(19) United States
(12) Patent Application Publication
(10) Pub. No.: US 2025/0090677 A1
(43) Pub. Date: Mar. 20, 2025

(54) ANTI-CDH6 ANTIBODIES AND ANTIBODY-DRUG CONJUGATES THEREOF

(71) Applicant: MULTITUDE THERAPEUTICS INC., Shanghai (CN)

(72) Inventors: Xun MENG, Shanghai (CN); Shu-Hui LIU, Shanghai (CN); Jing SHI, Shanghai (CN); Mingqiao WANG, Shanghai (CN); Rong PAN, Shanghai (CN); Yueyun JIANG, Shanghai (CN); Yiqiang WANG, Shanghai (CN); Zhaohui WANG, Shanghai (CN)

(21) Appl. No.: 18/715,982

(22) PCT Filed: Dec. 9, 2022
(86) PCT No.: PCT/CN2022/137932

§ 371 Date: Jun. 3, 2024

(30) Foreign Application Priority Data
Dec. 10, 2021 (CN) 202111507685.5
Dec. 10, 2021 (WO) PCT/CN2021/136994

(51) Int. Cl. A61K 47/68; A61K 39/00; A61P 35/00; C07K 16/28

(52) U.S. Cl. CPC A61K 47/68037; A61K 47/6849; A61P 35/00; C07K 16/28; A61K 2039/505; C07K 2317/24; C07K 2317/33; C07K 2317/565; C07K 2317/567; C07K 2317/73; C07K 2317/77; C07K 2317/92

(57) ABSTRACT

Provided is an antigen-binding protein having the following properties: (a) specifically binding to CDH6, and (b) having the activity of being internalized into CDH6-expressing cells by binding to CDH6. Further provided an immunoconjugate comprising an antibody or antigen binding fragment thereof that specifically binds to CDH6 and has internalization activity, a pharmaceutical product comprising the immunoconjugate and having therapeutic effects on a tumor, a method for treating a tumor using the immunoconjugate or the pharmaceutical product, and the like.

The present Application for Patent claims priority to Patent Cooperation Treaty application PCT/CN2021/136994, filed Dec. 10, 2021, and CN application CN2021115076855, filed Dec. 10, 2021. Priority is claimed to these applications and the disclosures of these prior applications are considered part of the disclosure of this application and the entire contents of the aforementioned applications are incorporated herein.

This application incorporates by reference a Sequence Listing with this application as an XML file entitled “0283-PA-002.xml” created on Nov. 1, 2022 and having a size of 149,920 bytes.

### BACKGROUND OF THE INVENTION

Cadherin-6 (CDH6) is specifically expressed in the brain or the kidney at the stage of development and has been reported to play an important role in the circuit formation of the central nervous system and nephron development in the kidney. The expression of CDH6 in the normal tissues of adult humans is localized to the tubules of the kidney, bile duct epithelial cells, and the like.

Meanwhile, it is known that CDH6 is specifically overexpressed at tumor sites in some types of human adult cancers. The correlation of CDH6 expression with poor prognosis and its applicability as a tumor marker has been reported with respect to human renal cell carcinoma, particularly, renal clear cell carcinoma and papillary renal cell carcinoma. The high expression of CDH6 has also been reported with respect to human ovarian cancer. It has also been reported that CDH6 is involved in the epithelial-mesenchymal transition of human thyroid cancer. Furthermore, it has been reported that CDH6 is also expressed in human bile duct cancer and human small-cell lung cancer.

Antibody drug conjugates (“ADCs”) have been used for the local delivery of cytotoxic agents in the treatment of cancer (see e.g., Lambert, Curr. Opinion In Pharmacology 5:543-549, 2005). ADCs allow targeted delivery of a drug moiety where maximum efficacy with minimal toxicity may be achieved. Although the recent FDA approval of six new antibody-drug conjugates (ADCs) is promising, attrition of ADCs during clinical development remains high.

DS-6000a, developed by Daiichi Sankyo, is the only reported CDH6 directed antibody drug conjugate (ADC) under investigation (phase I clinical stage). DS-6000a is comprised of a humanized anti-CDH6 IgG1 monoclonal antibody attached to a topoisomerase I inhibitor payload, an exatecan derivative, via a tetrapeptide-based cleavable linker. DS-6000a has not been approved for any indication in any country and the safety and efficacy need to be established.

Therefore, there remains a need for improved methods for treating cancers which are, or which have a high tendency to become, resistant to tyrosine kinase inhibitors, serine/threonine kinase inhibitors and/or chemotherapy, particularly using CDH6-ADCs.

### SUMMARY OF THE DISCLOSURE

The present application provides an antigen-binding protein having one or more of the following properties:
i) being capable of binding to CDH6 protein with a sensitivity of less than 12.5 ng/mL in an ELISA assay;
ii) being capable of binding to the CDH6 protein with a KD value below about 3.1×10⁻⁹M in an ELISA assay; and
iii) having capable of being internalized by an CDH6-expressing cell upon binding to CDH6.

In some embodiments, the CDH6 is a mammalian CDH6 protein.

In some embodiments, the CDH6 is human CDH6 protein.

In some embodiments, the antigen-binding protein competes for binding to CDH6 protein with a reference antibody, wherein the reference antibody comprises a light chain variable region (VL) and a heavy chain variable region (VH); wherein:
the VH comprises an amino acid sequence as set forth in SEQ ID NO: 18, and the VL comprises an amino acid sequence as set forth in SEQ ID NO: 23; or
the VH comprises an amino acid sequence as set forth in SEQ ID NO: 41, and the VL comprises an amino acid sequence as set forth in SEQ ID NO: 46.

In some embodiments, the antigen-binding protein comprises an antibody or an antigen-binding fragment thereof.

In some embodiments, the antibody comprises a monoclonal antibody, a polyclonal antibody, a dimer, a multimer, an intact antibody, a human antibody, a humanized antibody and/or a chimeric antibody.

In some embodiments, the antigen binding fragment comprises Fab, Fab′, Fv fragment, F(ab′)₂, scFv, di-scFv and/or dAb.

In some embodiments, the antibody is a monoclonal antibody.

In some embodiments, the antibody is a chimeric antibody, a humanized antibody and/or a human antibody.

In some embodiments, the antibody or the antigen binding fragment comprises at least one CDR (Complementarity Determining Region) of a heavy chain variable region (VH), the VH comprises the amino acid sequence as set forth in SEQ ID NO: 18, SEQ ID NO: 66, SEQ ID NO: 41, SEQ ID NO: 67, SEQ ID NO: 95 or SEQ ID NO: 103.

In some embodiments, the antibody or the antigen binding fragment comprises at least one CDR of a light chain variable region (VL), the VL comprises the amino acid sequence as set forth in SEQ ID NO: 23, SEQ ID NO: 68, SEQ ID NO: 46, SEQ ID NO: 69, SEQ ID NO: 96 or SEQ ID NO: 104.

In some embodiments, the antigen-binding protein comprises VH, wherein the VH comprises HCDR1, HCDR2, HCDR3, the HCDR1 comprises the amino acid sequence as set forth in SEQ ID NO: 1.

In some embodiments, the antigen-binding protein comprises VH, wherein the VH comprises HCDR1, HCDR2, HCDR3, the HCDR1 comprises the amino acid sequence as set forth in SEQ ID NO: 2, SEQ ID NO: 8, SEQ ID NO: 89 or SEQ ID NO: 97.

In some embodiments, the antigen-binding protein comprises VH, wherein the VH comprises HCDR1, HCDR2, HCDR3, the HCDR2 comprises the amino acid sequence as set forth in SEQ ID NO: 3, SEQ ID NO: 9, SEQ ID NO: 90 or SEQ ID NO: 98.

In some embodiments, the antigen-binding protein comprises VH, wherein the VH comprises HCDR1, HCDR2, HCDR3, the HCDR3 comprises the amino acid sequence as set forth in SEQ ID NO: 4, SEQ ID NO: 10, SEQ ID NO: 91 or SEQ ID NO: 99.

In some embodiments, the antigen-binding protein comprises VH, wherein the VH comprises HCDR1, HCDR2, HCDR3, the HCDR1 comprises the amino acid sequence as set forth in SEQ ID NO: 1, the HCDR2 comprises the amino acid sequence as set forth in SEQ ID NO: 3 or SEQ ID NO: 9, and the HCDR3 comprises the amino acid sequence as set forth in SEQ ID NO: 4 or SEQ ID NO: 10.

In some embodiments, the antigen-binding protein comprises VH, wherein the VH comprises HCDR1, HCDR2, HCDR3, wherein the HCDR1 comprises the amino acid sequence as set forth in SEQ ID NO: 2, the HCDR2 comprises the amino acid sequence as set forth in SEQ ID NO: 3, and the HCDR3 comprises the amino acid sequence as set forth in SEQ ID NO: 4; or
wherein the HCDR1 comprises the amino acid sequence as set forth in SEQ ID NO: 8, the HCDR2 comprises the amino acid sequence as set forth in SEQ ID NO: 9, and the HCDR3 comprises the amino acid sequence as set forth in SEQ ID NO: 10; or
wherein the HCDR1 comprises the amino acid sequence as set forth in SEQ ID NO: 89, the HCDR2 comprises the amino acid sequence as set forth in SEQ ID NO: 90, and the HCDR3 comprises the amino acid sequence as set forth in SEQ ID NO: 91; or
wherein the HCDR1 comprises the amino acid sequence as set forth in SEQ ID NO: 97, the HCDR2 comprises the amino acid sequence as set forth in SEQ ID NO: 98, and the HCDR3 comprises the amino acid sequence as set forth in SEQ ID NO: 99.

In some embodiments, the antigen-binding protein comprises a VH, wherein the VH comprises a framework region HFR1, the C-terminus of the HFR1 is directly or indirectly connected to the N-terminus of the HCDR1, and the HFR1 comprises an amino acid sequence set forth in SEQ ID NO: 14, SEQ ID NO: 24, SEQ ID NO: 37, SEQ ID NO: 47 or SEQ ID NO: 52.

In some embodiments, the antigen-binding protein comprises a VH, wherein the VH comprises a framework region HFR2, the HFR2 is positioned between the HCDR1 and the HCDR2, and the HFR2 comprises an amino acid sequence set forth in SEQ ID NO: 15, SEQ ID NO: 28, SEQ ID NO: 38 or SEQ ID NO: 48.

In some embodiments, the antigen-binding protein comprises a VH, wherein the VH comprises a framework region HFR3, the HFR3 is positioned between the HCDR2 and the HCDR3, and the HFR3 comprises an amino acid sequence set forth in SEQ ID NO: 16, SEQ ID NO: 25, SEQ ID NO: 39, SEQ ID NO: 49 or SEQ ID NO: 53.

In some embodiments, the antigen-binding protein comprises a VH, wherein the VH comprises a framework region HFR4, the N-terminus of the HFR4 is directly or indirectly connected to the C-terminus of the HCDR3, and the HFR4 comprises an amino acid sequence set forth in SEQ ID NO: 17, SEQ ID NO: 26, SEQ ID NO: 40 or SEQ ID NO: 50.

In some embodiments, the antigen-binding protein comprises a VH, wherein the VH comprises framework regions HFR1, HFR2, HFR3 and HFR4, the C-terminus of the HFR1 is directly or indirectly connected to the N-terminus of the HCDR1, the HFR2 is positioned between the HCDR1 and the HCDR2, the HFR3 is positioned between the HCDR2 and the HCDR3, and the N-terminus of the HFR4 is directly or indirectly connected to the C-terminus of the HCDR3; wherein the HFR1 comprises an amino acid sequence set forth in SEQ ID NO: 14, the HFR2 comprises an amino acid sequence set forth in SEQ ID NO: 15, the HFR3 comprises an amino acid sequence set forth in SEQ ID NO: 16, and the HFR4 comprises an amino acid sequence set forth in SEQ ID NO: 17; or
the HFR1 comprises an amino acid sequence set forth in SEQ ID NO: 24, the HFR2 comprises an amino acid sequence set forth in SEQ ID NO: 15, the HFR3 comprises an amino acid sequence set forth in SEQ ID NO: 25, and the HFR4 comprises an amino acid sequence set forth in SEQ ID NO: 26; or
the HFR1 comprises an amino acid sequence set forth in SEQ ID NO: 24, the HFR2 comprises an amino acid sequence set forth in SEQ ID NO: 28, the HFR3 comprises an amino acid sequence set forth in SEQ ID NO: 25, and the HFR4 comprises an amino acid sequence set forth in SEQ ID NO: 26; or
the HFR1 comprises an amino acid sequence set forth in SEQ ID NO: 37, the HFR2 comprises an amino acid sequence set forth in SEQ ID NO: 38, the HFR3 comprises an amino acid sequence set forth in SEQ ID NO: 39, and the HFR4 comprises an amino acid sequence set forth in SEQ ID NO: 40; or
the HFR1 comprises an amino acid sequence set forth in SEQ ID NO: 47, the HFR2 comprises an amino acid sequence set forth in SEQ ID NO: 48, the HFR3 comprises an amino acid sequence set forth in SEQ ID NO: 49, and the HFR4 comprises an amino acid sequence set forth in SEQ ID NO: 50; or
the HFR1 comprises an amino acid sequence set forth in SEQ ID NO: 52, the HFR2 comprises an amino acid sequence set forth in SEQ ID NO: 38, the HFR3 comprises an amino acid sequence set forth in SEQ ID NO: 53, and the HFR4 comprises an amino acid sequence set forth in SEQ ID NO: 26.

In some embodiments, the antigen-binding protein comprises VH, wherein the VH comprises the amino acid sequence as set forth in SEQ ID NO: 18, SEQ ID NO: 66, SEQ ID NO: 41, SEQ ID NO: 67, SEQ ID NO: 95 or SEQ ID NO: 103.

In some embodiments, the antigen-binding protein comprises an antibody heavy chain constant region.

In some embodiments, the antibody heavy chain constant region comprises a constant region derived from human IgG; optionally, the antibody heavy chain constant region comprises a constant region derived from human IgG1, IgG2, IgG3 or IgG4.

In some embodiments, the antibody heavy chain constant region comprises the amino acid sequence as set forth in SEQ ID NO: 70.

In some embodiments, the antibody heavy chain comprises the amino acid sequence as set forth in SEQ ID NO: 72, SEQ ID NO: 85, SEQ ID NO: 80 or SEQ ID NO: 87.

In some embodiments, the antigen-binding protein comprises VL, wherein the VL comprises LCDR1, LCDR2, LCDR3, the LCDR1 comprises the amino acid sequence as set forth in SEQ ID NO: 5, SEQ ID NO: 11, SEQ ID NO: 92 or SEQ ID NO: 100.

In some embodiments, the antigen-binding protein comprises VL, wherein the VL comprises LCDR1, LCDR2, LCDR3, the LCDR2 comprises the amino acid sequence as set forth in SEQ ID NO: 6, SEQ ID NO: 12, SEQ ID NO: 93 or SEQ ID NO: 101.

In some embodiments, the antigen-binding protein comprises VL, wherein the VL comprises LCDR1, LCDR2, LCDR3, the LCDR3 comprises the amino acid sequence as set forth in SEQ ID NO: 7, SEQ ID NO: 13, SEQ ID NO: 94 or SEQ ID NO: 102.

In some embodiments, the antigen-binding protein comprises VL, wherein the VL comprises LCDR1, LCDR2, LCDR3, the LCDR1 comprises the amino acid sequence as set forth in SEQ ID NO: 5, the LCDR2 comprises the amino acid sequence as set forth in SEQ ID NO: 6, and the LCDR3 comprises the amino acid sequence as set forth in SEQ ID NO: 7; or
wherein the LCDR1 comprises the amino acid sequence as set forth in SEQ ID NO: 11, the LCDR2 comprises the amino acid sequence as set forth in SEQ ID NO: 12, and the LCDR3 comprises the amino acid sequence as set forth in SEQ ID NO: 13; or
wherein the LCDR1 comprises the amino acid sequence as set forth in SEQ ID NO: 92, the LCDR2 comprises the amino acid sequence as set forth in SEQ ID NO: 93, and the LCDR3 comprises the amino acid sequence as set forth in SEQ ID NO: 94; or
wherein the LCDR1 comprises the amino acid sequence as set forth in SEQ ID NO: 100, the LCDR2 comprises the amino acid sequence as set forth in SEQ ID NO: 101, and the LCDR3 comprises the amino acid sequence as set forth in SEQ ID NO: 102.

In some embodiments, the antigen-binding protein comprises VH and VL, wherein the VH comprises HCDR1, HCDR2, HCDR3, and the VL comprises LCDR1, LCDR2, LCDR3, wherein the HCDR1 comprises the amino acid sequence as set forth in SEQ ID NO: 1, the HCDR2 comprises the amino acid sequence as set forth in SEQ ID NO: 3, and the HCDR3 comprises the amino acid sequence as set forth in SEQ ID NO: 4, the LCDR1 comprises the amino acid sequence as set forth in SEQ ID NO: 5, the LCDR2 comprises the amino acid sequence as set forth in SEQ ID NO: 6, and the LCDR3 comprises the amino acid sequence as set forth in SEQ ID NO: 7; or wherein the HCDR1 comprises the amino acid sequence as set forth in SEQ ID NO: 1, the HCDR2 comprises the amino acid sequence as set forth in SEQ ID NO: 9, and the HCDR3 comprises the amino acid sequence as set forth in SEQ ID NO: 10 the LCDR1 comprises the amino acid sequence as set forth in SEQ ID NO: 11, the LCDR2 comprises the amino acid sequence as set forth in SEQ ID NO: 12, and the LCDR3 comprises the amino acid sequence as set forth in SEQ ID NO: 13.

In some embodiments, the antigen-binding protein comprises VH and VL, wherein the VH comprises HCDR1, HCDR2, HCDR3, and the VL comprises LCDR1, LCDR2, LCDR3, wherein the HCDR1 comprises the amino acid sequence as set forth in SEQ ID NO: 2, the HCDR2 comprises the amino acid sequence as set forth in SEQ ID NO: 3, and the HCDR3 comprises the amino acid sequence as set forth in SEQ ID NO: 4, the LCDR1 comprises the amino acid sequence as set forth in SEQ ID NO: 5, the LCDR2 comprises the amino acid sequence as set forth in SEQ ID NO: 6, and the LCDR3 comprises the amino acid sequence as set forth in SEQ ID NO: 7; or
wherein the HCDR1 comprises the amino acid sequence as set forth in SEQ ID NO: 8, the HCDR2 comprises the amino acid sequence as set forth in SEQ ID NO: 9, and the HCDR3 comprises the amino acid sequence as set forth in SEQ ID NO: 10, the LCDR1 comprises the amino acid sequence as set forth in SEQ ID NO: 11, the LCDR2 comprises the amino acid sequence as set forth in SEQ ID NO: 12, and the LCDR3 comprises the amino acid sequence as set forth in SEQ ID NO: 13; or
wherein the HCDR1 comprises the amino acid sequence as set forth in SEQ ID NO: 89, the HCDR2 comprises the amino acid sequence as set forth in SEQ ID NO: 90, and the HCDR3 comprises the amino acid sequence as set forth in SEQ ID NO: 91, the LCDR1 comprises the amino acid sequence as set forth in SEQ ID NO: 92, the LCDR2 comprises the amino acid sequence as set forth in SEQ ID NO: 93, and the LCDR3 comprises the amino acid sequence as set forth in SEQ ID NO: 94; or
wherein the HCDR1 comprises the amino acid sequence as set forth in SEQ ID NO: 97, the HCDR2 comprises the amino acid sequence as set forth in SEQ ID NO: 98, and the HCDR3 comprises the amino acid sequence as set forth in SEQ ID NO: 99, the LCDR1 comprises the amino acid sequence as set forth in SEQ ID NO: 100, the LCDR2 comprises the amino acid sequence as set forth in SEQ ID NO: 101, and the LCDR3 comprises the amino acid sequence as set forth in SEQ ID NO: 102.

In some embodiments, the antigen-binding protein comprises a VL, wherein the VL comprises a framework region LFR1, the C-terminus of the LFR1 is directly or indirectly connected to the N-terminus of the LCDR1, and the LFR1 comprises an amino acid sequence set forth in SEQ ID NO: 19, SEQ ID NO: 30, SEQ ID NO: 42, SEQ ID NO: 55, SEQ ID NO: 59 or SEQ ID NO: 64.

In some embodiments, the antigen-binding protein comprises a VL, wherein the VL comprises a framework region LFR2, the LFR2 is positioned between the LCDR1 and the LCDR2, and the LFR2 comprises an amino acid sequence set forth in SEQ ID NO: 20, SEQ ID NO: 31, SEQ ID NO: 43, SEQ ID NO: 56 or SEQ ID NO: 60.

In some embodiments, the antigen-binding protein comprises a VL, wherein the VL comprises a framework region LFR3, the LFR3 is positioned between the LCDR2 and the LCDR3, and the LFR3 comprises an amino acid sequence set forth in SEQ ID NO: 21, SEQ ID NO: 32, SEQ ID NO: 35, SEQ ID NO: 44, SEQ ID NO: 57 or SEQ ID NO: 61.

In some embodiments, the antigen-binding protein comprises a VL, wherein the VL comprises a framework region LFR4, the N-terminus of the LFR4 is directly or indirectly connected to the C-terminus of the LCDR3, and the LFR4 comprises an amino acid sequence set forth in SEQ ID NO: 22, SEQ ID NO: 33, SEQ ID NO: 45 or SEQ ID NO: 62.

In some embodiments, the antigen-binding protein comprises a VL, wherein the VL comprises framework regions LFR1, LFR2, LFR3 and LFR4, wherein the C-terminus of the LFR1 is directly or indirectly connected to the N-terminus of the LCDR1, the LFR2 is positioned between the LCDR1 and the LCDR2, the LFR3 is positioned between the LCDR2 and the LCDR3, and the N-terminus of the LFR4 is directly or indirectly connected to the C-terminus of the LCDR3; wherein the LFR1 comprises an amino acid sequence set forth in SEQ ID NO: 19, the LFR2 comprises an amino acid sequence set forth in SEQ ID NO: 20, the LFR3 comprises an amino acid sequence set forth in SEQ ID NO: 21, and the LFR4 comprises an amino acid sequence set forth in SEQ ID NO: 22; or
the LFR1 comprises an amino acid sequence set forth in SEQ ID NO: 30, the LFR2 comprises an amino acid sequence set forth in SEQ ID NO: 31, the LFR3 comprises an amino acid sequence set forth in SEQ ID NO: 32, and the LFR4 comprises an amino acid sequence set forth in SEQ ID NO: 33; or
the LFR1 comprises an amino acid sequence set forth in SEQ ID NO: 30, the LFR2 comprises an amino acid sequence set forth in SEQ ID NO: 31, the LFR3 comprises an amino acid sequence set forth in SEQ ID NO: 35, and the LFR4 comprises an amino acid sequence set forth in SEQ ID NO: 33; or
the LFR1 comprises an amino acid sequence set forth in SEQ ID NO: 42, the LFR2 comprises an amino acid sequence set forth in SEQ ID NO: 43, the LFR3 comprises an amino acid sequence set forth in SEQ ID NO: 44, and the LFR4 comprises an amino acid sequence set forth in SEQ ID NO: 45; or
the LFR1 comprises an amino acid sequence set forth in SEQ ID NO: 55, the LFR2 comprises an amino acid sequence set forth in SEQ ID NO: 56, the LFR3 comprises an amino acid sequence set forth in SEQ ID NO: 57, and the LFR4 comprises an amino acid sequence set forth in SEQ ID NO: 45; or
the LFR1 comprises an amino acid sequence set forth in SEQ ID NO: 59, the LFR2 comprises an amino acid sequence set forth in SEQ ID NO: 60, the LFR3 comprises an amino acid sequence set forth in SEQ ID NO: 61, and the LFR4 comprises an amino acid sequence set forth in SEQ ID NO: 62; or
the LFR1 comprises an amino acid sequence set forth in SEQ ID NO: 64, the LFR2 comprises an amino acid sequence set forth in SEQ ID NO: 60, the LFR3 comprises an amino acid sequence set forth in SEQ ID NO: 61, and the LFR4 comprises an amino acid sequence set forth in SEQ ID NO: 62.

In some embodiments, the antigen-binding protein comprises VL, wherein the VL comprises the amino acid sequence as set forth in SEQ ID NO: 23, SEQ ID NO: 68, SEQ ID NO: 46, SEQ ID NO: 69, SEQ ID NO: 96 or SEQ ID NO: 104.

In some embodiments, the antigen-binding protein comprises VH and VL, wherein the VH comprises the amino acid sequence as set forth in SEQ ID NO: 18, and the VL comprises the amino acid sequence as set forth in SEQ ID NO: 23; or
wherein the VH comprises the amino acid sequence as set forth in SEQ ID NO: 41, and the VL comprises the amino acid sequence as set forth in SEQ ID NO: 46; or
wherein the VH comprises the amino acid sequence as set forth in SEQ ID NO: 66, and the VL comprises the amino acid sequence as set forth in SEQ ID NO: 68; or
wherein the VH comprises the amino acid sequence as set forth in SEQ ID NO: 67, and the VL comprises the amino acid sequence as set forth in SEQ ID NO: 69; or
wherein the VH comprises the amino acid sequence as set forth in SEQ ID NO: 95, and the VL comprises the amino acid sequence as set forth in SEQ ID NO: 96; or
wherein the VH comprises the amino acid sequence as set forth in SEQ ID NO: 103, and the VL comprises the amino acid sequence as set forth in SEQ ID NO: 104.

In some embodiments, the antigen-binding protein comprises an antibody light chain constant region.

In some embodiments, the antibody light chain constant region comprises a human Igκ constant region or a human Igλ constant region.

In some embodiments, the antibody light chain constant region comprises the amino acid sequence as set forth in SEQ ID NO: 71.

In some embodiments, the antibody light chain comprises the amino acid sequence as set forth in SEQ ID NO: 73, SEQ ID NO: 86, SEQ ID NO: 81 or SEQ ID NO: 88.

In some embodiments, the antigen-binding protein comprises an antibody heavy chain and an antibody light chain, wherein the antibody heavy chain comprises the amino acid sequence as set forth in SEQ ID NO: 72, and the antibody light chain comprises the amino acid sequence as set forth in SEQ ID NO: 73; or
wherein the antibody heavy chain comprises the amino acid sequence as set forth in SEQ ID NO: 85, and the antibody light chain comprises the amino acid sequence as set forth in SEQ ID NO: 86; or
wherein the antibody heavy chain comprises the amino acid sequence as set forth in SEQ ID NO: 80, and the antibody light chain comprises the amino acid sequence as set forth in SEQ ID NO: 81; or
wherein the antibody heavy chain comprises the amino acid sequence as set forth in SEQ ID NO: 87, and the antibody light chain comprises the amino acid sequence as set forth in SEQ ID NO: 88.

In another aspect, the present application provides an antigen-binding protein that specifically binds CDH6, comprising
(i) a VL comprising VL CDR1, CDR2, and CDR3 from a VL having an amino acid sequence consisting of SEQ ID NO: 18; and/or a VH comprising VH CDR1, CDR2, and CDR3 from a VH having an amino acid sequence consisting of SEQ ID NO: 23;
(ii) a VL comprising VL CDR1, CDR2, and CDR3 from a VL having an amino acid sequence consisting of SEQ ID NO: 41; and/or a VH comprising VH CDR1, CDR2, and CDR3 from a VH having an amino acid sequence consisting of SEQ ID NO: 46;
(iii) a VL comprising VL CDR1, CDR2, and CDR3 from a VL having an amino acid sequence consisting of SEQ ID NO: 66; and/or a VH comprising VH CDR1, CDR2, and CDR3 from a VH having an amino acid sequence consisting of SEQ ID NO: 68;
(iv) a VL comprising VL CDR1, CDR2, and CDR3 from a VL having an amino acid sequence consisting of SEQ ID NO: 67; and/or a VH comprising VH CDR1, CDR2, and CDR3 from a VH having an amino acid sequence consisting of SEQ ID NO: 69;
(v) a VL comprising VL CDR1, CDR2, and CDR3 from a VL having an amino acid sequence consisting of SEQ ID NO: 95; and/or a VH comprising VH CDR1, CDR2, and CDR3 from a VH having an amino acid sequence consisting of SEQ ID NO: 96; or
(vi) a VL comprising VL CDR1, CDR2, and CDR3 from a VL having an amino acid sequence consisting of SEQ ID NO: 103; and/or a VH comprising VH CDR1, CDR2, and CDR3 from a VH having an amino acid sequence consisting of SEQ ID NO: 104.

In another aspect, the present application provides an antigen-binding protein that competes with the antigen-binding protein provided herein for binding to CDH6.

In another aspect, the present application provides a polypeptide comprising the antigen-binding protein described above.

In another aspect, the present application provides a nucleic acid molecule or molecules encoding the antigen-binding protein described above or the polypeptide described above.

In another aspect, the present application provides a vector comprising the nucleic acid molecule described above.

In another aspect, the present application provides a cell comprising the nucleic acid molecule described above or the vector described above or expressing the antigen-binding protein described above or the polypeptide described above.

In another aspect, the present application provides a method for preparing the antigen-binding protein described above, wherein the method comprises culturing the cell described above under a condition that the antigen-binding protein described above is expressed.

In another aspect, the present application provides a pharmaceutical composition comprising the antigen-binding protein described above, the polypeptide described above, the nucleic acid molecule described above, the vector described above and/or the cell described above, and optionally a pharmaceutically acceptable carrier.

In another aspect, the present application provides a kit comprising the antigen-binding protein described above, the polypeptide described above or the pharmaceutical composition described above.

In another aspect, the present application provides use of the antigen-binding protein described above, the polypeptide described above, the nucleic acid molecule described above, the vector described above and/or the cell described above, and/or the pharmaceutical composition described above in preparing a medicament for preventing and/or treating a disease or disorder related to CDH6.

In some embodiments, the disease or disorder related to CDH6 comprises a tumor.

In some embodiments, the tumor comprises a CDH6-expressing tumor.

In some embodiments, the tumor comprises renal cell carcinoma, renal clear cell carcinoma, papillary renal cell carcinoma, ovarian cancer, ovarian serous adenocarcinoma, thyroid cancer, bile duct cancer, lung cancer, small cell lung cancer, liver cancer, glioblastoma, mesothelioma, uterine cancer, pancreatic cancer, Wilm's tumor or neuroblastoma.

In some embodiments, the medicament further comprises an additional therapeutic agent.

In another aspect, the present application provides an immunoconjugate or a pharmaceutically acceptable salt or a solvate thereof comprising the antigen-binding protein described above.

In some embodiments, the immunoconjugate or a pharmaceutically acceptable salt or a solvate thereof further comprises an active moiety conjugated to the antibody or the antigen binding fragment thereof.

In some embodiments, the active moiety comprises a drug moiety and/or a label.

In some embodiments, the drug moiety is selected from the group consisting of a cytotoxic agent, a cytokine, a nucleic acid, a nucleic acid-associated molecule, a radionuclide, a chemokine, an immuno(co)-stimulatory molecule, an immunosuppressive molecule, a death ligand, an apoptosis-inducing protein, a kinase, a prodrug-converting enzyme, a RNase, an agonistic antibody or antibody fragment, an antagonistic antibody or antibody fragment, a growth factor, a hormone, a coagulation factor, a fibrinolytic protein, peptides mimicking these, and fragments, fusion proteins or derivatives thereof.

In some embodiments, the label selected from the group consisting of a radiolabel, a fluorophore, a chromophore, an imaging agent, and a metal ion.

In some embodiments, the cytotoxic agent comprises a microtubule disrupting drug and/or a DNA-damaging agent.

In some embodiments, the cytotoxic agent comprises a tubulin inhibitor and/or a topoisomerase inhibitor.

In some embodiments, the cytotoxic agent comprises a topoisomerase I inhibitor.

In some embodiments, the cytotoxic agent comprises camptothecin (CPT) or a derivative thereof.

In some embodiments, the cytotoxic agent comprises the following structure of formula II or a tautomer, mesomer, racemate, enantiomer, or diastereomer thereof, or mixtures thereof, or a pharmaceutically acceptable salt or a solvate thereof:

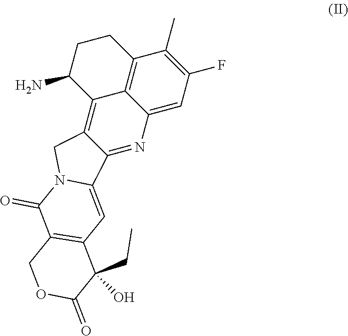 [SMILES: CC[C@@]1(O)C(=O)OCc2c1cc1n(c2=O)Cc2c-1nc1cc(F)c(C)c3c1c2[C@@H](N)CC3]

In some embodiments, the immunoconjugate or a pharmaceutically acceptable salt or a solvate thereof comprises an antibody drug conjugate (ADC) of formula (I):

Ab−(L−(D)ₘ)ₙ, (I)
or a pharmaceutically acceptable salt thereof; wherein
Ab is the antigen-binding protein described above;
L is a linker;
D is a drug moiety;
m is an integer from 1 to 8; and
n is any number from 1 to 10.

In some embodiments, the L is selected from: a cleavable linker and a non-cleavable linker.

In some embodiments, the L comprises cleavable peptide.

In some embodiments, the cleavable peptide is cleavable by an enzyme.

In some embodiments, the enzyme comprises Cathepsin B.

In some embodiments, the cleavable peptide or L comprises an amino acid unit.

In some embodiments, the amino acid unit comprises a dipeptide, tripeptide, tetrapeptide or pentapeptide.

In some embodiments, the amino acid unit is selected from: Val-Cit, Val-Ala (VA), Glu-Val-Cit, Ala-Ala-Asn (AAN), Gly-Val-Cit, Gly-Gly-Gly(GGG) and Gly-Gly-Phe-Gly(GGFG).

In some embodiments, the L comprises a spacer.

In some embodiments, the spacer comprises self-immolative spacers.

In some embodiments, the self-immolative spacer comprises p-aminobenzoxy carbonyl (PABC) or p-aminobenzyl (PAB).

In some embodiments, the cleavable peptide is directly spliced to the spacer.

In some embodiments, the L comprises: -Val-Cit-PABC-, -Val-Ala-PABC-, -Glu-Val-Cit-PABC-, -Ala-Ala-Asn-PABC-, -Gly-Val-Cit-PABC-, -Gly-Gly-Gly-PABC-, -Gly-Gly-Phe-Gly-PABC-, -Val-Cit-PAB-, -Val-Ala-PAB-, -Glu-Val-Cit-PAB-, -Ala-Ala-Asn-PAB-, -Gly-Val-Cit-PAB-, -Gly-Gly-Gly-PAB- or -Gly-Gly-Phe-Gly-PAB-.

In some embodiments, the spacer comprises the structure shown in —NH—(CH₂)n¹-La-Lb-Lc-, where La denotes —O— or a single bond; Lb denotes —CR²(—CR³)— or a single bond, where R² and R³ each independently denote C₁-C₆ alkyl, —(CH₂)n^{a}-NH₂, —(CH₂)n^{b}-COOH or —(CH₂)n¹-OH, n¹ denotes an integer from 0 to 6, n^{a}, n^{b} and n^{c} each independently denote an integer from 1 to 4, but R² and R³ are not the same when n^{a} is 0, and Lc denotes —C(═O)—.

In some embodiments, the spacer comprises —NH—(CH₂)₃—C(═O)—, —NH—CH₂—O—CH₂—C(═O)— or —NH—(CH₂)₂—O—CH₂—C(═O)—.

In some embodiments, the L comprises the structure shown in -L₁-L₂-L₃-, where L₁ denotes -(succinimidyl-3-yl-N)—(CH₂)n²-C(═O)—, —CH₂—C(═O)—NH—(CH₂)n³-C(═O)— or —C(═O)—(CH₂)n⁴-C(═O)—, where n² denotes an integer from 2 to 8, n³ denotes an integer from 1 to 8, and n⁴ denotes an integer from 1 to 8; L₂ denotes amino acid unit; L₃ denotes a self-degradable spacer.

In some embodiments, the L is selected from:
-(succinimidyl-3-yl-N)—CH₂CH₂—C(═O)-GGFG-PABC-;
-(succinimidyl-3-yl-N)—CH₂CH₂CH₂CH₂CH₂—C(═O)-GGFG-PABC-;
-(succinimidyl-3-yl-N)—CH₂CH₂CH₂CH₂CH₂—C(═O)-GGFG-NH-PABC-;
-(succinimidyl-3-yl-N)—CH₂CH₂—C(═O)—NH—CH₂CH₂O—CH₂CH₂O—CH₂CH₂—C(═O)-GGFG-PABC-;
-(succinimidyl-3-yl-N)—CH₂CH₂—C(═O)—NH—CH₂CH₂O—CH₂CH₂O—CH₂CH₂O—CH₂CH₂O—
CH₂CH₂—C(═O)-GGFG-PABC-;
—CH₂—C(═O)—NH—CH₂CH₂—C(═O)-GGFG-PABC-;
—C(═O)—CH₂CH₂CH₂CH₂CH₂CH₂—C(═O)-GGFG-PABC-;
-(succinimidyl-3-yl-N)—CH₂CH₂—C(═O)-GGFG-NH—CH₂CH₂—C(═O)—;
-(succinimidyl-3-yl-N)—CH₂CH₂—C(═O)-GGFG-NH—CH₂CH₂CH₂—C(═O)—;
-(succinimidyl-3-yl-N)—CH₂CH₂CH₂CH₂CH₂—C(═O)-GGFG-NH—CH₂CH₂—C(═O)—;
-(succinimidyl-3-yl-N)—CH₂CH₂CH₂CH₂CH₂—C(═O)-GGFG-NH—CH₂CH₂CH₂—C(═O)—;
-(succinimidyl-3-yl-N)—CH₂CH₂CH₂CH₂CH₂—C(═O)-GGFG-NH—CH₂CH₂CH₂CH₂CH₂—C(═O)—;
-(succinimidyl-3-yl-N)—CH₂CH₂CH₂CH₂CH₂—C(═O)-GGFG-NH—CH₂—O—CH₂—C(═O)—;
-(succinimidyl-3-yl-N)—CH₂CH₂CH₂CH₂CH₂—C(═O)-GGFG-NH—CH₂CH₂—O—CH₂—C(═O)—;
-(succinimidyl-3-yl-N)—CH₂CH₂—C(═O)—NH—CH₂CH₂O—CH₂CH₂O—CH₂CH₂—C(═O)-GGFG-NH—CH₂CH₂CH₂—C(═O)—;
-(succinimidyl-3-yl-N)—CH₂CH₂—C(═O)—NH—CH₂CH₂O—CH₂CH₂O—CH₂CH₂—C(═O)-GGFG-NH—CH₂CH₂—C(═O)—;
-(succinimidyl-3-yl-N)—CH₂CH₂—C(═O)—NH—CH₂CH₂O—CH₂CH₂O—CH₂CH₂O—CH₂CH₂O—CH₂CH₂—C(═O)-GGFG-NH—CH₂CH₂CH₂—C(═O)—;
-(succinimidyl-3-yl-N)—CH₂CH₂—C(═O)—NH—CH₂CH₂O—CH₂CH₂O—CH₂CH₂O—CH₂CH₂O—CH₂CH₂—C(═O)-GGFG-NH—CH₂CH₂—C(═O)—;
—CH₂—C(═O)—NH—CH₂CH₂—C(═O)-GGFG-NH—CH₂CH₂CH₂—C(═O)—;
—C(═O)—CH₂CH₂CH₂CH₂CH₂CH₂—C(═O)-GGFG-NH—CH₂CH₂CH₂—C(═O)—;
-(succinimidyl-3-yl-N)—CH₂CH₂—C(═O)—VA-PABC-;
-(succinimidyl-3-yl-N)—CH₂CH₂CH₂CH₂CH₂—C(═O)—VA-PABC-;
-(succinimidyl-3-yl-N)—CH₂CH₂CH₂CH₂CH₂—C(═O)—VA-NH-PABC-;
-(succinimidyl-3-yl-N)—CH₂CH₂—C(═O)—NH—CH₂CH₂O—CH₂CH₂O—CH₂CH₂—C(═O)—VA-PABC-;
-(succinimidyl-3-yl-N)—CH₂CH₂—C(═O)—NH—CH₂CH₂O—CH₂CH₂O—CH₂CH₂O—CH₂CH₂O—CH₂CH₂—C(═O)—VA-PABC-;
—CH₂—C(═O)—NH—CH₂CH₂—C(═O)—VA-PABC-;
—C(═O)—CH₂CH₂CH₂CH₂CH₂CH₂—C(═O)—VA-PABC-;
-(succinimidyl-3-yl-N)—CH₂CH₂—C(═O)—VA-NH—CH₂CH₂—C(═O)—;
-(succinimidyl-3-yl-N)—CH₂CH₂—C(═O)—VA-NH—CH₂CH₂CH₂—C(═O)—;
-(succinimidyl-3-yl-N)—CH₂CH₂CH₂CH₂CH₂—C(═O)—VA-NH—CH₂CH₂—C(═O)—;
-(succinimidyl-3-yl-N)—CH₂CH₂CH₂CH₂CH₂—C(═O)—VA-NH—CH₂CH₂CH₂—C(═O)—;
-(succinimidyl-3-yl-N)—CH₂CH₂CH₂CH₂CH₂—C(═O)—VA-NH—CH₂CH₂CH₂CH₂CH₂—C(═O)—;
-(succinimidyl-3-yl-N)—CH₂CH₂CH₂CH₂CH₂—C(═O)—VA-NH—CH₂—O—CH₂—C(═O)—;
-(succinimidyl-3-yl-N)—CH₂CH₂CH₂CH₂CH₂—C(═O)—VA-NH—CH₂CH₂—O—CH₂—C(═O)—;
-(succinimidyl-3-yl-N)—CH₂CH₂—C(═O)—NH—CH₂CH₂O—CH₂CH₂O—CH₂CH₂—C(═O)—VA-NH—CH₂CH₂CH₂—C(═O)—;
-(succinimidyl-3-yl-N)—CH₂CH₂—C(═O)—NH—CH₂CH₂O—CH₂CH₂O—CH₂CH₂—C(═O)—VA-NH—CH₂CH₂—C(═O)—;
-(succinimidyl-3-yl-N)—CH₂CH₂—C(═O)—NH—CH₂CH₂O—CH₂CH₂O—CH₂CH₂O—CH₂CH₂O—CH₂CH₂—C(═O)—VA-NH—CH₂CH₂CH₂—C(═O)—;
-(succinimidyl-3-yl-N)—CH₂CH₂—C(═O)—NH—CH₂CH₂O—CH₂CH₂O—CH₂CH₂O—CH₂CH₂O—CH₂CH₂—C(═O)—VA-NH—CH₂CH₂—C(═O)—;
—CH₂—C(═O)—NH—CH₂CH₂—C(═O)—VA-NH—CH₂CH₂CH₂—C(═O)—; and
—C(═O)—CH₂CH₂CH₂CH₂CH₂CH₂—C(═O)—VA-NH—CH₂CH₂CH₂—C(═O)—.

In some embodiments, the p-aminobenzoxy carbonyl (PABC) or p-aminobenzyl (PAB) comprises a polysarcosine (poly-N-methylglycine) residue.

In some embodiments, the L is selected from the following structure:

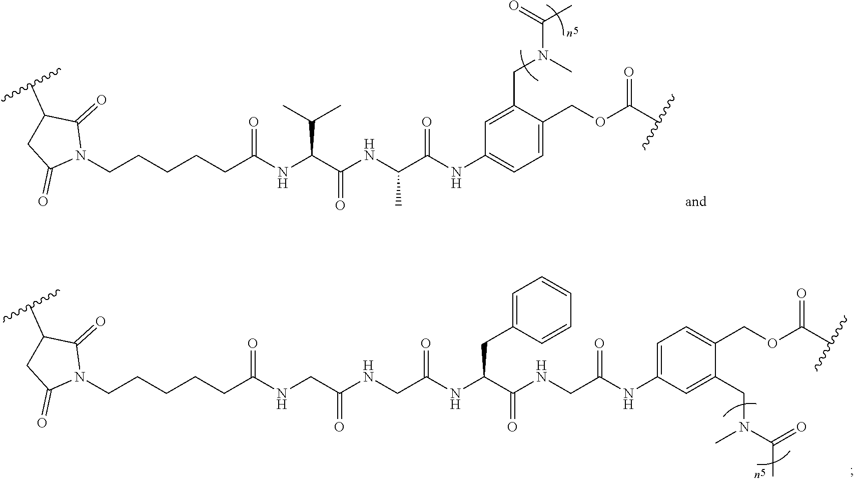

wherein n⁵ denotes an integer from 0 to 20.

In some embodiments, the n⁵ denotes an integer from 1 to 15.

In some embodiments, the n⁵ denotes an integer from 1 to 7.

In some embodiments, the n⁵ denotes an integer from 8 to 15.

In some embodiments, the L is selected from the following structure:

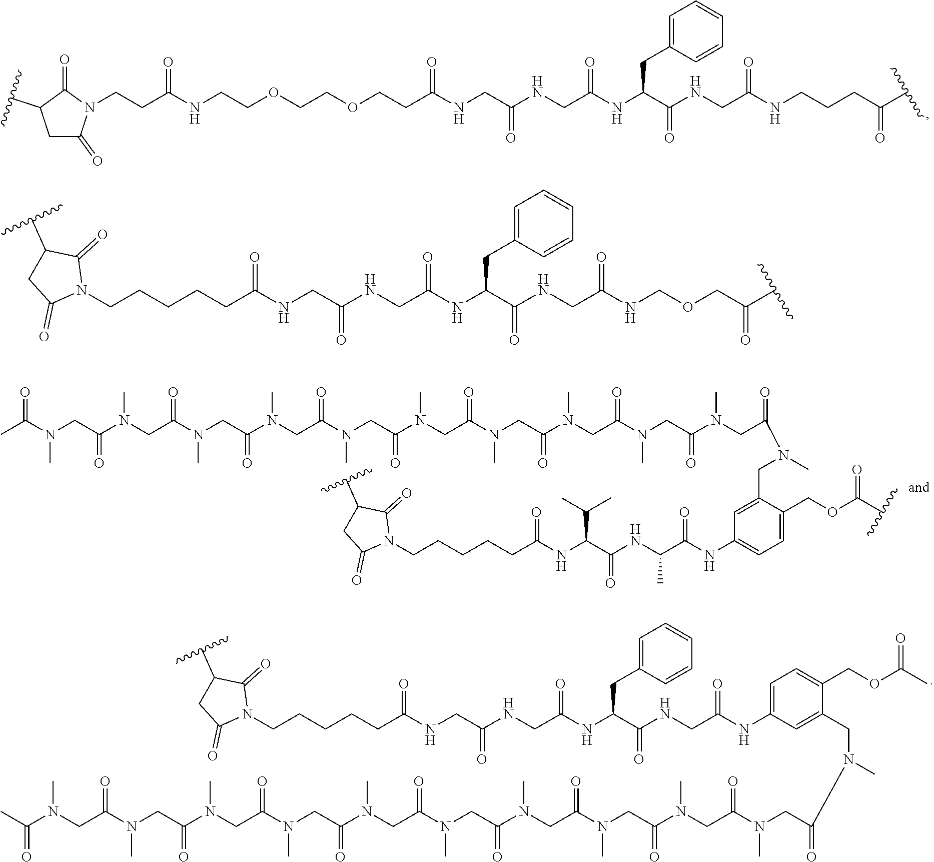

In some embodiments, the antibody drug conjugate is selected from the following structure:

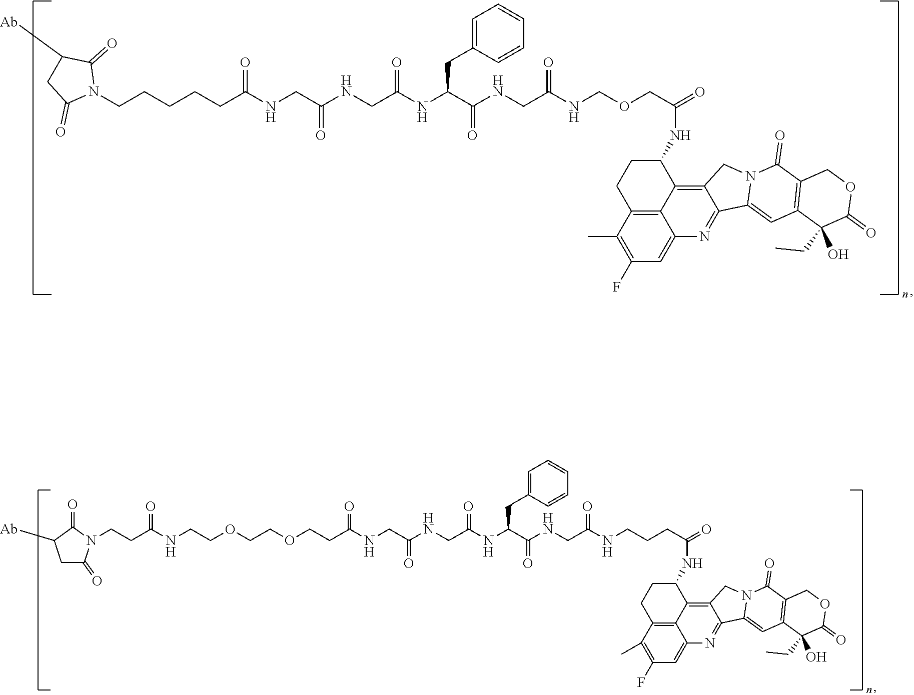

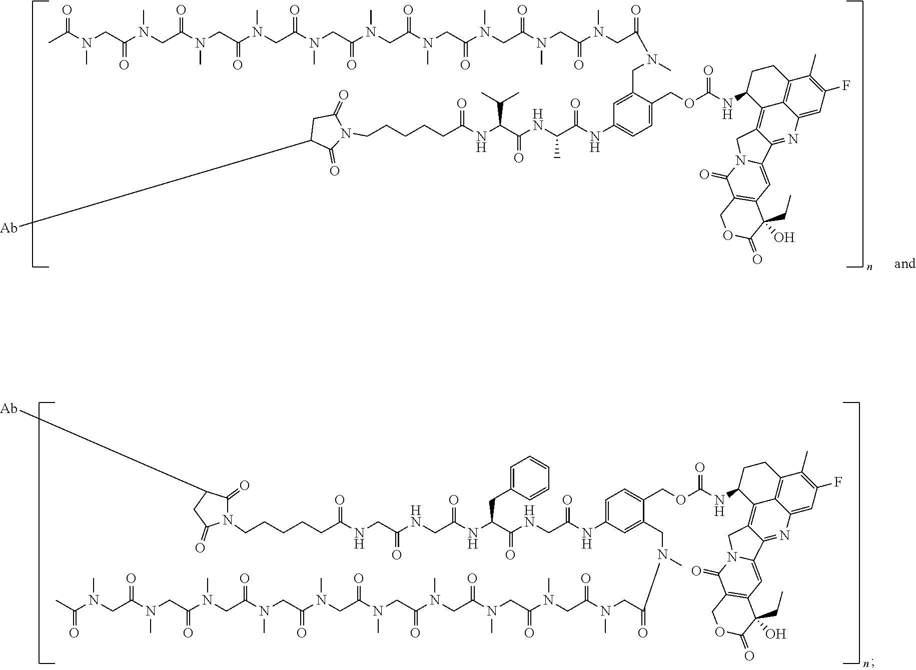

wherein n is any number from 1 to 10.

In some embodiments, the n is any number from 2 to 9.

The present application further provides a method of preparing an immunoconjugate of the present application, comprising the step of reacting an antibody or antigen binding fragment thereof of the present application with a drug-linker intermediate compound.

The present application further provides a pharmaceutical composition comprising the immunoconjugate of the present application, a salt thereof or a hydrate of the conjugate or the salt.

In some embodiments, the pharmaceutical composition further comprises a pharmaceutically acceptable carrier or excipient.

The present application further provides use of the immunoconjugate of the present application or the pharmaceutical composition of the present application in the manufacture of a medicament for treating tumors.

In some embodiments, the tumor is a tumor expressing CDH6.

In some embodiments, the tumor comprises renal cell carcinoma, renal clear cell carcinoma, papillary renal cell carcinoma, ovarian cancer, ovarian serous adenocarcinoma, thyroid cancer, bile duct cancer, lung cancer, small-cell lung cancer, glioblastoma, mesothelioma, uterine cancer, pancreatic cancer, Wilms' tumor or neuroblastoma.

The present application further provides a method for treating a tumor, which comprises administering the immunoconjugate of the present application, a salt thereof, and/or a hydrate of the conjugate or the salt to a subject.

In some embodiments, the tumor is a tumor expressing CDH6.

In some embodiments, the tumor comprises renal cell carcinoma, renal clear cell carcinoma, papillary renal cell carcinoma, ovarian cancer, ovarian serous adenocarcinoma, thyroid cancer, bile duct cancer, lung cancer, small-cell lung cancer, glioblastoma, mesothelioma, uterine cancer, pancreatic cancer, Wilms' tumor or neuroblastoma.

The present application further provides a method for treating a tumor, which comprises administering a pharmaceutical composition comprising at least one component selected from the immunoconjugate of the present application, a salt thereof, and a hydrate of the conjugate or the salt, and at least one antitumor drug to a subject, simultaneously, separately or sequentially.

### Advantageous Effects of Disclosure

An anti-CDH6 antibody drug conjugate comprising the anti-CDH6 antibody of the present disclosure conjugated to a drug exerting toxicity in cells via a linker having a specific structure can be expected to achieve an excellent antitumor effect and safety by administration to patients having cancer cells expressing CDH6. Specifically, the anti-CDH6 antibody drug conjugate of the present disclosure is useful as an antitumor agent.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1A-1B show flow cytometry results of examining the binding of an anti-CDH6 chimeric antibody (Ch069707) and its humanized variants (CL069707-H1L1, CL069707-H1L2, CL069707-H2L1, CL069707-H2L2) to ovarian cancer cell line OVCAR-3.

FIG. 2 shows flow cytometry results of examining the binding of an anti-CDH6 chimeric antibody (Ch069463) and its humanized variants (CL069463-H1L1, CL069463-H2L2, CL069463-H2L3) to ovarian cancer cell line OVCAR-3.

FIG. 3 shows ELISA results of examining the binding of CL069707-H1L1 to human CDH6-ECD-His, CDH9-ECD-His and CDH10-ECD-His recombinant protein.

FIG. 4 shows ELISA results of examining the binding of CL069707-H1L1 to human, cyno monkey, mouse and rat CDH6-ECD-His recombinant protein.

FIG. 5A shows the SEC chromatogram of CL069707-H1L1 measured by common procedure G.

FIG. 5B shows the HIC chromatogram of CL069707-H1L1 measured by common procedure H.

FIG. 6A shows the SEC chromatogram of the antibody-drug conjugate CL069707-H1L1-LP1 measured by common procedure G.

FIG. 6B shows the HIC chromatogram of the antibody-drug conjugate CL069707-H1L1-LP1 measured by common procedure H.

FIG. 7A shows the SEC chromatogram of the antibody-drug conjugate CL069707-H1L1-LP2 measured by common procedure G.

FIG. 7B shows the HIC chromatogram of the antibody-drug conjugate CL069707-H1L1-LP2 measured by common procedure H.

FIG. 8A shows the SEC chromatogram of the antibody-drug conjugate CL069707-H1L1-GGFG-DXd measured by common procedure G.

FIG. 8B shows the HIC chromatogram of the antibody-drug conjugate CL069707-H1L1-GGFG-DXd measured by common procedure H.

FIG. 9A shows the SEC chromatogram of the antibody-drug conjugate DS-6000a measured by common procedure G.

FIG. 9B shows the HIC chromatogram of the antibody-drug conjugate DS-6000a measured by common procedure H.

FIG. 10A shows the SEC chromatogram of the antibody-drug conjugate Reference Antibody-LP1 measured by common procedure G.

FIG. 10B shows the HIC chromatogram of the antibody-drug conjugate Reference Antibody-LP1 measured by common procedure H.

FIG. 11A shows the SEC chromatogram of the antibody-drug conjugate Human IgG-LP1 measured by common procedure G.

FIG. 11B shows the HIC chromatogram of the antibody-drug conjugate Human IgG-LP1 measured by common procedure H.

FIG. 12A shows the SEC chromatogram of the antibody-drug conjugate Human IgG-LP2 measured by common procedure G.

FIG. 12B shows the HIC chromatogram of the antibody-drug conjugate Human IgG-LP2 measured by common procedure H.

FIG. 13A shows the SEC chromatogram of the antibody-drug conjugate Human IgG-GGFG-DXd measured by common procedure G.

FIG. 13B shows the HIC chromatogram of the antibody-drug conjugate Human IgG-GGFG-DXd measured by common procedure H.

FIG. 14 shows the internalization of CL069707-H1L1-LP1 by PA-1, OVCAR-3 and 786-O cells.

FIG. 15A-15D show the results of evaluating the in vitro cell growth inhibition activity of anti-CDH6 antibody drug conjugates (CL069707-H1L1-LP1, CL069707-H1L1-LP2, CL069707-H1L1-GGFG-DXd, CL069707-H1L2-GGFG-DXd, CL069707-H2L1-GGFG-DXd, CL069707-H2L2-GGFG-DXd) against CDH6-positive tumor cell line OVCAR-3 and PA-1.

FIG. 16A shows the results of the in vitro cell killing activity of anti-CDH6 antibodies (CL069707, CL069439, CL069066) conjugated to vc-MMAE.

FIG. 16B shows the results of the in vitro cell killing activity of anti-CDH6 antibodies (CL069707, CL069439, CL069066) conjugated to GGFG-DXd.

FIG. 17A shows the in vivo antitumor effects of 3 humanized anti-CDH6 antibody drug conjugates (CL069707-H1L1-LP1, CL069707-H1L1-LP2, CL069707-H1L1-GGFG-DXd).

FIG. 17B shows the effect on body weight of mice of 3 humanized anti-CDH6 antibody drug conjugates (CL069707-H1L1-LP1, CL069707-H1L1-LP2, CL069707-H1L1-GGFG-DXd). The evaluation was conducted using animal models in which CDH6-positive human ovarian teratoma cell line PA-1 was inoculated into immunodeficient mice.

FIG. 18A shows CL069707-H1L1-LP1, CL069707-H1L1-LP2 exerted dose-dependent tumor regression after a single dose administration. FIG. 18B shows the effect of different doses of CL069707-H1L1-LP1, CL069707-H1L1-LP2 on body weight of mice. The evaluation was conducted using animal models in which CDH6-positive human ovarian teratoma cell line PA-1 was inoculated into immunodeficient mice.

FIG. 19A shows the in vivo antitumor effects of CL069707-H1L1-LP1. FIG. 19B shows the effect of a single dose of CL069707-H1L1-LP1 on body weight of mice. The evaluation was conducted using animal models in which CDH6-positive tumor cell line OVCAR-3 was inoculated into immunodeficient mice.

FIG. 20A shows the in vivo antitumor effects of CL069707-H1L1-LP1. FIG. 20B shows the effect on body weight of mice of CL069707-H1L1-LP1. The evaluation was conducted using animal models in which CDH6-positive tumor cell line 786-O was inoculated into immunodeficient mice.

FIG. 21A shows the in vivo antitumor effects of CL069707-H1L1-LP1. FIG. 21B shows the effect on body weight of mice of CL069707-H1L1-LP1. The evaluation was conducted using a Renal Carcinoma Patient-Derived Xenograft (PDX) Model.

FIG. 22A-22C shows the structure of the CL069707-H1L1-LP1, CL069707-H1L1-LP2 and CL069707-H1L1-GGFG-DXd.

FIG. 23 shows the detection sensitivity of the binding of the antibody described in the present application to CDH6 antigen.

FIG. 24 shows the specific recognition of CDH6 antigen expressed on the cell surface by the antibody described in the present application.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Unless stated otherwise, the following terms and phrases as used herein are intended to have the following meanings:

In the present application, the term “alkyl” generally refers to a monovalent saturated hydrocarbon chain having the specified number of carbon atoms. For example, C1-C6 alkyl refers to an alkyl group having from 1 to 6 carbon atoms. Alkyl groups may be straight or branched. Representative branched alkyl groups have one, two or three branches. Examples of alkyl groups include, but are not limited to, methyl, ethyl, propyl (n-propyl and isopropyl), butyl (n-butyl, isobutyl, sec-butyl, and t-butyl), pentyl (n-pentyl, isopentyl, and neopentyl), and hexyl.

In the present application, the term “antibody” as used herein generally refers to a polypeptide of the immunoglobulin family that is capable of binding a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antibody” includes, but is not limited to, monoclonal antibodies, human antibodies, humanized antibodies, camelid antibodies, and chimeric antibodies. The antibodies can be of any isotype/class (e.g., IgG, IgE, IgM, IgD, IgA and IgY) or subclass (e.g., IgG1, IgG2, IgG3, IgG4, IgA1 and IgA2).

In the present application, the term “Complementarity determining domains” is used herein interchangeably with the term “complementary determining regions” (“CDRs”), and generally refers to the hypervariable regions of VL and VH. The CDRs are the target protein-binding site of the antibody chains that harbors specificity for such target protein. There are three CDRs (CDR1-3, numbered sequentially from the N-terminus) in each human VL or VH, constituting about 15-20% of the variable domains. CDRs can be referred to by their region and order. For example, “VH CDR1” or “HCDR1” both refer to the first CDR of the heavy chain variable region. The CDRs are structurally complementary to the epitope of the target protein and are thus directly responsible for the binding specificity. The remaining stretches of the VL or VH, the so-called framework regions, exhibit less variation in amino acid sequence (Kuby, Immunology, 4th ed., Chapter 4. W.H. Freeman & Co., New York, 2000). In the art, the CDR of the antibody can be defined by various methods, such as a Kabat definition rule based on sequence variability (see Kabat et al., protein sequence in immunology, 5th edition, National Institutes of Health, Bethesda, Maryland (1991)), a Chothia definition rule based on the position of a structural loop region (see Al-Lazikani et al., J Mol Biol 273:927-48, 1997) and a IMGT definition rule based on the concept of IMGT-ONTOLOGY and IMGT Scientific chart rules. In certain embodiments, the present application uses the IMGT rules to define the CDRs of an antibody. The definition rules of Martin, PyIgClassify and Combined definition rules of Kabat, Chothia, IMGT, Martin and PyIgClassify are also included in this application. (see Mark L. Chiu et al., Antibodies 8(4), 55, 2019).

TABLE A
Numbering systems for the amino acids to each region (in Chothia numbering)

| CDR | Kabat | Chothia | IMGT | Martin | PyIgClassify | Combined |
|---|---|---|---|---|---|---|
| LCDR1 | L24-L34 | L24-L34 | L27-L32 | L24-L34 | L24-L34 | L24-L34 |
| LCDR2 | L50-L56 | L50-L56 | L50-L52 | L50-L56 | L49-L56 | L49-L56 |
| LCDR3 | L89-L97 | L89-L97 | L89-L97 | L89-L97 | L89-L97 | L89-L97 |
| HCDR1 | H31-H35 | H26-H32 | H26-H33 | H26-H35 | H23-H35 | H23-H35 |
| HCDR2 | H50-H65 | H52-H56 | H51-H57 | H50-H58 | H50-H58 | H50-H65 |
| HCDR3 | H95-H102 | H95-H102 | H93-H102 | H95-H102 | H93-H102 | H93-H102 |

Wherein, Laa-Lbb or Haa-Hbb may refer to, from N-terminal, the amino acids sequence from NO.aa to NO.bb of light chain or heavy chain respectively. For example, L24-L34 refers to the amino acid sequence from NO.24 to NO.34 of light chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention, the numbering of the constant region domains increases as they become more distal from the antigen binding site or amino-terminus of the antibody. The N-terminus is a variable region and at the C-terminus is a constant region; the CH3 and CL domains actually comprise the carboxy-terminal domains of the heavy and light chain, respectively.

In the present application, the term “antigen binding fragment”, as used herein, generally refers to a polypeptide including one or more portions of an antibody that retain the ability to specifically interact with (e.g., by binding, steric hindrance, stabilizing/destabilizing, spatial distribution) an epitope of an antigen. Examples of binding fragments include, but are not limited to, single-chain Fvs (scFv), disulfide-linked Fvs (sdFv), Fab fragments, F(ab′) fragments, a monovalent fragment consisting of the VL, VH, CL and CHI domains; a F(ab′)2 fragment, a bivalent fragment comprising two Fab fragments linked by a disulfide bridge at the hinge region; a Fd fragment consisting of the VH and CHI domains; a Fv fragment consisting of the VL and VH domains of a single arm of an antibody; a dAb fragment (Ward et al., Nature 341:544-546, 1989), which consists of a VH domain; and an isolated complementarity determining region (CDR) or other epitope-binding fragments of an antibody.

Antigen binding fragments also comprises single domain antibodies, maxibodies, minibodies, nanobodies, intrabodies, diabodies, triabodies, tetrabodies and bis-scFv (see, e.g., Hollinger and Hudson, Nature Biotechnology 23: 1126-1136, 2005). Antigen binding fragments can also comprise single chain molecules comprising a pair of tandem Fv segments (VH-CH1-VH—CH1) which, together with complementary light chain polypeptides, form a pair of antigen binding regions (Zapata et al, Protein Eng. 8: 1057-1062, 1995). A fragment of a conventional antibody may also be a single domain antibody, such as a heavy chain antibody or VHH.

In the present application, the term “monoclonal antibody” as used herein generally refers to polypeptides, including antibodies and antigen binding fragments that have substantially identical amino acid sequence or are derived from the same genetic source. This term also includes preparations of antibody molecules of single molecular composition. A monoclonal antibody composition displays a single binding specificity and affinity for a particular epitope.

In the present application, the term “humanized antibody” generally refers to an antibody which includes sequences of heavy chain variable regions and light chain variable regions derived from non-human species (e.g., mice), but in which at least a portion of the VH and/or VL sequences have been changed to be similar to the human germline variable sequences. For example, the term “humanized antibody” is an antibody or a variant, derivative, analogue or fragment thereof that can bind to a related antigen with immune specificity and includes a framework region (FR) which includes substantially an amino acid sequence of a human antibody and a complementary determining region (CDR) which includes substantially an amino acid sequence of a non-human antibody. In the context of CDR, the term “substantially” means that the amino acid sequence of CDR has at least 80%, e.g., at least 85%, at least 90%, at least 95%, at least 98% or at least 99% identity with an amino acid sequence of CDR of a non-human antibody. The humanized antibodies include substantially at least one, typically two variable domains (Fab, Fab′, F(ab′)2, Fab, Fv), wherein all or substantially all CDR regions correspond to the CDR region of a non-human immunoglobulin and all or substantially all framework regions are frame regions with consensus sequence of human immunoglobulin. In some embodiments, the humanized antibody can further include at least a portion of a constant region of immunoglobulin (Fe), typically a constant region of human immunoglobulin.

In the present application, the term “human antibody” generally refers to an antibody with variable and constant regions derived from the sequence of immunoglobulin of human germ line. Human antibodies are well known in the prior art (e.g., please refer to van Dijk, M. A. and van de Winkel, J. G., Curr. Opin. Chem. Biol. 5(2001)368-374). The human antibodies can also be generated in transgenic animals (e.g., mice) which can generate a complete or selected set of human antibodies in absence of generated endogenous immunoglobulin after immunization (e.g., Jakobovits, A. et al., Proc. Natl. Acad. Sci. USA 90(1993)2551-2555; Jakobovits, A. et al., Nature 362(1993)255-258; Brueggemann, M. et al., Year Immunol. 7(1993)33-40). The human antibodies can also be generated in a phage display library (e.g., Hoogenboom, H. R. and Winter, G., J. Mol. Biol. 227(1992)381-388; Marks, J. D. et al., J. Mol. Biol. 222(1991)581-597). The term “human antibody” can also include antibodies modified in the constant regions.

In the present application, the term “chimeric antibody” generally refers to an engineered antibody which in its broadest sense contains one or more regions from one antibody and one or more regions from one or more other antibody(ies). In particular a chimeric antibody comprises a VH domain and a VL domain of an antibody derived from a non-human animal, in association with a CH domain and a CL domain of another antibody, in particular a human antibody. As the non-human animal, any animal such as mouse, rat, hamster, rabbit or the like can be used. A chimeric antibody may also denote a multispecific antibody having specificity for at least two different antigens.

By “purified” and “isolated” it is meant, when referring to a polypeptide (i.e. the antibody of the invention) or a nucleotide sequence, that the indicated molecule is present in the substantial absence of other biological macromolecules of the same type. The term “purified” as used herein in particular means at least 75%, 85%, 95% or 98% by weight, of biological macromolecules of the same type are present. An “isolated” nucleic acid molecule that encodes a particular polypeptide refers to a nucleic acid molecule that is substantially free of other nucleic acid molecules that do not encode the subject polypeptide; however, the molecule may include some additional bases or moieties, which do not deleteriously affect the basic characteristics of the composition. The present invention may contain, for example, an isolated antigen-binding protein, an isolated antibody or an antigen-binding fragment thereof, an isolated polypeptide, an isolated nucleic acid molecule or molecules.

In the present application, the term “Affinity” is generally defined by the equilibrium association between the whole antibody and the antigen. Affinity may be expressed for example in half-maximal effective concentration (EC50) or the equilibrium dissociation constant (KD). Affinity can be experimentally assessed by a variety of known methods, such as measuring association and dissociation rates with surface Plasmon resonance or measuring the EC50 in an immunochemical assay (ELISA, FACS).

“Half maximal effective concentration” or “EC50 “generally refers to the concentration of a drug, antibody or toxicant which induces a response halfway between the baseline and maximum after a specified exposure time. EC50 and affinity are inversely related, the lower the EC50 value the higher the affinity of the antibody.

“KD” is the equilibrium dissociation constant, a ratio of koff/kon, between the antibody and its antigen. KD and affinity are inversely related. The KD value relates to the concentration of antibody and the lower the KD value and the higher the affinity of the antibody. The antibodies of the present disclosure generally will have an equilibrium dissociation constant of less than about 10⁻⁷ M or 10⁻⁸ M, for example, less than about 10⁻⁹ M or 10⁻¹⁰ M, in some aspects, less than about 10⁻¹¹ M, 10⁻¹² M or 10⁻¹³ M.

The term “conservatively modified variant” applies to both amino acid and nucleic acid sequences. With respect to particular nucleic acid sequences, conservatively modified variant refers to those nucleic acids which encode identical or essentially identical amino acid sequences or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some aspects, the term “conservative sequence modifications” are used to refer to amino acid modifications that do not significantly affect or alter the binding characteristics of the antibody containing the amino acid sequence.

In the present application, the term “percent identical” or “percent identity,” in the context of two or more nucleic acids or polypeptide sequences, refers to the extent to which two or more sequences or subsequences that are the same. Two sequences are “identical” if they have the same sequence of amino acids or nucleotides over the region being compared. Two sequences are “substantially identical” if two sequences have a specified percentage of amino acid residues or nucleotides that are the same (i.e., 60% identity, optionally 65%, 70%, 75%, 80%, 85%, 90%, 95% or 99% identity over a specified region or, when not specified, over the entire sequence), when compared and aligned for maximum correspondence over a comparison window or designated region as measured using one of the following sequence comparison algorithms or by manual alignment and visual inspection. Optionally, the identity exists over a region that is at least about 30 nucleotides (or 10 amino acids) in length or more preferably over a region that is 100 to 500 or 1000 or more nucleotides (or 20, 50, 200 or more amino acids) in length. Two examples of algorithms that are suitable for determining percent sequence identity and sequence similarity are the BLAST and BLAST 2.0 algorithms, which are described in Altschul et al, Nuc. Acids Res. 25:3389-3402, 1977; and Altschul et al, J. Mol. Biol. 215:403-410, 1990, respectively.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions. Yet another indication that two nucleic acid sequences are substantially identical is that the same primers can be used to amplify the sequence.

In the present application, the term “nucleic acid” is used herein interchangeably with the term “polynucleotide” and generally refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

In the present application, the term “polypeptide” is used herein interchangeably with the term “protein” and refers to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

In the present application, the term “immunoconjugate” as used herein generally refers to the linkage of an antibody or an antigen binding fragment thereof with another agent, such as a payload, a drug moiety, a chemotherapeutic agent, a toxin, an immunotherapeutic agent, an imaging probe, and the like. The linkage can be a covalent bond or non-covalent interactions such as through electrostatic forces. Various linkers, known in the art, can be employed in order to form the immunoconjugate. Additionally, the immunoconjugate can be provided in the form of a fusion protein that may be expressed from a polynucleotide encoding the immunoconjugate. As used herein, “fusion protein” refers to a protein created through the joining of two or more genes or gene fragments which originally coded for separate proteins (including peptides and polypeptides). Translation of the fusion gene results in a single protein with functional properties derived from each of the original proteins.

The term “active moiety” or “payload” as used herein generally refers to the portion of a conjugated compound that constitutes an active agent, which mediates a pharmaceutical effect including but not limited to prophylactic, therapeutic, and/or diagnostic effects, for example, an anti-cancer, anti-inflammatory, anti-infective (e.g., anti-fungal, antibacterial, anti-parasitic, anti-viral) or an anesthetic agent. Methods for attaching each of these to a linker compatible with the antibodies and method of the present disclosure are known in the art. See, e.g., Singh et al., (2009) Therapeutic Antibodies: Methods and Protocols, vol. 525, 445-457. In addition, an “active moiety” or a “payload” can be a biophysical probe, a fluorophore, a spin label, an infrared probe, an affinity probe, a chelator, a spectroscopic probe, a radioactive probe, a lipid molecule, a polyethylene glycol, a polymer, DNA, RNA, a protein, a peptide, a surface, an antibody, an antibody fragment, a nanoparticle, a quantum dot, a liposome, a PLGA particle, a saccharide or a polysaccharide.

In the present application, the term “drug moiety” or “D” generally refers to any compound possessing a desired biological activity and a reactive functional group that may be used to incorporate the drug into the conjugate of the disclosure. In some embodiments, the drug moiety indicate a cytotoxic drug useful in cancer therapy; a protein or polypeptide possessing a desired biological activity, such as a toxin, e.g., abrin, ricin A, Pseudomonas exotoxin, and diphtheria toxin; other suitable proteins include tumor necrosis factor, a-interferon, 3-interferon, nerve growth factor, platelet derived growth factor, tissue plasminogen activator, and biological response modifiers, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF) or other growth factors. In some embodiments, the term “drug moiety” may be a chemical moiety. In certain aspects, a drug moiety is selected from a V-ATPase inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, an inhibitor of phosphoryl transfer reactions in mitochondria, a protein synthesis inhibitor, a kinase inhibitor, a CDK2 inhibitor, a CDK9 inhibitor, a proteasome inhibitor, a kinesin inhibitor, an HDAC inhibitor, a DNA damaging agent, a DNA alkylating agent, a DNA intercalator, a DNA minor groove binder and a DHFR inhibitor.

In one embodiment, the drug moiety can be microtubule disrupting drugs such as auristatin, e.g. monomethyl auristatin E (MMAE), monomethyl auristatin F (MMAF), and auristatin F (AF). In another embodiment, the drug moiety can be microtubule disrupting drugs such as maytansinoids, e.g. DM1, DM3, and DM4. In another embodiment, the drug moiety can be DNA damaging agents such as calicheamicins, duocarmycins, SN-38, and pyrrolo[2,1-c][1,4]benzodi-azepines (PBDs). Still in other embodiments, the drug moiety can be amanitins, anthracyclines, baccatins, camptothecins, cemadotins, colchicines, colcimids, combretastatins, cryptophycins, discodermolides, docetaxel, doxorubicin, echinomycins, eleutherobins, epothilones, estramustines, lexitropsins, maytansines, methotrexate, netropsins, puromycins, rhizoxins, taxanes, tubulysins or vinca alkaloids.

In the present application, the term “topoisomerase inhibitor” usually refers to a compound that inhibits topoiosmerase activity. Compounds known as topoisomerase I inhibitors have activity against topoisomerase I, and the topoiosmerase II inhibitors have activity against topoisomerase II. Some compounds have activity against both topoisomerase I and topoisomerase II and are known as topoisomerase I/II inhibitors. Preferred topoisomerase I inhibitors for use in the present invention are camptothecin and analogs of camptothecin. Camptothecin is a pentacyclic alkaloid initially isolated from the wood and bark of Camptotheca acuminata, a tree indigenous to China (Wall, M. E. et al, J. Am. Chem. Soc, 94:388 (1966)). Camptothecin exerts its pharmacological effects by irreversibly inhibiting topoisomerase I. Methods for the synthesis of camptothecin and camptothecin analogs or derivatives are known, and are summarized and as set forth in U.S. Pat. No. 5,244,903, which is herein incorporated by reference in its entirety.

In the present application, the term “camptothecin” generally includes camptothecin and camptothecin derivatives including irinotecan, topotecan, lurtotecan, silatecan, etirinotecan pegol, TAS 103, 9-aminocamptothecin, 7-ethylcamptothecin, 10-hydroxycamptothecin, 9-nitrocamptothecin, 10,11-methylenedioxycamptothecin, 9-amino-10,11-methylenedioxycamptothecin, 9-chloro-10,11-methylenedioxycamptothecin, 7-(4-methylpiperazinomethylene)-10,11-ethylenedioxy-20(S)-camptothecin, 7-(4-methylpiperazinomethylene)-10,11-methylenedioxy-20(S)-camptothecin, and 7-(2-(N-isopropylamino)ethyl)-(20S)-camptothecin, and stereoisomers, salts and esters thereof.

It should be noted that the drugs are not limited to above-mentioned categories and include all that could be used in ADCs.

In the present application, the term “linker” described in the present disclosure includes a cleavable linker or a noncleavable linker. Cleavable linkers can be chemically labile and enzyme-labile linkers. Due to the high plasma stability and good intracellular cleaving selectivity and efficiency, enzyme-labile linkers are broadly selected as cleavable linker candidates in ADCs. In some embodiments, enzyme-labile linkers may include a peptide unit (-AAs-) selected from a group consisting of -valline-citruline- (-Val-Cit-), -valline-lysine- (-Val-Lys-), -valline-arginine- (-Val-Arg-), -phenylalanine-citruline- (-Phe-Cit-), -phenylalanine-lysine-(-Phe-Lys-), and -phenylalanine-arginine- (-Phe-Arg-). Typical enzyme-labile linkers include -Val-Cit- and -Phe-Lys-, which can be recognized by cathepsin B. In some embodiments, the noncleavable linker may be linkers that are capable of increasing the hydrophilicity of the resulting ADC. In one embodiment, the noncleavable linker may include one or more poly(ethylene glycol)(PEG). In other embodiment, the noncleavable linker may be PEG, PEG diamine (NH2-PEG-NH2), amine-PEG-hydroxyl (NH2-PEG-OH), amine-PEG-COGH (NH2-PEG-COOH), diethylene triamine or a combination thereof. In some embodiments, PEG may be represented by —(CH₂CH₂O)ₓ—, wherein x may be an integer ranging from 1 to 20.

In the present application, the term “toxin,” “cytotoxin” or “cytotoxic agent” as used herein, generally refers to any agent that is detrimental to the growth and proliferation of cells and may act to reduce, inhibit or destroy a cell or malignancy.

In the present application, the term “anti-tumor agent” or “antitumor drug” as used herein generally refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

In the present application, the term “tumor” or “cancer” generally refers to neoplastic cell growth and proliferation, whether malignant or benign, and all pre-cancerous and cancerous cells and tissues.

In the present application, the term “anti-tumor activity” means a reduction in the rate of tumor cell proliferation, viability or metastatic activity. A possible way of showing anti-tumor activity is to show a decline in growth rate of tumor cells, tumor size stasis or tumor size reduction. Such activity can be assessed using accepted in vitro or in vivo tumor models, including but not limited to xenograft models, allograft models, MMTV models, and other known models known in the art to investigate anti-tumor activity.

In the present application, the term “Cadherin 6” or “CDH6” generally refers to a single-pass transmembrane protein composed of 790 amino acids, which is classified into the type II cadherin family, and this protein has N-terminal extracellular and C-terminal intracellular domains. The human CDH6 gene was cloned for the first time in 1995, and its sequence can be referred to under, for example, accession Nos. NM_004932 and NP_004923 (NCBI). Moreover, a protein which consists of an amino acid sequence comprising a substitution, deletion and/or addition of one or several amino acids in the above-described amino acid sequence of CDH6, and has a biological activity equivalent to that of the CDH6 protein, is also included within the term “CDH6”.

In the present description, the term “epitope” is generally used to mean the partial peptide or partial three-dimensional structure of CDH6, to which a specific anti-CDH6 antibody binds.

The terms “CDH6 expressing tumor “or “CDH6 positive tumor “generally refers to a tumor that express CDH6 and/or a mutant form of CDH6 on the surface of tumor cells.

In the present application, the term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

In the present application, the term “pharmaceutically acceptable” generally refers to one or more non-toxic substances that do not interfere with the effectiveness of the biological activity of the active ingredient. Such formulations may typically contain salts, buffers, preservatives, compatible carriers, and optionally other therapeutic agents. Such pharmaceutically acceptable formulations may also typically contain compatible solid or liquid fillers, diluents, or encapsulating materials suitable for administration to humans. When used in medicine, the salt should be a pharmaceutically acceptable salt, but non-pharmaceutically acceptable salts can be conveniently used to prepare pharmaceutically acceptable salts and cannot be excluded from the scope of the present invention. Such pharmacologically and pharmaceutically acceptable salts include, but are not limited to, salts prepared from the following acids: hydrochloric acid, hydrobromic acid, sulfuric acid, nitric acid, phosphoric acid, maleic acid, acetic acid, salicylic acid, citric acid, boric acid, formic acid, malonic acid, succinic acid, etc. Pharmacologically acceptable salts can also be prepared as alkali metal salts or alkaline earth metal salts, such as sodium, potassium or calcium salts. The term “solvate” is used herein in the conventional sense to refer to a complex of a solute (e.g., an active compound, a salt of an active compound) and a solvent. Solvates usually do not significantly alter the physiological activity or toxicity of the compound and therefore can act as pharmacological equivalents. If the solvent is water, the solvent compound may be conveniently referred to as a hydrate, e.g., monohydrate, dihydrate, trihydrate, etc.

In the present application, the terms “treat,” “treating,” or “treatment” of any disease or disorder generally refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter) or both. In yet another aspect, “treat,” “treating,” or “treatment” refers to preventing or delaying the onset or development or progression of the disease or disorder.

In the present application, the term “therapeutically acceptable amount” or “therapeutically effective dose” interchangeably refers to an amount sufficient to effect the desired result (i.e., a reduction in tumor size, inhibition of tumor growth, prevention of metastasis, inhibition or prevention of viral, bacterial, fungal or parasitic infection). In some aspects, a therapeutically acceptable amount does not induce or cause undesirable side effects. A therapeutically acceptable amount can be determined by first administering a low dose, and then incrementally increasing that dose until the desired effect is achieved. A “prophylactically effective dosage,” and a “therapeutically effective dosage,” of the molecules of the present disclosure can prevent the onset of or result in a decrease in severity of, respectively, disease symptoms, including symptoms associated with cancer.

In the present application, the term “co-administer” generally refers to the simultaneous presence of two active agents in the blood of an individual. Active agents that are co-administered can be concurrently or sequentially delivered.

In the present application, the terms “comprising”, “containing”, “having”, “include”, and “including” are to be construed as “including, but not limited to” unless otherwise noted. The terms “a,” “an,” and “the” and similar referents in the context of describing the invention and, specifically, in the context of the appended claims, are to be construed to cover both the singular and the plural unless otherwise noted. The use of any and all examples or exemplary language (“for example”, “e.g.”, “such as”) is intended merely to illustrate aspects or embodiments of the invention, and is not to be construed as limiting the scope thereof, unless otherwise claimed.

In the present application, the term “about” when referring to a measurable value such as an amount, a temporal duration, and the like, is meant to encompass variations of ±20% or in some instances ±10% or in some instances ±5% or in some instances ±1% or in some instances ±0.1% from the specified value, as such variations are appropriate to perform the disclosed methods.

### Anti-CDH6 Antibody

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The anti-CDH6 antibody of the present invention may be derived from any species. Preferred examples of the species can include humans, monkeys, rats, mice and rabbits. When the anti-CDH6 antibody of the present invention is derived from a species other than humans, it is preferred to chimerize or humanize the anti-CDH6 antibody by a well-known technique. The antibody of the present invention may be a polyclonal antibody or may be a monoclonal antibody, and a monoclonal antibody is preferred.

The anti-CDH6 antibody of the present invention is an antibody that can target tumor cells. Specifically, the anti-CDH6 antibody of the present invention possesses the property of being able to recognize tumor cells, the property of being able to bind to tumor cells, and/or the property of being internalized into tumor cells by cellular uptake, and the like. Accordingly, the anti-CDH6 antibody of the present invention can be conjugated to an active moiety via a linker to prepare an immunoconjugate (e.g., the anti-CDH6 antibody of the present invention can be conjugated to a compound having antitumor activity via a linker to prepare antibody drug conjugate).

The anti-CDH6 antibody can be obtained by immunizing an animal with a polypeptide serving as an antigen by a method usually performed in this field, and then collecting and purifying an antibody produced in a living body thereof. The origin of the antigen is not limited to a human, and thus, an animal can also be immunized with an antigen derived from a non-human animal such as a mouse or a rat. In this case, an antibody applicable to the disease of a human can be selected by examining the cross-reactivity of the obtained antibody binding to the heterologous antigen with the human antigen.

Furthermore, antibody-producing cells that produce an antibody against the antigen can be fused with myeloma cells according to a known method (e.g., Kohler and Milstein, Nature (1975) 256, 495-497; and Kennet, R. ed., Monoclonal Antibodies, 365-367, Plenum Press, N. Y. (1980)) to establish hybridomas, so as to obtain a monoclonal antibody.

The anti-CDH6 antibody used in the present invention is not particularly limited. For example, an antibody specified by an amino acid sequence as set forth in the sequence listing of the present application can be suitably used. The anti-CDH6 antibody used in the present invention is desirably an antibody having the following properties: (a) specifically binding to CDH6, and (b) having the activity of being internalized into CDH6 expressing cells by binding to CDH6; wherein the CDH6 can be human CDH6.

Hereinafter, the method for obtaining an antibody against CDH6 will be specifically described.
(a) the extracellular region of CDH6 (Ser 54-Ala 615) can be used as the immunogen (ACRO Biosystems, CA6-H5229), and directly administering the antigen to an animal (e.g., a rat or a mouse) to be immunized. The administration of the antigen may be performed one or more times, preferably a plurality of times, if necessary for enhancing antibody titer;
(b) collection of tissue (e.g., a lymph node) containing antibody-producing cells from the aforementioned animal in which the immune response has been induced;
(c) preparation of myeloma cells (hereinafter, referred to as “myelomas”) (e.g., mouse myeloma SP2/0-ag14 cells);
(d) cell fusion between the antibody-producing cells and the myelomas;
(e) selection of a hybridoma group producing an antibody of interest;
(f) division into single cell clones (cloning);
(g) optionally, the culture of hybridomas for the mass production of monoclonal antibodies or the breeding of animals into which the hybridomas are inoculated; and/or
(h) study of the physiological activity (internalization activity) and binding specificity of the monoclonal antibody thus produced or examination of the properties of the antibody as a labeling reagent.

Examples of the method for measuring the antibody titer used herein can include, but are not limited to, flow cytometry and Cell-ELISA.

The specific CDR sequences defined herein are generally based on the IMGT definition. However, it is understood that reference to a heavy chain CDR or CDRs and/or a light chain CDR or CDRs of a specific antibody encompass all CDR definitions as known to those of skill in the art.

In some embodiments, anti-CDH6 antibodies or antigen-binding fragments provided herein comprise one, two, three, four, five, and/or six CDRs of any one of the antibodies described herein. In some embodiments, anti-CDH6 antibodies or antigen-binding fragments provided herein comprise a VL comprising one, two, and/or three, VL CDRs from Table 1. In some embodiments, anti-CDH6 antibodies or antigen-binding fragments provided herein comprise a VH comprising one, two, and/or three VH CDRs from Table 1. In some embodiments, anti-CDH6 antibodies or antigen-binding fragments provided herein comprise one, two, and/or three VL CDRs from Table 1 and one, two, and/or three VH CDRs from Table 1.

In some embodiments, an anti-CDH6 antibody or antigen-binding fragment thereof comprises a VL CDR1, VL CDR2, VL CDR3, VH CDR1, VH CDR2, and/or VH CDR3 from an antibody or antigen-binding fragment described herein. In some embodiments, an anti-CDH6 antibody or antigen-binding fragment thereof comprises a variant of an anti-CDH6 antibody or antigen-binding fragment described herein. In some embodiments, a variant of an anti-CDH6 antibody or antigen-binding fragment comprises one to 30 amino acid substitutions, additions, and/or deletions in the anti-CDH6 antibody or antigen-binding fragment. In some embodiments, a variant of an anti-CDH6 antibody or antigen-binding fragment comprises one to 25 amino acid substitutions, additions, and/or deletions in the anti-CDH6 antibody or antigen-binding fragment. In some embodiments, a variant of an anti-CDH6 antibody or antigen-binding fragment comprises one to 20 substitutions, additions, and/or deletions in the anti-CDH6 antibody or antigen-binding fragment. In some embodiments, a variant of an anti-CDH6 antibody or antigen-binding fragment comprises one to 15 substitutions, additions, and/or deletions in the anti-CDH6 antibody or antigen-binding fragment. In some embodiments, a variant of an anti-CDH6 antibody or antigen-binding fragment comprises one to 10 substitutions, additions, and/or deletions in the anti-CDH6 antibody or antigen-binding fragment. In some embodiments, a variant of an anti-CDH6 antibody or antigen-binding fragment comprises one to five conservative amino acid substitutions, additions, and/or deletions in the anti-CDH6 antibody or antigen-binding fragment. In some embodiments, a variant of an anti-CDH6 antibody or antigen-binding fragment comprises one to three amino acid substitutions, additions, and/or deletions in the anti-CDH6 antibody or antigen-binding fragment. In some embodiments, the amino acid substitutions, additions, and/or deletions are conservative amino acid substitutions. In some embodiments, the conservative amino acid substitution(s) is in a CDR of the antibody or antigen-binding fragment. In some embodiments, the conservative amino acid substitution(s) is not in a CDR of the antibody or antigen-binding fragment. In some embodiments, the conservative amino acid substitution(s) is in a framework region of the antibody or antigen-binding fragment.

In some embodiments, provided herein are antibodies or antigen-binding fragments thereof that specifically bind CDH6 (e.g., human CDH6) comprising:
a) a heavy chain variable region (VH) comprising (1) a heavy chain CDR1 (HCDR1) having an amino acid sequence of SEQ ID NO: 2; (2) a heavy chain CDR2 (HCDR2) having an amino acid sequence of SEQ ID NO: 3; or (3) a heavy chain CDR3 (HCDR3) having an amino acid sequence of SEQ ID NO: 4; or a variant thereof having up to about 3, about 5, about 8, about 10, about 12, or about 15 amino acid substitutions, additions, and/or deletions in the HCDRs;
and/or b) a light chain variable region (VL) comprising (1) a light chain CDR1 (LCDR1) having an amino acid sequence of SEQ ID NO: 5; (2) a light chain CDR2 (LCDR2) having an amino acid sequence of SEQ ID NO: 6; or (3) a light chain CDR3 (LCDR3) having an amino acid sequence of SEQ ID NO: 7; or a variant thereof having up to about 3, about 5, about 8, about 10, about 12, or about 15 amino acid substitutions, additions, and/or deletions in the LCDRs.

In some embodiments, the variant has up to about 5 amino acid substitutions, additions, and/or deletions in the HCDRs, and/or the variant has up to about 5 amino acid substitutions, additions, and/or deletions in the LCDRs.

In some embodiments, provided herein are antibodies or antigen-binding fragments thereof that specifically bind CDH6 (e.g., human CDH6) comprising: a) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence of SEQ ID NO: 18; and/or b) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence of SEQ ID NO: 23.

In some embodiments, provided herein are antibodies or antigen-binding fragments thereof that specifically bind CDH6 (e.g., human CDH6) comprising:
a) a heavy chain variable region (VH) comprising (1) a heavy chain CDR1 (HCDR1) having an amino acid sequence of SEQ ID NO: 8; (2) a heavy chain CDR2 (HCDR2) having an amino acid sequence of SEQ ID NO: 9; or (3) a heavy chain CDR3 (HCDR3) having an amino acid sequence of SEQ ID NO: 10; or a variant thereof having up to about 3, about 5, about 8, about 10, about 12, or about 15 amino acid substitutions, additions, and/or deletions in the HCDRs;
and/or b) a light chain variable region (VL) comprising (1) a light chain CDR1 (LCDR1) having an amino acid sequence of SEQ ID NO: 11; (2) a light chain CDR2 (LCDR2) having an amino acid sequence of SEQ ID NO: 12; or (3) a light chain CDR3 (LCDR3) having an amino acid sequence of SEQ ID NO: 13; or a variant thereof having up to about 3, about 5, about 8, about 10, about 12, or about 15 amino acid substitutions, additions, and/or deletions in the LCDRs.

In some embodiments, the variant has up to about 5 amino acid substitutions, additions, and/or deletions in the HCDRs, and/or the variant has up to about 5 amino acid substitutions, additions, and/or deletions in the LCDRs.

In some embodiments, provided herein are antibodies or antigen-binding fragments thereof that specifically bind CDH6 (e.g., human CDH6) comprising: a) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence of SEQ ID NO: 41; and/or b) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence of SEQ ID NO: 46.

In some embodiments, provided herein are antibodies or antigen-binding fragments thereof that specifically bind CDH6 (e.g., human CDH6) comprising:
a) a heavy chain variable region (VH) comprising (1) a heavy chain CDR1 (HCDR1) having an amino acid sequence of SEQ ID NO: 89; (2) a heavy chain CDR2 (HCDR2) having an amino acid sequence of SEQ ID NO: 90; or (3) a heavy chain CDR3 (HCDR3) having an amino acid sequence of SEQ ID NO: 91; or a variant thereof having up to about 3, about 5, about 8, about 10, about 12, or about 15 amino acid substitutions, additions, and/or deletions in the HCDRs;
and/or b) a light chain variable region (VL) comprising (1) a light chain CDR1 (LCDR1) having an amino acid sequence of SEQ ID NO: 92; (2) a light chain CDR2 (LCDR2) having an amino acid sequence of SEQ ID NO: 93; or (3) a light chain CDR3 (LCDR3) having an amino acid sequence of SEQ ID NO: 94; or a variant thereof having up to about 3, about 5, about 8, about 10, about 12, or about 15 amino acid substitutions, additions, and/or deletions in the LCDRs.

In some embodiments, the variant has up to about 5 amino acid substitutions, additions, and/or deletions in the HCDRs, and/or the variant has up to about 5 amino acid substitutions, additions, and/or deletions in the LCDRs.

In some embodiments, provided herein are antibodies or antigen-binding fragments thereof that specifically bind CDH6 (e.g., human CDH6) comprising: a) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence of SEQ ID NO: 95; and/or b) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence of SEQ ID NO: 96.

In some embodiments, provided herein are antibodies or antigen-binding fragments thereof that specifically bind CDH6 (e.g., human CDH6) comprising:
a) a heavy chain variable region (VH) comprising (1) a heavy chain CDR1 (HCDR1) having an amino acid sequence of SEQ ID NO: 97; (2) a heavy chain CDR2 (HCDR2) having an amino acid sequence of SEQ ID NO: 98; or (3) a heavy chain CDR3 (HCDR3) having an amino acid sequence of SEQ ID NO: 99; or a variant thereof having up to about 3, about 5, about 8, about 10, about 12, or about 15 amino acid substitutions, additions, and/or deletions in the HCDRs;
and/or b) a light chain variable region (VL) comprising (1) a light chain CDR1 (LCDR1) having an amino acid sequence of SEQ ID NO: 100; (2) a light chain CDR2 (LCDR2) having an amino acid sequence of SEQ ID NO: 101; or (3) a light chain CDR3 (LCDR3) having an amino acid sequence of SEQ ID NO: 102; or a variant thereof having up to about 3, about 5, about 8, about 10, about 12, or about 15 amino acid substitutions, additions, and/or deletions in the LCDRs.

In some embodiments, the variant has up to about 5 amino acid substitutions, additions, and/or deletions in the HCDRs, and/or the variant has up to about 5 amino acid substitutions, additions, and/or deletions in the LCDRs.

In some embodiments, provided herein are antibodies or antigen-binding fragments thereof that specifically bind CDH6 (e.g., human CDH6) comprising: a) a VH having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence of SEQ ID NO: 103; and/or b) a VL having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence of SEQ ID NO: 104.

Examples of the hybridoma strain thus established can include anti-CDH6 antibody-producing hybridomas 707 and 463. It is to be noted that, in the present description, an antibody produced by the anti-CDH6 antibody-producing hybridoma 707 is referred to as a “707 antibody” or simply “707”, an antibody produced by the hybridoma 463 is referred to as a “463 antibody” or simply “463”, an antibody produced by the hybridoma 066 is referred to as a “066 antibody” or simply “066”, and an antibody produced by the hybridoma 439 is referred to as a “439 antibody” or simply “439”.

The heavy chain variable region of the 707 antibody has HCDR1 consisting of the amino acid sequence as set forth in SEQ ID NO: 2, HCDR2 consisting of the amino acid sequence as set forth in SEQ ID NO: 3, and HCDR3 consisting of the amino acid sequence as set forth in SEQ ID NO: 4. The light chain variable region of the 707 antibody has LCDR1 comprising the amino acid sequence as set forth in SEQ ID NO: 5, LCDR2 comprising the amino acid sequence as set forth in SEQ ID NO: 6, and LCDR3 comprising the amino acid sequence as set forth in SEQ ID NO: 7.

Further, the heavy chain variable region of the 707 antibody consists of the amino acid sequence as set forth in SEQ ID NO: 18. The light chain variable region of the 707 antibody consists of the amino acid sequence as set forth in SEQ ID NO: 23.

The heavy chain variable region of the 463 antibody has HCDR1 consisting of the amino acid sequence as set forth in SEQ ID NO: 8, HCDR2 consisting of the amino acid sequence as set forth in SEQ ID NO: 9, and HCDR3 consisting of the amino acid sequence as set forth in SEQ ID NO: 10. The light chain variable region of the 463 antibody has LCDR1 comprising the amino acid sequence as set forth in SEQ ID NO: 11, LCDR2 comprising the amino acid sequence as set forth in SEQ ID NO: 12, and LCDR3 comprising the amino acid sequence as set forth in SEQ ID NO: 13.

Further, the heavy chain variable region of the 463 antibody consists of the amino acid sequence as set forth in SEQ ID NO: 41. The light chain variable region of the 463 antibody consists of the amino acid sequence as set forth in SEQ ID NO: 46.

The heavy chain variable region of the 066 antibody has HCDR1 consisting of the amino acid sequence as set forth in SEQ ID NO: 89, HCDR2 consisting of the amino acid sequence as set forth in SEQ ID NO: 90, and HCDR3 consisting of the amino acid sequence as set forth in SEQ ID NO: 91. The light chain variable region of the 066 antibody has LCDR1 comprising the amino acid sequence as set forth in SEQ ID NO: 92, LCDR2 comprising the amino acid sequence as set forth in SEQ ID NO: 93, and LCDR3 comprising the amino acid sequence as set forth in SEQ ID NO: 94.

Further, the heavy chain variable region of the 066 antibody consists of the amino acid sequence as set forth in SEQ ID NO: 95. The light chain variable region of the 066 antibody consists of the amino acid sequence as set forth in SEQ ID NO: 96.

The heavy chain variable region of the 439 antibody has HCDR1 consisting of the amino acid sequence as set forth in SEQ ID NO: 97, HCDR2 consisting of the amino acid sequence as set forth in SEQ ID NO: 98, and HCDR3 consisting of the amino acid sequence as set forth in SEQ ID NO: 99. The light chain variable region of the 439 antibody has LCDR1 comprising the amino acid sequence as set forth in SEQ ID NO: 100, LCDR2 comprising the amino acid sequence as set forth in SEQ ID NO: 101, and LCDR3 comprising the amino acid sequence as set forth in SEQ ID NO: 102.

Further, the heavy chain variable region of the 439 antibody consists of the amino acid sequence as set forth in SEQ ID NO: 103. The light chain variable region of the 439 antibody consists of the amino acid sequence as set forth in SEQ ID NO: 104.

### Other Antibodies

The antibody of the present invention also includes genetically recombinant antibodies that have been artificially modified for the purpose of reducing heterogenetic antigenicity to humans, such as a chimeric antibody, a humanized antibody and a human antibody, as well as the above-described monoclonal antibody against CDH6. These antibodies can be produced by known methods.

In some embodiments, an anti-CDH6 antibody or antigen-binding fragment is a human antibody or antigen-binding fragment. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology can be used to produce human antibodies and antibody fragments in vitro, from immunoglobulin variable region gene repertoires from unimmunized donors. Techniques for the generation and use of antibody phage libraries are well-known in the art. Once antibodies are identified, affinity maturation strategies known in the art, including but not limited to, chain shuffling and site-directed mutagenesis, can be employed to generate higher affinity human antibodies. In some embodiments, human antibodies are produced in transgenic mice that contain human immunoglobulin loci. Upon immunization these mice are capable of producing the full repertoire of human antibodies in the absence of endogenous immunoglobulin production.

Example of the chimeric antibody can include antibodies in which a variable region and a constant region are heterologous to each other, such as a chimeric antibody formed by conjugating the variable region of a mouse- or rat-derived antibody to a human-derived constant region (see Proc. Natl. Acad. Sci. U.S.A., 81, 6851-6855, (1984)).

Examples of the chimeric antibody derived from the mouse anti-human CDH6 antibody include an antibody consisting of a light chain comprising the light chain variable region of each mouse anti-human CDH6 antibody described in the present description (e.g., the 707 antibody or the 463 antibody) and a human-derived constant region, and a heavy chain comprising the heavy chain variable region thereof and a human-derived constant region.

Other examples of the chimeric antibody derived from the mouse anti-human CDH6 antibody include an antibody consisting of a light chain comprising a light chain variable region having a substitution of one to several residues, 1 to 3 residues, 1 or 2 residues, preferably 1 residue, of amino acids in the light chain variable region of each mouse anti-human CDH6 antibody described in the present description (e.g., the 707 antibody or the 463 antibody) with other amino acid residues, and a heavy chain comprising a heavy chain variable region having a substitution of one to several residues, 1 to 3 residues, 1 or 2 residues, preferably 1 residue, of amino acids in the heavy chain variable region thereof with other amino acid residues. This antibody may have any given human-derived constant region.

Other examples of the chimeric antibody derived from the mouse anti-human CDH6 antibody include an antibody consisting of a light chain comprising a light chain variable region having a substitution of 1 or 2 residues, preferably 1 residue, of amino acids in any 1 to 3 CDRs in the light chain variable region of each mouse anti-human CDH6 antibody described in the present description (e.g., the 707 antibody or the 463 antibody) with other amino acid residues, and a heavy chain comprising a heavy chain variable region having a substitution of 1 or 2 residues, preferably 1 residue, of amino acids in any 1 to 3 CDRs in the heavy chain variable region thereof with other amino acid residues. This antibody may have any given human-derived constant region.

It is known in the art that the constant region(s) of an antibody mediates several effector functions and these effector functions can vary depending on the isotype of the antibody. In some embodiments, anti-CDH6 antibody or antigen-binding fragment described herein comprise at least one constant region of a human IgA antibody. In some embodiments, anti-CDH6 antibody or antigen-binding fragment described herein comprise at least one constant region of a human IgD antibody. In some embodiments, anti-CDH6 antibody or antigen-binding fragment described herein comprise at least one constant region of a human IgE antibody. In some embodiments, anti-CDH6 antibody or antigen-binding fragment described herein comprise at least one constant region of a human IgG antibody. In some embodiments, anti-CDH6 antibody or antigen-binding fragment described herein comprise at least one constant region of a human IgM antibody. In some embodiments, anti-CDH6 antibody or antigen-binding fragment described herein comprise at least one constant region of a human IgG1 antibody. In some embodiments, anti-CDH6 antibody or antigen-binding fragment described herein comprise at least one constant region of a human IgG2 antibody. In some embodiments, anti-CDH6 antibody or antigen-binding fragment described herein comprise at least one constant region of a human IgG3 antibody. In some embodiments, anti-CDH6 antibody or antigen-binding fragment described herein comprise at least one constant region of a human IgG4 antibody. In some embodiments, the Fc region is fused via a hinge. The hinge can be an IgG1 hinge, an IgG2 hinge, or an IgG3 hinge. The amino acid sequences of the Fc region of human IgG1, IgG2, IgG3, and IgG4 are known to those of ordinary skill in the art. In some cases, Fc regions with amino acid variations have been identified in native antibodies.

Examples of the chimeric antibody derived from the 707 antibody include an antibody consisting of a heavy chain comprising a heavy chain variable region comprising the amino acid sequence as set forth in SEQ ID NO: 18, and a light chain comprising a light chain variable region comprising the amino acid sequence as set forth in SEQ ID NO: 23. This antibody may have any given human-derived constant region. Optionally, the heavy chain constant region can be human IgG1 constant region and the light chain constant region can be human Igκ constant region.

In one embodiment, the heavy chain can comprise the amino acid sequence as set forth in SEQ ID NO: 72, and the light chain can comprise the amino acid sequence as set forth in SEQ ID NO: 73, and the chimeric antibody is named Ch069707.

Examples of the chimeric antibody derived from the 463 antibody include an antibody consisting of a heavy chain comprising a heavy chain variable region comprising the amino acid sequence as set forth in SEQ ID NO: 41, and a light chain comprising a light chain variable region comprising the amino acid sequence as set forth in SEQ ID NO: 46. This antibody may have any given human-derived constant region. Optionally, the heavy chain constant region can be human IgG1 constant region (SEQ ID NO: 70) and the light chain constant region can be human Igκ constant region (SEQ ID NO: 71).

In one embodiment, the heavy chain can comprise the amino acid sequence as set forth in SEQ ID NO: 80, and the light chain can comprise the amino acid sequence as set forth in SEQ ID NO: 81, and the chimeric antibody is named Ch069463.

Examples of the humanized antibody can include an antibody formed by incorporating only complementarity determining regions (CDRs) into a human-derived antibody (see Nature (1986) 321, p. 522-525), an antibody formed by incorporating the amino acid residues from some frameworks, as well as CDR sequences, into a human antibody according to a CDR grafting method (International Publication No. WO90/07861), and an antibody formed by modifying the amino acid sequences of some CDRs while maintaining antigen-binding ability.

In the present description, the humanized antibody derived from the 707 antibody, the Ch069707 antibody, the 463 antibody or the Ch069463 antibody is not limited to a specific humanized antibody as long as the humanized antibody retains all 6 CDR sequences unique to the 707 antibody, the Ch069707 antibody, the 463 antibody or the Ch069463 antibody and has internalization activity. The amino acid sequences of some CDRs of this humanized antibody may be further modified as long as it has internalization activity.

Concrete examples of the humanized antibody of the Ch069707 antibody can include any given combination of: a heavy chain comprising a heavy chain variable region consisting of any one amino acid sequence selected from the group consisting of (1) the amino acid sequence as set forth in SEQ ID NO: 66, (2) an amino acid sequence having an identity of at least 95% or more (preferably an amino acid sequence having a sequence identity of at least 95% or more to the sequence of a framework region other than at each CDR sequence) to the above-described amino acid sequence (1), and (3) an amino acid sequence comprising a deletion, substitution or addition of one or several amino acids in the above-described amino acid sequence (1); and a light chain comprising a light chain variable region consisting of any one amino acid sequence selected from the group consisting of (4) the amino acid sequence as set forth in SEQ ID NO: 68, (5) an amino acid sequence having an identity of at least 95% or more (preferably an amino acid sequence having a sequence identity of at least 95% or more to the sequence of a framework region other than at each CDR sequence) to the above-described amino acid sequence (4), and (6) an amino acid sequence comprising a deletion, substitution or addition of one or several amino acids in the above-described amino acid sequence (4). Optionally, the heavy chain constant region can be human IgG1 constant region (SEQ ID NO: 70) and the light chain constant region can be human Igκ constant region (SEQ ID NO: 71), and certain humanized antibodies are named CL069707-H1L1, CL069707-H1L2, CL069707-H2L1 and CL069707-H2L2, respectively, as further described below.

In some embodiments, the heavy chain can comprise the amino acid sequence as set forth in SEQ ID NO: 85 or an amino acid sequence having an identity of at least 95% or more to the SEQ ID NO: 85, and the light chain can comprise the amino acid sequence as set forth in SEQ ID NO: 86 or an amino acid sequence having an identity of at least 95% or more to the SEQ ID NO: 86.

In some embodiments, the humanized antibody of the Ch069707 antibody can include any given combination of: a heavy chain comprising a heavy chain variable region consisting of any one amino acid sequence selected from the group consisting of the amino acid sequence as set forth in SEQ ID NOs: 27 and 29; and a light chain comprising a light chain variable region consisting of any one amino acid sequence selected from the group consisting of the amino acid sequence as set forth in SEQ ID NOs: 34 and 36.

In one embodiment, the heavy chain can comprise the amino acid sequence as set forth in SEQ ID NO: 74, and the light chain can comprise the amino acid sequence as set forth in SEQ ID NO: 76, and the humanized antibody is named CL069707-H1L1.

In one embodiment, the heavy chain can comprise the amino acid sequence as set forth in SEQ ID NO: 74, and the light chain can comprise the amino acid sequence as set forth in SEQ ID NO: 77, and the humanized antibody is named CL069707-H1L2.

In one embodiment, the heavy chain can comprise the amino acid sequence as set forth in SEQ ID NO: 75, and the light chain can comprise the amino acid sequence as set forth in SEQ ID NO: 76, and the humanized antibody is named CL069707-H2L1.

In one embodiment, the heavy chain can comprise the amino acid sequence as set forth in SEQ ID NO: 75, and the light chain can comprise the amino acid sequence as set forth in SEQ ID NO: 77, and the humanized antibody is named CL069707-H2L2.

Concrete examples of the humanized antibody of the Ch069463 antibody can include any given combination of: a heavy chain comprising a heavy chain variable region consisting of any one amino acid sequence selected from the group consisting of (1) the amino acid sequence as set forth in SEQ ID NO: 67, (2) an amino acid sequence having an identity of at least 95% or more (preferably an amino acid sequence having a sequence identity of at least 95% or more to the sequence of a framework region other than at each CDR sequence) to the above-described amino acid sequence (1), and (3) an amino acid sequence comprising a deletion, substitution or addition of one or several amino acids in the above-described amino acid sequence (1); and a light chain comprising a light chain variable region consisting of any one amino acid sequence selected from the group consisting of (4) the amino acid sequence as set forth in SEQ ID NO: 69, (5) an amino acid sequence having an identity of at least 95% or more (preferably an amino acid sequence having a sequence identity of at least 95% or more to the sequence of a framework region other than at each CDR sequence) to the above-described amino acid sequence (4), and (6) an amino acid sequence comprising a deletion, substitution or addition of one or several amino acids in the above-described amino acid sequence (4). Optionally, the heavy chain constant region can be human IgG1 constant region (SEQ ID NO: 70) and the light chain constant region can be human Igκ constant region (SEQ ID NO: 71), and the humanized antibodies are named CL069463-H1L1, CL069463-H1L2, CL069463-H1L3, CL069463-H2L1, CL069463-H2L2, and CL069463-H2L3.

In some embodiments, the humanized antibody of the Ch069463 antibody can include any given combination of: a heavy chain comprising a heavy chain variable region consisting of any one amino acid sequence selected from the group consisting of the amino acid sequence as set forth in SEQ ID NOs: 51 and 54; and a light chain comprising a light chain variable region consisting of any one amino acid sequence selected from the group consisting of the amino acid sequence as set forth in SEQ ID NOs: 58, 63 and 65.

In some embodiments, the heavy chain can comprise the amino acid sequence as set forth in SEQ ID NO: 87 or an amino acid sequence having an identity of at least 95% or more to the SEQ ID NO: 87, and the light chain can comprise the amino acid sequence as set forth in SEQ ID NO: 88 or an amino acid sequence having an identity of at least 95% or more to the SEQ ID NO: 88.

In one embodiment, the heavy chain can comprise the amino acid sequence as set forth in SEQ ID NO: 78, and the light chain can comprise the amino acid sequence as set forth in SEQ ID NO: 82, and the humanized antibody is named CL069463-H1L1.

In one embodiment, the heavy chain can comprise the amino acid sequence as set forth in SEQ ID NO: 78, and the light chain can comprise the amino acid sequence as set forth in SEQ ID NO: 83, and the humanized antibody is named CL069463-H1L2.

In one embodiment, the heavy chain can comprise the amino acid sequence as set forth in SEQ ID NO: 78, and the light chain can comprise the amino acid sequence as set forth in SEQ ID NO: 84, and the humanized antibody is named CL069463-H1L3.

In one embodiment, the heavy chain can comprise the amino acid sequence as set forth in SEQ ID NO: 79, and the light chain can comprise the amino acid sequence as set forth in SEQ ID NO: 82, and the humanized antibody is named CL069463-H2L1.

In one embodiment, the heavy chain can comprise the amino acid sequence as set forth in SEQ ID NO: 79, and the light chain can comprise the amino acid sequence as set forth in SEQ ID NO: 83, and the humanized antibody is named CL069463-H2L2.

In one embodiment, the heavy chain can comprise the amino acid sequence as set forth in SEQ ID NO: 79, and the light chain can comprise the amino acid sequence as set forth in SEQ ID NO: 84, and the humanized antibody is named CL069463-H-2L3.

TABLE 1
The sequence of the mouse antibody, chimeric antibody, and humanized antibody

| ID | NOTE | SEQ |
|---|---|---|
| 1 | CL069707 HCDR1; CL069463 | GFTF[NT]TYA |
| | HCDR1; Ch069707 HCDR1; | |
| | Ch069463 HCDR1; | |
| 2 | CL069707 HCDR1; Ch069707 | GFTFTTYA |
| | HCDR1; | |
| 3 | CL069707 HCDR2; Ch069707 | IRIKNNNYAT |
| | HCDR2; | |
| 4 | CL069707 HCDR3; Ch069707 | VRPDYGNYGLAY |
| | HCDR3; | |
| 5 | CL069707 LCDR1; Ch069707 | SSISSHY |
| | LCDR1; | |
| 6 | CL069707 LCDR2; Ch069707 | RSF |
| | LCDR2; | |
| 7 | CL069707 LCDR3; Ch069707 | QQDIVPLT |
| | LCDR3; | |
| 8 | CL069463 HCDR1; Ch069463 | GFTFNTYA |
| | HCDR1; | |
| 9 | CL069463 HCDR2; Ch069463 | IKSKNKDYET |
| | HCDR2; | |
| 10 | CL069463 HCDR3; Ch069463 | VRQNWNYFDY |
| | HCDR3; | |
| 11 | CL069463 LCDR1; Ch069463 | ENIYYS |
| | LCDR1; | |
| 12 | CL069463 LCDR2; Ch069463 | NAN |
| | LCDR2; | |
| 13 | CL069463 LCDR3; Ch069463 | QQAYDVPPT |
| | LCDR3; | |
| 14 | CL069707 HFR1; Ch069707 | EVHLVESGGGLVQPKGSLKLSCVAS |
| | HFR1; | |
| 15 | CL069707 HFR2; Ch069707 | MNWVRQAPGKGLEWVAR |
| | HFR2; CL069707-H1 HFR2 | |
| 16 | CL069707 HFR3; Ch069707 | YYADSVTDRFTISRDDSQSMLYLQMNNLKTEDTAMYYC |
| | HFR3 | |
| 17 | CL069707 HFR4; Ch069707 | WGQGTLVTVSA |
| | HFR4 | |
| 18 | CL069707 VH; Ch069707 VH | EVHLVESGGGLVQPKGSLKLSCVASGFTFTTYAMNWVRQAPGKGLEWV |
| | | ARIRIKNNNYATYYADSVTDRFTISRDDSQSMLYLQMNNLKTEDTAMYY |
| | | CVRPDYGNYGLAYWGQGTLVTVSA |
| 19 | CL069707 LFR1; Ch069707 | EIVLTQSPTTMAASPGEKITITCSAS |
| | LFR1 | |
| 20 | CL069707 LFR2; Ch069707 | LHWYQQKPGFSPKLLIY |
| | LFR2 | |
| 21 | CL069707 LFR3; Ch069707 | YLASGVPLRFTGSGSGTSYSLTIGTMEAEDVATYYC |
| | LFR3 | |
| 22 | CL069707 LFR4; Ch069707 | FGAGTKLELK |
| | LFR4 | |
| 23 | CL069707 VL; Ch069707 VL | EIVLTQSPTTMAASPGEKITITCSASSSISSHYLHWYQQKPGFSPKLLIYRSF |
| | | YLASGVPLRFTGSGSGTSYSLTIGTMEAEDVATYYCQQDIVPLTFGAGTKL |
| | | ELK |
| 24 | CL069707-H1 HFR1; CL069707- | EVQLVESGGGLVQPGGSLRLSCAAS |
| | H2 HFR1 | |
| 25 | CL069707-H1 HFR3; CL069707- | YYADSVTDRFTISRDDSKNSLYLQMNSLKTEDTAVYYC |
| | H2 HFR3 | |
| 26 | CL069707-H1 HFR4; CL069707- | WGQGTLVTVSS |
| | H2 HFR4; CL069463-H2 HFR4 | |
| 27 | CL069707-H1 VH | EVQLVESGGGLVQPGGSLRLSCAASGFTFTTYAMNWVRQAPGKGLEWV |
| | | ARIRIKNNNYATYYADSVTDRFTISRDDSKNSLYLQMNSLKTEDTAVYYC |
| | | VRPDYGNYGLAYWGQGTLVTVSS |
| 28 | CL069707-H2 HFR2 | MNWVRQAPGKGLEWVGR |
| 29 | CL069707-H2 VH | EVQLVESGGGLVQPGGSLRLSCAASGFTFTTYAMNWVRQAPGKGLEWV |
| | | GRIRIKNNNYATYYADSVTDRFTISRDDSKNSLYLQMNSLKTEDTAVYYC |
| | | VRPDYGNYGLAYWGQGTLVTVSS |
| 30 | CL069707-L1 LFR1; CL069707- | DIQLTQSPSSLSASVGDRVTITCSAS |
| | L2 LFR1 | |
| 31 | CL069707-L1 LFR2; CL069707- | LHWYQQKPGKSPKLLIY |
| | L2 LFR2 | |
| 32 | CL069707-L1 LFR3 | YLASGVPSRFSGSGSGTDFTLTISSLQPEDFATYYC |
| 33 | CL069707-L1 LFR4; CL069707- | FGQGTKLEIK |
| | L2 LFR4 | |
| 34 | CL069707-L1 VL | DIQLTQSPSSLSASVGDRVTITCSASSSISSHYLHWYQQKPGKSPKLLIYRS |
| | | FYLASGVPSRFSGSGSGTDFTLTISSLQPEDFATYYCQQDIVPLTFGQGTKL |
| | | EIK |
| 35 | CL069707-L2 LFR3 | YLASGVPLRFSGSGSGTDFTLTISSLQPEDFATYYC |
| 36 | CL069707-L2 VL | DIQLTQSPSSLSASVGDRVTITCSASSSISSHYLHWYQQKPGKSPKLLIYRS |
| | | FYLASGVPLRFSGSGSGTDFTLTISSLQPEDFATYYCQQDIVPLTFGQGTKL |
| | | EIK |
| 37 | CL069463 HFR1; Ch069463 | EVQLVESGGGLVQPKGSLKLSCAAS |
| | HFR1; | |
| 38 | CL069463 HFR2; Ch069463 | LTWVRQAPGKGLEWVGR |
| | HFR2; CL069463-H2 HFR2 | |
| 39 | CL069463 HFR3; Ch069463 | YYADSVKDRFTISRNDSQSLLYLQMNNLKTEDTAIYYC |
| | HFR3 | |
| 40 | CL069463 HFR4; Ch069463 | WGQGTTLTVSS |
| | HFR4 | |
| 41 | CL069463 VH; Ch069463 VH | EVQLVESGGGLVQPKGSLKLSCAASGFTFNTYALTWVRQAPGKGLEWV |
| | | GRIKSKNKDYETYYADSVKDRFTISRNDSQSLLYLQMNNLKTEDTAIYYC |
| | | VRQNWNYFDYWGQGTTLTVSS |
| 42 | CL069463 LFR1; Ch069463 | DIQMTQSPASLAASVGETVTITCRAS |
| | LFR1 | |
| 43 | CL069463 LFR2; Ch069463 | LAWYQQRQGKSPQLLIY |
| | LFR2 | |
| 44 | CL069463 LFR3; Ch069463 | SLKDGVPSRFSGSGSGTQYSMKINSMQPEDTATYFC |
| | LFR3 | |
| 45 | CL069463 LFR4; Ch069463 | FGGGTKLEIK |
| | LFR4 CL069463-L1 LFR4 | |
| 46 | CL069463 VL; Ch069463 VL | DIQMTQSPASLAASVGETVTITCRASENIYYSLAWYQQRQGKSPQLLIYN |
| | | ANSLKDGVPSRFSGSGSGTQYSMKINSMQPEDTATYFCQQAYDVPPTFG |
| | | GGTKLEIK |
| 47 | CL069463-H1 HFR1 | EVQLVESGGGLVQPSGSLKLSCAAS |
| 48 | CL069463-H1 HFR2 | LTWVRQASGKGLEWVGR |
| 49 | CL069463-H1 HFR3 | YYADSVKDRFTISRDDSISTLYLQMNSLKTEDTALYYC |
| 50 | CL069463-H1 HFR4 | WGQGTMLTVSS |
| 51 | CL069463-H1 VH | EVQLVESGGGLVQPSGSLKLSCAASGFTFNTYALTWVRQASGKGLEWVG |
| | | RIKSKNKDYETYYADSVKDRFTISRDDSISTLYLQMNSLKTEDTALYYCV |
| | | RQNWNYFDYWGQGTMLTVSS |
| 52 | CL069463-H2 HFR1 | EVQLVESGGGLVQPGGSLKLSCAAS |
| 53 | CL069463-H2 HFR3 | YYADSVKDRFTISRDDSKNTLYLQMNSLKTEDTAVYYC |
| 54 | CL069463-H2 VH | EVQLVESGGGLVQPGGSLKLSCAASGFTFNTYALTWVRQAPGKGLEWV |
| | | GRIKSKNKDYETYYADSVKDRFTISRDDSKNTLYLQMNSLKTEDTAVYY |
| | | CVRQNWNYFDYWGQGTLVTVSS |
| 55 | CL069463-L1 LFR1 | DIQMTQSPSSLSASVGQRVTITCRAS |
| 56 | CL069463-L1 LFR2 | LAWYQQKPGKPPRLLIY |
| 57 | CL069463-L1 LFR3 | SLKDGVPSRFSGSGSGTQYTMTITSMQPEDFATYYC |
| 58 | CL069463-L1 VL | DIQMTQSPSSLSASVGQRVTITCRASENIYYSLAWYQQKPGKPPRLLIYNA |
| | | NSLKDGVPSRFSGSGSGTQYTMTITSMQPEDFATYYCQQAYDVPPTFGGG |
| | | TKLEIK |
| 59 | CL069463-L2 LFR1 | DIQMTQSPSSLSASVGDRVTITCRAS |
| 60 | CL069463-L2 LFR2; CL069463- | LAWYQQKPGKAPKLLIY |
| | L3 LFR2 | |
| 61 | CL069463-L2 LFR3; CL069463- | SLKDGVPSRFSGSGSGTDYTMTISSMQPEDFATYYC |
| | L3 LFR3 | |
| 62 | CL069463-L2 LFR4; CL069463- | FGGGTKVEIK |
| | L3 LFR4 | |
| 63 | CL069463-L2 VL | DIQMTQSPSSLSASVGDRVTITCRASENIYYSLAWYQQKPGKAPKLLIYN |
| | | ANSLKDGVPSRFSGSGSGTDYTMTISSMQPEDFATYYCQQAYDVPPTFGG |
| | | GTKVEIK |
| 64 | CL069463-L3 LFR1 | DIQMTQSPSSLSASVGERVTITCRAS |
| 65 | CL069463-L3 VL | DIQMTQSPSSLSASVGERVTITCRASENIYYSLAWYQQKPGKAPKLLIYN |
| | | ANSLKDGVPSRFSGSGSGTDYTMTISSMQPEDFATYYCQQAYDVPPTFGG |
| | | GTKVEIK |
| 66 | CL069707-H1 VH; CL069707-H2 | EVQLVESGGGLVQPGGSLRLSCAASGFTFTTYAMNWVRQAPGKGLEWV[ |
| | VH | AG]RIRIKNNNYATYYADSVTDRFTISRDDSKNSLYLQMNSLKTEDTAVYY |
| | | CVRPDYGNYGLAYWGQGTLVTVSS |
| 67 | CL069463-H1 VH; CL069463-H2 | EVQLVESGGGLVQP[GS]GSLKLSCAASGFTFNTYALTWVRQA[PS]GKGL |
| | VH | EWVGRIKSKNKDYETYYADSVKDRFTISRDDS[IK][NS]TLYLQMNSLKTE |
| | | DTA[LV]YYCVRQNWNYFDYWGQGT[LM][LV]TVSS |
| 68 | CL069707-L1 VL; CL069707-L2 | DIQLTQSPSSLSASVGDRVTITCSASSSISSHYLHWYQQKPGKSPKLLIYRS |
| | VL | FYLASGVP[LS]RFSGSGSGTDFTLTISSLQPEDFATYYCQQDIVPLTFGQGT |
| | | KLEIK |
| 69 | CL069463-L1 VL; CL069463-L2 | DIQMTQSPSSLSASVG[DEQ]RVTITCRASENIYYSLAWYQQKPGK[AP]P[K |
| | VL; CL069463-L3 VL | RJLLIYNANSLKDGVPSRFSGSGSGT[DQ]YTMTI[ST]SMQPEDFATYYCQ |
| | | QAYDVPPTFGGGTK[LV]EIK |
| 70 | heavy chain constant region | ASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNG |
| | | KEYKCKVSNKALPAPIEKTISKAKGQPREPQVYTLPPSRDELTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPGK |
| 71 | light chain constant region | RTVAAPSVFIFPPSDEQLKSGTASVVCLLNNFYPREAKVQWKVDNALQSG |
| | | NSQESVTEQDSKDSTYSLSSTLTLSKADYEKHKVYACEVTHQGLSSPVTK |
| | | SFNRGEC |
| 72 | Ch069707 HC | EVHLVESGGGLVQPKGSLKLSCVASGFTFTTYAMNWVRQAPGKGLEWV |
| | | ARIRIKNNNYATYYADSVTDRFTISRDDSQSMLYLQMNNLKTEDTAMYY |
| | | CVRPDYGNYGLAYWGQGTLVTVSAASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAK |
| | | GQPREPQVYTLPPSRDELTKNQVSLTCLVKGFYPSDIAVEWESNGQPENN |
| | | YKTTPPVLDSDGSFFLYSKLTVDKSRWQQGNVFSCSVMHEALHNHYTQK |
| | | SLSLSPGK |
| 73 | Ch069707 LC | EIVLTQSPTTMAASPGEKITITCSASSSISSHYLHWYQQKPGFSPKLLIYRSF |
| | | YLASGVPLRFTGSGSGTSYSLTIGTMEAEDVATYYCQQDIVPLTFGAGTKL |
| | | ELKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSP |
| | | VTKSFNRGEC |
| 74 | CL069707-H1 | EVQLVESGGGLVQPGGSLRLSCAASGFTFTTYAMNWVRQAPGKGLEWV |
| | | ARIRIKNNNYATYYADSVTDRFTISRDDSKNSLYLQMNSLKTEDTAVYYC |
| | | VRPDYGNYGLAYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLFP |
| | | PKPKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKAKG |
| | | QPREPQVYTLPPSRDELTKNQVSLTCLVKGFYPSDIAVEWESNGQPENNY |
| | | KTTPPVLDSDGSFFLYSKLTVDKSRWQQGNVFSCSVMHEALHNHYTQKS |
| | | LSLSPGK |
| 75 | CL069707-H2 | EVQLVESGGGLVQPGGSLRLSCAASGFTFTTYAMNWVRQAPGKGLEWV |
| | | GRIRIKNNNYATYYADSVTDRFTISRDDSKNSLYLQMNSLKTEDTAVYYC |
| | | VRPDYGNYGLAYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLFP |
| | | PKPKDTLMISRTPEVTCVVVDVSHEDPEVKFNWYVDGVEVHNAKTKPR |
| | | EEQYNSTYRVVSVLTVLHQDWLNGKEYKCKVSNKALPAPIEKTISKAKG |
| | | QPREPQVYTLPPSRDELTKNQVSLTCLVKGFYPSDIAVEWESNGQPENNY |
| | | KTTPPVLDSDGSFFLYSKLTVDKSRWQQGNVFSCSVMHEALHNHYTQKS |
| | | LSLSPGK |
| 76 | CL069707-L1 | DIQLTQSPSSLSASVGDRVTITCSASSSISSHYLHWYQQKPGKSPKLLIYRS |
| | | FYLASGVPSRFSGSGSGTDFTLTISSLQPEDFATYYCQQDIVPLTFGQGTKL |
| | | EIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSP |
| | | VTKSFNRGEC |
| 77 | CL069707-L2 | DIQLTQSPSSLSASVGDRVTITCSASSSISSHYLHWYQQKPGKSPKLLIYRS |
| | | FYLASGVPLRFSGSGSGTDFTLTISSLQPEDFATYYCQQDIVPLTFGQGTKL |
| | | EIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSP |
| | | VTKSFNRGEC |
| 78 | CL069463-H1 | EVQLVESGGGLVQPSGSLKLSCAASGFTFNTYALTWVRQASGKGLEWVG |
| | | RIKSKNKDYETYYADSVKDRFTISRDDSISTLYLQMNSLKTEDTALYYCV |
| | | RQNWNYFDYWGQGTMLTVSSASTKGPSVFPLAPSSKSTSGGTAALGCLV |
| | | KDYFPEPVTVSWNSGALTSGVHTFPAVLQSSGLYSLSSVVTVPSSSLGTQT |
| | | YICNVNHKPSNTKVDKKVEPKSCDKTHTCPPCPAPELLGGPSVFLFPPKP |
| | | KDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPR |
| | | EPQVYTLPPSRDELTKNQVSLTCLVKGFYPSDIAVEWESNGQPENNYKTT |
| | | PPVLDSDGSFFLYSKLTVDKSRWQQGNVFSCSVMHEALHNHYTQKSLSL |
| | | SPGK |
| 79 | CL069463-H2 | EVQLVESGGGLVQPGGSLKLSCAASGFTFNTYALTWVRQAPGKGLEWV |
| | | GRIKSKNKDYETYYADSVKDRFTISRDDSKNTLYLQMNSLKTEDTAVYY |
| | | CVRQNWNYFDYWGQGTLVTVSSASTKGPSVFPLAPSSKSTSGGTAALGC |
| | | LVKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPRE |
| | | EQYNSTYRVVSVLTVLHQDWLNGKEYKCKVSNKALPAPIEKTISKAKGQ |
| | | PREPQVYTLPPSRDELTKNQVSLTCLVKGFYPSDIAVEWESNGQPENNYK |
| | | TTPPVLDSDGSFFLYSKLTVDKSRWQQGNVFSCSVMHEALHNHYTQKSL |
| | | SLSPGK |
| 80 | Ch069463 HC | EVQLVESGGGLVQPKGSLKLSCAASGFTFNTYALTWVRQAPGKGLEWV |
| | | GRIKSKNKDYETYYADSVKDRFTISRNDSQSLLYLQMNNLKTEDTAIYYC |
| | | VRQNWNYFDYWGQGTTLTVSSASTKGPSVFPLAPSSKSTSGGTAALGCL |
| | | VKDYFPEPVTVSWNSGALTSGVHTFPAVLQSSGLYSLSSVVTVPSSSLGTQ |
| | | TYICNVNHKPSNTKVDKKVEPKSCDKTHTCPPCPAPELLGGPSVFLFPPKP |
| | | KDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPR |
| | | EPQVYTLPPSRDELTKNQVSLTCLVKGFYPSDIAVEWESNGQPENNYKTT |
| | | PPVLDSDGSFFLYSKLTVDKSRWQQGNVFSCSVMHEALHNHYTQKSLSL |
| | | SPGK |
| 81 | Ch069463 LC | DIQMTQSPASLAASVGETVTITCRASENIYYSLAWYQQRQGKSPQLLIYN |
| | | ANSLKDGVPSRFSGSGSGTQYSMKINSMQPEDTATYFCQQAYDVPPTFG |
| | | GGTKLEIKRTVAAPSVFIFPPSDEQLKSGTASVVCLLNNFYPREAKVQWK |
| | | VDNALQSGNSQESVTEQDSKDSTYSLSSTLTLSKADYEKHKVYACEVTH |
| | | QGLSSPVTKSFNRGEC |
| 82 | CL069463-L1 | DIQMTQSPSSLSASVGQRVTITCRASENIYYSLAWYQQKPGKPPRLLIYNA |
| | | NSLKDGVPSRFSGSGSGTQYTMTITSMQPEDFATYYCQQAYDVPPTFGGG |
| | | TKLEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQG |
| | | LSSPVTKSFNRGEC |
| 83 | CL069463-L2 | DIQMTQSPSSLSASVGDRVTITCRASENIYYSLAWYQQKPGKAPKLLIYN |
| | | ANSLKDGVPSRFSGSGSGTDYTMTISSMQPEDFATYYCQQAYDVPPTFGG |
| | | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQ |
| | | GLSSPVTKSFNRGEC |
| 84 | CL069463-L3 | DIQMTQSPSSLSASVGERVTITCRASENIYYSLAWYQQKPGKAPKLLIYN |
| | | ANSLKDGVPSRFSGSGSGTDYTMTISSMQPEDFATYYCQQAYDVPPTFGG |
| | | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQ |
| | | GLSSPVTKSFNRGEC |
| 85 | CL069707-H1; CL069707-H2 | EVQLVESGGGLVQPGGSLRLSCAASGFTFTTYAMNWVRQAPGKGLEWV[ |
| | | AG]RIRIKNNNYATYYADSVTDRFTISRDDSKNSLYLQMNSLKTEDTAVYY |
| | | CVRPDYGNYGLAYWGQGTLVTVSSASTKGPSVFPLAPSSKSTSGGTAAL |
| | | GCLVKDYFPEPVTVSWNSGALTSGVHTFPAVLQSSGLYSLSSVVTVPSSSL |
| | | GTQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAK |
| | | GQPREPQVYTLPPSRDELTKNQVSLTCLVKGFYPSDIAVEWESNGQPENN |
| | | YKTTPPVLDSDGSFFLYSKLTVDKSRWQQGNVFSCSVMHEALHNHYTQK |
| | | SLSLSPGK |
| 86 | CL069707-L1; CL069707-L2 | DIQLTQSPSSLSASVGDRVTITCSASSSISSHYLHWYQQKPGKSPKLLIYRS |
| | | FYLASGVP[LS]RFSGSGSGTDFTLTISSLQPEDFATYYCQQDIVPLTFGQGT |
| | | KLEIKRTVAAPSVFIFPPSDEQLKSGTASVVCLLNNFYPREAKVQWKVDN |
| | | ALQSGNSQESVTEQDSKDSTYSLSSTLTLSKADYEKHKVYACEVTHQGLS |
| | | SPVTKSFNRGEC |
| 87 | CL069463-H1; CL069463-H2 | EVQLVESGGGLVQP[GS]GSLKLSCAASGFTFNTYALTWVRQA[PS]GKGL |
| | | EWVGRIKSKNKDYETYYADSVKDRFTISRDDS[IK][NS]TLYLQMNSLKTE |
| | | DTA[LV]YYCVRQNWNYFDYWGQGT[LM][LV]TVSSASTKGPSVFPLAPS |
| | | SKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPA |
| | | PELLGGPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNKALP |
| | | APIEKTISKAKGQPREPQVYTLPPSRDELTKNQVSLTCLVKGFYPSDIAVE |
| | | WESNGQPENNYKTTPPVLDSDGSFFLYSKLTVDKSRWQQGNVFSCSVMH |
| | | EALHNHYTQKSLSLSPGK |
| 88 | CL069463-L3; CL069463-L2; | DIQMTQSPSSLSASVG[DEQ]RVTITCRASENIYYSLAWYQQKPGK[AP]P[K |
| | CL069463-L1 | RJLLIYNANSLKDGVPSRFSGSGSGT[DQ]YTMTI[ST]SMQPEDFATYYCQ |
| | | QAYDVPPTFGGGTK[LV]EIKRTVAAPSVFIFPPSDEQLKSGTASVVCLLNN |
| | | FYPREAKVQWKVDNALQSGNSQESVTEQDSKDSTYSLSSTLTLSKADYE |
| | | KHKVYACEVTHQGLSSPVTKSFNRGEC |
| 89 | CL069066 HCDR1; Ch069066 | GFDFSRYW |
| | HCDR1 | |
| 90 | CL069066 HCDR2; Ch069066 | INPDSSTI |
| | HCDR2 | |
| 91 | CL069066 HCDR3; Ch069066 | TRRPHYYGYFDS |
| | HCDR3 | |
| 92 | CL069066 LCDR1; Ch069066 | ESVEYYGTTL |
| | LCDR1 | |
| 93 | CL069066 LCDR2; Ch069066 | AGS |
| | LCDR2 | |
| 94 | CL069066 LCDR3; Ch069066 | QQSRKVPWT |
| | LCDR3 | |
| 95 | CL069066 VH; Ch069066 VH | EVKLLESGGGLVQPGGSLKLSCAASGFDFSRYWMTWVRQAPGKGLEWI |
| | | GEINPDSSTINYTPSLKDKFIISRDNAKNTLYLQMNKVTSEDTALYYCTRR |
| | | PHYYGYFDSWGQGTTLTVSS |
| 96 | CL069066 VL; Ch069066 VL | DIVLTQSPASLAVSLGQRATISCRASESVEYYGTTLMQWYQQKPGQPPQL |
| | | LIYAGSNVESGVPARFSGSGSGTEFSLNIHPVEEDDIGMYFCQQSRKVPWT |
| | | FGGGTKLEIK |
| 97 | CL069439 HCDR1; Ch069439 | GYSFTGYY |
| | HCDR1 | |
| 98 | CL069439 HCDR2; Ch069439 | ISCFNGDT |
| | HCDR2 | |
| 99 | CL069439 HCDR3; Ch069439 | VRGRYGNFNYFDY |
| | HCDR3 | |
| 100 | CL069439 LCDR1; Ch069439 | ESVEFYGTSL |
| | LCDR1 | |
| 101 | CL069439 LCDR2; Ch069439 | ATS |
| | LCDR2 | |
| 102 | CL069439 LCDR3; Ch069439 | QQSRRIPWT |
| | LCDR3 | |
| 103 | CL069439 VH; Ch069439 VH | EVQLQQSGTELVRTGASVKISCKASGYSFTGYYIHWIKQSHGESLEWIGYI |
| | | SCFNGDTSYNQNFKDRATFNVDTSSSTAYMQFISLTSEDSAVYYCVRGRY |
| | | GNFNYFDYWGQGTTLTVSS |
| 104 | CL069439 VL; Ch069439 VL | DIVLTQSPASLAVSLGQRATISCRASESVEFYGTSLMQWFQQKPGHPPQLL |
| | | IYATSNVDSGVPARFSASGSGTDFSLNIHPVEEADIAMYFCQQSRRIPWTF |
| | | GGGTKLEIK |
| 105 | H01L02 VH | EVQLVQSGAEVKKPGASVKVSCKASGYTFTRNFMHWVRQAPGQGLEW |
| | | MGWIYPGDGETEYAQKFQGRVTITADTSTSTAYMELSSLRSEDTAVYYCA |
| | | RGVYGGFAGGYFDFWGQGTLVTVSS |
| 106 | H01L02 VL | DIQMTQSPSSLSASVGDRVTITCKASQNIYKNLAWYQQKPGKAPKLLIYD |
| | | ANTLQTGVPSRFSGSGSGSDFTLTISSLQPEDFATYFCQQYYSGWAFGQGT |
| | | KVEIK |

The amino acid substitution in the present description is preferably a conservative amino acid substitution. The conservative amino acid substitution is a substitution occurring within an amino acid group associated with certain amino acid side chains. Preferred amino acid groups are the following: acidic group=aspartic acid and glutamic acid; basic group=lysine, arginine, and histidine; non-polar group=alanine, valine, leucine, isoleucine, proline, phenylalanine, methionine, and tryptophan; and uncharged polar family=glycine, asparagine, glutamine, cysteine, serine, threonine, and tyrosine. Other preferred amino acid groups are the following: aliphatic hydroxy group=serine and threonine; amide-containing group=asparagine and glutamine; aliphatic group=alanine, valine, leucine and isoleucine; and aromatic group=phenylalanine, tryptophan and tyrosine. Such amino acid substitution is preferably carried out without impairing the properties of a substance having the original amino acid sequence.

By combining together sequences showing a high identity to the above-described heavy chain amino acid sequences and light chain amino acid sequences, it is possible to select an antibody having a biological activity equivalent to that of each of the above-described antibodies. Such an identity is an identity of generally 80% or more, preferably 90% or more, more preferably 95% or more, and most preferably 99% or more. Moreover, also by combining amino acid sequences of a heavy chain and a light chain comprising a substitution, deletion or addition of one or several amino acid residues thereof with respect to the amino acid sequence of a heavy chain or a light chain, it is possible to select an antibody having a biological activity equivalent to that of each of the above-described antibodies.

The anti-CDH6 human antibody can also be obtained by transforming eukaryotic cells with cDNA encoding each of the heavy chain and light chain of such a human antibody or preferably with a vector comprising the cDNA, according to genetic recombination techniques, and then culturing the transformed cells producing a genetically modified human monoclonal antibody, so that the antibody can be obtained from the culture supernatant. In this context, eukaryotic cells, and preferably, mammalian cells such as CHO cells, lymphocytes or myelomas can, for example, be used as a host.

Furthermore, a method of obtaining a phage display-derived human antibody that has been selected from a human antibody library (see Wormstone, I. M. et al., Investigative Ophthalmology & Visual Science. (2002) 43 (7), p. 2301-2308; Carmen, S. et al., Briefings in Functional Genomics and Proteomics (2002), 1 (2), p. 189-203; Siriwardena, D. et al., Ophthalmology (2002) 109 (3), p. 427-431; etc.) is also known.

### Immunoconjugate

The anti-CDH6 antibody of present application can be conjugated to an active moiety, and the active moiety can comprise a drug moiety and/or a label.

In some embodiments, the drug moiety is selected from the group consisting of a cytotoxic agent, a cytokine, a nucleic acid, a nucleic acid-associated molecule, a radionuclide, a chemokine, an immuno(co)-stimulatory molecule, an immunosuppressive molecule, a death ligand, an apoptosis-inducing protein, a kinase, a prodrug-converting enzyme, a RNase, an agonistic antibody or antibody fragment, an antagonistic antibody or antibody fragment, a growth factor, a hormone, a coagulation factor, a fibrinolytic protein, peptides mimicking these, and fragments, fusion proteins and derivatives thereof.

In some embodiments, the label selected from the group consisting of a radiolabel, a fluorophore, a chromophore, an imaging agent, and a metal ion.

In some embodiments, the cytotoxic agent comprises a microtubule disrupting drug and/or a DNA-damaging agent.

In some embodiments, the cytotoxic agent can be selected from: Paclitaxel; cytochalasin B; short bacteriocin D; ethidium bromide; emetine; mitomycin; etoposide; onychothioside; thienoside; vincristine; colchicine; doxorubicin; erythromycin; dihydroxycarbamycin dione; microtubulin inhibitors; mitoxantrone; actinomycin D; 1-dehydrotestosterone; glucocorticoids; procaine; bupivacaine; lidocaine; propranolol; puromycin; kallikrein or its analogs or derivatives; antimetabolites; alkylating agents; antibiotics; antimitotic agents; diphtheria toxin and related molecules and their active fragments and heterodimeric molecules, ricin toxin, cholera toxin, shiga-like toxin, LT toxin, C3 toxin, shiga toxin, pertussis toxin, tetanus toxin, soybean Bowman-Birk protease inhibitor, Pseudomonas exotoxin, alorin, fucoside, capsidin, gelanin, phaseolus toxin chain A, capsidin chain A, alpha-bromotoxin, oleuropein, staphylin protein, American commercial protein, bitter melon inhibitor, jatropha toxin, croton toxin, soapwort inhibitor, white tree toxin, mitogellin, limiting trichothecene, phenomycin and enoxycin Toxins; Ribonuclease (RNase); DNase I, staphylococcal endotoxin A; American commercial land antiviral protein; diphtheria toxin; and Pseudomonas endotoxin.

In some embodiments, the microtubulin inhibitor can be medensin I or an analogue or derivative thereof; the antimetabolite is aminopterin, 6-mercaptopurine, 6-thioguanine, cytarabine, fludarabine, 5-fluorouracil, dacarbazine, hydroxyurea, asparaginase, gemcitabine or cladribine; the alkylating agent is azacitidine, thiophan, azacitidine benzoate, melphalan, carazacitidine (BSNU), lomustine (CCNU), cyclophosphamide, leucovorin, dibromomannan, streptozotocin, dacarbazine (DTIC), procarbazine, mitomycin C, cis-molybdenum, carbomolybdenum, duocarmycin A, duocarmycin SA, rachamycin (CC-1065) or analogues or derivatives thereof; the antibiotics being bleomycin, adriamycin, idarubicin (or mitomycin, mitoxantrone, puccamycin, antrixin (AMC)); the antimitotic agent is monomethyl auristatin E or F; the diphtheria toxin-related molecule is diphtheria A chain; the ricin toxin is ricin A or deglycosylated ricin A chain toxin; the shiga-like toxin is SLT I, SLT II and SLT IIV; the American commercial protein is PAPI, PAPII and PAP S.

In some embodiments, the radionuclide comprises: At²¹¹, I¹³¹, I¹²⁵, I¹²³, Y⁹⁰, Re¹⁸⁶, Re¹⁸⁸, Sm¹⁵³, Bi²¹², P³², Pb²¹², Tc⁹⁹, S³⁵, F¹⁹, N¹⁵, C¹⁴, C¹³ or H³, optionally the radionuclide can be conjugated to the antibody via a chelating agent.

In some embodiments, the cytokine comprises: IL-2, IL-4, IL-6, IL-7, IL-10, IL-12, IL-13, IL-15, IL-18, IL-23, IL-24, IL-27, IL_28a, IL_28b, IL-29, KGF IFNα, IFNβ, IFNγ, GM-CSF, CD40L, Flt3 ligand, stem cell factor, anisidine or TNFα.

### Anti-CDH6 Antibody Drug Conjugate

The anti-CDH6 antibody of present application can be conjugated to a drug via a linker structure moiety to prepare an anti-CDH6 antibody drug conjugate. The drug is not particularly limited as long as it has a substituent or a partial structure that can be connected to a linker structure. The anti-CDH6 antibody drug conjugate can be used for various purposes according to the conjugated drug. Examples of such a drug can include substances having antitumor activity, substances effective for blood diseases, substances effective for autoimmune diseases, anti-inflammatory substances, antimicrobial substances, antifungal substances, antiparasitic substances, antiviral substances, and anti-anesthetic substances.

### Cytotoxic Agent

An example using a cytotoxic agent as a compound to be conjugated in the anti-CDH6 antibody drug conjugate of the present invention will be described below. The cytotoxic agent is not particularly limited as long as the compound has an antitumor effect and has a substituent or a partial structure that can be connected to a linker structure. Upon cleavage of a part or the whole of the linker in tumor cells, the cytotoxic agent is released so that the cytotoxic agent exhibits an antitumor effect. As the linker is cleaved at a connecting position with the drug, the cytotoxic agent is released in its original structure to exert its original antitumor effect.

In some embodiments, the cytotoxic agent comprises a tubulin inhibitor and/or a topoisomerase inhibitor. For example, the cytotoxic agent can comprise: monomethyl auristatin E (MMAE), monomethyl auristatin F (MMAF), medensin I, pyrrolobenzodiazepine (PBD), camptothecin, nemorubicin, PNU-159682, anthracyclines, betacamycin, perillyl alkaloids, paclitaxel, montelukast, elinafide, kallikrein, duocarmycin, rachamycin (CC-1065) or an analogue, a derivative or a prodrug thereof.

In some embodiments, the cytotoxic agent comprises a topoisomerase I inhibitor. For example, the cytotoxic agent can comprise camptothecin (CPT) or a derivative thereof.

As a non-restricted example, the camptothecin can comprise camptothecin and camptothecin derivatives including but not limited to irinotecan, topotecan, lurtotecan, silatecan, etirinotecan pegol, TAS 103, 9-aminocamptothecin, 7-ethylcamptothecin, 10-hydroxycamptothecin, 9-nitrocamptothecin, 10,11-methylenedioxycamptothecin, 9-amino- 10,11-methylenedioxycamptothecin, 9-chloro-10,11-methylenedioxycamptothecin, 7-(4-methylpiperazinomethylene)-10,11-ethylenedioxy-20(S)-camptothecin, 7-(4-methylpiperazinomethylene)-10,11-methylenedioxy-20(S)-camptothecin, and 7-(2-N-isopropylamino)ethyl)-(20S)-camptothecin, and stereoisomers, salts and esters thereof.

As one example of the cytotoxic agent used in the present invention, exatecan, a camptothecin derivative ((1S,9S)-1-amino-9-ethyl-5-fluoro-2,3-dihydro-9-hydroxy-4-methyl-1H,12H-benzo[de]pyrano[3′,4′:6,7]indolizino[1,2-b]quinoline-10,13(9H,15H)-dione represented by the following formula) can preferably be used.

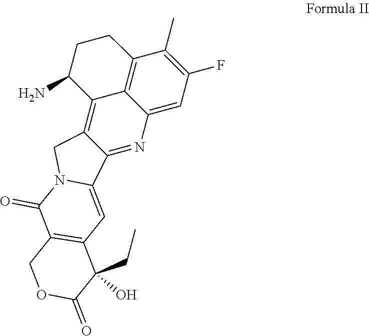 [SMILES: CC[C@@]1(O)C(=O)OCc2c1cc1n(c2=O)Cc2c-1nc1cc(F)c(C)c3c1c2[C@@H](N)CC3]

Exatecan can be easily obtained by, for example, a method described in U.S. Patent Publication No. US2016/0297890 or other known methods, and the amino group at position 1 can be preferably used as a connecting position to a linker structure.

Since exatecan has a camptothecin structure, it is known that the equilibrium shifts to a structure with a formed lactone ring (closed ring) in an acidic aqueous medium (e.g., of the order of pH 3) whereas the equilibrium shifts to a structure with an opened lactone ring (open ring) in a basic aqueous medium (e.g., of the order of pH 10). Formula II as discussed herein includes a tautomer, mesomer, racemate, enantiomer, or diastereomer thereof, or mixtures thereof, or a pharmaceutically acceptable salt or a solvate thereof. A drug conjugate into which exatecan residues corresponding to such a closed ring structure and/or an open ring structure have been introduced is also expected to have an equivalent antitumor effect, and it is needless to say that any of such drug conjugate is included within the scope of the present invention.

Other examples of the cytotoxic agent can include cytotoxic agents described in the literature (Pharmacological Reviews, 68, p. 3-19, 2016). Specific examples thereof can include doxorubicin, calicheamicin, dolastatin 10, auristatins such as monomethyl auristatin E (MMAE) and monomethyl auristatin F (MMAF), maytansinoids such as DM1 and DM4, a pyrrolobenzodiazepine dimer SG2000 (SJG-136), a camptothecin derivative SN-38, duocarmycins such as CC-1065, amanitin, daunorubicin, mitomycin C, bleomycin, cyclocytidine, vincristine, vinblastine, methotrexate, platinum-based antitumor agents (cisplatin and derivatives thereof), and Taxol and derivatives thereof.

In the antibody drug conjugate, the number of conjugated drug molecules per antibody molecule is a key factor having an influence on the efficacy and safety thereof. The production of the antibody drug conjugate is carried out by specifying reaction conditions such as the amounts of starting materials and reagents used for reaction, so as to attain a constant number of conjugated drug molecules. Unlike the chemical reaction of a low-molecular-weight compound, a mixture containing different numbers of conjugated drug molecules is usually obtained. The number of conjugated drug molecules per antibody molecule is defined and indicated as an average value, i.e., the average number of conjugated drug molecules. Unless otherwise specified, i.e., except in the case of representing an antibody drug conjugate having a specific number of conjugated drug molecules that is included in an antibody drug conjugate mixture having different numbers of conjugated drug molecules, the number of conjugated drug molecules according to the present invention also means an average value as a rule. The number of exatecan molecules conjugated to an antibody molecule is controllable, and as an average number of conjugated drug molecules per antibody, approximately 1 to 10 exatecan molecules can be conjugated. The number of exatecan molecules is preferably 2 to 8, 3 to 8, 4 to 8, 5 to 8, 6 to 8 or 7 to 8, more preferably 5 to 8, further preferably 7 to 8, still further preferably 8. It is to be noted that a person skilled in the art can design a reaction for conjugating a required number of drug molecules to an antibody molecule based on the description of Examples of the present application, and can obtain an antibody drug conjugate with a controlled number of conjugated exatecan molecules.

### Linker Structure

The linker structure which conjugates a drug moiety to the anti-CDH6 antibody in the anti-CDH6 antibody-drug conjugate of the present invention will be described. In the antibody-drug conjugate of the present application, the linker structure which conjugates the anti-CDH6 antibody to the drug is not particularly limited as long as the resulting antibody-drug conjugate can be used. The linker structure may be appropriately selected and used according to the purpose of use. One example of the linker structure can include a linker described in known literature (Pharmacol Rev 68: 3-19, January 2016, Protein Cell DOI 10.1007/s13238-016-0323-0, etc.).

Any linker structure given below can preferably be used. It is to be noted that the left terminus of the structure is a connecting position to the antibody, and the right terminus thereof is a connecting position to the drug. Furthermore, VA in the linker structures given below represents an amino acid sequence consisting of valine-alanine (VA) linked through peptide bonds, and GGFG in the linker structures given below represents an amino acid sequence consisting of glycine-glycine-phenylalanine-glycine (GGFG) linked through peptide bonds. -(succinimidyl-3-yl-N)—CH₂CH₂—C(═O)-GGFG-PABC-;
-(succinimidyl-3-yl-N)—CH₂CH₂CH₂CH₂CH₂—C(═O)-GGFG-PABC-;
-(succinimidyl-3-yl-N)—CH₂CH₂CH₂CH₂CH₂—C(═O)-GGFG-NH-PABC-;
-(succinimidyl-3-yl-N)—CH₂CH₂—C(═O)—NH—CH₂CH₂O—CH₂CH₂O—CH₂CH₂—C(═O)-GGFG-PABC-;
-(succinimidyl-3-yl-N)—CH₂CH₂—C(═O)—NH—CH₂CH₂O—CH₂CH₂O—CH₂CH₂O—CH₂CH₂O—CH₂CH₂—C(═O)-GGFG-PABC-;
—CH₂—C(═O)—NH—CH₂CH₂—C(═O)-GGFG-PABC-;
—C(═O)—CH₂CH₂CH₂CH₂CH₂CH₂—C(═O)-GGFG-PABC-;
-(succinimidyl-3-yl-N)—CH₂CH₂—C(═O)-GGFG-NH—CH₂CH₂—C(═O)—;
-(succinimidyl-3-yl-N)—CH₂CH₂—C(═O)-GGFG-NH—CH₂CH₂CH₂—C(═O)—;
-(succinimidyl-3-yl-N)—CH₂CH₂CH₂CH₂CH₂—C(═O)-GGFG-NH—CH₂CH₂—C(═O)—;
-(succinimidyl-3-yl-N)—CH₂CH₂CH₂CH₂CH₂—C(═O)-GGFG-NH—CH₂CH₂CH₂—C(═O)—;
-(succinimidyl-3-yl-N)—CH₂CH₂CH₂CH₂CH₂—C(═O)-GGFG-NH—CH₂CH₂CH₂CH₂CH₂—C(═O)—;
-(succinimidyl-3-yl-N)—CH₂CH₂CH₂CH₂CH₂—C(═O)-GGFG-NH—CH₂—O—CH₂—C(═O)—;
-(succinimidyl-3-yl-N)—CH₂CH₂CH₂CH₂CH₂—C(═O)-GGFG-NH—CH₂CH₂—O—CH₂—C(═O)—;
-(succinimidyl-3-yl-N)—CH₂CH₂—C(═O)—NH—CH₂CH₂O—CH₂CH₂O—CH₂CH₂—C(═O)-GGFG-NH—CH₂CH₂CH₂—C(═O)—;
-(succinimidyl-3-yl-N)—CH₂CH₂—C(═O)—NH—CH₂CH₂O—CH₂CH₂O—CH₂CH₂—C(═O)-GGFG-NH—CH₂CH₂—C(═O)—;
-(succinimidyl-3-yl-N)—CH₂CH₂—C(═O)—NH—CH₂CH₂O—CH₂CH₂O—CH₂CH₂O—CH₂CH₂O—CH₂CH₂—C(═O)-GGFG-NH—CH₂CH₂CH₂—C(═O)—;
-(succinimidyl-3-yl-N)—CH₂CH₂—C(═O)—NH—CH₂CH₂O—CH₂CH₂O—CH₂CH₂O—CH₂CH₂O—CH₂CH₂—C(═O)-GGFG-NH—CH₂CH₂—C(═O)—;
—CH₂—C(═O)—NH—CH₂CH₂—C(═O)-GGFG-NH—CH₂CH₂CH₂—C(═O)—;
—C(═O)—CH₂CH₂CH₂CH₂CH₂CH₂—C(═O)-GGFG-NH—CH₂CH₂CH₂—C(═O)—;
-(succinimidyl-3-yl-N)—CH₂CH₂—C(═O)—VA-PABC-;
-(succinimidyl-3-yl-N)—CH₂CH₂CH₂CH₂CH₂—C(═O)—VA-PABC-;
-(succinimidyl-3-yl-N)—CH₂CH₂CH₂CH₂CH₂—C(═O)—VA-NH-PABC-;
-(succinimidyl-3-yl-N)—CH₂CH₂—C(═O)—NH—CH₂CH₂O—CH₂CH₂O—CH₂CH₂—C(═O)—VA-PABC-;
-(succinimidyl-3-yl-N)—CH₂CH₂—C(═O)—NH—CH₂CH₂o-CH₂CH₂O—CH₂CH₂O—CH₂CH₂O—CH₂CH₂—C(═O)—VA-PABC-;
—CH₂—C(═O)—NH—CH₂CH₂—C(═O)—VA-PABC-;
—C(═O)—CH₂CH₂CH₂CH₂CH₂CH₂—C(═O)—VA-PABC-;
-(succinimidyl-3-yl-N)—CH₂CH₂—C(═O)—VA-NH—CH₂CH₂—C(═O)—;
-(succinimidyl-3-yl-N)—CH₂CH₂—C(═O)—VA-NH—CH₂CH₂CH₂—C(═O)—;
-(succinimidyl-3-yl-N)—CH₂CH₂CH₂CH₂CH₂—C(═O)—VA-NH—CH₂CH₂—C(═O)—;
-(succinimidyl-3-yl-N)—CH₂CH₂CH₂CH₂CH₂—C(═O)—VA-NH—CH₂CH₂CH₂—C(═O)—;
-(succinimidyl-3-yl-N)—CH₂CH₂CH₂CH₂CH₂—C(═O)—VA-NH—CH₂CH₂CH₂CH₂CH₂—C(═O)—;
-(succinimidyl-3-yl-N)—CH₂CH₂CH₂CH₂CH₂—C(═O)—VA-NH—CH₂—O—CH₂—C(═O)—;
-(succinimidyl-3-yl-N)—CH₂CH₂CH₂CH₂CH₂—C(═O)—VA-NH—CH₂CH₂—O—CH₂—C(═O)—;
-(succinimidyl-3-yl-N)—CH₂CH₂—C(═O)—NH—CH₂CH₂O—CH₂CH₂O—CH₂CH₂—C(═O)—VA-NH—CH₂CH₂CH₂—C(═O)—;
-(succinimidyl-3-yl-N)—CH₂CH₂—C(═O)—NH—CH₂CH₂O—CH₂CH₂O—CH₂CH₂—C(═O)—VA-NH—CH₂CH₂—C(═O)—;
-(succinimidyl-3-yl-N)—CH₂CH₂—C(═O)—NH—CH₂CH₂O—CH₂CH₂O—CH₂CH₂O—CH₂CH₂O—CH₂CH₂—C(═O)—VA-NH—CH₂CH₂CH₂—C(═O)—;
-(succinimidyl-3-yl-N)—CH₂CH₂—C(═O)—NH—CH₂CH₂O—CH₂CH₂O—CH₂CH₂O—CH₂CH₂O—CH₂CH₂—C(═O)—VA-NH—CH₂CH₂—C(═O)—;
—CH₂—C(═O)—NH—CH₂CH₂—C(═O)—VA-NH—CH₂CH₂CH₂—C(═O)—; and
—C(═O)—CH₂CH₂CH₂CH₂CH₂CH₂—C(═O)—VA-NH—CH₂CH₂CH₂—C(═O)—.

In some embodiments, the spacer comprises self-immolative spacers. For example, the self-immolative spacer can comprise p-aminobenzoxy carbonyl (PABC) or p-aminobenzyl (PAB).

In some embodiments, the cleavable peptide can be directly spliced to the spacer.

In some embodiments, the L can comprise: -Val-Cit-PABC-, -Val-Ala-PABC-, -Glu-Val-Cit-PABC-, -Ala-Ala-Asn-PABC-, -Gly-Val-Cit-PABC-, -Gly-Gly-Gly-PABC-, -Gly-Gly-Phe-Gly-PABC-, -Val-Cit-PAB-, -Val-Ala-PAB-, -Glu-Val-Cit-PAB-, -Ala-Ala-Asn-PAB-, -Gly-Val-Cit-PAB-, -Gly-Gly-Gly-PAB- or -Gly-Gly-Phe-Gly-PAB-.

For example, the L can be selected from the following structure:

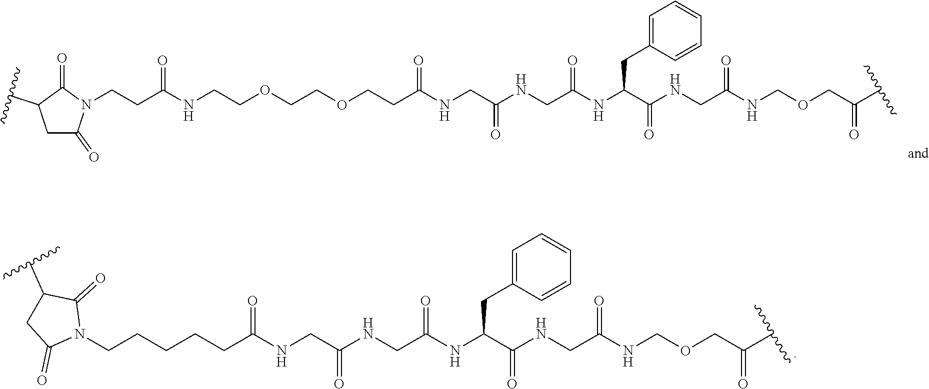 [SMILES: *C(=O)COCNC(=O)CNC(=O)[C@H](Cc1ccccc1)NC(=O)CNC(=O)CNC(=O)CCOCCOCCNC(=O)CCN1C(=O)CC(*)C1=O ; *C(=O)COCNC(=O)CNC(=O)[C@H](Cc1ccccc1)NC(=O)CNC(=O)CNC(=O)CCCCCN1C(=O)CC(*)C1=O]

In some embodiments, the p-aminobenzoxy carbonyl (PABC) or p-aminobenzyl (PAB) comprises a polysarcosine (poly-N-methylglycine) residue.

In some embodiments, the L is selected from the following structure:

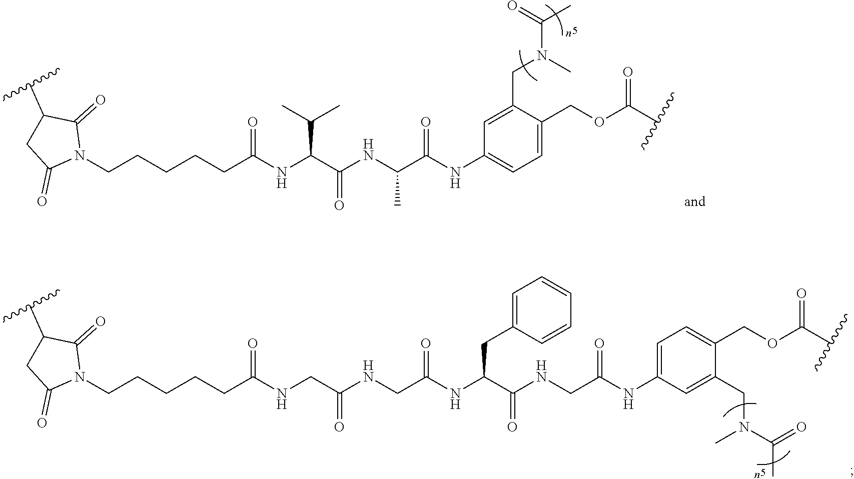

wherein n⁵ denotes an integer from 0 to 20, optionally the n⁵ can denote an integer from 1 to 15.

For example, the n⁵ can be 0, 1, 2, 3, 4, 5, 6, 7, 8, 9, 10, 11, 12, 13, 14, 15, 16, 17, 18, 19 or 20.

For example, the n⁵ can be denote an integer from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, 1 to 2, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 2 to 3, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, 3 to 4, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 4 to 7, 4 to 6, 4 to 5, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 5 to 7, 5 to 6, 6 to 20, 6 to 19, 6 to 18, 6 to 17, 6 to 16, 6 to 15, 6 to 14, 6 to 13, 6 to 12, 6 to 11, 6 to 10, 6 to 9, 6 to 8, 6 to 7, 7 to 20, 7 to 19, 7 to 18, 7 to 17, 7 to 16, 7 to 15, 7 to 14, 7 to 13, 7 to 12, 7 to 11, 7 to 10, 7 to 9, 7 to 8, 8 to 20, 8 to 19, 8 to 18, 8 to 17, 8 to 16, 8 to 15, 8 to 14, 8 to 13, 8 to 12, 8 to 11, 8 to 10, 8 to 9, 9 to 20, 9 to 19, 9 to 18, 9 to 17, 9 to 16, 9 to 15, 9 to 14, 9 to 13, 9 to 12, 9 to 11, 9 to 10, 10 to 20, 10 to 19, 10 to 18, 10 to 17, 10 to 16, 10 to 15, 10 to 14, 10 to 13, 10 to 12 or 10 to 11.

For example, the n⁵ can be denote an integer from 1 to 7. For example, the n⁵ can be denote an integer from 8 to 15.

For example, the L can be selected from the following structure:

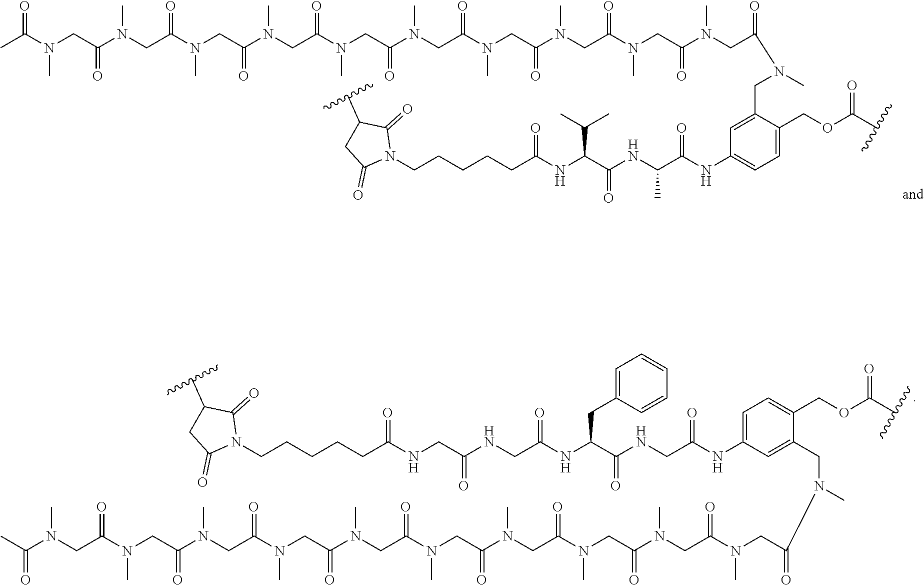 [SMILES: *C(=O)OCc1ccc(NC(=O)[C@H](C)NC(=O)[C@@H](NC(=O)CCCCCN2C(=O)CC(*)C2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O ; *C(=O)OCc1ccc(NC(=O)CNC(=O)[C@H](Cc2ccccc2)NC(=O)CNC(=O)CNC(=O)CCCCCN2C(=O)CC(*)C2=O)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O]

The antibody drug conjugate can be produced by reacting the compound obtainable by a known method (e.g., obtainable by a method described in the patent publication literature US2016/297890 (e.g., obtainable by a method described in the paragraphs [0336] to [0374])), with the antibody having a sulfhydryl group. The antibody having a sulfhydryl group can be obtained by a method well known to a person skilled in the art (Hermanson, G. T, Bioconjugate Techniques, pp. 56-136, pp. 456-493, Academic Press (1996)).

One specific example of the antibody-drug conjugate of the present invention can include an antibody-drug conjugate having a structure represented by the following formula:

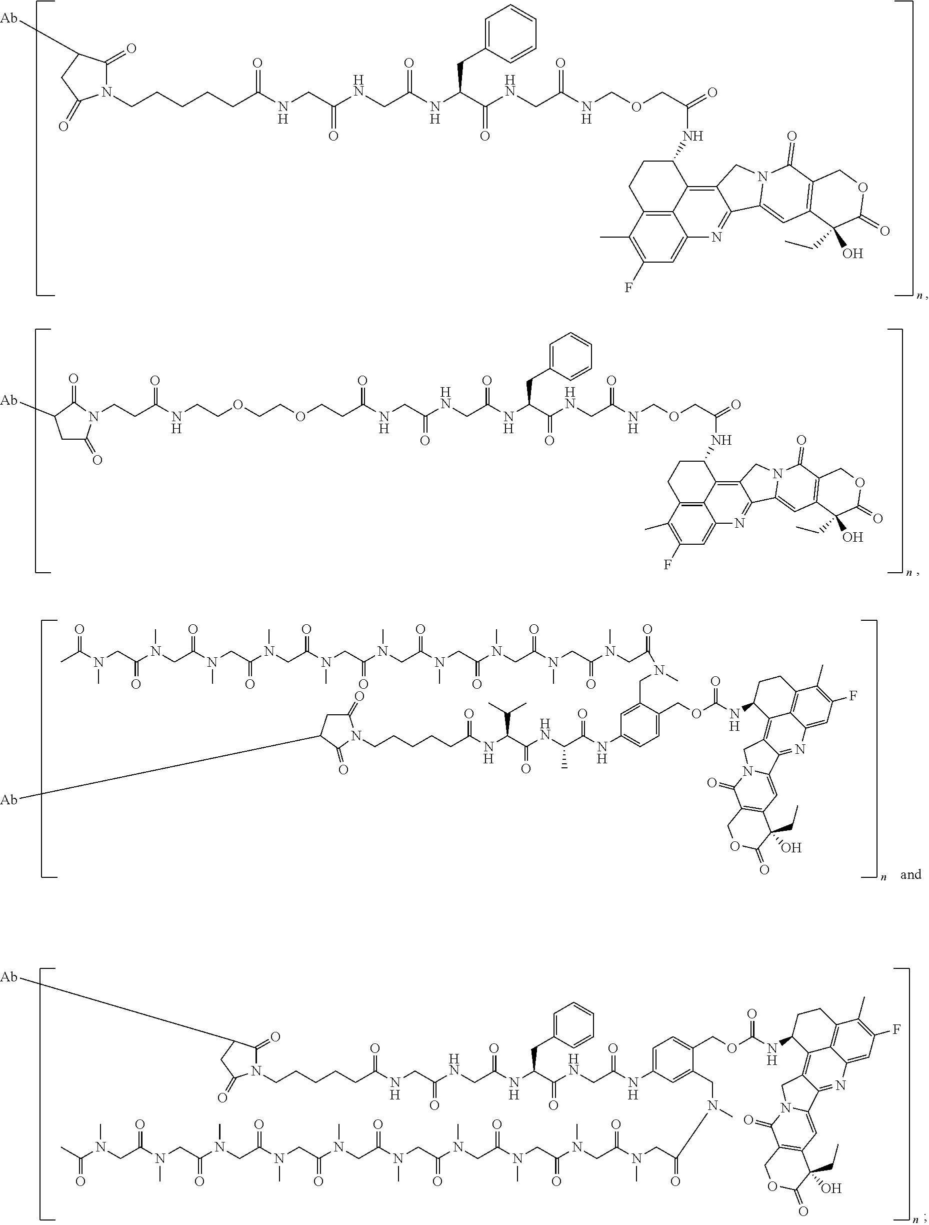

wherein n can be any number from 1 to 10. For example, the n can be any number from 1 to 9.5, 1 to 9, 1 to 8.5, 1 to 8, 1 to 7.5, 1 to 7, 1 to 6.5, 1 to 6, 1 to 5.5, 1 to 5, 1 to 4.5, 1 to 4, 1 to 3.5, 1 to 3, 1 to 2.5, 1 to 2, 1 to 1.5, 1.5 to 9.5, 1.5 to 9, 1.5 to 8.5, 1.5 to 8, 1.5 to 7.5, 1.5 to 7, 1.5 to 6.5, 1.5 to 6, 1.5 to 5.5, 1.5 to 5, 1.5 to 4.5, 1.5 to 4, 1.5 to 3.5, 1.5 to 3, 1.5 to 2.5, 1.5 to 2, 2 to 9.5, 2 to 9, 2 to 8.5, 2 to 8, 2 to 7.5, 2 to 7, 2 to 6.5, 2 to 6, 2 to 5.5, 2 to 5, 2 to 4.5, 2 to 4, 2 to 3.5, 2 to 3, 2 to 2.5, 2.5 to 9.5, 2.5 to 9, 2.5 to 8.5, 2.5 to 8, 2.5 to 7.5, 2.5 to 7, 2.5 to 6.5, 2.5 to 6, 2.5 to 5.5, 2.5 to 5, 2.5 to 4.5, 2.5 to 4, 2.5 to 3.5, 2.5 to 3, 3 to 9.5, 3 to 9, 3 to 8.5, 3 to 8, 3 to 7.5, 3 to 7, 3 to 6.5, 3 to 6, 3 to 5.5, 3 to 5, 3 to 4.5, 3 to 4, 3 to 3.5, 3.5 to 9.5, 3.5 to 9, 3.5 to 8.5, 3.5 to 8, 3.5 to 7.5, 3.5 to 7, 3.5 to 6.5, 3.5 to 6, 3.5 to 5.5, 3.5 to 5, 3.5 to 4.5, 3.5 to 4, 4 to 9.5, 4 to 9, 4 to 8.5, 4 to 8, 4 to 7.5, 4 to 7, 4 to 6.5, 4 to 6, 4 to 5.5, 4 to 5, 4 to 4.5, 4.5 to 9.5, 4.5 to 9, 4.5 to 8.5, 4.5 to 8, 4.5 to 7.5, 4.5 to 7, 4.5 to 6.5, 4.5 to 6, 4.5 to 5.5, 4.5 to 5, 5 to 9.5, 5 to 9, 5 to 8.5, 5 to 8, 5 to 7.5, 5 to 7, 5 to 6.5, 5 to 6, 5 to 5.5, 5.5 to 9.5, 5.5 to 9, 5.5 to 8.5, 5.5 to 8, 5.5 to 7.5, 5.5 to 7, 5.5 to 6.5, 5.5 to 6, 6 to 9.5, 6 to 9, 6 to 8.5, 6 to 8, 6 to 7.5, 6 to 7, 6 to 6.5, 6.5 to 9.5, 6.5 to 9, 6.5 to 8.5, 6.5 to 8, 6.5 to 7.5, 6.5 to 7, 7 to 9.5, 7 to 9, 7 to 8.5, 7 to 8, 7 to 7.5, 7.5 to 9.5, 7.5 to 9, 7.5 to 8.5, 7.5 to 8, 8 to 9.5, 8 to 9, 8 to 8.5, 8.5 to 9.5,or 8.5 to 9.

In this context, Ab represents the anti-CDH6 antibody disclosed in the present description, and the antibody is conjugated to the linker-payload via a sulfhydryl group stemming from the antibody. In this context, n has the same meaning as that of the so-called DAR (drug-to-antibody Ratio), and represents a drug-to-antibody ratio per antibody. Specifically, n represents the number of conjugated drug molecules per antibody molecule, which is a numeric value defined and indicated as an average value, i.e., the average number of conjugated drug molecules. In the present invention, n can be 2 to 8 and is preferably 5 to 8, more preferably 7 to 8, and still more preferably 8, in measurement by common procedure F.

One example of the antibody-drug conjugate of the present invention can include an antibody-drug conjugate having a structure represented by the above-described formula wherein the antibody represented by Ab comprises any one antibody selected from the group consisting of the following antibodies (a) to (k), a functional fragment of the antibody, and a pharmacologically acceptable salt of the antibody-drug conjugate:
(a) an antibody consisting of a heavy chain comprising the amino acid sequence as set forth in SEQ ID NO: 72 and a light chain comprising the amino acid sequence as set forth in SEQ ID NO: 73;
(b) an antibody consisting of a heavy chain comprising the amino acid sequence as set forth in SEQ ID NO: 74 and a light chain comprising the amino acid sequence as set forth in SEQ ID NO: 76;
(c) an antibody consisting of a heavy chain comprising the amino acid sequence as set forth in SEQ ID NO: 74 and a light chain comprising the amino acid sequence as set forth in SEQ ID NO: 77;
(d) an antibody consisting of a heavy chain comprising the amino acid sequence as set forth in SEQ ID NO: 75 and a light chain comprising the amino acid sequence as set forth in SEQ ID NO: 76;
(e) an antibody consisting of a heavy chain comprising the amino acid sequence as set forth in SEQ ID NO: 75 and a light chain comprising the amino acid sequence as set forth in SEQ ID NO: 77;
(f) an antibody consisting of a heavy chain comprising the amino acid sequence as set forth in SEQ ID NO: 80 and a light chain comprising the amino acid sequence as set forth in SEQ ID NO: 81;
(g) an antibody consisting of a heavy chain comprising the amino acid sequence as set forth in SEQ ID NO: 78 and a light chain comprising the amino acid sequence as set forth in SEQ ID NO: 82;
(h) an antibody consisting of a heavy chain comprising the amino acid sequence as set forth in SEQ ID NO: 78 and a light chain comprising the amino acid sequence as set forth in SEQ ID NO: 83;
(i) an antibody consisting of a heavy chain comprising the amino acid sequence as set forth in SEQ ID NO: 78 and a light chain comprising the amino acid sequence as set forth in SEQ ID NO: 84;
(j) an antibody consisting of a heavy chain comprising the amino acid sequence as set forth in SEQ ID NO: 79 and a light chain comprising the amino acid sequence as set forth in SEQ ID NO: 82;
(k) an antibody consisting of a heavy chain comprising the amino acid sequence as set forth in SEQ ID NO: 79 and a light chain comprising the amino acid sequence as set forth in SEQ ID NO: 83;
(l) an antibody consisting of a heavy chain comprising the amino acid sequence as set forth in SEQ ID NO: 79 and a light chain comprising the amino acid sequence as set forth in SEQ ID NO: 84;
(m) any one antibody selected from the group consisting of the antibodies (a) to (j), wherein the heavy chain or the light chain comprises one or two or more modifications selected from the group consisting of posttranslational modifications typified by N-linked glycosylation, O-linked glycosylation, N-terminal processing, C-terminal processing, deamidation, isomerization of aspartic acid, oxidation of methionine, addition of a methionine residue to the N-terminus, amidation of a proline residue, and conversion of N-terminal glutamine or N-terminal glutamic acid to pyroglutamic acid, and a deletion of one or two amino acids at the carboxyl terminus.

One example of the antibody-drug conjugate of the present invention can include an antibody-drug conjugate having a structure represented by the following formula:

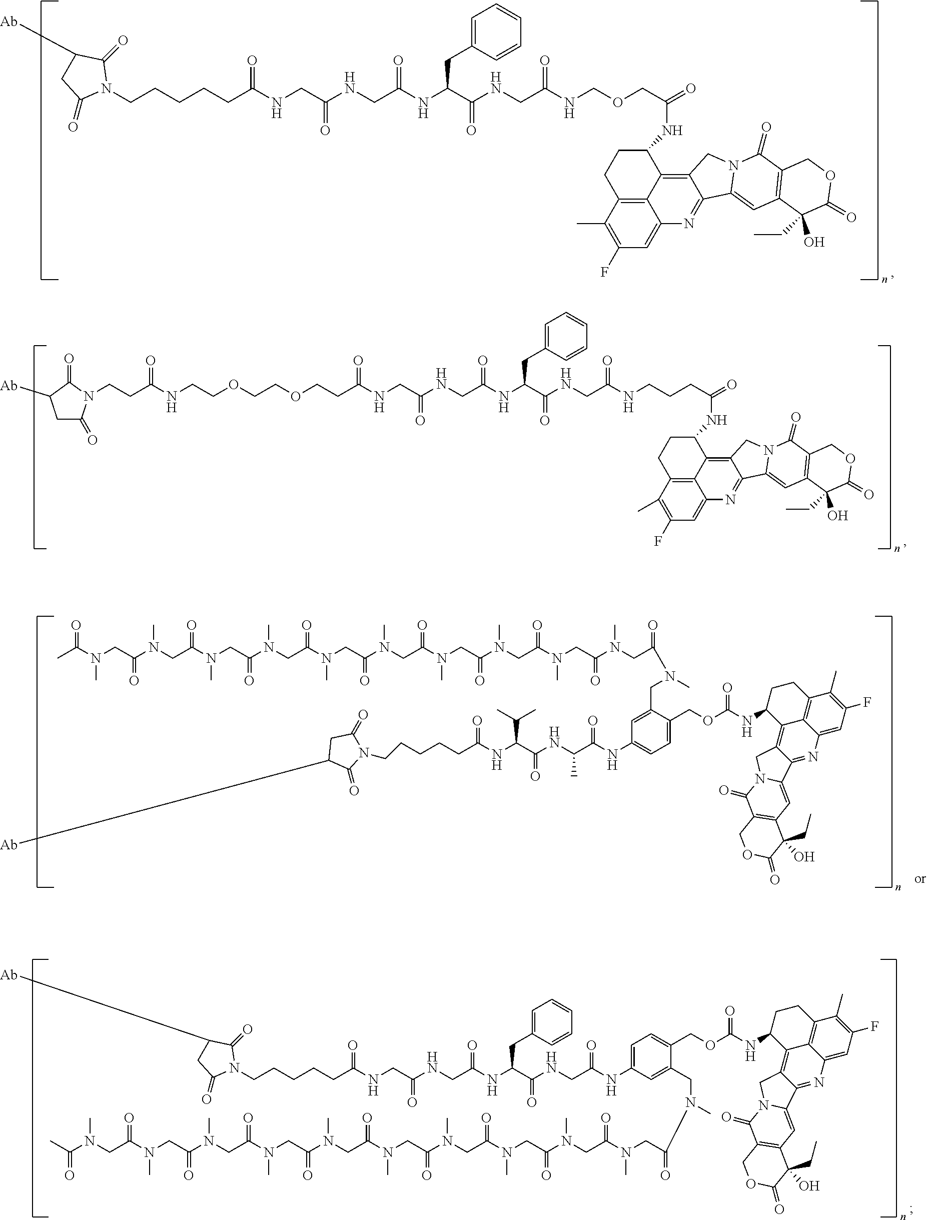

wherein n can be any number from 1 to 10;
wherein the antibody represented by Ab comprises a heavy chain variable region (VH) comprising (1) a heavy chain CDR1 (HCDR1) having an amino acid sequence of SEQ ID NO: 2; (2) a heavy chain CDR2 (HCDR2) having an amino acid sequence of SEQ ID NO: 3; and (3) a heavy chain CDR3 (HCDR3) having an amino acid sequence of SEQ ID NO: 4, and/or a light chain variable region comprising (1) a light chain CDR1 (LCDR1) having an amino acid sequence of SEQ ID NO: 5; (2) a light chain CDR2 (LCDR2) having an amino acid sequence of SEQ ID NO: 6; and (3) a light chain CDR3 (LCDR3) having an amino acid sequence of SEQ ID NO: 7; and wherein n is a number from 1 to 10 (e.g., 2 to 10, 4 to 10, 5 to 10, 6 to 10, 7 to 10, and 8 to 10). In some embodiments, the antibody represented by Ab comprises a heavy chain variable region comprising the amino acid sequence as set forth in SEQ ID NO: 18, and a light chain variable region comprising the amino acid sequence as set forth in SEQ ID NO: 23. In some embodiments, the antibody represented by Ab comprises a heavy chain variable region comprising the amino acid sequence as set forth in SEQ ID NO: 66, and a light chain variable region comprising the amino acid sequence as set forth in SEQ ID NO: 68. In some embodiments, the antibody represented by Ab comprises CL069707-H1L1, CL069707-H1L2, CL069707-H2L1 or CL069707-H2L2.

One example of the antibody-drug conjugate of the present invention can include an antibody-drug conjugate having a structure represented by the following formula:

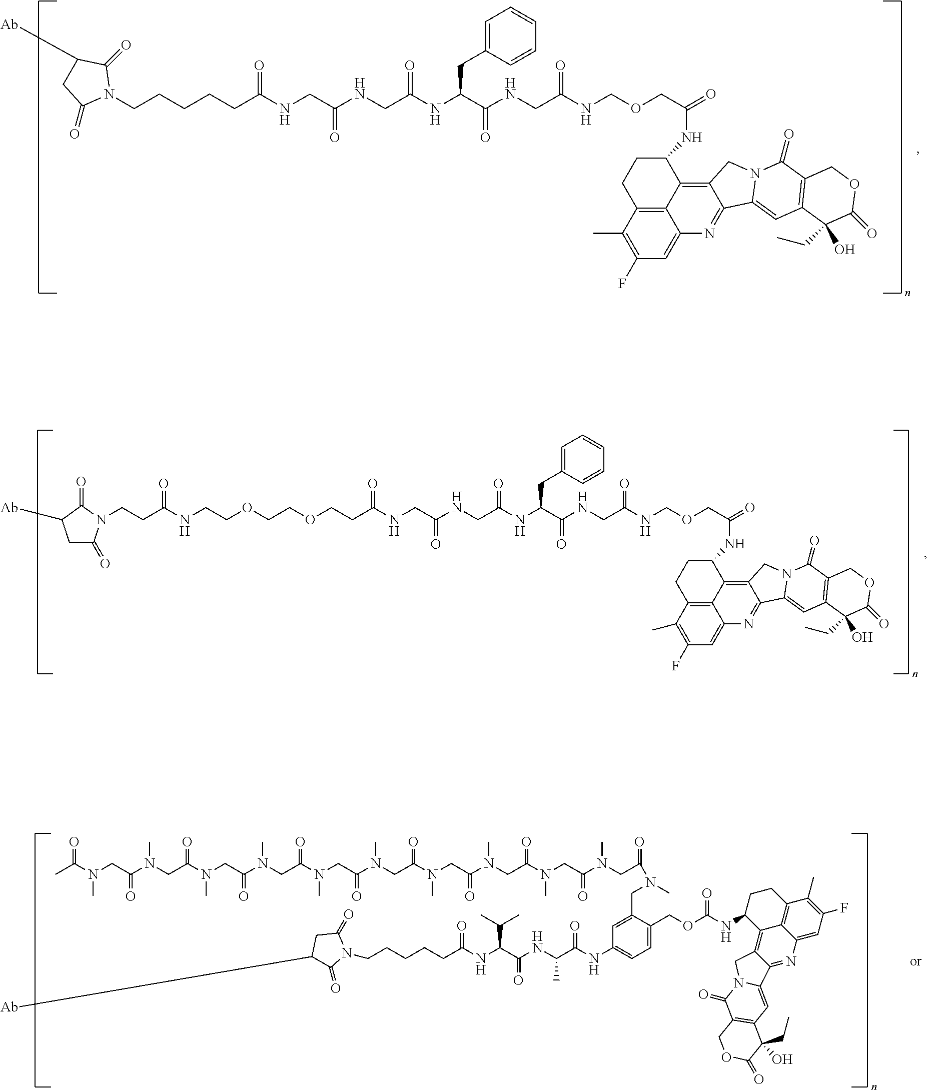

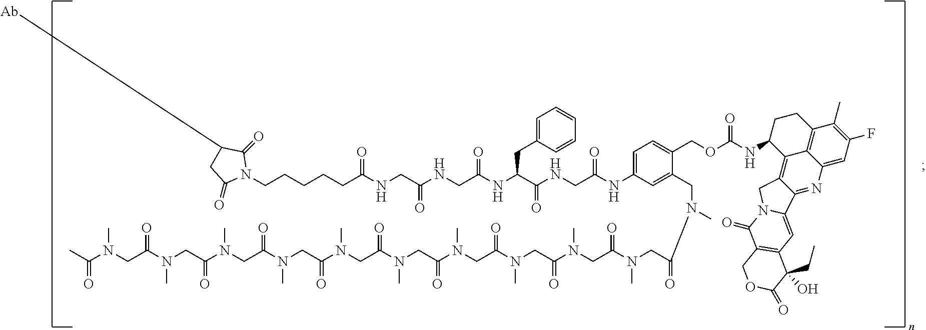

wherein n can be any number from 1 to 10;
wherein the antibody represented by Ab comprises a heavy chain variable region (VH) comprising (1) a heavy chain CDR1 (HCDR1) having an amino acid sequence of SEQ ID NO: 8; (2) a heavy chain CDR2 (HCDR2) having an amino acid sequence of SEQ ID NO: 9; and (3) a heavy chain CDR3 (HCDR3) having an amino acid sequence of SEQ ID NO: 10, and/or a light chain variable region comprising (1) a light chain CDR1 (LCDR1) having an amino acid sequence of SEQ ID NO: 11; (2) a light chain CDR2 (LCDR2) having an amino acid sequence of SEQ ID NO: 12; and (3) a light chain CDR3 (LCDR3) having an amino acid sequence of SEQ ID NO: 13; and wherein n is a number from 1 to 10 (e.g., 2 to 10, 4 to 10, 5 to 10, 6 to 10, 7 to 10, and 8 to 10). In some embodiments, the antibody represented by Ab comprises a heavy chain variable region comprising the amino acid sequence as set forth in SEQ ID NO: 41, and a light chain variable region comprising the amino acid sequence as set forth in SEQ ID NO: 46. In some embodiments, the antibody represented by Ab comprises a heavy chain variable region comprising the amino acid sequence as set forth in SEQ ID NO: 67, and a light chain variable region comprising the amino acid sequence as set forth in SEQ ID NO: 69. In some embodiments, the antibody represented by Ab comprises CL069463-H1L1, CL069463-H1L2, CL069463-H1L3, CL069463-H2L1, CL069463-H2L2, or CL069463-H2L3.

One example of the antibody-drug conjugate of the present invention can include an antibody-drug conjugate having a structure represented by the following formula:

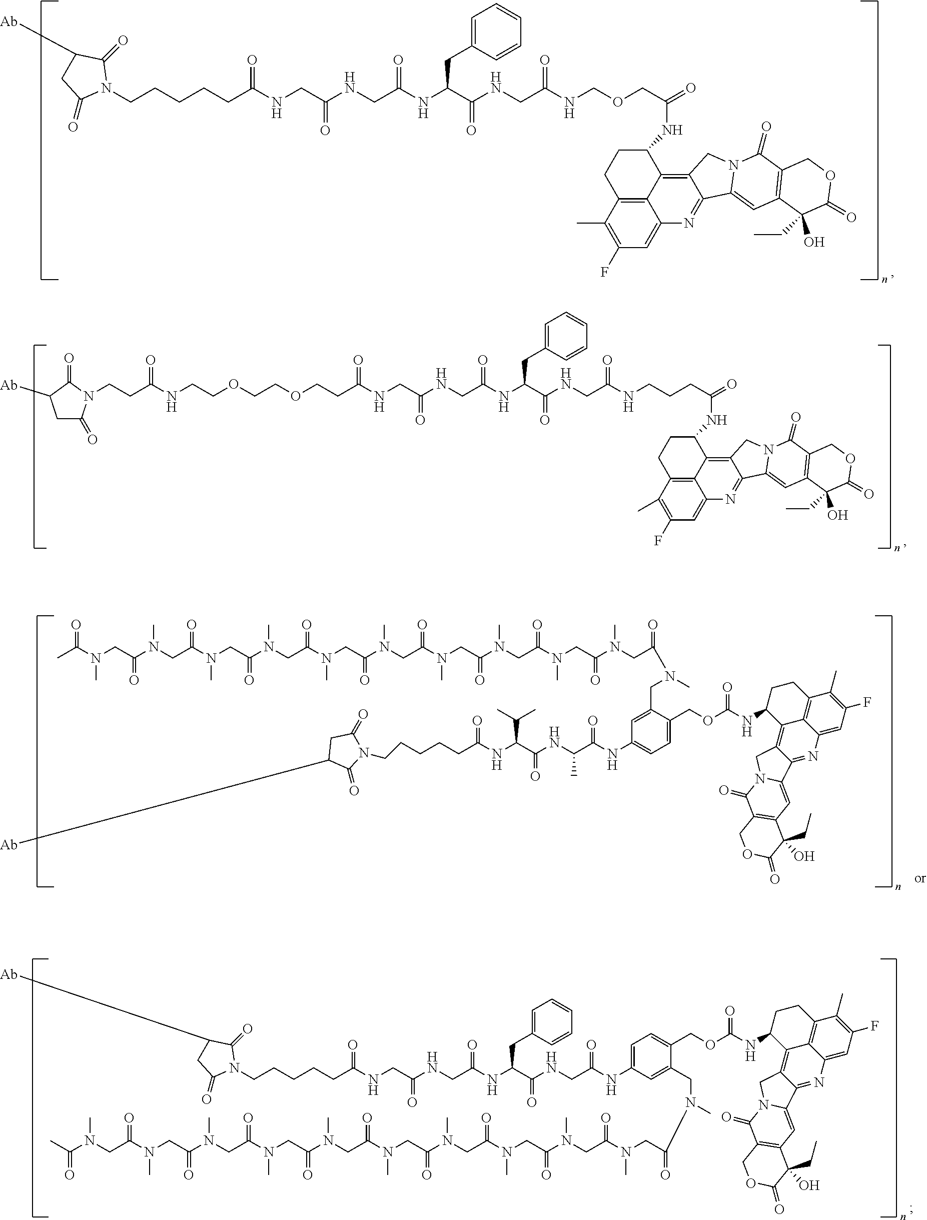

wherein n can be any number from 1 to 10;
wherein the antibody represented by Ab comprises a heavy chain variable region (VH) comprising (1) a heavy chain CDR1 (HCDR1) having an amino acid sequence of SEQ ID NO: 89; (2) a heavy chain CDR2 (HCDR2) having an amino acid sequence of SEQ ID NO: 90; and (3) a heavy chain CDR3 (HCDR3) having an amino acid sequence of SEQ ID NO: 91, and/or a light chain variable region comprising (1) a light chain CDR1 (LCDR1) having an amino acid sequence of SEQ ID NO: 92; (2) a light chain CDR2 (LCDR2) having an amino acid sequence of SEQ ID NO: 93; and (3) a light chain CDR3 (LCDR3) having an amino acid sequence of SEQ ID NO: 94; and wherein n is a number from 1 to 10 (e.g., 2 to 10, 4 to 10, 5 to 10, 6 to 10, 7 to 10, and 8 to 10). In some embodiments, the antibody represented by Ab comprises a heavy chain variable region comprising the amino acid sequence as set forth in SEQ ID NO: 95, and a light chain variable region comprising the amino acid sequence as set forth in SEQ ID NO: 96.

One example of the antibody-drug conjugate of the present invention can include an antibody-drug conjugate having a structure represented by the following formula:

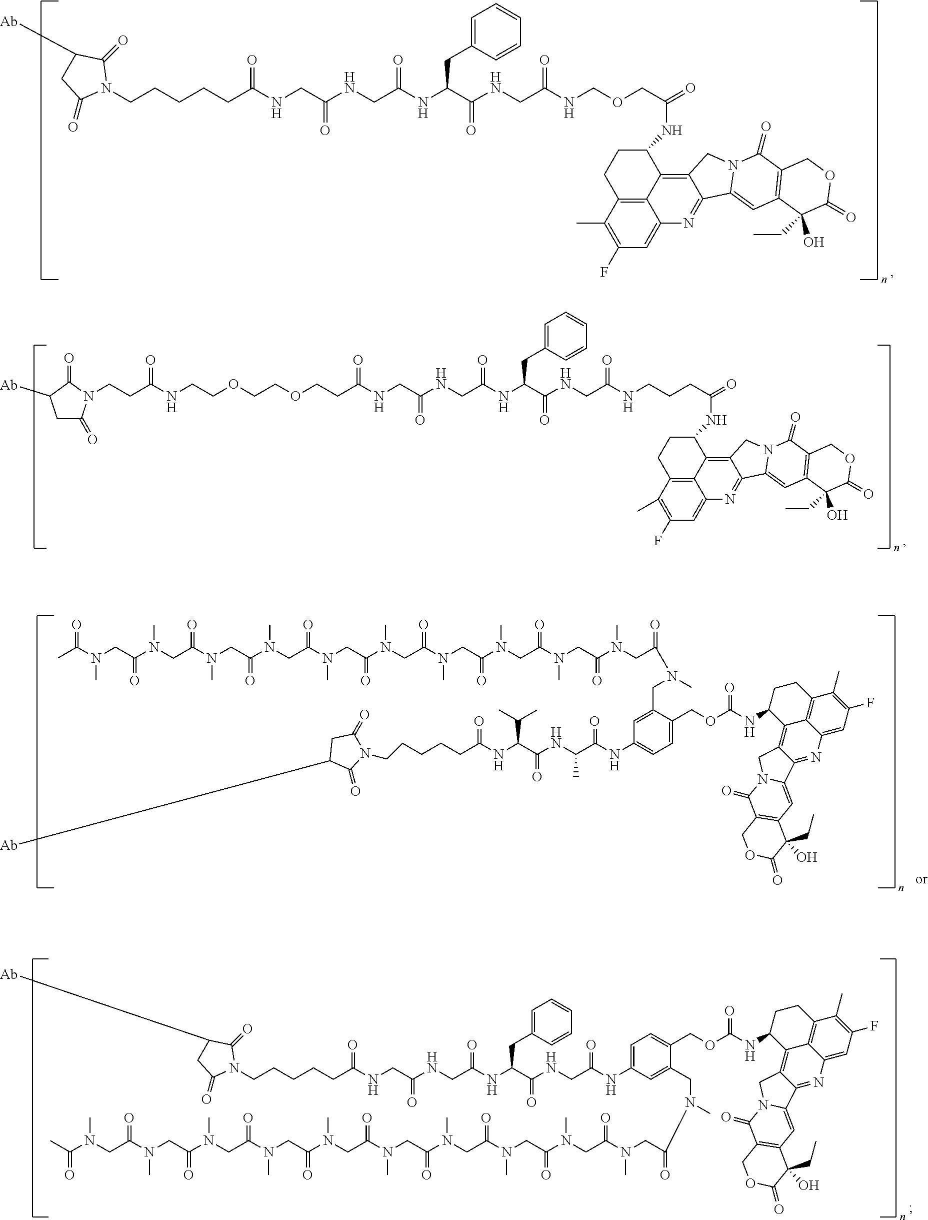

wherein n can be any number from 1 to 10;
wherein the antibody represented by Ab comprises a heavy chain variable region (VH) comprising (1) a heavy chain CDR1 (HCDR1) having an amino acid sequence of SEQ ID NO: 97; (2) a heavy chain CDR2 (HCDR2) having an amino acid sequence of SEQ ID NO: 98; and (3) a heavy chain CDR3 (HCDR3) having an amino acid sequence of SEQ ID NO: 99, and/or a light chain variable region comprising (1) a light chain CDR1 (LCDR1) having an amino acid sequence of SEQ ID NO: 100; (2) a light chain CDR2 (LCDR2) having an amino acid sequence of SEQ ID NO: 101; and (3) a light chain CDR3 (LCDR3) having an amino acid sequence of SEQ ID NO: 102; and wherein n is a number from 1 to 10 (e.g., 2 to 10, 4 to 10, 5 to 10, 6 to 10, 7 to 10, and 8 to 10). In some embodiments, the antibody represented by Ab comprises a heavy chain variable region comprising the amino acid sequence as set forth in SEQ ID NO: 103, and a light chain variable region comprising the amino acid sequence as set forth in SEQ ID NO: 104.

One example of the antibody-drug conjugate of the present invention can include an antibody-drug conjugate having a structure represented by the following formula:

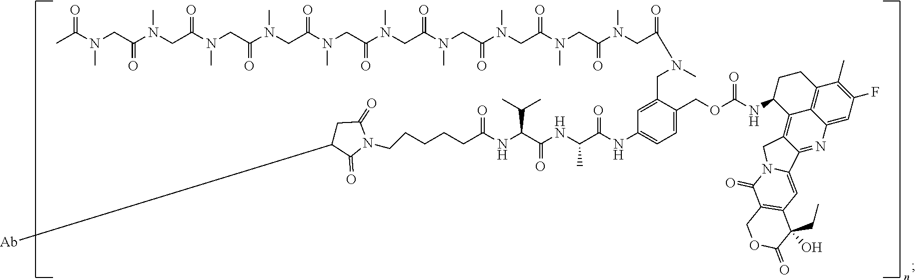

wherein n can be any number from 1 to 10;
wherein the antibody represented by Ab comprises a heavy chain variable region (VH) comprising (1) a heavy chain CDR1 (HCDR1) having an amino acid sequence of SEQ ID NO: 2; (2) a heavy chain CDR2 (HCDR2) having an amino acid sequence of SEQ ID NO: 3; and (3) a heavy chain CDR3 (HCDR3) having an amino acid sequence of SEQ ID NO: 4, and/or a light chain variable region comprising (1) a light chain CDR1 (LCDR1) having an amino acid sequence of SEQ ID NO: 5; (2) a light chain CDR2 (LCDR2) having an amino acid sequence of SEQ ID NO: 6; and (3) a light chain CDR3 (LCDR3) having an amino acid sequence of SEQ ID NO: 7; and wherein n is a number from 1 to 10 (e.g., 2 to 10, 4 to 10, 5 to 10, 6 to 10, 7 to 10, and 8 to 10). In some embodiments, the antibody represented by Ab comprises a heavy chain variable region comprising the amino acid sequence as set forth in SEQ ID NO: 18, and a light chain variable region comprising the amino acid sequence as set forth in SEQ ID NO: 23. In some embodiments, the antibody represented by Ab comprises a heavy chain variable region comprising the amino acid sequence as set forth in SEQ ID NO: 66, and a light chain variable region comprising the amino acid sequence as set forth in SEQ ID NO: 68. In some embodiments, the antibody represented by Ab comprises CL069707-H1L1.

### Medicament

Anti-CDH6 antibody-drug conjugate in the Examples is a conjugate of the anti-CDH6 antibody and/or the functional fragment of the antibody having internalization activity, and the drug having antitumor activity such as cytotoxic activity. Since this anti-CDH6 antibody-drug conjugate exhibits antitumor activity against cancer cells expressing CDH6, it can be used as a medicament, and in particular, as a therapeutic agent and/or a prophylactic agent for cancer.

The anti-CDH6 antibody-drug conjugate of the present invention may absorb moisture or have adsorption water, for example, to turn into a hydrate when it is left in air or subjected to recrystallization or purification procedures. Such a compound or a pharmacologically acceptable salt containing water is also included in the present invention. The present invention can also include an anti-CDH6 antibody-drug conjugate in which one or more atoms constituting the antibody-drug conjugate are replaced with isotopes of the atoms. A composition comprising the antibody-drug conjugate labeled with such an isotope is useful as, for example, a therapeutic agent, a prophylactic agent, a research reagent, an assay reagent, a diagnostic agent, and an in vivo diagnostic imaging agent.

The type of cancer to which the anti-CDH6 antibody-drug conjugate of the present invention is applied is not particularly limited as long as the cancer expresses CDH6 in cancer cells to be treated. Examples thereof can include renal cell carcinoma (e.g., renal clear cell carcinoma or papillary renal cell carcinoma), ovarian cancer, ovarian serous adenocarcinoma, thyroid cancer, bile duct cancer, lung cancer (e.g., small-cell lung cancer or non-small cell lung cancer), glioblastoma, mesothelioma, uterine cancer, pancreatic cancer, Wilms' tumor and neuroblastoma, though the cancer is not limited thereto as long as the cancer expresses CDH6. More preferred examples of the cancer can include renal cell carcinoma (e.g., renal clear cell carcinoma and papillary renal cell carcinoma) and ovarian cancer.

The anti-CDH6 antibody-drug conjugate of the present invention can preferably be administered to a mammal, and more preferably to a human.

A substance used in a pharmaceutical composition comprising the anti-CDH6 antibody-drug conjugate of the present invention can be appropriately selected from pharmaceutical additives and others usually used in this field, in terms of the applied dose or the applied concentration, and then used.

The anti-CDH6 antibody-drug conjugate of the present invention can be administered as a pharmaceutical composition comprising one or more pharmaceutically compatible components. For example, the pharmaceutical composition typically comprises one or more pharmaceutical carriers (e.g., sterilized liquids (e.g., water and oil (including petroleum oil and oil of animal origin, plant origin or synthetic origin (e.g., peanut oil, soybean oil, mineral oil, and sesame oil)))). Water is a more typical carrier when the pharmaceutical composition is intravenously administered. An aqueous saline solution, an aqueous dextrose solution, and an aqueous glycerol solution can also be used as a liquid carrier, in particular, for an injection solution. Suitable pharmaceutical vehicles are known in the art. If desired, the composition may also comprise a trace amount of a moisturizing agent, an emulsifying agent or a pH buffering agent.

Various delivery systems are known, and they can be used for administering the anti-CDH6 antibody-drug conjugate of the present invention. Examples of the administration route can include, but are not limited to, intradermal, intramuscular, intraperitoneal, intravenous, and subcutaneous routes. The administration can be made by injection or bolus injection, for example. According to a specific preferred embodiment, the administration of the above-described antibody-drug conjugate is performed by injection. Parenteral administration is a preferred administration route.

### The Instant Application Discloses the Following Specific Embodiments

Embodiment 1: An immunoconjugate or a pharmaceutically acceptable salt or a solvate thereof comprising an antibody or antigen binding fragment thereof that specifically binds to an epitope of Cadherin-6 (CDH6), the antibody or the antigen binding fragment thereof has one or more of the following properties:
i) is capable of binding to the CDH6 protein with a KD value below about 3.1×10⁻⁹M in an ELISA assay;
ii) is capable of being internalized by an CDH6-expressing cell upon binding to CDH6.

Embodiment 2: The immunoconjugate or a pharmaceutically acceptable salt or a solvate thereof of embodiment 1, wherein the CDH6 is a mammalian CDH6 protein.

Embodiment 3: The immunoconjugate or a pharmaceutically acceptable salt or a solvate thereof of any one of embodiments 1-2, wherein the CDH6 is human CDH6 protein.

Embodiment 4: The immunoconjugate or a pharmaceutically acceptable salt or a solvate thereof of any one of embodiments 1-3, wherein the antibody comprises a monoclonal antibody, a polyclonal antibody, a dimer, a multimer, an intact antibody, an antibody fragment, a human antibody, a humanized antibody and/or a chimeric antibody.

Embodiment 5: The immunoconjugate or a pharmaceutically acceptable salt or a solvate thereof of any one of embodiments 1-4, wherein the antigen binding fragment comprises Fab, Fab′, Fv fragment, F(ab′)₂, scFv, di-scFv and/or dAb.

Embodiment 6: The immunoconjugate or a pharmaceutically acceptable salt or a solvate thereof of any one of embodiments 1-5, wherein the antibody is a monoclonal antibody.

Embodiment 7: The immunoconjugate or a pharmaceutically acceptable salt or a solvate thereof of any one of embodiments 1-6, wherein the antibody is a chimeric antibody, a humanized antibody and/or a human antibody.

Embodiment 8: The immunoconjugate or a pharmaceutically acceptable salt or a solvate thereof of any one of embodiments 1-7, wherein the antibody or the antigen binding fragment comprises at least one CDR (Complementarity Determining Region) of a heavy chain variable region (VH), the VH comprises the amino acid sequence as set forth in SEQ ID NO: 18, SEQ ID NO: 66, SEQ ID NO: 41, SEQ ID NO: 67, SEQ ID NO: 95 or SEQ ID NO: 103.

Embodiment 9: The immunoconjugate or a pharmaceutically acceptable salt or a solvate thereof of any one of embodiments 1-8, wherein the antibody or the antigen binding fragment comprises at least one CDR of a light chain variable region (VL), the VL comprises the amino acid sequence as set forth in SEQ ID NO: 23, SEQ ID NO: 68, SEQ ID NO: 46, SEQ ID NO: 69, SEQ ID NO: 96 or SEQ ID NO: 104.

Embodiment 10: The immunoconjugate or a pharmaceutically acceptable salt or a solvate thereof of any one of embodiments 1-9, comprising VH, wherein the VH comprises HCDR1, HCDR2, HCDR3, the HCDR1 comprises the amino acid sequence as set forth in SEQ ID NO: 1.

Embodiment 11: The immunoconjugate or a pharmaceutically acceptable salt or a solvate thereof of any one of embodiments 1-10, comprising VH, wherein the VH comprises HCDR1, HCDR2, HCDR3, the HCDR1 comprises the amino acid sequence as set forth in SEQ ID NO: 2, SEQ ID NO: 8, SEQ ID NO: 89 or SEQ ID NO: 97.

Embodiment 12: The immunoconjugate or a pharmaceutically acceptable salt or a solvate thereof of any one of embodiments 1-11, comprising VH, wherein the VH comprises HCDR1, HCDR2, HCDR3, the HCDR2 comprises the amino acid sequence as set forth in SEQ ID NO: 3, SEQ ID NO: 9, SEQ ID NO: 90 or SEQ ID NO: 98.

Embodiment 13: The immunoconjugate or a pharmaceutically acceptable salt or a solvate thereof of any one of embodiments 1-12, comprising VH, wherein the VH comprises HCDR1, HCDR2, HCDR3, the HCDR3 comprises the amino acid sequence as set forth in SEQ ID NO: 4, SEQ ID NO: 10, SEQ ID NO: 91 or SEQ ID NO: 99.

Embodiment 14: The immunoconjugate or a pharmaceutically acceptable salt or a solvate thereof of any one of embodiments 1-13, comprising VH, wherein the VH comprises HCDR1, HCDR2, HCDR3, the HCDR1 comprises the amino acid sequence as set forth in SEQ ID NO: 1, the HCDR2 comprises the amino acid sequence as set forth in SEQ ID NO: 3 or SEQ ID NO: 9, and the HCDR3 comprises the amino acid sequence as set forth in SEQ ID NO: 4 or SEQ ID NO: 10.

Embodiment 15: The immunoconjugate or a pharmaceutically acceptable salt or a solvate thereof of any one of embodiments 1-14, comprising VH, wherein the VH comprises HCDR1, HCDR2, HCDR3, wherein the HCDR1 comprises the amino acid sequence as set forth in SEQ ID NO: 2, the HCDR2 comprises the amino acid sequence as set forth in SEQ ID NO: 3, and the HCDR3 comprises the amino acid sequence as set forth in SEQ ID NO: 4; or
wherein the HCDR1 comprises the amino acid sequence as set forth in SEQ ID NO: 8, the HCDR2 comprises the amino acid sequence as set forth in SEQ ID NO: 9, and the HCDR3 comprises the amino acid sequence as set forth in SEQ ID NO: 10; or
wherein the HCDR1 comprises the amino acid sequence as set forth in SEQ ID NO: 89, the HCDR2 comprises the amino acid sequence as set forth in SEQ ID NO: 90, and the HCDR3 comprises the amino acid sequence as set forth in SEQ ID NO: 91; or
wherein the HCDR1 comprises the amino acid sequence as set forth in SEQ ID NO: 97, the HCDR2 comprises the amino acid sequence as set forth in SEQ ID NO: 98, and the HCDR3 comprises the amino acid sequence as set forth in SEQ ID NO: 99.

Embodiment 16: The immunoconjugate or a pharmaceutically acceptable salt or a solvate thereof of any one of embodiments 1-15, comprising VH, wherein the VH comprises the amino acid sequence as set forth in SEQ ID NO: 18, SEQ ID NO: 66, SEQ ID NO: 41, SEQ ID NO: 67, SEQ ID NO: 95 or SEQ ID NO: 103.

Embodiment 17: The immunoconjugate or a pharmaceutically acceptable salt or a solvate thereof of any one of embodiments 1-16, comprising an antibody heavy chain constant region.

Embodiment 18: The immunoconjugate or a pharmaceutically acceptable salt or a solvate thereof of embodiment 17, wherein the antibody heavy chain constant region comprises a constant region derived from human IgG.

Embodiment 19: The immunoconjugate or a pharmaceutically acceptable salt or a solvate thereof of any one of embodiments 17-18, wherein the antibody heavy chain constant region comprises the amino acid sequence as set forth in SEQ ID NO: 70.

Embodiment 20: The immunoconjugate or a pharmaceutically acceptable salt or a solvate thereof of any one of embodiments 1-16, comprising an antibody heavy chain, wherein the antibody heavy chain comprises the amino acid sequence as set forth in SEQ ID NO: 72, SEQ ID NO: 85, SEQ ID NO: 80 or SEQ ID NO: 87.

Embodiment 21: The immunoconjugate or a pharmaceutically acceptable salt or a solvate thereof of any one of embodiments 1-20, comprising VL, wherein the VL comprises LCDR1, LCDR2, LCDR3, the LCDR1 comprises the amino acid sequence as set forth in SEQ ID NO: 5, SEQ ID NO: 11, SEQ ID NO: 92 or SEQ ID NO: 100.

Embodiment 22: The immunoconjugate or a pharmaceutically acceptable salt or a solvate thereof of any one of embodiments 1-21, comprising VL, wherein the VL comprises LCDR1, LCDR2, LCDR3, the LCDR2 comprises the amino acid sequence as set forth in SEQ ID NO: 6, SEQ ID NO: 12, SEQ ID NO: 93 or SEQ ID NO: 101.

Embodiment 23: The immunoconjugate or a pharmaceutically acceptable salt or a solvate thereof of any one of embodiments 1-22, comprising VL, wherein the VL comprises LCDR1, LCDR2, LCDR3, the LCDR3 comprises the amino acid sequence as set forth in SEQ ID NO: 7, SEQ ID NO: 13, SEQ ID NO: 94 or SEQ ID NO: 102.

Embodiment 24: The immunoconjugate or a pharmaceutically acceptable salt or a solvate thereof of any one of embodiments 1-23, comprising VL, wherein the VL comprises LCDR1, LCDR2, LCDR3, wherein the LCDR1 comprises the amino acid sequence as set forth in SEQ ID NO: 5, the LCDR2 comprises the amino acid sequence as set forth in SEQ ID NO: 6, and the LCDR3 comprises the amino acid sequence as set forth in SEQ ID NO: 7; or
wherein the LCDR1 comprises the amino acid sequence as set forth in SEQ ID NO: 11, the LCDR2 comprises the amino acid sequence as set forth in SEQ ID NO: 12, and the LCDR3 comprises the amino acid sequence as set forth in SEQ ID NO: 13; or
wherein the LCDR1 comprises the amino acid sequence as set forth in SEQ ID NO: 92, the LCDR2 comprises the amino acid sequence as set forth in SEQ ID NO: 93, and the LCDR3 comprises the amino acid sequence as set forth in SEQ ID NO: 94; or
wherein the LCDR1 comprises the amino acid sequence as set forth in SEQ ID NO: 100, the LCDR2 comprises the amino acid sequence as set forth in SEQ ID NO: 101, and the LCDR3 comprises the amino acid sequence as set forth in SEQ ID NO: 102.

Embodiment 25: The immunoconjugate or a pharmaceutically acceptable salt or a solvate thereof of any one of embodiments 1-24, comprising VH and VL, wherein the VH comprises HCDR1, HCDR2, HCDR3, and the VL comprises LCDR1, LCDR2, LCDR3, wherein the HCDR1 comprises the amino acid sequence as set forth in SEQ ID NO: 1, the HCDR2 comprises the amino acid sequence as set forth in SEQ ID NO: 3, and the HCDR3 comprises the amino acid sequence as set forth in SEQ ID NO: 4, the LCDR1 comprises the amino acid sequence as set forth in SEQ ID NO: 5, the LCDR2 comprises the amino acid sequence as set forth in SEQ ID NO: 6, and the LCDR3 comprises the amino acid sequence as set forth in SEQ ID NO: 7; or
wherein the HCDR1 comprises the amino acid sequence as set forth in SEQ ID NO: 1, the HCDR2 comprises the amino acid sequence as set forth in SEQ ID NO: 9, and the HCDR3 comprises the amino acid sequence as set forth in SEQ ID NO: 10 the LCDR1 comprises the amino acid sequence as set forth in SEQ ID NO: 11, the LCDR2 comprises the amino acid sequence as set forth in SEQ ID NO: 12, and the LCDR3 comprises the amino acid sequence as set forth in SEQ ID NO: 13.

Embodiment 26: The immunoconjugate or a pharmaceutically acceptable salt or a solvate thereof of any one of embodiments 1-25, comprising VH and VL, wherein the VH comprises HCDR1, HCDR2, HCDR3, and the VL comprises LCDR1, LCDR2, LCDR3, wherein the HCDR1 comprises the amino acid sequence as set forth in SEQ ID NO: 2, the HCDR2 comprises the amino acid sequence as set forth in SEQ ID NO: 3, and the HCDR3 comprises the amino acid sequence as set forth in SEQ ID NO: 4, the LCDR1 comprises the amino acid sequence as set forth in SEQ ID NO: 5, the LCDR2 comprises the amino acid sequence as set forth in SEQ ID NO: 6, and the LCDR3 comprises the amino acid sequence as set forth in SEQ ID NO: 7; or
wherein the HCDR1 comprises the amino acid sequence as set forth in SEQ ID NO: 8, the HCDR2 comprises the amino acid sequence as set forth in SEQ ID NO: 9, and the HCDR3 comprises the amino acid sequence as set forth in SEQ ID NO: 10, the LCDR1 comprises the amino acid sequence as set forth in SEQ ID NO: 11, the LCDR2 comprises the amino acid sequence as set forth in SEQ ID NO: 12, and the LCDR3 comprises the amino acid sequence as set forth in SEQ ID NO: 13; or
wherein the HCDR1 comprises the amino acid sequence as set forth in SEQ ID NO: 89, the HCDR2 comprises the amino acid sequence as set forth in SEQ ID NO: 90, and the HCDR3 comprises the amino acid sequence as set forth in SEQ ID NO: 91, the LCDR1 comprises the amino acid sequence as set forth in SEQ ID NO: 92, the LCDR2 comprises the amino acid sequence as set forth in SEQ ID NO: 93, and the LCDR3 comprises the amino acid sequence as set forth in SEQ ID NO: 94; or
wherein the HCDR1 comprises the amino acid sequence as set forth in SEQ ID NO: 97, the HCDR2 comprises the amino acid sequence as set forth in SEQ ID NO: 98, and the HCDR3 comprises the amino acid sequence as set forth in SEQ ID NO: 99, the LCDR1 comprises the amino acid sequence as set forth in SEQ ID NO: 100, the LCDR2 comprises the amino acid sequence as set forth in SEQ ID NO: 101, and the LCDR3 comprises the amino acid sequence as set forth in SEQ ID NO: 102.

Embodiment 27: The immunoconjugate or a pharmaceutically acceptable salt or a solvate thereof of any one of embodiments 1-26, comprising VL, wherein the VL comprises the amino acid sequence as set forth in SEQ ID NO: 23, SEQ ID NO: 68, SEQ ID NO: 46, SEQ ID NO: 69, SEQ ID NO: 96 or SEQ ID NO: 104.

Embodiment 28: The immunoconjugate or a pharmaceutically acceptable salt or a solvate thereof of any one of embodiments 1-27, comprising VH and VL, wherein the VH comprises the amino acid sequence as set forth in SEQ ID NO: 18, and the VL comprises the amino acid sequence as set forth in SEQ ID NO: 23; or
wherein the VH comprises the amino acid sequence as set forth in SEQ ID NO: 41, and the VL comprises the amino acid sequence as set forth in SEQ ID NO: 46; or
wherein the VH comprises the amino acid sequence as set forth in SEQ ID NO: 66, and the VL comprises the amino acid sequence as set forth in SEQ ID NO: 68; or
wherein the VH comprises the amino acid sequence as set forth in SEQ ID NO: 67, and the VL comprises the amino acid sequence as set forth in SEQ ID NO: 69; or
wherein the VH comprises the amino acid sequence as set forth in SEQ ID NO: 95, and the VL comprises the amino acid sequence as set forth in SEQ ID NO: 96; or
wherein the VH comprises the amino acid sequence as set forth in SEQ ID NO: 103, and the VL comprises the amino acid sequence as set forth in SEQ ID NO: 104.

Embodiment 29: The immunoconjugate or a pharmaceutically acceptable salt or a solvate thereof of any one of embodiments 1-28, comprising an antibody light chain constant region.

Embodiment 30: The immunoconjugate or a pharmaceutically acceptable salt or a solvate thereof of any one of embodiments 1-29, wherein the antibody light chain constant region comprises the amino acid sequence as set forth in SEQ ID NO: 71.

Embodiment 31: The immunoconjugate or a pharmaceutically acceptable salt or a solvate thereof of any one of embodiments 1-30, comprising an antibody light chain, wherein the antibody light chain comprises the amino acid sequence as set forth in SEQ ID NO: 73, SEQ ID NO: 86, SEQ ID NO: 81 or SEQ ID NO: 88.

Embodiment 32: The immunoconjugate or a pharmaceutically acceptable salt or a solvate thereof of any one of embodiments 1-31, comprising an antibody heavy chain and an antibody light chain, wherein the antibody heavy chain comprises the amino acid sequence as set forth in SEQ ID NO: 72, and the antibody light chain comprises the amino acid sequence as set forth in SEQ ID NO: 73; or
wherein the antibody heavy chain comprises the amino acid sequence as set forth in SEQ ID NO: 85, and the antibody light chain comprises the amino acid sequence as set forth in SEQ ID NO: 86; or
wherein the antibody heavy chain comprises the amino acid sequence as set forth in SEQ ID NO: 80, and the antibody light chain comprises the amino acid sequence as set forth in SEQ ID NO: 81; or
wherein the antibody heavy chain comprises the amino acid sequence as set forth in SEQ ID NO: 87, and the antibody light chain comprises the amino acid sequence as set forth in SEQ ID NO: 88.

Embodiment 33: The immunoconjugate or a pharmaceutically acceptable salt or a solvate thereof of any one of embodiments 1-32, further comprising an active moiety conjugated to the antibody or the antigen binding fragment thereof.

Embodiment 34: The immunoconjugate or a pharmaceutically acceptable salt or a solvate thereof of embodiment 33, wherein the active moiety comprises a drug moiety and/or a label.

Embodiment 35: The immunoconjugate or a pharmaceutically acceptable salt or a solvate thereof of embodiment 34, wherein the drug moiety is selected from the group consisting of a cytotoxic agent, a cytokine, a nucleic acid, a nucleic acid-associated molecule, a radionuclide, a chemokine, an immuno(co)-stimulatory molecule, an immunosuppressive molecule, a death ligand, an apoptosis-inducing protein, a kinase, a prodrug-converting enzyme, a RNase, an agonistic antibody or antibody fragment, an antagonistic antibody or antibody fragment, a growth factor, a hormone, a coagulation factor, a fibrinolytic protein, peptides mimicking these, and fragments, fusion proteins and derivatives thereof.

Embodiment 36: The immunoconjugate or a pharmaceutically acceptable salt or a solvate thereof of embodiment 34, wherein the label is selected from the group consisting of a radiolabel, a fluorophore, a chromophore, an imaging agent, and a metal ion.

Embodiment 37: The immunoconjugate or a pharmaceutically acceptable salt or a solvate thereof of any one of embodiments 35-36, wherein the cytotoxic agent comprises a microtubule disrupting drug and/or a DNA damaging agent.

Embodiment 38: The immunocomplex or a pharmaceutically acceptable salt or a solvate thereof of any one of embodiments 35-37, wherein the cytotoxic agent comprises a tubulin inhibitor and/or a topoisomerase inhibitor.

Embodiment 39: The immunoconjugate or a pharmaceutically acceptable salt or a solvate thereof of any one of embodiments 35-38, wherein the cytotoxic agent comprises a topoisomerase I inhibitor.

Embodiment 40: The immunoconjugate or a pharmaceutically acceptable salt or a solvate thereof of any one of embodiments 35-39, wherein the cytotoxic agent comprises camptothecin or a derivative thereof.

Embodiment 41: The immunoconjugate or a pharmaceutically acceptable salt or a solvate thereof of any one of embodiments 35-40, wherein the cytotoxic agent comprises the following structure of formula II:

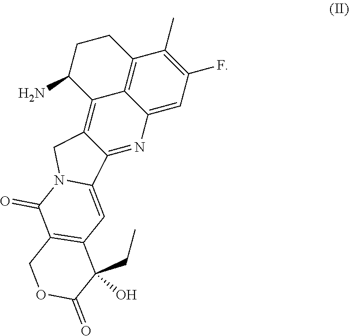 [SMILES: CC[C@@]1(O)C(=O)OCc2c1cc1n(c2=O)Cc2c-1nc1cc(F)c(C)c3c1c2[C@@H](N)CC3]

Embodiment 42: The immunoconjugate or a pharmaceutically acceptable salt or a solvate thereof of any one of embodiments 1-41, comprising an antibody drug conjugate (ADC) of formula (I):

Ab−(L−(D)ₘ)ₙ, (I)
wherein Ab is an antibody or antigen binding fragment thereof of any one of embodiments 1-41;
L is a linker;
D is a drug moiety;
m is an integer from 1 to 8; and
n is any number from 1 to 10.

Embodiment 43: The immunoconjugate or a pharmaceutically acceptable salt or a solvate thereof of embodiment 42, wherein the L is selected from: cleavable linker and a non-cleavable linker.

Embodiment 44: The immunoconjugate or a pharmaceutically acceptable salt or a solvate thereof of any one of embodiments 42-43, wherein the L comprises a cleavable peptide.

Embodiment 45: The immunoconjugate or a pharmaceutically acceptable salt or a solvate thereof of embodiment 44, wherein the cleavable peptide is cleavable by an enzyme.

Embodiment 46: The immunoconjugate of embodiment 45, wherein the enzyme comprises Cathepsin B.

Embodiment 47: The immunoconjugate or a pharmaceutically acceptable salt or a solvate thereof of any one of embodiments 44-46, wherein the cleavable peptide or L comprises an amino acid unit.

Embodiment 48: The immunoconjugate or a pharmaceutically acceptable salt or a solvate thereof of embodiment 47, wherein the amino acid unit comprises a dipeptide, tripeptide, tetrapeptide or pentapeptide.

Embodiment 49: The immunoconjugate or a pharmaceutically acceptable salt or a solvate thereof of any one of embodiments 47-48, wherein the amino acid unit is selected from: Val-Cit, Val-Ala, Glu-Val-Cit, Ala-Ala-Asn, Gly-Val-Cit, Gly-Gly-Gly and Gly-Gly-Phe-Gly.

Embodiment 50: The immunoconjugate or a pharmaceutically acceptable salt or a solvate thereof of any one of embodiments 42-49, wherein the L comprises a spacer.

Embodiment 51: The immunoconjugate or a pharmaceutically acceptable salt or a solvate thereof of embodiment 50, wherein the spacer comprises self-immolative spacers.

Embodiment 52: The immunoconjugate of embodiment 51, wherein the self-immolative spacer comprises p-aminobenzoxy carbonyl (PABC) or p-aminobenzyl (PAB).

Embodiment 53: The immunoconjugate of any one of embodiments 44-52, wherein the cleavable peptide is directly spliced to the spacer.

Embodiment 54: The immunoconjugate or a pharmaceutically acceptable salt or a solvate thereof of any one of embodiments 42-53, wherein the L comprises: -Val-Cit-PABC-, -Val-Ala-PABC-, -Glu-Val-Cit-PABC-, -Ala-Ala-Asn-PABC-, -Gly-Val-Cit-PABC-, -Gly-Gly-Gly-PABC-, -Gly-Gly-Phe-Gly-PABC-, -Val-Cit-PAB-, -Val-Ala-PAB-, -Glu-Val-Cit-PAB-, -Ala-Ala-Asn-PAB-, -Gly-Val-Cit-PAB-, -Gly-Gly-Gly-PAB- or -Gly-Gly-Phe-Gly-PAB-.

Embodiment 55: The immunoconjugate or a pharmaceutically acceptable salt or a solvate thereof of any one of embodiments 42-54, wherein the spacer comprises the structure shown in —NH—(CH₂)n¹-La-Lb-Lc-, where La denotes —O— or a single bond; Lb denotes —CR²(—CR³)— or a single bond, where R² and R³ each independently denote C₁-C₆ alkyl, —(CH₂)n^{a}-NH₂, —(CH₂)n^{b}-COOH or —(CH₂)n^{c}-OH, n¹ denotes an integer from 0 to 6, n^{a}, n^{b} and n^{c} each independently denote an integer from 1 to 4, but R² and R³ are not the same when n^{a} is 0, and Lc denotes —C(═O)—.

Embodiment 56: The immunoconjugate or a pharmaceutically acceptable salt or a solvate thereof of embodiment 55, wherein the spacer comprises —NH—(CH₂)3-C(═O)—, —NH—CH₂—O—CH₂—C(═O)— or —NH—(CH₂)₂—O—CH₂—C(═O)—.

Embodiment 57: The immunoconjugate or a pharmaceutically acceptable salt or a solvate thereof of any one of embodiments 42-56, wherein the L comprises the structure shown in -L₁-L₂-L₃-, where L₁ denotes -(succinimidyl-3-yl-N)—(CH₂)n²-C(═O)—, —CH₂—C(═O)—NH—(CH₂)n³-C(═O)— or —C(═O)—(CH₂)n⁴-C(═O)—, where n² denotes an integer from 2 to 8, n³ denotes an integer from 1 to 8, and n⁴ denotes an integer from 1 to 8; L₂ denotes amino acid unit; L₃ denotes a self-degradable spacer.

Embodiment 58: The immunoconjugate or a pharmaceutically acceptable salt or a solvate thereof of any one of embodiments 42-57, wherein the L is selected from:
-(succinimidyl-3-yl-N)—CH₂CH₂—C(═O)-GGFG-PABC-;
-(succinimidyl-3-yl-N)—CH₂CH₂CH₂CH₂CH₂—C(═O)-GGFG-PABC-;
-(succinimidyl-3-yl-N)—CH₂CH₂CH₂CH₂CH₂—C(═O)-GGFG-NH-PABC-;
-(succinimidyl-3-yl-N)—CH₂CH₂—C(═O)—NH—CH₂CH₂O—CH₂CH₂O—CH₂CH₂—C(═O)-GGFG-PABC-;
-(succinimidyl-3-yl-N)—CH₂CH₂—C(═O)—NH—CH₂CH₂O—CH₂CH₂O—CH₂CH₂O—CH₂CH₂O—CH₂CH₂—C(═O)-GGFG-PABC-;
—CH₂—C(═O)—NH—CH₂CH₂—C(═O)-GGFG-PABC-;
—C(═O)—CH₂CH₂CH₂CH₂CH₂CH₂—C(═O)-GGFG-PABC-;
-(succinimidyl-3-yl-N)—CH₂CH₂—C(═O)-GGFG-NH—CH₂CH₂—C(═O)—;
-(succinimidyl-3-yl-N)—CH₂CH₂—C(═O)-GGFG-NH—CH₂CH₂CH₂—C(═O)—;
-(succinimidyl-3-yl-N)—CH₂CH₂CH₂CH₂CH₂—C(═O)-GGFG-NH—CH₂CH₂—C(═O)—;
-(succinimidyl-3-yl-N)—CH₂CH₂CH₂CH₂CH₂—C(═O)-GGFG-NH—CH₂CH₂CH₂—C(═O)—;
-(succinimidyl-3-yl-N)—CH₂CH₂CH₂CH₂CH₂—C(═O)-GGFG-NH—CH₂CH₂CH₂CH₂CH₂—C(═O)—;
-(succinimidyl-3-yl-N)—CH₂CH₂CH₂CH₂CH₂—C(═O)-GGFG-NH—CH₂—O—CH₂—C(═O)—;
-(succinimidyl-3-yl-N)—CH₂CH₂CH₂CH₂CH₂—C(═O)-GGFG-NH—CH₂CH₂—O—CH₂—C(═O)—;
-(succinimidyl-3-yl-N)—CH₂CH₂—C(═O)—NH—CH₂CH₂O—CH₂CH₂O—CH₂CH₂—C(═O)-GGFG-NH—CH₂CH₂CH₂—C(═O)—;
-(succinimidyl-3-yl-N)—CH₂CH₂—C(═O)—NH—CH₂CH₂O—CH₂CH₂O—CH₂CH₂—C(═O)-GGFG-NH—CH₂CH₂—C(═O)—;
-(succinimidyl-3-yl-N)—CH₂CH₂—C(═O)—NH—CH₂CH₂O—CH₂CH₂O—CH₂CH₂O—CH₂CH₂O—CH₂CH₂—C(═O)-GGFG-NH—CH₂CH₂CH₂—C(═O)—;
-(succinimidyl-3-yl-N)—CH₂CH₂—C(═O)—NH—CH₂CH₂O—CH₂CH₂O—CH₂CH₂O—CH₂CH₂O—CH₂CH₂—C(═O)-GGFG-NH—CH₂CH₂—C(═O)—;
—CH₂—C(═O)—NH—CH₂CH₂—C(═O)-GGFG-NH—CH₂CH₂CH₂—C(═O)—;
—C(═O)—CH₂CH₂CH₂CH₂CH₂CH₂—C(═O)-GGFG-NH—CH₂CH₂CH₂—C(═O)—;
-(succinimidyl-3-yl-N)—CH₂CH₂—C(═O)—VA-PABC-;
-(succinimidyl-3-yl-N)—CH₂CH₂CH₂CH₂CH₂—C(═O)—VA-PABC-;
-(succinimidyl-3-yl-N)—CH₂CH₂CH₂CH₂CH₂—C(═O)—VA-NH-PABC-;
-(succinimidyl-3-yl-N)—CH₂CH₂—C(═O)—NH—CH₂CH₂O—CH₂CH₂O—CH₂CH₂—C(═O)—VA-PABC-;
-(succinimidyl-3-yl-N)—CH₂CH₂—C(═O)—NH—CH₂CH₂o-CH₂CH₂O—CH₂CH₂O—CH₂CH₂O—CH₂CH₂—C(═O)—VA-PABC-;
—CH₂—C(═O)—NH—CH₂CH₂—C(═O)—VA-PABC-;
—C(═O)—CH₂CH₂CH₂CH₂CH₂CH₂—C(═O)—VA-PABC-;
-(succinimidyl-3-yl-N)—CH₂CH₂—C(═O)—VA-NH—CH₂CH₂—C(═O)—;
-(succinimidyl-3-yl-N)—CH₂CH₂—C(═O)—VA-NH—CH₂CH₂CH₂—C(═O)—;
-(succinimidyl-3-yl-N)—CH₂CH₂CH₂CH₂CH₂—C(═O)—VA-NH—CH₂CH₂—C(═O)—;
-(succinimidyl-3-yl-N)—CH₂CH₂CH₂CH₂CH₂—C(═O)—VA-NH—CH₂CH₂CH₂—C(═O)—;
-(succinimidyl-3-yl-N)—CH₂CH₂CH₂CH₂CH₂—C(═O)—VA-NH—CH₂CH₂CH₂CH₂CH₂—C(═O)—;
-(succinimidyl-3-yl-N)—CH₂CH₂CH₂CH₂CH₂—C(═O)—VA-NH—CH₂—O—CH₂—C(═O)—;
-(succinimidyl-3-yl-N)—CH₂CH₂CH₂CH₂CH₂—C(═O)—VA-NH—CH₂CH₂—O—CH₂—C(═O)—;
-(succinimidyl-3-yl-N)—CH₂CH₂—C(═O)—NH—CH₂CH₂O—CH₂CH₂O—CH₂CH₂—C(═O)—VA-NH—CH₂CH₂CH₂—C(═O)—;
-(succinimidyl-3-yl-N)—CH₂CH₂—C(═O)—NH—CH₂CH₂O—CH₂CH₂O—CH₂CH₂—C(═O)—VA-NH—CH₂CH₂—C(═O)—;
-(succinimidyl-3-yl-N)—CH₂CH₂—C(═O)—NH—CH₂CH₂O—CH₂CH₂O—CH₂CH₂O—CH₂CH₂O—CH₂CH₂—C(═O)—VA-NH—CH₂CH₂CH₂—C(═O)—;
-(succinimidyl-3-yl-N)—CH₂CH₂—C(═O)—NH—CH₂CH₂O—CH₂CH₂O—CH₂CH₂O—CH₂CH₂O—CH₂CH₂—C(═O)—VA-NH—CH₂CH₂—C(═O)—;
—CH₂—C(═O)—NH—CH₂CH₂—C(═O)—VA-NH—CH₂CH₂CH₂—C(═O)—; and
—C(═O)—CH₂CH₂CH₂CH₂CH₂CH₂—C(═O)—VA-NH—CH₂CH₂CH₂—C(═O)—.

Embodiment 59: The immunoconjugate or a pharmaceutically acceptable salt or a solvate thereof of any one of embodiments 52-58, wherein the p-aminobenzoxy carbonyl (PABC) or p-aminobenzyl (PAB) comprises a polysarcosine (poly-N-methylglycine) residue.

Embodiment 60: The immunoconjugate or a pharmaceutically acceptable salt or a solvate thereof of any one of embodiments 42-59, wherein the L is selected from the following structure:

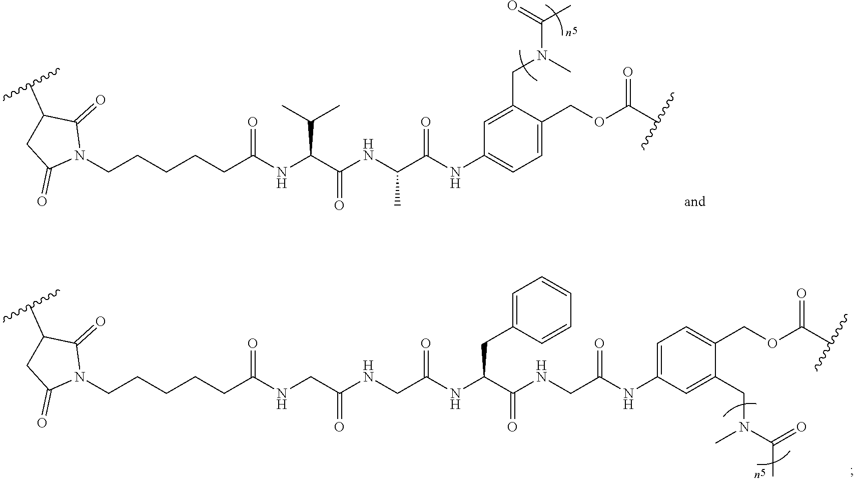

wherein n⁵ denotes an integer from 0 to 20.

Embodiment 61: The immunoconjugate or a pharmaceutically acceptable salt or a solvate thereof of embodiment 60, wherein n⁵ denotes an integer from 8 to 15.

Embodiment 62: The immunoconjugate or a pharmaceutically acceptable salt or a solvate thereof of any one of embodiments 42-61, wherein the L is selected from the following structure:

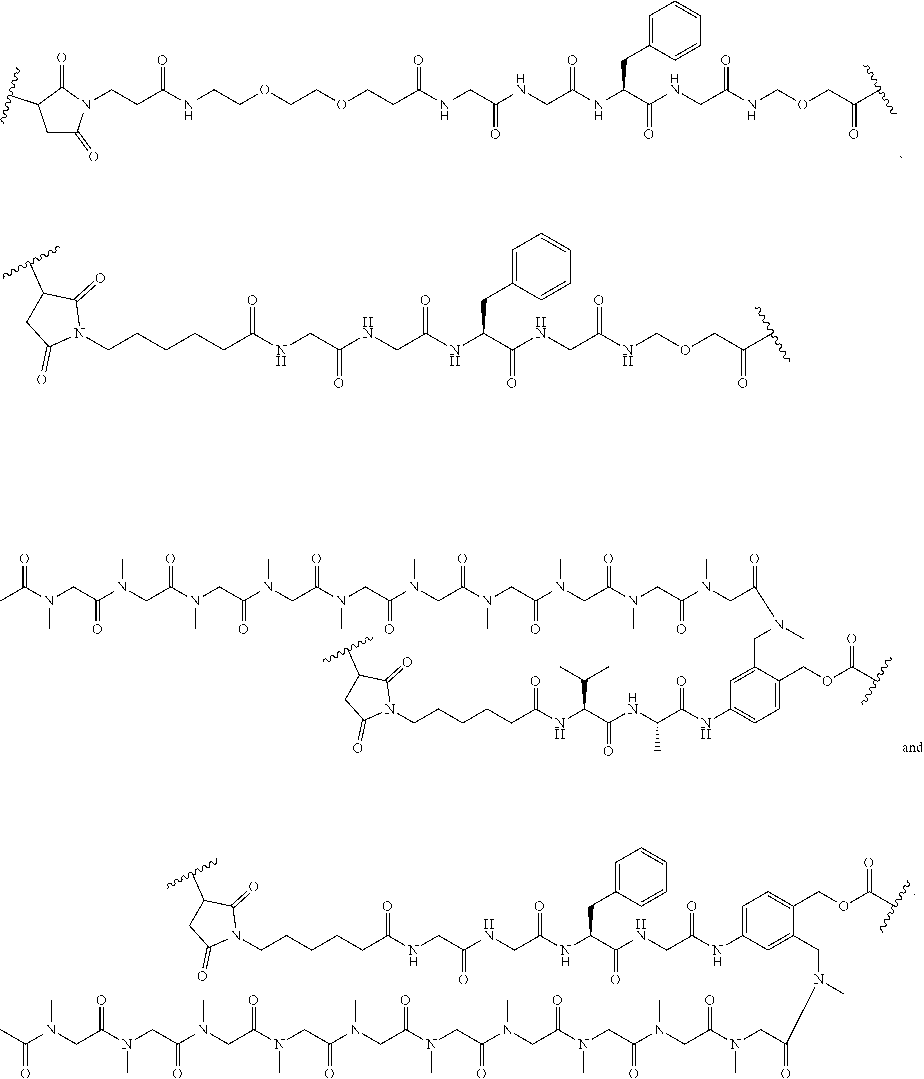

Embodiment 63: The immunoconjugate or a pharmaceutically acceptable salt or a solvate thereof of any one of embodiments 42-62, wherein the antibody drug conjugate is selected from the following structure:

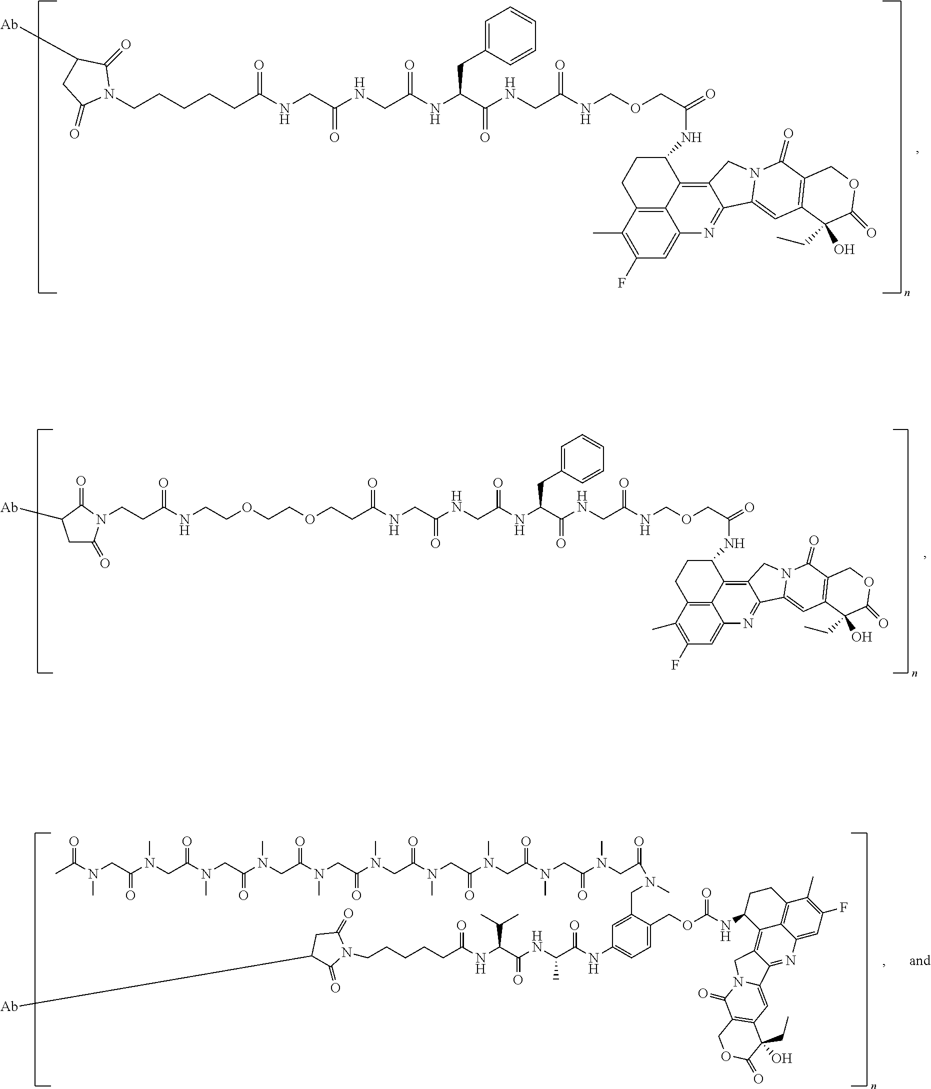

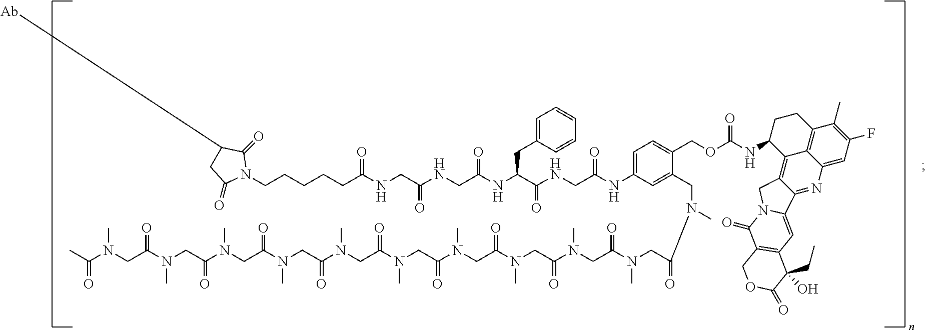

wherein n is any number from 1 to 10.

Embodiment 64: The immunoconjugate or a pharmaceutically acceptable salt or a solvate thereof of embodiment 63, wherein n is any number from 2 to 8.

Embodiment 65: A method of preparing an immunoconjugate or a pharmaceutically acceptable salt or a solvate thereof of any one of embodiments 1-64, comprising the step of reacting an antibody or antigen binding fragment thereof of any one of embodiments 1-32 with a drug-linker intermediate compound.

Embodiment 66: A pharmaceutical composition comprising the immunoconjugate or a pharmaceutically acceptable salt or a solvate thereof of any one of embodiments 1-64.

Embodiment 67: The pharmaceutical composition of embodiment 66, which further comprises a pharmaceutically acceptable carrier or excipient.

Embodiment 68: Use of the immunoconjugate or a pharmaceutically acceptable salt or a solvate thereof of any one of embodiments 1-64 or the pharmaceutical composition of any one of embodiments 66-67 in the manufacture of a medicament for treating tumors.

Embodiment 69: The use of embodiment 68, wherein the tumor is a tumor expressing CDH6.

Embodiment 70: The use of any one of embodiments 68-69, wherein the tumor comprises renal cell carcinoma, renal clear cell carcinoma, papillary renal cell carcinoma, ovarian cancer, ovarian serous adenocarcinoma, thyroid cancer, bile duct cancer, lung cancer, small-cell lung cancer, glioblastoma, mesothelioma, uterine cancer, pancreatic cancer, Wilms' tumor or neuroblastoma.

Embodiment 71: A method for treating a tumor, which comprises administering the immunoconjugate or a pharmaceutically acceptable salt or a solvate thereof of any one of embodiments 1-64 to a subject.

Embodiment 72: The method of embodiment 71, wherein the tumor is a tumor expressing CDH6.

Embodiment 73: The method of any one of embodiments 71-72, wherein the tumor comprises renal cell carcinoma, renal clear cell carcinoma, papillary renal cell carcinoma, ovarian cancer, ovarian serous adenocarcinoma, thyroid cancer, bile duct cancer, lung cancer, small-cell lung cancer, glioblastoma, mesothelioma, uterine cancer, pancreatic cancer, Wilms' tumor or neuroblastoma.

Embodiment 74: A method for treating a tumor, which comprises administering a pharmaceutical composition comprising at least one component selected from the immunoconjugate or a pharmaceutically acceptable salt or a solvate thereof of any one of embodiments 1-64, and at least one antitumor drug to a subject, simultaneously, separately or sequentially.

### Examples

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1 Preparation of Linker-Payload

### 1.1 Preparation of Compound LP-1

### Step 1: Synthesis of Intermediate 11-1

To the solution of compound 11-1A (Mc-Val-Ala-OH, purchased from MedChemExpress Shanghai, 2.4 g, 6.29 mmol) and 11-1B(3.18 g, 6.29 mmol) in the mixture of DCM:MeOH (v:v=2:1, 90 mL), EEDQ (1.86 g, 7.55 mmol) was added at room temperature. The reaction was stirred at rt for 24 hours, solvent was removed in vacuo, the crude residue was further purified by flash chromatography to give compound 11-1 (3.9 g, 71%). LC-MS (ESI, m/z): 868.49 (M+H).

### Step 2: Synthesis of Intermediate 11-2

Compound 11-1(2 g, 2.3 mmol) was dissolved in anhydrous THF (50 mL), hydrogen fluoride-pyridine (4.6 g, 46 mmol) was added under a argon atmosphere at 0° C., and then the reaction mixture was stirred at 0° C. for 2 hours. The reaction was quenched by addition of water, extracted with DCM, and the organic phase dried, and concentrated. The residue was purified by silica gel chromatography to afford compound 11-2 (1.1 g, 76%). LC-MS (ESI, m/z): 630.31 (M+H).

### Step 3: Synthesis of Intermediate 11-3

Compound 11-2(700 mg, 1.11 mmol) was dissolved in anhydrous DMF (4 mL), DIPEA (0.39 ml, 2.23 mmol) and 4,4′-dinitrodiphenyl carbonate (406 mg, 1.33 mmol) were added under a argon atmosphere at room temperature, and then the reaction mixture was stirred at ambient temperature for overnight. The solvent was removed by concentration, the product was precipitated from MTBE, the yellow solid was collected by filtration, washed with dietheyl ether, and dried to give compound 11-3, used in next step directly. LC-MS (ESI, m/z): 795.41 (M+H).

### Step 4: Synthesis of Intermediate 11-4

Compound 11-3 (300 mg, 0.44 mmol) was dissolved in anhydrous DMF (4 mL), dried pyridine (1 mL) was added, followed by exatecan mesylate (purchased from MedChemExpress Shanghai, 234 mg, 0.44 mmol) and HOBt (60 mg, 0.44 mmol). The reaction mixture was stirred at rt under argon overnight. The product was purified by pre-HPLC to afford intermediate 11-4 (230 mg, 48%). LC-MS (ESI, m/z): 1091.53 (M+H).

### Step 5: Synthesis of Intermediate 11-5

Compound 11-4 (200 mg, 0.183 mmol) was dissolved in 1 mL anhydrous DCM, 300 μL TFA was added at 0° C., the reaction mixture was stirred at rt for 30 min, the solvent was removed by concentration, to afford the intermediate 11-5 as TFA salt, which is used in next step without further purification. LC-MS (ESI, m/z): 991.47 (M+H).

### Step 6: Synthesis of Compound LP-1

Compound 11-5 (120 mg, 0.109 mmol) was dissolved in 1 mL anhydrous DMF, Ac-Sar10-COOH (84 mg, 0.109 mmol) was added, followed by HATU (50 mg, 0.130 mmol) and DIPEA (38 μL, 0.22 mmol), the reaction mixture was stirred at rt overnight, the solvent was removed by concentration, the crude product was purified by pre-HPLC to afford compound LP-1 (74 mg, 38%). LC-MS (ESI, m/z): 1743.85 (M+H).

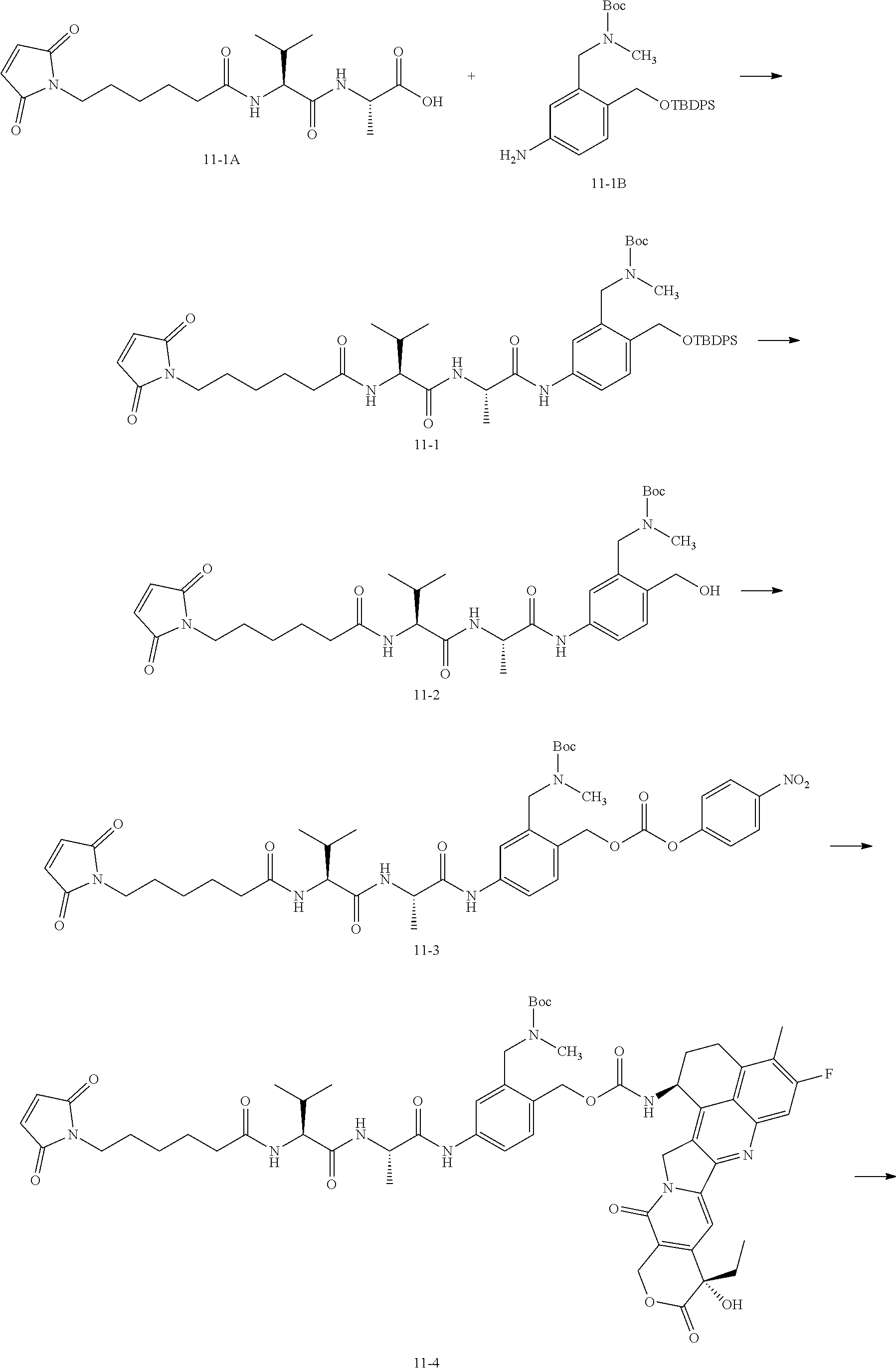

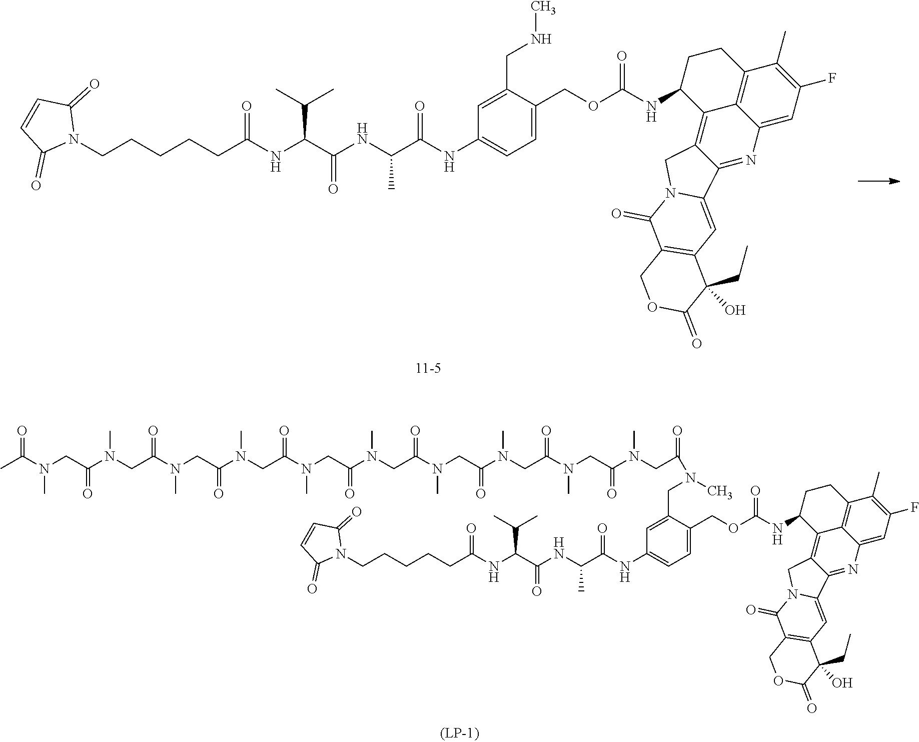 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)CCCCCN2C(=O)C=CC2=O)C(C)C)cc1CNC)CC3 ; CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)CCCCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O)CC3]

### 1.2 Preparation of Compound LP-2

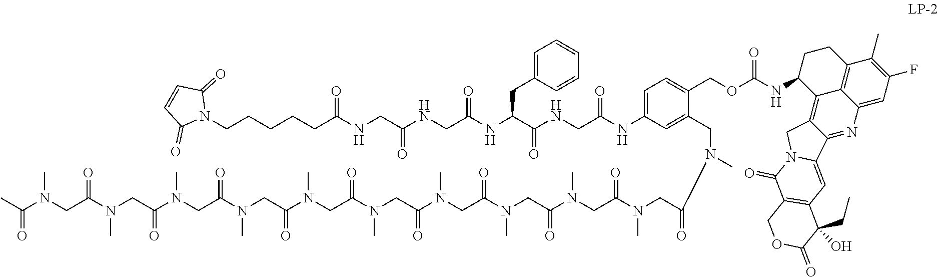 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)CNC(=O)[C@H](Cc2ccccc2)NC(=O)CNC(=O)CNC(=O)CCCCCN2C(=O)C=CC2=O)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O)CC3]

The synthesis of compound LP-2 is according to the procedure of compound LP-1, the starting material 11-1A was replaced by Mc-GGFG-OH (purchased from MedChemExpress Shanghai), compound LP-2 was afforded as beige amphorous solid. LC-MS (ESI, m/z): 1891.90 (M+H).

### Example 2 Production and Screening of Humanized Anti-CDH6 Antibody

The mouse Anti-CDH6 antibody CL069707 was humanized by CDR grafting (Proc. Natl. Acad. Sci. USA 86, 10029-10033(1989). The CDR regions of CL069707 were grafted to the most similar human germ line sequence. Some of the key residues that are important were back mutated based on, for example, the criteria given by Queen et al. (Proc. Natl. Acad. Sci. USA 86, 10029-10033(1989)).

Two humanized heavy chain variants were named CL069707-H1, CL069707-H2. The amino acid sequence of the CL069707-H1 heavy chain is shown in SEQ ID NO: 74. The amino acid sequence of the CL069707-H2 heavy chain is shown in SEQ ID NO: 75. Two humanized light chain variants were named CL069707-L1, CL069707-L2. The amino acid sequence of the CL069707-L1 light chain is shown in SEQ ID NO: 76. The amino acid sequence of the CL069707-L2 light chain is shown in SEQ ID NO: 77.

The DNA sequence encoding the full length CL069707-H1, CL069707-H2, CL069707-L1, CL069707-L2 amino acid sequences were synthesized and constructed into pCDNA3.1 expression vector. The antibodies CL069707-H1L1, CL069707-H1L2, CL069707-H2L1 and CL069707-H2L2 were produced in 293 cell by the combination of the heavy chain and the light chain.

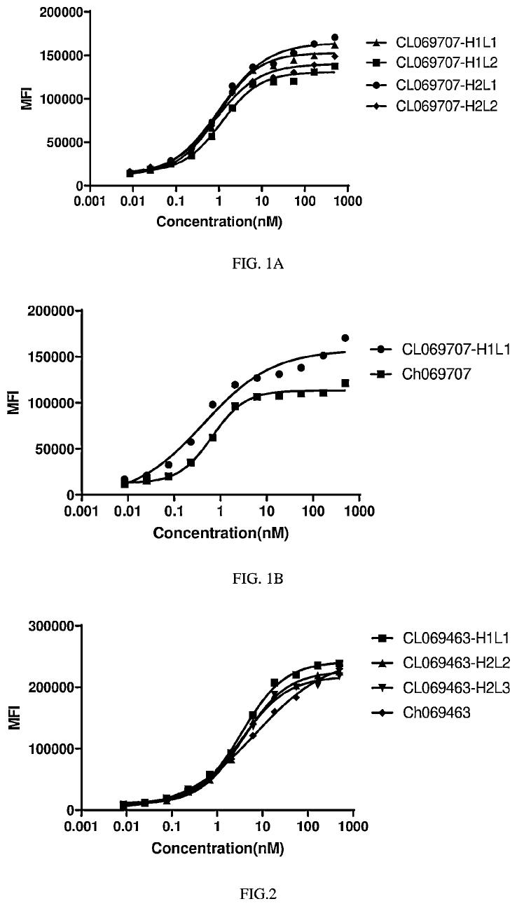

The binding EC50 of the humanized antibodies on tumor cell were tested as follow. Human ovarian cancer cell line OVCAR-3(purchased from National Collection of Authenticated Cell Cultures) cell culture was maintained in vitro as independent monolayer cultures at 37° C. in an atmosphere of 5% CO₂. The cells were harvested using trypsin-EDTA partial digestion followed by centrifugation at 1000 rpm for 5 minutes. The cell was resuspended in cold PBS and serial dilution of Ch069707 and CL069707-H1L1, CL069707-H1L2, CL069707-H2L1 and CL069707-H2L2 antibodies were added. Ch069707 antibody was a chimeric antibody. The variable regions were the same as those of CL069707, and the constant region was the same as human IgG1 constant regions (SEQ ID NO: 70 and SEQ ID NO: 71). The cell solutions were mixed, incubated at 4° C. and washed with cold PBS prior to addition of secondary antibody conjugates (for detection purposes). After incubation at 4° C., the cells were washed with PBS, and then subjected to flow cytometry (FACS) analysis. FIG. 1A shows that CL069707-H1L1, CL069707-H1L2, CL069707-H2L1 and CL069707-H2L2 had similar EC50 on OVCAR-3 cell. FIG. 1B shows that CL069707-H1L1 had slightly lower EC50 as that of Ch069707 on OVCAR-3 cell.

The mouse Anti-CDH6 antibody CL069463 was humanized by the same method. The results are shown in FIG. 2. CL069463-H1L1, CL069463-H2L2, CL069463-H2L3 had similar EC50 as Ch069463 on OVCAR-3 cell. Ch069463 antibody was a chimeric antibody. The variable regions were the same as those of CL069463, and the constant region was the same as human IgG1 constant regions (SEQ ID NO: 70 and SEQ ID NO: 71).

### Example 3 In Vitro Evaluation of Humanized CL069707-H1L1

### 3.1 Analysis of Binding of CL069707-H1L1 on Human CDH6, CDH9 and CDH10

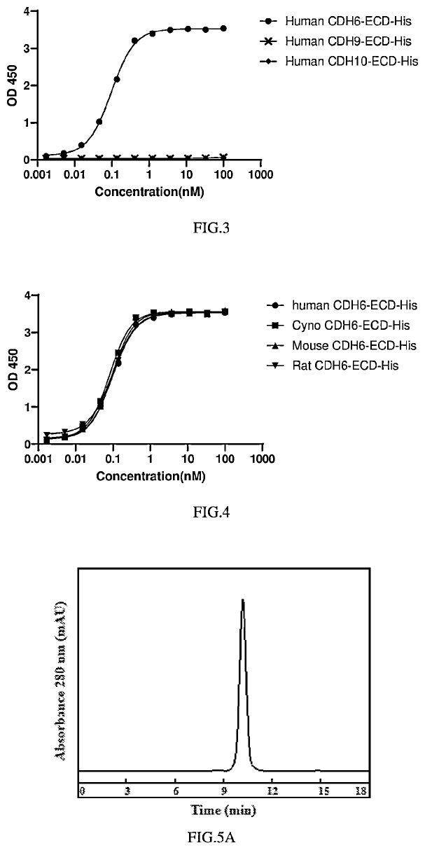

CDH9 and CDH1RO are the most closely related family members to CDH6. Binding of CL069707-H1L1 on human CDH6, CDH9 and CDH10 ECD domains were analyzed by ELISA Binding Assay. Human CDH6-EC6D-His, CDH9-ECD-His and CDH10-ECD-His antigens were obtained from Acro Biosystems, Inc. 100 μL of 1 μg/mL of the antigens diluted in PBS were added to 96 well plates and incubated in 4° C. overnight. The plates were washed 3 times with 300 μL of washing buffer. Then 100 μL/well of 1% BSA blocking reagent were added to the plates, and incubated at 37° C. for 1 h then the blocking reagent were discarded. A 3-fold serial dilution of CL069707-H1L1 was added to the plates and then incubated at 37° C. for 1 h. Then the plates were washed 3 times with 300 μL of washing buffer. 100 μL/well of HRP-conjugated anti-Human antibody diluted in 1% BSA were added to the plates and then incubated at 37° C. for 1 h. The plates were washed 3 times with 300 μL of washing buffer. 100 μL/well of TMB substrate were added to the plates and incubated for about 10 min in room temperature. Then the reaction was stopped with 100 μL/well of stopping solution. OD450 was read with a plate reader and the data were processed. The results are shown in FIG. 3. CL069707-H1L1 binds to human CDH6-ECD-His with a EC50 of 0.1 nM, CL069707-H1L1 does not bind to CDH9-ECD-His or CDH10-ECD-His protein.

### 3.2 Evaluation of ELISA Binding EC50 of CL069707-H1L1 on Human, Cyno Monkey, Mouse and Rat CDH6 Antigen

Binding of CL069707-H1L1 on human, cyno monkey, mouse or rat CDH6 antigen were analyzed by ELISA. Human, cyno monkey, mouse and rat CDH6-ECD-His antigens were obtained from Acro Biosystems, Inc. ELISA were done with the same methods as in example 3.1. The results were shown in FIG. 4. CL069707-H1L1 binds to human, cyno monkey, mouse and rat CDH6-ECD-His antigens with similar EC50.

### 3.3 Evaluation of Binding Constants of CL069707-H1L1 and Ch069707 on Human and Cyno Monkey CDH6 Antigen

Binding affinities of CL069707-H1L1 and Ch069707 were measured using the Octet® system based on Bio-Layer Interferometry (BLI) technique. Antibodies were loaded to AHC (Anti-hIgG Fc Capture) at 100 nM concentration. Then 3.13 nM, 6.25 nM, 12.5 nM, 25 nM, 50 nM and 100 nM of human CDH6-ECD-His or cyno monkey CDH6-ECD-His antigens were incubated with the antibody-conjugated biosensor tip to measure the association between antigen and antibody. From the six different binding curves obtained from each antigen concentration, binding constants (KD) were calculated based on the 1:1 binding model. The results are shown in Table 2. CL069707-H1L1 and Ch069707 binds to human CDH6-ECD-His antigen with similar affinity. CL069707-H1L1 binds to human CDH6-ECD-His and cyno monkey CDH6-ECD-His antigens with similar affinity.

TABLE 2

| Antibody | Antigen | KD(M) |
|---|---|---|
| Ch069707 | Cyno CDH6-ECD-His | 2.19E−09 |
| Ch069707 | Human CDH6-ECD-His | 2.28E−9 |
| CL069707-H1L1 | Cyno CDH6-ECD-His | 2.69E−9 |
| CL069707-H1L1 | Human CDH6-ECD-His | 3.08E−9 |

### Example 4 Production of Antibody-Drug Conjugate

### General Method for Producing Antibody-Drug Conjugate

The antibody that can be used in the antibody-drug conjugate of the present invention is not particularly limited as long as it is an anti-CDH6 antibody having internalization activity or a functional fragment of the antibody.

Next, a typical method for producing the antibody-drug conjugate of the present invention will be described. It is to be noted that, in the description below, “compound No.” shown in each reaction scheme is used to represent a compound. Specifically, each compound is referred to as a “compound of formula (1)”, “compound (1)” or the like. The same holds true for the other compound Nos.

The antibody-drug conjugate represented by formula (1) given below in which the anti-CDH6 antibody is connected to the linker structure via a thioether can be produced by reacting an antibody having a sulfhydryl group converted from a disulfide bond by the reduction of the anti-CDH6 antibody, with the compound (2). This antibody-drug conjugate can be produced by the following method, for example.

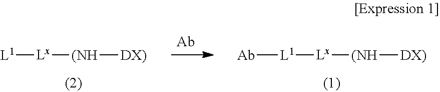

Wherein Ab represents an antibody with a sulfhydryl group, wherein L¹ has a structure represented by -(Succinimid-3-yl-N)—, and L^{1′} represents a maleimidyl group represented by the following formula.

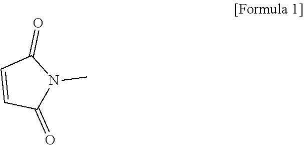

Wherein (NH-DX) has a structure represented by the following formula.

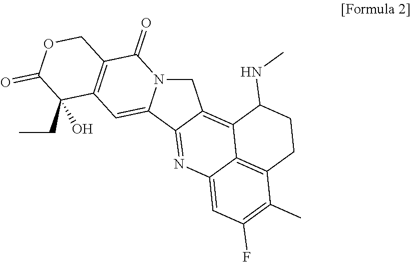 [SMILES: CC[C@@]1(O)C(=O)OCc2c1cc1n(c2=O)Cc2c-1nc1cc(F)c(C)c3c1c2C(NC)CC3]

-L¹-L^{x}- has a structure represented by any of the following formulas:
-(succinimidyl-3-yl-N)—CH2CH2-C(═O)-GGFG-PABC-;
-(succinimidyl-3-yl-N)—CH2CH2CH2CH2CH2-C(═O)-GGFG-PABC-;
-(succinimidyl-3-yl-N)—CH2CH2CH2CH2CH2-C(═O)-GGFG-NH-PABC-;
-(succinimidyl-3-yl-N)—CH2CH2-C(═O)—NH—CH2CH2O—CH2CH2O—CH2CH2-C(═O)-GGFG-PABC-;
-(succinimidyl-3-yl-N)—CH2CH2-C(═O)—NH—CH2CH2O—CH2CH2O—CH2CH2O—CH2CH2O—CH2CH2-C(═O)-GGFG-PABC-;
—CH₂—C(═O)—NH—CH2CH2-C(═O)-GGFG-PABC-;
—C(═O)—CH2CH2CH2CH2CH2CH2-C(═O)-GGFG-PABC-;
-(succinimidyl-3-yl-N)—CH2CH2-C(═O)-GGFG-NH—CH2CH2-C(═O)—;
-(succinimidyl-3-yl-N)—CH2CH2-C(═O)-GGFG-NH—CH2CH2CH2-C(═O)—;
-(succinimidyl-3-yl-N)—CH2CH2CH2CH2CH2-C(═O)-GGFG-NH—CH2CH2-C(═O)—;
-(succinimidyl-3-yl-N)—CH2CH2CH2CH2CH2-C(═O)-GGFG-NH—CH2CH2CH2-C(═O)—;
-(succinimidyl-3-yl-N)—CH2CH2CH2CH2CH2-C(═O)-GGFG-NH—CH2CH2CH2CH2CH2-C(═O)—;
-(succinimidyl-3-yl-N)—CH2CH2CH2CH2CH2-C(═O)-GGFG-NH—CH2-CH2-C(═O)—;
-(succinimidyl-3-yl-N)—CH2CH2CH2CH2CH2-C(═O)-GGFG-NH—CH2CH2-O—CH2-C(═O)—;
-(succinimidyl-3-yl-N)—CH2CH2-C(═O)—NH—CH2CH2O—CH2CH2O—CH2CH2-C(═O)-GGFG-NH—CH2CH2CH2-C(═O)—;
-(succinimidyl-3-yl-N)—CH2CH2-C(═O)—NH—CH2CH2O—CH2CH2O—CH2CH2-C(═O)-GGFG-NH—CH2CH2-C(═O)—;
-(succinimidyl-3-yl-N)—CH2CH2-C(═O)—NH—CH2CH2O—CH2CH2O—CH2CH2O—CH2CH2O—CH2CH2-C(═O)-GGFG-NH—CH2CH2CH2-C(═O)—;
-(succinimidyl-3-yl-N)—CH2CH2-C(═O)—NH—CH2CH2O—CH2CH2O—CH2CH2O—CH2CH2O—CH2CH2-C(═O)-GGFG-NH—CH2CH2-C(═O)—;
—CH2-C(═O)—NH—CH2CH2-C(═O)-GGFG-NH—CH2CH2CH2-C(═O)—;
—C(═O)—CH2CH2CH2CH2CH2CH2-C(═O)-GGFG-NH—CH2CH2CH2-C(═O)—;
-(succinimidyl-3-yl-N)—CH2CH2-C(═O)—VA-PABC-;
-(succinimidyl-3-yl-N)—CH2CH2CH2CH2CH2-C(═O)—VA-PABC-;
-(succinimidyl-3-yl-N)—CH2CH2CH2CH2CH2-C(═O)—VA-NH-PABC-;
-(succinimidyl-3-yl-N)—CH2CH2-C(═O)—NH—CH2CH2O—CH2CH2O—CH2CH2-C(═O)—VA-PABC-;
-(succinimidyl-3-yl-N)—CH2CH2-C(═O)—NH—CH2CH2o-CH2CH2O—CH2CH2O—CH2CH2O—CH2CH2-C(═O)—VA-PABC-;
—CH2-C(═O)—NH—CH2CH2-C(═O)—VA-PABC-;
—C(═O)—CH2CH2CH2CH2CH2CH2-C(═O)—VA-PABC-;
-(succinimidyl-3-yl-N)—CH2CH2-C(═O)—VA-NH—CH2CH2-C(═O)—;
-(succinimidyl-3-yl-N)—CH2CH2-C(═O)—VA-NH—CH2CH2CH2-C(═O)—;
-(succinimidyl-3-yl-N)—CH2CH2CH2CH2CH2-C(═O)—VA-NH—CH2CH2-C(═O)—;
-(succinimidyl-3-yl-N)—CH2CH2CH2CH2CH2-C(═O)—VA-NH—CH2CH2CH2-C(═O)—;
-(succinimidyl-3-yl-N)—CH2CH2CH2CH2CH2-C(═O)—VA-NH—CH2CH2CH2CH2CH2-C(═O)—;
-(succinimidyl-3-yl-N)—CH2CH2CH2CH2CH2-C(═O)—VA-NH—CH2-O—CH2-C(═O)—;
-(succinimidyl-3-yl-N)—CH2CH2CH2CH2CH2-C(═O)—VA-NH—CH2CH2-O—CH2-C(═O)—;
-(succinimidyl-3-yl-N)—CH2CH2-C(═O)—NH—CH2CH2O—CH2CH2O—CH2CH2-C(═O)—VA-NH—CH2CH2CH2-C(═O)—;
-(succinimidyl-3-yl-N)—CH2CH2-C(═O)—NH—CH2CH2O—CH2CH2O—CH2CH2-C(═O)—VA-NH—CH2CH2-C(═O)—;
-(succinimidyl-3-yl-N)—CH2CH2-C(═O)—NH—CH2CH2O—CH2CH2O—CH2CH2O—CH2CH2O—CH2CH2-C(═O)—VA-NH—CH2CH2CH2-C(═O)—;
-(succinimidyl-3-yl-N)—CH2CH2-C(═O)—NH—CH2CH2O—CH2CH2O—CH2CH2O—CH2CH2O—CH2CH2-C(═O)—VA-NH—CH2CH2-C(═O)—;
—CH2-C(═O)—NH—CH2CH2-C(═O)—VA-NH—CH2CH2CH2-C(═O)—; and
—C(═O)—CH2CH2CH2CH2CH2CH2-C(═O)—VA-NH—CH2CH2CH2-C(═O)—.

In the above-described reaction scheme, the antibody-drug conjugate (1) can be understood as having a structure in which one structure moiety from the drug to the linker terminus is connected to one antibody. However, this description is given for the sake of convenience, and there are actually many cases in which a plurality of the aforementioned structure moieties is connected to one antibody molecule. The same holds true for the explanation of the production method described below.

The antibody having a sulfhydryl group can be obtained by a method well known to a person skilled in the art (Hermanson, G. T, Bioconjugate Techniques, pp. 56-136, pp. 456-493, Academic Press (1996)). Examples of the method can include, but are not limited to: Traut's reagent being reacted with the amino group of the antibody; N-succinimidyl S-acetylthioalkanoates being reacted with the amino group of the antibody followed by reaction with hydroxylamine; N-succinimidyl 3-(pyridyldithio)propionate being reacted with the antibody, followed by reaction with a reducing agent; the antibody being reacted with a reducing agent such as dithiothreitol, 2-mercaptoethanol or tris(2-carboxyethyl)phosphine hydrochloride (TCEP) to reduce the interchain disulfide bond in the antibody, so as to form a sulfhydryl group.

Specifically, an antibody with interchain disulfide bonds partially or completely reduced can be obtained by using 0.3 to 3 molar equivalents of TCEP as a reducing agent per interchain disulfide bond in the antibody, and reacting the reducing agent with the antibody in a buffer solution containing a chelating agent. Examples of the chelating agent can include ethylenediaminetetraacetic acid (EDTA) and diethylenetriaminepentaacetic acid (DTPA). The chelating agent can be used at a concentration of 1 mM to 20 mM. A solution of sodium phosphate, sodium borate, sodium acetate or the like can be used as the buffer solution.

It is to be noted that by carrying out an addition reaction of a sulfhydryl group to a drug-linker moiety, the drug-linker moiety can be conjugated by a thioether bond.

Then, using 2 to 20 molar equivalents of the compound (2) per antibody having a sulfhydryl group, the antibody-drug conjugate (1) in which 2 to 8 drug molecules are conjugated per antibody can be produced. Specifically, a solution containing the compound (2) dissolved therein may be added to a buffer solution containing the antibody having a sulfhydryl group for the reaction. In this context, a sodium acetate solution, sodium phosphate, sodium borate or the like can be used as the buffer solution. pH for the reaction is 5 to 9, and more preferably, the reaction may be performed near pH 7. An organic solvent such as dimethyl sulfoxide (DMSO), dimethylformamide (DMF), dimethylacetamide (DMA) or N-methyl-2-pyrrolidone (NMP) can be used as a solvent for dissolving the compound (2). The reaction may be performed by adding the solution containing the compound (2) dissolved in the organic solvent at 1 to 20% v/v to a buffer solution containing the antibody having a sulfhydryl group. The reaction temperature is 0 to 37° C., more preferably 10 to 25° C., and the reaction time is 0.5 to 2 hours. The reaction can be terminated by deactivating the reactivity of unreacted compound (2) with a thiol-containing reagent. The thiol-containing reagent is, for example, cysteine or N-acetyl-L-cysteine (NAC). More specifically, the reaction can be terminated by adding 1 to 2 molar equivalents of NAC to the compound (2) used, and incubating the obtained mixture at room temperature for 10 to 30 minutes.

### Identification of Antibody-Drug Conjugate

The produced antibody-drug conjugate (1) can be subjected to concentration, buffer exchange, purification, and measurement of antibody concentration and the average number of conjugated drug molecules per antibody molecule according to common procedures described below, to identify the antibody-drug conjugate (1).

### Common Procedure A: Concentration of Aqueous Solution of Antibody or Antibody-Drug Conjugate

To an Amicon Ultra (50,000 MWCO, Millipore Corporation) container, a solution of an antibody or an antibody-drug conjugate was added, and the solution of the antibody or the antibody-drug conjugate was concentrated by centrifugation (centrifugation at 2000 G to 3800 G for 5 to 20 minutes).

### Common Procedure B: Measurement of Antibody Concentration

Using a Microplate reader (Multiskan GO, Thermo Fisher Scientific Inc.), measurement of the antibody absorption was carried out according to the method defined by the manufacturer. In this respect, 280 nm absorption coefficient differing among antibodies (1.3 mLmg⁻¹ cm⁻¹ to 1.8 mLmg⁻¹ cm⁻¹) was used.

### Common Procedure C: Buffer Exchange for Antibody

Zeba™ Spin Desalting columns (5 mL, 40K MWCO, Thermo Scientific™) were equilibrated with a phosphate buffer (50 mM, pH 7.0) (referred to as PBS7.0/EDTA in the present description) containing sodium chloride (50 mM) and EDTA (2 mM) according to the method defined by the manufacturer. An aqueous solution of the antibody was applied in an amount of 2 mL per Zeba™ (5 mL) column, and thereafter, a fraction (about 2 mL) was collected by centrifuging the desalting column (centrifugation at 1000 G for 4 minutes). This fraction can be concentrated by common procedure A. After measurement of the concentration of the antibody using common procedure B, the antibody concentration was adjusted to more than 10 mg/mL using PBS7.0/EDTA.

### Common Procedure D: Purification of Antibody-Drug Conjugate

Zeba™ Spin Desalting columns (5 mL, 40K MWCO, Thermo Scientific™) were equilibrated with any available formulation buffer solution such as a histidine-acetate buffer (20 mM histidine, pH 5.5) containing sodium chloride (150 mM) or a phosphate buffer (50 mM, pH 7.0) containing sodium chloride (50 mM). An aqueous reaction solution of the antibody-drug conjugate (approximately 2 mL) was applied to the ZebaT^{M} column (5 mL), and thereafter, an antibody fraction (about 2 mL) was collected by centrifuging the desalting column (centrifugation at 1000 G for 4 minutes). A gel filtration purification process, in which the collected fraction was applied again to the Zeba™ desalting column, and elution was carried out with centrifugation, was repeated a total of 2 times to obtain the antibody-drug conjugate excluding non-conjugated linker-payload and low-molecular-weight compounds (tris(2-carboxyethyl) phosphine hydrochloride (TCEP), N-acetyl-L-cysteine (NAC), and dimethyl sulfoxide).

### Common Procedure E: Measurement of Antibody Concentration in Antibody-Drug Conjugate and Average Number of Conjugated Drug Molecules Per Antibody Molecule

The conjugated drug concentration in the antibody-drug conjugate can be calculated by measuring UV absorbance of an aqueous solution of the antibody-drug conjugate at two wavelengths of 280 nm and 370 nm, and thereafter performing the calculation shown below.

The total absorbance at any given wavelength is equal to the sum of the absorbance of all light-absorbing chemical species that are present in a system [additivity of absorbance]. Therefore, based on the hypothesis that the molar absorption coefficients of the antibody and the drug do not vary between before and after conjugation between the antibody and the drug, the antibody concentration and the drug concentration in the antibody-drug conjugate are represented by the following equations.

$${A}_{280} = {A}_{D , 280} + {A}_{A , 280} = {ε}_{D , 280} {C}_{D} + {ε}_{A , 280} {C}_{A} \qquad Equation ( 1 )$$

$${A}_{370} = {A}_{D , 370} + {A}_{A , 370} = {ε}_{D , 370} {C}_{D} + {ε}_{A , 370} {C}_{A} \qquad Equation ( 2 )$$

In this context, A₂₈₀ represents the absorbance of an aqueous solution of the antibody-drug conjugate at 280 nm, A₃₇₀ represents the absorbance of an aqueous solution of the antibody-drug conjugate at 370 nm, A_{A,280} represents the absorbance of the antibody at 280 nm, A_{A,370} represents the absorbance of the antibody at 370 nm, A_{D,280} represents the absorbance of a conjugate precursor at 280 nm, A_{D,370} represents the absorbance of a conjugate precursor at 370 nm, ε_{A,280} represents the molar absorption coefficient of the antibody at 280 nm, ε_{A,370} represents the molar absorption coefficient of the antibody at 370 nm, ε_{D,280} represents the molar absorption coefficient of a conjugate precursor at 280 nm, ε_{D,370} represents the molar absorption coefficient of a conjugate precursor at 370 nm, C_{A} represents the antibody concentration in the antibody-drug conjugate, and C_{D} represent the drug concentration in the antibody-drug conjugate.

In this context, with regard to ε_{A,280}, ε_{A,370}, ε_{D,280}, and ε_{D,370}, preliminarily prepared values (estimated values based on calculation or measurement values obtained by UV measurement of the compound) are used. For example, ε_{A,280} can be estimated from the amino acid sequence of the antibody by a known calculation method (Protein Science, 1995, vol. 4, 2411-2423). ε_{A,370} is generally zero. ε_{D,280} and ε_{D,370} can be obtained according to Lambert-Beer's law (Absorbance Molar concentration×Molar absorption coefficient×Cell path length) by measuring the absorbance of a solution in which the conjugate precursor used is dissolved at a certain molar concentration. C_{A} and C_{D} can be determined by measuring A₂₈₀ and A₃₇₀ of an aqueous solution of the antibody-drug conjugate, and then solving the simultaneous equations (1) and (2) by substitution of these values. Further, by dividing C_{D} by C_{A}, the average number of conjugated drug molecules per antibody can be determined.

### Common Procedure F: Measurement of Average Number of Conjugated Drug Molecules Per Antibody Molecule-(2)

The average number of conjugated drug molecules per antibody molecule in the antibody-drug conjugate can also be determined by liquid chromatograph-mass spectrometer (LC-MS) analysis using the following method, in addition to the aforementioned “Common procedure E”. Hereinafter, the method for measuring the average number of conjugated drug molecules by LC-MS when the antibody is conjugated to the linker-payload by a disulfide bond will be described. A person skilled in the art is capable of appropriately measuring the average number of conjugated drug molecules by LC-MS, depending on the connecting manner between the antibody and the linker-payload, with reference to this method.

### Preparation of Sample for LC-MS Analysis (Reduction of Antibody-Drug Conjugate)

An antibody-drug conjugate solution (approximately 5 mg/mL, 6 L) is mixed with an aqueous solution of dithiothreitol (DTT) (100 mM, 3 μL) and 21 μL water. By incubating the mixture at 37° C. for 30 minutes, the disulfide bond between the light chain and heavy chain of the antibody-drug conjugate is cleaved. The resulting sample is used in LC-MS analysis.

### HPLC Parameters

Column: Agilent PLRP-S, 1000 Å, 50×2.1 mm, 8 μm.
Detection wavelength: 280 nm
Band width: 4 nm
Column Oven Temperature: 80° C.
Autosampler thermostat: 5° C.
Flow Rate: 0.5 mL/min
Inject Volume: 5 μL
Mobile phase A: 0.05% TFA, H2O
Mobile phase B: 0.05% TFA, ACN
Gradient program (B %): 25%-34% (0 min-0.7 min), 34%-45% (0.7 min-5 min), 45%-90% (5 min-6 min), 90% (6 min-7 min), 90%-25% (7 min-7.10 min), 25% (7.10 min-10 min)

### MS Parameters

Gas Temp.: 350° C.
Drying Gas: 13 L/min
Nebulizer: 45 psig
V Cap: 5000 V
Fragmentor: 350 V
Mass Range: 500-8000 m/z
Acquisition Rate: 1 spectra/s

### Data Analysis

According to ESI scan gram, a light chain bound to drug molecule(s) (light chain bound to i drug molecule(s): Li) and a heavy chain bound to drug molecule(s) (heavy chain bound to i drug molecule(s): Hi) can be detected.

The peak area ratio (%) of each chain is calculated for the total of the corrected values of peak areas according to the following expression.

Peak area ratio of light chain bound to

$$i drug molecule ( s ) = 100 \% \times {A}_{Li} / ( {A}_{L 0} + {A}_{L 1} )$$

Peak area ratio of heavy chain bound to

$$i drug molecule ( s ) = 100 \% \times {A}_{Hi} / ( {A}_{H 0} + {A}_{H 1} + {A}_{H 2} + {A}_{H 3} )$$

The average number of conjugated drug molecules per antibody molecule in the antibody-drug conjugate is calculated according to the following expression.

Average number of conjugated drug molecules=(L0 peak area ratio×0+L1 peak area ratio×1+H0 peak area ratio×0+H1 peak area ratio×1+H2 peak area ratio×2+H3 peak area ratio×3)×2

### Common Procedure G: Measurement of Antibody Aggregation in Antibody-Drug Conjugate

The aggregation in the antibody-drug conjugate can be determined by size exclusion chromatography (SEC) in high-performance liquid chromatography (HPLC) analysis using the following method.
HPLC system: Agilent 1260 Infinity II HPLC system (Agilent Technologies, Inc.)
Detector: Ultraviolet absorption spectrometer (measurement wavelength: 280 nm)
SEC-Column: TOSOH TSKgel G3000SWXL (7.8×300 mm, 5 μm)
Mobile phase: 200 mmol/L KHPO₄, 150 mmol/L NaCl, 15% (v/v) isopropanol, pH7.0
Flow rate: 0.75 mL/min
Isocratic Operation: 18 min
Column temperature: room temperature
Sample injection: 50 μg

### Data Analysis

The SEC chromatogram of quality control (QC, CL069707-H1L1) was shown in FIG. 5A, it is confirmed that the molecular weight of the main QC product is about 150 kDa and the monomer retention time is around 9.5-10.5 min. Meanwhile, the retention time of aggregation is earlier than that of monomer.

### Common Procedure H: Comparison of Antibody Hydrophilicity in Antibody-Drug Conjugate

The Hydrophilicity of the antibody-drug conjugate can be determined by hydrophobic interaction chromatography (HIC) in high-performance liquid chromatography (HPLC) analysis using the following method.
HPLC system: Agilent 1260 Infinity II HPLC system (Agilent Technologies, Inc.)
Detector: Ultraviolet absorption spectrometer (measurement wavelength: 280 nm)
HIC-Column: TOSOH TSKgel Butyl-NPR (4.6 mm I.D.×3.5 cm, 2.5 μm)
Mobile phase A: 1.5 mol/L (NH₄)₂SO₄, 50 mmol/L KHPO₄, pH7.0
Mobile phase B: 50 mmol/L KHPO₄, 25% (v/v) isopropanol, pH7.0
Isocratic Operation: 25 min
Column temperature: room temperature
Gradient program (B %): 0%-25% (0 min-1 min), 25% (1 min-3 min), 25%-80% (3 min-13 min), 80% (13 min-17 min), 80%-0% (17 min-17.10 min), 0% (17.10 min-25 min)
Sample injection: 10 μL

Data Analysis: The HIC chromatogram of quality control (QC, CL069707-H1L1) was shown in FIG. 5B. Compared with non-conjugated antibody, antibody-drug conjugates exhibit higher hydrophobicity and thus have a larger retention time. It is confirmed that the shorter the retention time, the better of the sample's hydrophilicity

### 4.1: Production of CL069707-H1L1-Drug Conjugate

### 4.1.1 Production of Antibody-Drug Conjugate CL069707-H1L1-LP1

### Step 1: Antibody-Drug Conjugate

Reduction of antibody: CL069707-H1L1 was adjusted to 9 mg/mL with PBS7.0/EDTA by using common procedures B (using 1.423 mLmg⁻¹ cm⁻¹ as 280 nm absorption coefficient) and C described in production method. To this solution (1.099 mL), an aqueous solution of 5 mM TCEP (0.101 mL; 7.0 equivalents per antibody molecule). After confirming that the solution had a pH within 7.0±0.1, the interchain disulfide bond in the antibody was reduced by incubating the solution at 37° C. for 2 hours.

Conjugation between antibody and linker-payload: The above-described solution was incubated at 4° C. for 10 minutes. Subsequently, a 10 mM solution of LP1 in dimethylacetamide (0.108 mL; 15 equivalents per antibody molecule) was added thereto, and the obtained mixture was incubated at 22° C. for 30 minutes to conjugate the linker-payload to the antibody.

Purification: The above-described solution was purified by common procedure D described in production method to obtain 1.2 mL of a solution containing the title antibody-drug conjugate “CL069707-H1L1-LP1”.

Characterization: Using common procedure E (using _{εD,280}=6384 and _{εD,370}=16180), procedure F, procedure G and procedure H described in production method, the following characteristic values were obtained.

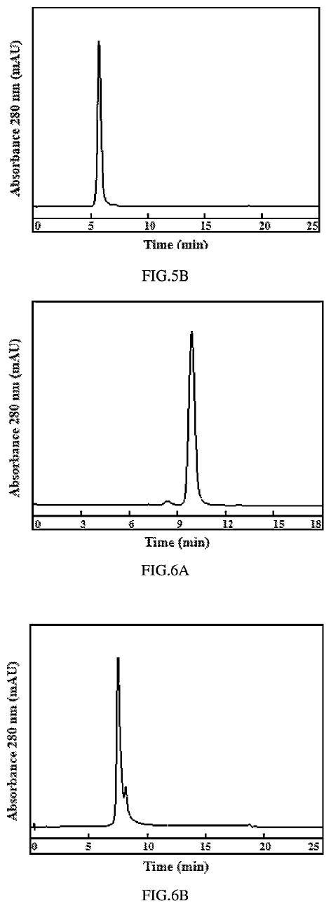

Antibody concentration: 7.82 mg/mL, average number of conjugated drug molecules (n) per antibody molecule measured by common procedure E: 8.55. Average number of conjugated drug molecules (n) per antibody molecule measured by common procedure F: 7.65. The aggregation of the antibody-drug conjugate measured by common procedure G: 2.83% (FIG. 6A). The retention time of the antibody-drug conjugate measured by common procedure H: 7.505 min (FIG. 6B).

### 4.1.2 Production of Antibody-Drug Conjugate CL069707-H1L1-LP2

### Step 1: Antibody-Drug Conjugate

Reduction of antibody: CL069707-H1L1 was adjusted to 9 mg/mL with PBS7.0/EDTA by using common procedures B (using 1.423 mLmg⁻¹ cm⁻¹ as 280 nm absorption coefficient) and C described in production method. To this solution (1.099 mL), an aqueous solution of 5 mM TCEP (0.101 mL; 7.0 equivalents per antibody molecule). After confirming that the solution had a pH within 7.0±0.1, the interchain disulfide bond in the antibody was reduced by incubating the solution at 37° C. for 2 hours.

Conjugation between antibody and linker-payload: The above-described solution was incubated at 4° C. for 10 minutes. Subsequently, a 10 mM solution of LP2 in dimethylacetamide (0.108 mL; 15 equivalents per antibody molecule) was added thereto, and the obtained mixture was incubated at 22° C. for 30 minutes to conjugate the linker-payload to the antibody.

Purification: The above-described solution was purified by common procedure D described in production method to obtain 1.2 mL of a solution containing the title antibody-drug conjugate “CL069707-H1L1-LP2”.

Characterization: Using common procedure E (using _{εD,280}=5814 and _{εD,370}=14742), procedure F, procedure G and procedure H described in production method, the following characteristic values were obtained.

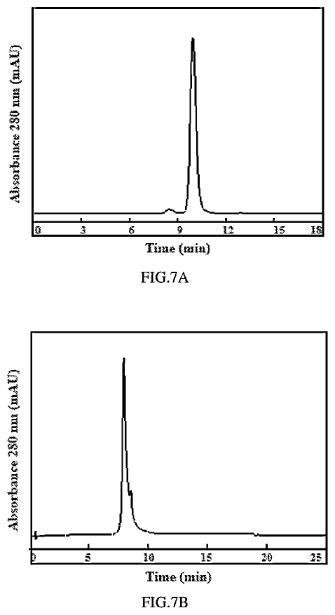

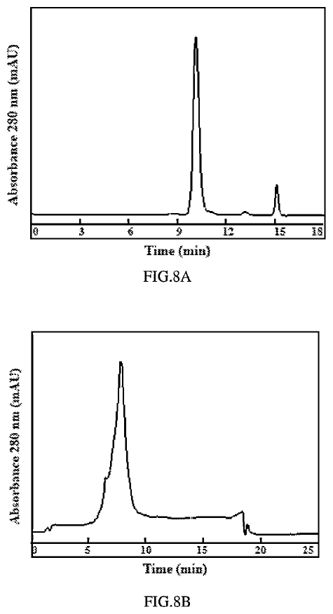

Antibody concentration: 7.37 mg/mL, average number of conjugated drug molecules (n) per antibody molecule measured by common procedure E: 9.27. Average number of conjugated drug molecules (n) per antibody molecule measured by common procedure F: 7.72. The aggregation of the antibody-drug conjugate measured by common procedure G: 2.96% (FIG. 7A). The retention time of the antibody-drug conjugate measured by common procedure H: 7.820 min (FIG. 7B).

### 4.1.3 Production of Antibody-Drug Conjugate CL069707-H1L1-GGFG-DXd

### Step 1: Antibody-Drug Conjugate

Reduction of antibody: CL069707-H1L1 was adjusted to 9 mg/mL with PBS7.0/EDTA by using common procedures B (using 1.423 mLmg⁻¹ cm⁻¹ as 280 nm absorption coefficient) and C described in production method. To this solution (0.275 mL), an aqueous solution of 5 mM TCEP (0.025 mL; 7.0 equivalents per antibody molecule). After confirming that the solution had a pH within 7.0±0.1, the interchain disulfide bond in the antibody was reduced by incubating the solution at 37° C. for 2 hours.

Conjugation between antibody and linker-payload: The above-described solution was incubated at 4° C. for 10 minutes. Subsequently, a 10 mM solution of GGFG-DXd (purchased from DC Chemicals, DC7556) in dimethylacetamide (0.027 mL; 15 equivalents per antibody molecule) was added thereto, and the obtained mixture was incubated at 22° C. for 30 minutes to conjugate the linker-payload to the antibody.

Purification: The above-described solution was purified by common procedure D described in production method to obtain 0.3 mL of a solution containing the title antibody-drug conjugate “CL069707-H1L1-GGFG-DXd”.

Characterization: Using common procedure E (using _{εD,280}=5178 and _{εD,370}=20217), procedure G and procedure H described in production method, the following characteristic values were obtained.

Antibody concentration: 6.78 mg/mL, average number of conjugated drug molecules (n) per antibody molecule measured by common procedure E: 6.88. The aggregation of the antibody-drug conjugate measured by common procedure G: 0.92% (FIG. 8A). The retention time of the antibody-drug conjugate measured by common procedure H: 8.802 min (FIG. 8B).

### 4.1.4 Production of Antibody-Drug Conjugate DS-6000a (Referred to Herein as Reference ADC)

### Step 1: Antibody-Drug Conjugate

The sequence of DS-6000a mAb (referred to herein as Reference Antibody) was disclosed in US 2020/0390900 Å1(antibody H01L02). The sequence of heavy chain variable region of Reference Antibody is shown in SEQ ID NO: 105. The sequence of light chain variable region of Reference Antibody is shown in SEQ ID NO: 106. The sequences of heavy chain constant region and light chain constant region of the Reference Antibody are shown respectively in SEQ ID NO: 70 and SEQ ID NO: 71.

(SEQ ID NO: 109)
EVQLVQSGAEVKKPGASVKVSCKASGYTFTRNFMHWVRQAPGQGLEWMG
WIYPGDGETEYAQKFQGRVTITADTSTSTAYMELSSLRSEDTAVYYCAR
GVYGGFAGGYFDFWGQGTLVTVSS.
(SEQ ID NO: 110)
DIQMTQSPSSLSASVGDRVTITCKASQNIYKNLAWYQQKPGKAPKLLIY
DANTLQTGVPSRFSGSGSGSDFTLTISSLQPEDFATYFCQQYYSGWAFG
QGTKVEIK.

Reduction of antibody: The Reference Antibody was adjusted to 14.8 mg/mL with PBS7.0/EDTA by using common procedures B (using 1.678 mLmg⁻¹ cm⁻¹ as 280 nm absorption coefficient) and C described in production method. To this solution (0.659 mL), an aqueous solution of 5 mM TCEP (0.130 mL; 10.0 equivalents per antibody molecule). After confirming that the solution had a pH within 7.0±0.1, the interchain disulfide bond in the antibody was reduced by incubating the solution at 37° C. for 2 hours.

Conjugation between antibody and linker-payload: The above-described solution was incubated at 4° C. for 10 minutes. Subsequently, a 10 mM solution of GGFG-DXd in dimethylacetamide (0.098 mL; 15 equivalents per antibody molecule) was added thereto, and the obtained mixture was incubated at 22° C. for 30 minutes to conjugate the linker-payload to the antibody.

Purification: The above-described solution was purified by common procedure D described in production method to obtain 1.4 mL of a solution containing the title antibody-drug conjugate “Reference ADC”.

Characterization: Using common procedure E (using _{εD,280}=5178 and _{εD,370}=20217), procedure G and procedure H described in production method, the following characteristic values were obtained.

Antibody concentration: 4.46 mg/mL, average number of conjugated drug molecules (n) per antibody molecule measured by common procedure E: 7.02. The aggregation of the antibody-drug conjugate measured by common procedure G: 2.68% (FIG. 9A). The retention time of the antibody-drug conjugate measured by common procedure H: 7.672 min (FIG. 9B).

### 4.1.5 Production of Antibody-Drug Conjugate Reference Antibody-LP1

### Step 1: Antibody-Drug Conjugate

Reduction of antibody: Reference Antibody was adjusted to 12.7 mg/mL with PBS7.0/EDTA by using common procedures B (using 1.678 mLmg⁻¹ cm⁻¹ as 280 nm absorption coefficient) and C described in production method. To this solution (0.106 mL), an aqueous solution of 5 mM TCEP (12.6 uL; 7 equivalents per antibody molecule). After confirming that the solution had a pH within 7.0±0.1, the interchain disulfide bond in the antibody was reduced by incubating the solution at 37° C. for 2 hours.

Conjugation between antibody and linker-payload: The above-described solution was incubated at 4° C. for 10 minutes. Subsequently, a 10 mM solution of LP1 in dimethylacetamide (13.5 uL; 15 equivalents per antibody molecule) was added thereto, and the obtained mixture was incubated at 22° C. for 30 minutes to conjugate the linker-payload to the antibody.

Purification: The above-described solution was purified by common procedure D described in production method to obtain 200 μL of a solution containing the title antibody-drug conjugate “Reference Antibody-LP1”.

Characterization: Using common procedure E (using _{εD,280}=6384 and _{εD,370}=16180), procedure G and procedure H described in production method, the following characteristic values were obtained.

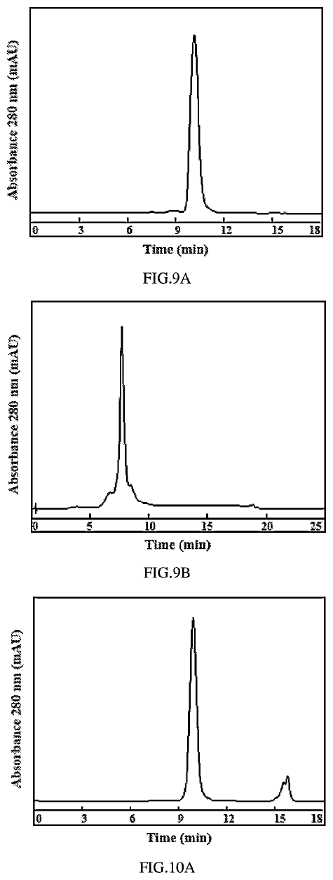

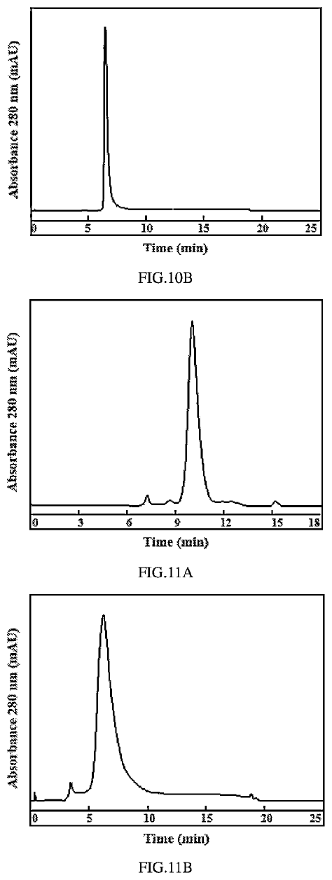

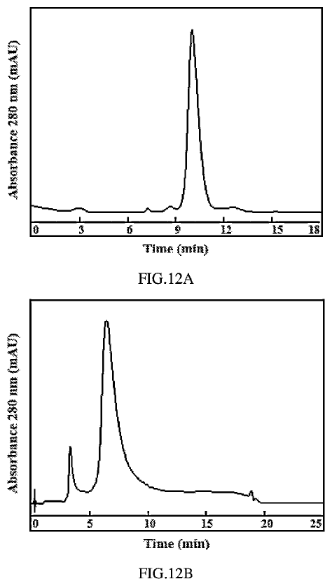

Antibody concentration: 8.07 mg/mL, average number of conjugated drug molecules (n) per antibody molecule measured by common procedure E: 7.11. The aggregation of the antibody-drug conjugate measured by common procedure G: 0.89% (FIG. 10A). The retention time of the antibody-drug conjugate measured by common procedure H: 6.422 min (FIG. 10B).

### 4.1.6 Production of Antibody-Drug Conjugate Human IgG-LP1

### Step 1: Antibody-Drug Conjugate

Reduction of antibody: Human IgG (purchase from Solarbio) was adjusted to 10 mg/mL with PBS7.0/EDTA by using common procedures B (using 1.35 mLmg⁻¹ cm⁻¹ as 280 nm absorption coefficient) and C described in production method. To this solution (1.33 mL), an aqueous solution of 5 mM TCEP (0.267 mL; 10 equivalents per antibody molecule). After confirming that the solution had a pH within 7.0±0.1, the interchain disulfide bond in the antibody was reduced by incubating the solution at 37° C. for 2 hours.

Conjugation between antibody and linker-payload: The above-described solution was incubated at 4° C. for 10 minutes. Subsequently, a 10 mM solution of LP1 in dimethylacetamide (0.2 mL; 15 equivalents per antibody molecule) was added thereto, and the obtained mixture was incubated at 22° C. for 30 minutes to conjugate the linker-payload to the antibody.

Purification: The above-described solution was purified by common procedure D described in production method to obtain 2 mL of a solution containing the title antibody-drug conjugate “Human IgG-LP1”.

Characterization: Using common procedure E (using _{εD,280}=6384 and _{εD,370}=16180), procedure G and procedure H described in production method, the following characteristic values were obtained.

Antibody concentration: 11.08 mg/mL, average number of conjugated drug molecules (n) per antibody molecule measured by common procedure E: 9.15. The aggregation of the antibody-drug conjugate measured by common procedure G: 4.38% (FIG. 11A). The retention time of the antibody-drug conjugate measured by common procedure H: 6.213 min (FIG. 11B).

### 4.1.7 Production of Antibody-Drug Conjugate Human IgG-LP2

### Step 1: Antibody-Drug Conjugate

Reduction of antibody: Human IgG (purchase from Solarbio) was adjusted to 10 mg/mL with PBS7.0/EDTA by using common procedures B (using 1.35 mLmg⁻¹ cm⁻¹ as 280 nm absorption coefficient) and C described in production method. To this solution (0.467 mL), an aqueous solution of 5 mM TCEP (0.093 mL; 10 equivalents per antibody molecule). After confirming that the solution had a pH within 7.0±0.1, the interchain disulfide bond in the antibody was reduced by incubating the solution at 37° C. for 2 hours.

Conjugation between antibody and linker-payload: The above-described solution was incubated at 4° C. for 10 minutes. Subsequently, a 10 mM solution of LP2 in dimethylacetamide (0.07 mL; 15 equivalents per antibody molecule) was added thereto, and the obtained mixture was incubated at 22° C. for 30 minutes to conjugate the linker-payload to the antibody.

Purification: The above-described solution was purified by common procedure D described in production method to obtain 1 mL of a solution containing the title antibody-drug conjugate “Human IgG-LP2”.

Characterization: Using common procedure E (using _{εD,280}=5814 and _{εD,370}=14742), procedure G and procedure H described in production method, the following characteristic values were obtained.

Antibody concentration: 4.68 mg/mL, average number of conjugated drug molecules (n) per antibody molecule measured by common procedure E: 9.18. The aggregation of the antibody-drug conjugate measured by common procedure G: 2.92% (FIG. 12A). The retention time of the antibody-drug conjugate measured by common procedure H: 6.506 min (FIG. 12B).

### 4.1.8 Production of Antibody-Drug Conjugate Human IgG-GGFG-DXd

### Step 1: Antibody-Drug Conjugate

Reduction of antibody: Human IgG (purchase from Solarbio) was adjusted to 10 mg/mL with PBS7.0/EDTA by using common procedures B (using 1.35 mLmg⁻¹ cm⁻¹ as 280 nm absorption coefficient) and C described in production method. To this solution (2.45 mL), an aqueous solution of 5 mM TCEP (0.327 mL; 10 equivalents per antibody molecule). After confirming that the solution had a pH within 7.0±0.1, the interchain disulfide bond in the antibody was reduced by incubating the solution at 37° C. for 2 hours.

Conjugation between antibody and linker-payload: The above-described solution was incubated at 4° C. for 10 minutes. Subsequently, a 10 mM solution of GGFG-DXd in dimethylacetamide (0.294 mL; 18 equivalents per antibody molecule) was added thereto, and the obtained mixture was incubated at 22° C. for 30 minutes to conjugate the linker-payload to the antibody.

Purification: The above-described solution was purified by common procedure D described in production method to obtain 1 mL of a solution containing the title antibody-drug conjugate “Human IgG-GGFG-DXd”.

Characterization: Using common procedure E (using _{εD,280}=5178 and _{εD,370}=20217), procedure G and procedure H described in production method, the following characteristic values were obtained.

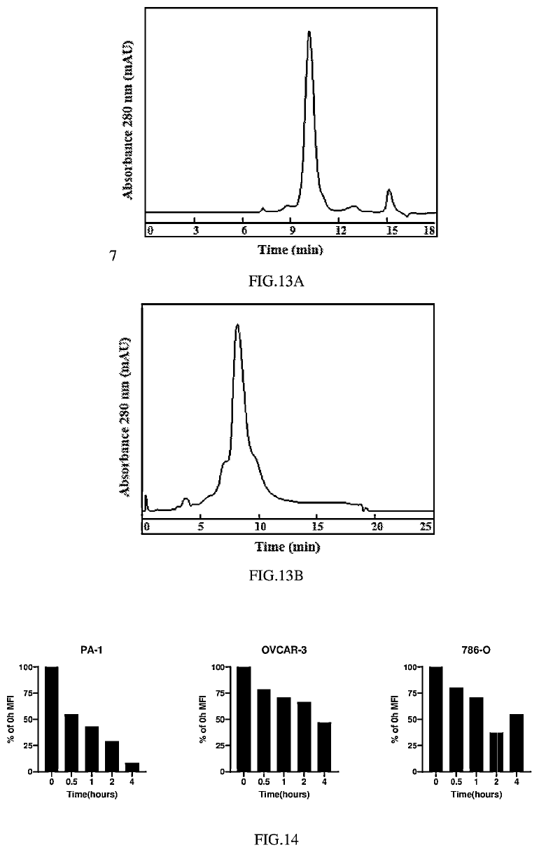

Antibody concentration: 8.20 mg/mL, average number of conjugated drug molecules (n) per antibody molecule measured by common procedure E: 7.08. The aggregation of the antibody-drug conjugate measured by common procedure G: 4.25% (FIG. 13A). The retention time of the antibody-drug conjugate measured by common procedure H: 8.222 min (FIG. 13B).

The production of other Antibody-Drug Conjugates ‘CL069707-MMAE’, ‘CL069707-DXd’, ‘CL069066-MMAE’, ‘CL069066-DXd’, ‘CL069439-MMAE’ and ‘CL069439-DXd’ were the same as samples above.

### Example 5 In Vitro Evaluation of Antibody-Drug Conjugate

### 5.1 Internalization Activity of Antibody-Drug Conjugate

Human ovarian cancer cell line PA-1(purchased from Zhejiang Meisen Cell Technology Co., Ltd.), OVCAR-3(purchased from National Collection of Authenticated Cell Cultures) and human renal carcinoma cell line 786-O (purchased from Shanghai Xunqing Biotechnology Co., Ltd.) cell culture were maintained in vitro as independent monolayer cultures at 37° C. in an atmosphere of 5% CO₂. The cells were harvested using trypsin-EDTA partial digestion followed by centrifugation at 1000 rpm for 5 minutes. The cell was resuspended in cold PBS and 50 ug/mL of CL069707-H1L1-LP1 were added. The cell solutions were mixed, incubated at 4° C. for 30 min, then the cells were washed with PBS, and then aliquoted into 5 portions and incubated at 37° C. for 0 h, 0.5 h, 1, 2 h, and 4 h. At each time point, one sample was taken out and washed with cold PBS prior to addition of secondary antibody conjugates (for detection purposes). After incubation at 4° C., the cells were washed with PBS, and then subjected to flow cytometry (FACS) analysis. The results are shown in FIG. 14. CL069707-H1L1-LP1 was internalized into PA-1, OVCAR-3 and 786-O in a time dependent manner.

### 5.2 In Vitro Tumor Growth Inhibition Activity of Antibody-Drug Conjugates

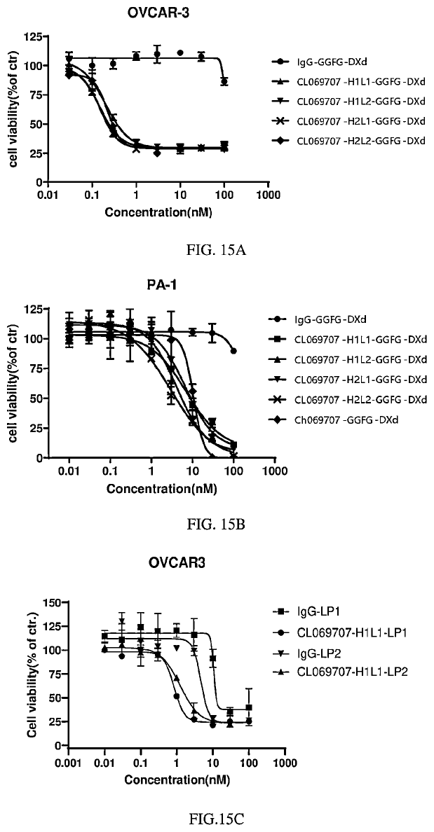

CDH6-positive human ovarian cancer cell line OVCAR-3(purchased from National Collection of Authenticated Cell Cultures) and PA-1(purchased from Zhejiang Meisen Cell Technology Co., Ltd.) were seeded over a 96 well plate at 2000 cells/100 uL/well in culture medium, the cells were then cultured overnight. On the next day, each of the conjugates was added to the cells such that the final concentrations were from 0.01 nM to 100 nM. After culture for 5 days, media were removed from the cells and the CCK-8 (Cell Counting Kit-8) viability assays were performed according to the manufacturer's instructions. Cell viability was calculated as % of control wells with only tumor cells. FIG. 15A-15D shows concentration-dependent cell growth inhibition activity when each of the antibody-drug conjugate was added to CDH6 positive OVCAR-3 and PA-1 cells. All the humanized CL069707-GGFG-DXd (H1L1, H1L2, H2L1 and H2L2) showed similar excellent tumor cell growth inhibition activity on OVCAR-3 cells and PA-1 cells in FIG. 15A and FIG. 15B. All the two positive antibody-drug conjugates showed excellent tumor cell growth inhibition activity in FIG. 15C and FIG. 15D, among which CL069707-H1L1-LP1 showed the most significant inhibition activity on OVCAR-3 cells, followed by CL069707-H1L1-LP2, and among which CL069707-H1L1-LP1 and CL069707-H1L1-LP2 showed comparable inhibition activity on PA-1 cells.

### Example 6 In Vitro Cell Killing Activity of Mouse Anti-CDH6 Antibodies CL069707, CL069439, CL069066 Conjugated to Vc-MMAE or GGFG-DXd

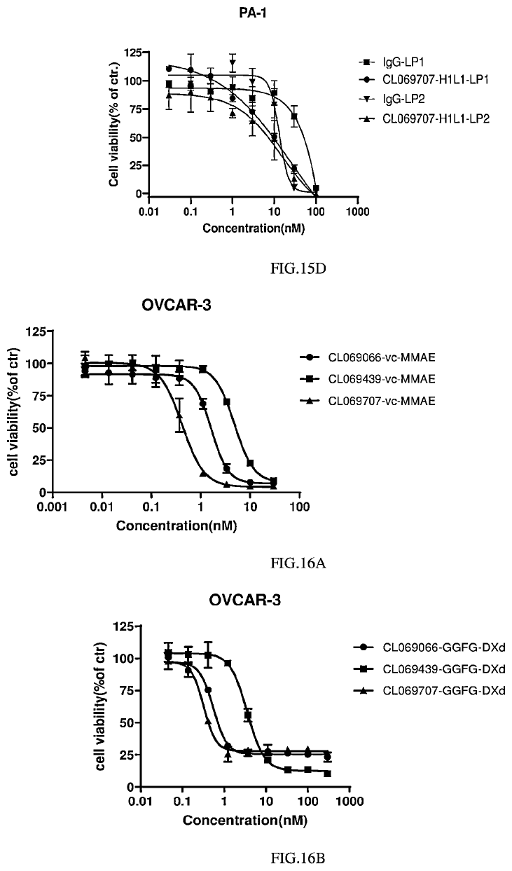

CDH6-positive human ovarian cancer cell line OVCAR-3(purchased from National Collection of Authenticated Cell Cultures) was seeded over a 96 well plate at 2000 cells/100 uL/well in culture medium, the cells were then cultured overnight. On the next day, each of CL069707-vc-MMAE, CL069439-vc-MMAE and CL069066-vc-MMAE was added to the cells such that the final concentrations were from 0.005 nM to 30 nM; and each of CL069707-GGFG-DXd, CL069439-GGFG-DXd and CL069066-GGFG-DXd was added to the cells such that the final concentrations were from 0.05 nM to 300 nM. After culturing for 5 days, media were removed from the cells and the CCK-8 (Cell Counting Kit-8) viability assays were performed according to the manufacturer's instructions. Cell viability was calculated as % of control wells with only tumor cells. FIG. 16A shows that all the 3 conjugates had concentration-dependent cell growth inhibition activity against OVCAR-3 cell. Moreover, CL069707-vc-MMAE showed stronger cell killing activity than that of CL069439-vc-MMAE and CL069066-vc-MMAE. FIG. 16B shows that all the 3 conjugates had concentration-dependent cell growth inhibition activity against OVCAR-3 cell. Moreover, CL069707-GGFG-DXd showed stronger cell killing activity than that of CL069439-GGFG-DXd and CL069066-GGFG-DXd.

### Example 7 In Vivo Antitumor Effect of Antibody-Drug Conjugate in PA-1 Xenograft Model

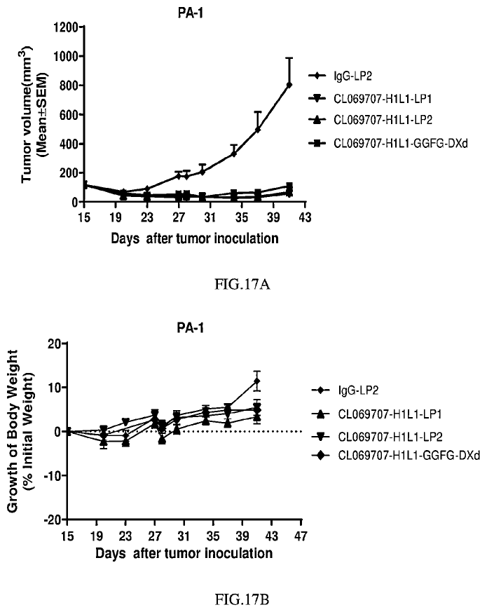

6 weeks female Balb/c mice were purchased from JSJ Laboratory (Shanghai, China). Human ovarian cancer cell line PA-1(purchased from Zhejiang Meisen Cell Technology Co., Ltd.) cells were mixed with matrigel, then implanted subcutaneously at 10 million cells/animal. On day 15 (mean tumor size ˜150 mm³) following implanting of PA-1 cells, mice were injected intravenous with 10 mg/kg of one of the following substances: IgG-LP2, CL069707-H1L1-LP1, CL069707-H1L1-LP2 or CL069707-H1L1-GGFG-DXd. Tumor volumes were measured two times per week from day of treatment initiation by Caliper and calculated as follows: TV=(Width*Length)²/2. The results are shown in FIG. 17A-17B. All the antibody-drug conjugates decreased tumor volume significantly after a single dose administration without affecting mice body weight significantly.

### Example 8 In Vivo Antitumor Effect of Antibody-Drug Conjugate in PA-1 Xenograft Model

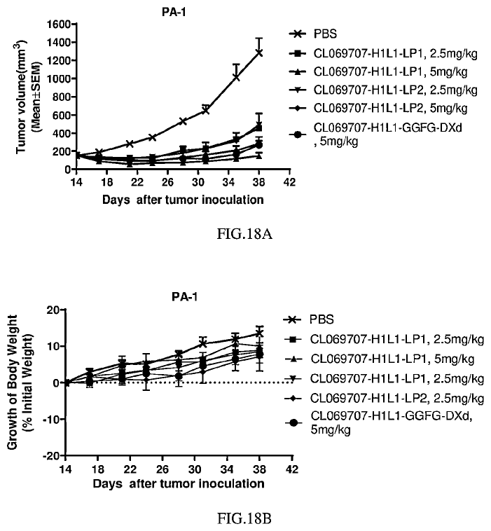

6 weeks female Balb/c mice were purchased from JSJ Laboratory (Shanghai, China). Human ovarian cancer cell line PA-1(purchased from Zhejiang Meisen Cell Technology Co., Ltd.) cells were mixed with matrigel, then implanted subcutaneously at 10 million cells/animal. On day 14 (mean tumor size ˜150 mm³) following implanting of PA-I cells, mice were injected intravenous with one of the following substances: PBS, CL069707-H1L1-LP1 (2.5 mg/kg and 5 mg/kg), CL069707-H1L1-LP2 (2.5 mg/kg and 5 mg/kg) or CL069707-H1L1-GGFG-DXd(5 mg/kg). Tumor volumes were measured two times per week from day of treatment initiation by Caliper and calculated as follows: TV=(Width*Length)²/2. The results are shown in FIG. 18A-18B. All the antibody-drug conjugates decreased tumor volume in a dose dependent manner after a single dose administration without affecting mice body weight significantly. Notably, 5 mg/kg CL067707-H1L1-LP1 had better tumor inhibition effect than CL069707-H1L1-LP2 and CL067707-H1L1-GGFG-DXd.

### Example 9 In Vivo Antitumor Effect of Antibody-Drug Conjugate in OVCAR-3 Xenograft Model

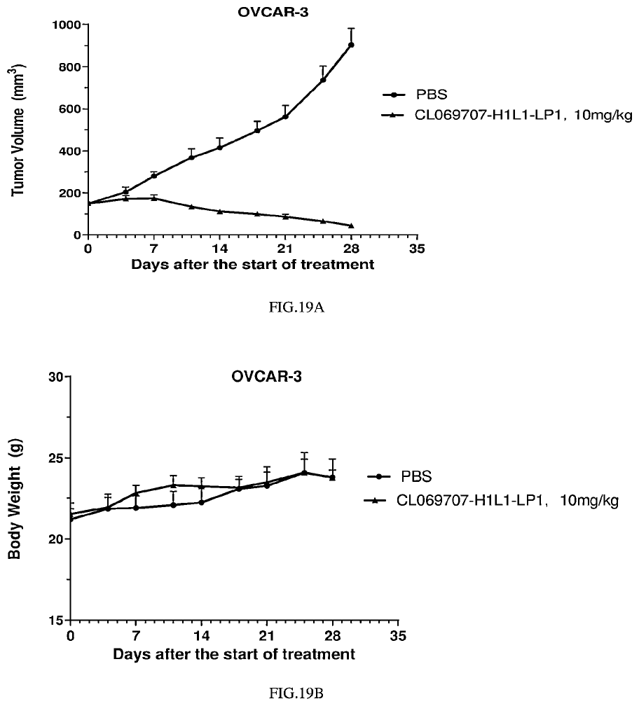

6-8 weeks Balb/c mice were purchased from Shanghai Lingchang Biotechnology Co., Ltd. (Shanghai, China). Human ovarian cancer cell line OVCAR-3(ATCC-HTB-161) cells were mixed with matrigel, then implanted subcutaneously at 10 million cells/animal. On day 35 (mean tumor size ˜150 mm³) following implanting of OVCAR-3 cells, mice were injected intravenous with one of the following substances: PBS and 10 mg/kg CL069707-H1L1-LP1. Tumor volumes were measured two times per week from day of treatment initiation by Caliper and calculated as follows: TV=(Width*Length)²/2. The results are shown in FIG. 19A-19B. A single dose of CL069707-H1L1-LP1 decreased tumor volume significantly without affecting mice body weight.

### Example 10 In Vivo Antitumor Effect of Antibody-Drug Conjugate in 786-O Xenograft Model

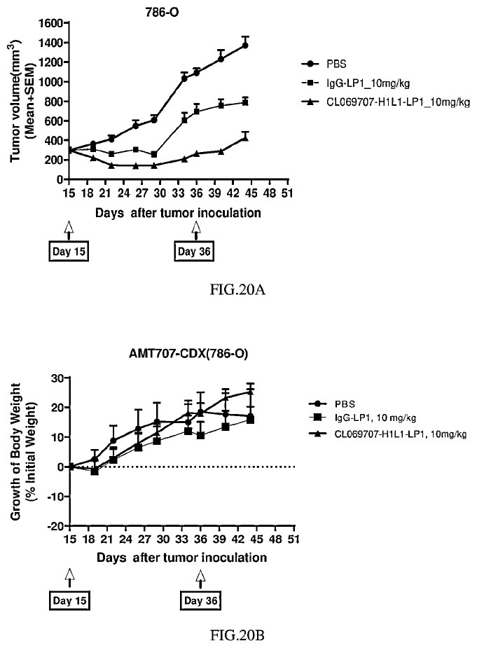

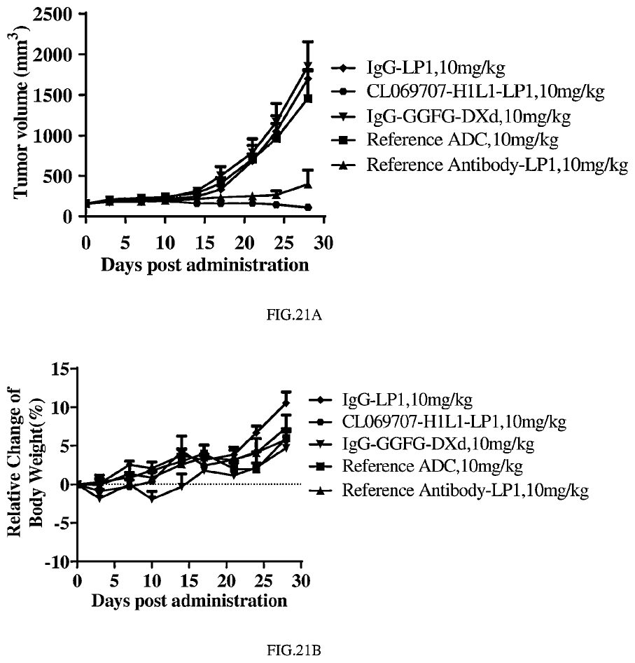

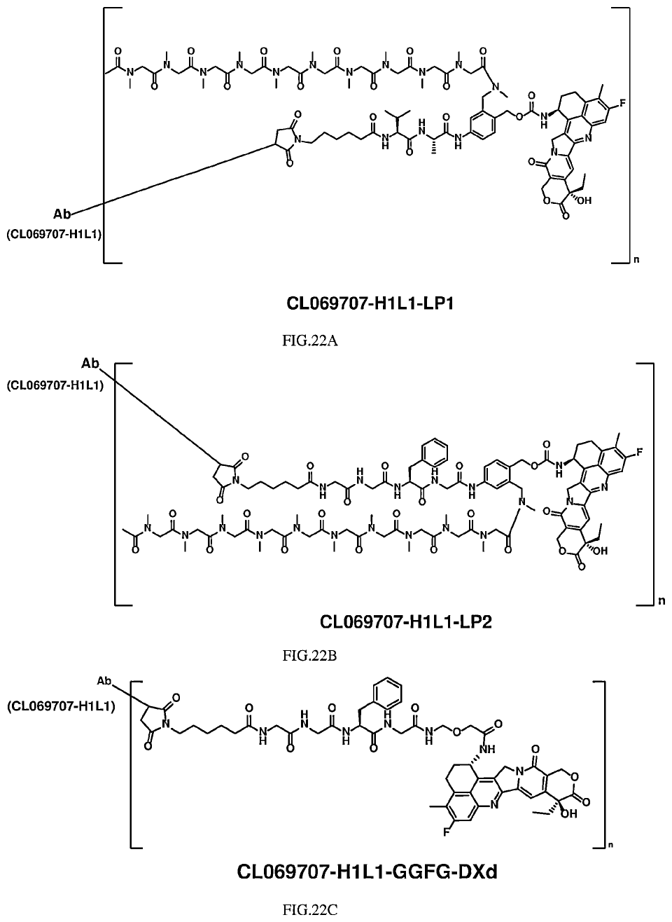

6 weeks NCG mice were purchased from gempharmatech (Suzhou, China).Human renal carcinoma cell line 786-O (purchased from Shanghai Xunqing Biotechnology Co., Ltd.) cells were resuspended in serum-free medium, then implanted subcutaneously at 6.5 million cells/animal. On day 15 (mean tumor size ˜150 mm³) and day 36 following implanting of 786-O cells, mice were injected intravenous with one of the following substances: PBS, 10 mg/kg IgG-LP1 and 10 mg/kg CL069707-H1L1-LP1. Tumor volumes were measured two times per week from day of treatment initiation by Caliper and calculated as follows: TV=(Width*Length)²/2. The results are shown in FIG. 20A-20B. CL069707-H1L1-LP1 decreased tumor volume significantly when compare with IgG-LP1, without affecting mice body weight.

### Example 11 In Vivo Antitumor Effect of Antibody-Drug Conjugate in a Renal Carcinoma Patient-Derived Xenograft (PDX) Model

6-8 weeks Nu/Nu mice were purchased from Zhejiang Charles River Co., Ltd. The tumors were sliced into 3 mm×3 mm×3 mm (about 50-90 mg) fragments and implanted subcutaneously into the right flank of mice. Then the animals were checked daily for tumor growth and body weight. The mice were randomized into 5 groups when mean tumor volume reaches ˜150 mm³. The grouping day was regarded as day 0. Mice were then injected intravenous with 10 mg/kg of one of the following substances:10 mg/kg IgG-LP1, CL069707-H1L1-LP1, IgG-GGFG-DXd, Reference ADC and Reference Antibody-LP1. Tumor volumes were measured two times per week from day of treatment initiation by Caliper and calculated as follows: TV=(Width*Length)²/2. The results are shown in FIG. 21A-21B. CL069707-H1L1-LP1 decreased tumor volume significantly followed by Reference Antibody-LP1 when compared with IgG-LP1, without affecting mice body weight. Reference ADC showed no tumor growth inhibition activity when comparing with IgG-GGFG-DXd.

### Example 12 Preparation of Mouse Monoclonal Antibody Hybridoma Cells of CDH6 Protein

### 12.1 Preparation of antigens

CDH6 extracellular region (Ser 54-Ala 615) was used as an immunogen, and the antigen was purchased from ACRO Biosystems with Cat. No. C_{A}6-H5229.

### 12.2 Immunization of Mice

Each group of antigens would be used to immunize 12 Balb/c mice (8-12 weeks old) and the serum titers of the immunized mice would be monitored to determine the optimal number of immunizations. The optimized adjuvant and immunization method could produce antibodies (IgG subtype) with high affinity for most antigenic polypeptides. After the initial immunization, 3 to 4 enhanced immunizations were performed, and after the enhanced immunizations, mouse serum was taken to detect the titer (recombinant protein was used as antigen coating). Mice with serum at qualified titer would be enhanced-immunized for fusion, while mice with serum at unqualified titer would continue to be enhanced-immunized one to two times to the highest titer before fusion.

### 12.3 Serum Detection and Screening

The immunized mice were subjected to blood collection from the eye orbit, and the serum titers were detected by ELISA (recombinant protein was used as antigen coating). Serum titers was required to be greater than 10 K, otherwise the enhanced immunization was continued.

### 12.4 Fusion and Screening

The whole spleen and ½ lymph node were taken and fused with myeloma SP2/0 cell line. The process was the optimized PEG fusion. The fused cells were plated in 4 384-well plates (10² to 10⁴ cells per well) and cultured. The supernatant from all wells was collected, positive cell strains were screened by ELISA to detect the response of the cell supernatant to CDH6 recombinant protein, and wells from which cells were mirrored were transferred to 96-well plates for further culture. After several days of growth, the supernatant from all wells was collected and detected for reactivity with CDH6 recombinant protein by ELISA. Positive wells were further detected for the binding ability of cell supernatant at various dilutions to CDH6 recombinant protein for affinity ranking, and 120 parental clones with the highest affinity were selected for subcloning.

### 12.5 Subcloning and Screening

Subcloning was performed by a limiting dilution method and ELISA screening to obtain monoclonal hybridoma cells. Cells were plated in a 96-well plate and cultured to cover approximately ⅙ bottom of the plate. The response of the supernatant in each well to the CDH6 recombinant protein was detected by ELISA, and two wells with high OD values and good cell status were used for the next round of subcloning. The above steps were repeated until the positive rate of the cell strain in the wells was 100%. Then monoclonal cell strains were obtained. After the last round of subcloning, all positive cells were immediately expanded, one part was frozen for later use, and the other part was prepared as ascites.

### 12.6 Ascites Preparation and Antibody Purification

Finally, 13 monoclonal cell strains were obtained and injected by abdomen into F1 mice for antibody production. The produced ascites was purified with Protein A/G and used for subsequent detection.

### Example 13 Binding Activity of Antibodies to Antigens

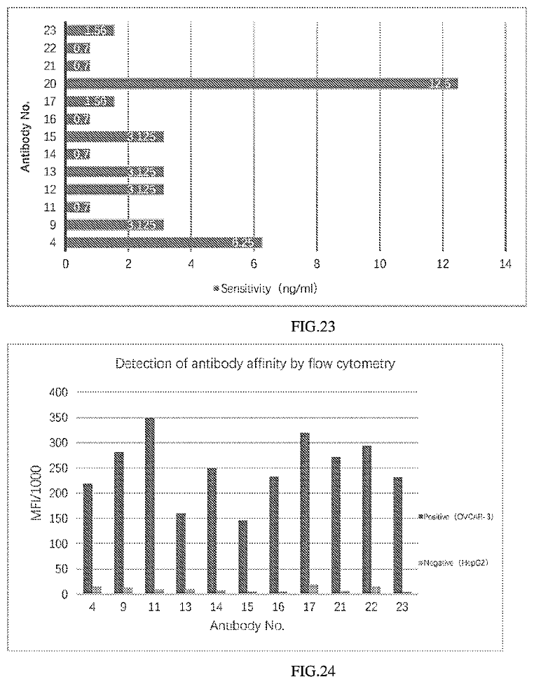

The recombinant protein CDH6 was coated on ELISA plates at 1 μg/mL overnight at 4° C.; after being washed with PBST, the plate was added with 10% fetal bovine serum, and the mixture was blocked at 37° C. for 1 h; the plate was added with 13 monoclonal antibodies of CDH6 with different dilutions (0.1 ng/mL to 100 ng/mL), and the mixture was reacted at 37° C. for 1 h; after being washed with PBST, the plate was added with horseradish peroxidase-labeled goat anti-human IgG secondary antibody (Goat anti Mouse (HRP), Thermo Fisher Scientific), and the mixture was reacted at 37° C. for 30 min; after the plate was repeatedly washed with PBST 5 times, the remaining droplets on the absorbent paper were patted as much as possible; each well of the plate was added with 100 μL of TMB (eBioscience) and placed in the dark at room temperature (20±5° C.) for 1.5 min; each well of the plate was added with 100 μL of 2 N H₂SO₄ stop buffer for stopping substrate reaction, OD values at 450 nm were read using a microplate reader, and the binding ability of the antibody to the target antigen CDH6 was analyzed. As shown in Table 1 and FIG. 23, the sensitivity of the anti-CDH6 antibody and CDH6 recombinant protein of the present application was not more than 12.5 ng/mL, wherein #13 (CL069463 antibody) was not more than 3.125 ng/mL, #23 (CL069707 antibody) was not more than 1.56 ng/mL, #4 (CL069066 antibody) was not more than 6.3 ng/mL, and #16 (CL069439 antibody) was not more than 1.0 ng/mL.

TABLE 3
Detection of binding activity of the antibody to CDH6 antigen by ELISA

| No. | 100 | 50 | 25 | 12.5 | 6.25 | 3.13 | 1.56 | 0.78 | 0.39 | 0.20 | 0.10 | NC |
|---|---|---|---|---|---|---|---|---|---|---|---|---|
| 4 | 2.382 | 2.111 | 1.685 | 1.076 | 0.577 | 0.300 | 0.170 | 0.094 | 0.063 | 0.050 | 0.048 | 0.056 |
| 9 | 3.553 | 3.267 | 2.930 | 2.249 | 1.644 | 1.081 | 0.470 | 0.255 | 0.135 | 0.077 | 0.057 | 0.050 |
| 11 | 3.900 | 3.900 | 3.900 | 3.672 | 3.194 | 2.490 | 1.467 | 0.796 | 0.457 | 0.183 | 0.102 | 0.049 |
| 12 | 3.732 | 3.254 | 2.818 | 2.159 | 1.554 | 0.865 | 0.456 | 0.299 | 0.157 | 0.097 | 0.072 | 0.053 |
| 13 | 2.619 | 2.320 | 1.798 | 1.697 | 0.975 | 0.596 | 0.374 | 0.211 | 0.139 | 0.086 | 0.073 | 0.061 |
| 14 | 3.872 | 3.872 | 3.763 | 3.161 | 2.787 | 1.588 | 0.929 | 0.558 | 0.279 | 0.157 | 0.107 | 0.057 |
| 15 | 3.536 | 3.411 | 3.536 | 2.258 | 1.448 | 0.812 | 0.391 | 0.202 | 0.120 | 0.078 | 0.072 | 0.060 |
| 16 | 3.900 | 3.888 | 3.691 | 3.619 | 2.824 | 1.873 | 1.113 | 0.647 | 0.255 | 0.181 | 0.106 | 0.061 |
| 17 | 3.778 | 3.334 | 3.213 | 2.618 | 2.088 | 1.323 | 0.615 | 0.349 | 0.176 | 0.106 | 0.071 | 0.072 |
| 20 | 2.052 | 1.266 | 0.893 | 0.698 | 0.363 | 0.205 | 0.120 | 0.078 | 0.062 | 0.049 | 0.057 | 0.048 |
| 21 | 3.567 | 3.759 | 3.634 | 3.315 | 2.638 | 1.580 | 1.169 | 0.635 | 0.299 | 0.171 | 0.086 | 0.054 |
| 22 | 2.405 | 2.439 | 2.230 | 2.337 | 2.240 | 1.539 | 0.800 | 0.508 | 0.262 | 0.174 | 0.090 | 0.051 |
| 23 | 1.409 | 1.326 | 1.183 | 1.273 | 1.011 | 0.831 | 0.577 | 0.305 | 0.165 | 0.101 | 0.069 | 0.064 |

### Example 14 Specific Recognition of CDH6 Antigens Expressed on the Cell Surface by Antibodies

The binding of 13 CDH6 monoclonal antibodies to CDH6 on the cell surface was analyzed by Flow cytometry (FCM) using OVCAR-3 cells (human ovarian cancer cells) as positive cells and HepG2 cells (human hepatoma cells) as negative cells. Cells in the logarithmic growth phase were collected, adjusted to a cell density of 5×10⁶ cells/mL, and pre-cooled on ice. 13 CDH6 monoclonal antibodies were diluted to 20 μg/mL in 2% FBS-containing pre-cooled saline. 100 μL of cells were taken and added with an equal volume of the diluted CDH6 monoclonal antibodies described above, and the mixture was reacted at 4° C. for 30 min in the dark. After the reaction was completed, the cells were washed twice with 2% PBS-containing pre-cooled saline (6000 rpm, 45 s). The secondary antibody PE anti-mouse IgG (BD Pharmingen) was diluted at 1:500 with 2% FBS-containing pre-cooled saline, and each of the washed cells were resuspended with 100 μL of diluted secondary antibody and reacted for 30 min at 4° C. in the dark. After the reaction was completed, the cells were washed twice with 2% FBS-containing pre-cooled saline (6000 rpm, 45 s). The cells were resuspended in 400 μL of saline. The flow cytometer (BD Calibur) was used to analyze the binding ability of antibodies to cell surface antigens.

The results are shown in FIG. 24. The results showed that a total of 11 antibodies including CL069463, CL069707 and the like were capable of specifically recognizing OVCAR-3 cells (human ovarian cancer cells) (FIG. 24), while no binding was observed in negative cells HepG2 cells (human hepatoma cells).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables.

It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### REFERENCES

(1) Wheelock, M. J. and Johnson, K. R. (2003) Annu Rev Cell Dev Biol 19, 207-35.
(2) Christofori, G. (2003) EMBO J 22, 2318-23.
(3) Hazan, R. B. et al. (2004) Ann N Y Acad Sci 1014, 155-63.
(4) Bryant, D. M. and Stow, J. L. (2004) Trends Cell Biol 14, 427-34.
(5) Rabascio, C. et al. (2004) Cancer Res 64, 4373-7.
(6) Yamaoka-Tojo, M. et al. (2006) Arterioscler Thromb Vasc Biol 26, 1991-7.
(7) Patel, I. S. et al. (2003) Int J Cancer 106, 172-7.
(8) Sanders, D. S. et al. (2000) J Pathol 190, 526-30.
(9) Goeppert, B. et al. (2016) Epigenetics 11, 780-790.
(10) Zhao, L. et al. (2016) Clin Endocrinol (Oxf) 84, 748-55.
(11) Sancisi, V. et al. (2013) PLoS One 8, e75489.
(12) Gugnoni, M. et al. (2017) Oncogene 36, 667-677.
(13) Bialucha, C. U. et al. (2017) Cancer Discov 7, 1030-1045.

1. An antigen-binding protein having one or more of the following properties:
i) being capable of binding to a CDH6 protein, optionally a mammalian CDH6 protein or human CDH6 protein, with a sensitivity of less than 12.5 ng/mL in an ELISA assay;
ii) being capable of binding to the CDH6 protein, optionally a mammalian CDH6 protein or human CDH6 protein, with a KD value below about 3.1×10⁻⁹M in an ELISA assay; and
iii) having the activity of being internalized into a CDH6-expressing cell by binding to CDH6, optionally a mammalian CDH6 protein or human CDH6 protein.

2-3. (canceled)

4. The antigen-binding protein according to claim 1, competing for binding to CDH6 protein with a reference antibody, wherein the reference antibody comprises a light chain variable region (VL) and a heavy chain variable region (VH); wherein:
the VH comprises an amino acid sequence as set forth in SEQ ID NO: 18, and the VL comprises an amino acid sequence as set forth in SEQ ID NO: 23; or
the VH comprises an amino acid sequence as set forth in SEQ ID NO: 41, and the VL comprises an amino acid sequence as set forth in SEQ ID NO: 46.

5. The antigen-binding protein according to claim 1, comprising an antibody or an antigen-binding fragment thereof;
optionally, wherein the antibody or antigen-binding fragment thereof comprises a monoclonal antibody, a polyclonal antibody, a dimer, a polymer, a multispecific antibody, an intact antibody, a human antibody, a humanized antibody or a chimeric antibody;
optionally, wherein the antigen-binding fragment comprises Fab, Fab′, an Fv fragment, F(ab′)2, scFv, di-scFv and/or dAb;
optionally, wherein the antibody comprises a chimeric antibody, a humanized antibody and/or a human antibody; and
optionally, wherein the antibody is a monoclonal antibody.

6-9. (canceled)

10. The antigen-binding protein according to claim 1, comprising:
(a) at least one CDR of a heavy chain variable region (VH), wherein the VH comprises an amino acid sequence as set forth in SEQ ID NO: 18, SEQ ID NO: 66, SEQ ID NO: 41, SEQ ID NO: 67, SEQ ID NO: 95 or SEQ ID NO: 103; and/or
(b) at least one CDR of a light chain variable region (VL), wherein the VL comprises an amino acid sequence as set forth in SEQ ID NO: 23, SEQ ID NO: 68, SEQ ID NO: 46, SEQ ID NO: 69, SEQ ID NO: 96 or SEQ ID NO: 104; and/or
(c) a VH, wherein the VH comprises an HCDR1, an HCDR2 and an HCDR3, and the HCDR1 comprises an amino acid sequence as set forth in SEQ ID NO: 1; and/or
(d) a VH, wherein the VH comprises an HCDR1, an HCDR2 and an HCDR3, and the HCDR1 comprises an amino acid sequence as set forth in SEQ ID NO: 2, SEQ ID NO: 8, SEQ ID NO: 89 or SEQ ID NO: 97; and/or
(e) a VH, wherein the VH comprises an HCDR1, an HCDR2 and an HCDR3, and the HCDR2 comprises an amino acid sequence as set forth in SEQ ID NO: 3, SEQ ID NO: 9, SEQ ID NO: 90 or SEQ ID NO: 98; and/or
(f) a VH, wherein the VH comprises an HCDR1, an HCDR2 and an HCDR3, and the HCDR3 comprises an amino acid sequence as set forth in SEQ ID NO: 4, SEQ ID NO: 10, SEQ ID NO: 91 or SEQ ID NO: 99; and/or
(g) a VH, wherein the VH comprises an HCDR1, an HCDR2 and an HCDR3, wherein the HCDR1 comprises an amino acid sequence as set forth in SEQ ID NO: 1, the HCDR2 comprises an amino acid sequence as set forth in SEQ ID NO: 3 or SEQ ID NO: 9, and the HCDR3 comprises an amino acid sequence as set forth in SEQ ID NO: 4 or SEQ ID NO: 10; and/or
(h) a VH, wherein the VH comprises an HCDR1, an HCDR2 and an HCDR3, wherein the HCDR1 comprises an amino acid sequence as set forth in SEQ ID NO: 1, the HCDR2 comprises an amino acid sequence as set forth in SEQ ID NO: 3, and the HCDR3 comprises an amino acid sequence as set forth in SEQ ID NO: 4; or
the HCDR1 comprises an amino acid sequence as set forth in SEQ ID NO: 1, the HCDR2 comprises an amino acid sequence as set forth in SEQ ID NO: 9, and the HCDR3 comprises an amino acid sequence as set forth in SEQ ID NO: 10; and/or
(i) a VH, wherein the VH comprises an HCDR1, an HCDR2 and an HCDR3, wherein the HCDR1 comprises an amino acid sequence as set forth in SEQ ID NO: 2, the HCDR2 comprises an amino acid sequence as set forth in SEQ ID NO: 3, and the HCDR3 comprises an amino acid sequence as set forth in SEQ ID NO: 4; or
the HCDR1 comprises an amino acid sequence as set forth in SEQ ID NO: 8, the HCDR2 comprises an amino acid sequence as set forth in SEQ ID NO: 9, and the HCDR3 comprises an amino acid sequence as set forth in SEQ ID NO: 10;
wherein the HCDR1 comprises the amino acid sequence as set forth in SEQ ID NO: 89, the HCDR2 comprises the amino acid sequence as set forth in SEQ ID NO: 90, and the HCDR3 comprises the amino acid sequence as set forth in SEQ ID NO: 91; or
wherein the HCDR1 comprises the amino acid sequence as set forth in SEQ ID NO: 97, the HCDR2 comprises the amino acid sequence as set forth in SEQ ID NO: 98, and the HCDR3 comprises the amino acid sequence as set forth in SEQ ID NO: 99;
(j) a VH, wherein the VH comprises a framework region HFR1, the C-terminus of the HFR1 is directly or indirectly connected to the N-terminus of the HCDR1, and the HFR1 comprises an amino acid sequence as set forth in SEQ ID NO: 14, SEQ ID NO: 24, SEQ ID NO: 37, SEQ ID NO: 47 or SEQ ID NO: 52; and/or
(k) a VH, wherein the VH comprises a framework region HFR2, the HFR2 is positioned between the HCDR1 and the HCDR2, and the HFR2 comprises an amino acid sequence as set forth in SEQ ID NO: 15, SEQ ID NO: 28, SEQ ID NO: 38 or SEQ ID NO: 48; and/or
(l) a VH, wherein the VH comprises a framework region HFR3, the HFR3 is positioned between the HCDR2 and the HCDR3, and the HFR3 comprises an amino acid sequence as set forth in SEQ ID NO: 16, SEQ ID NO: 25, SEQ ID NO: 39, SEQ ID NO: 49 or SEQ ID NO: 53; and/or
(m) a VH, wherein the VH comprises a framework region HFR4, the N-terminus of the HFR4 is directly or indirectly connected to the C-terminus of the HCDR3, and the HFR4 comprises an amino acid sequence as set forth in SEQ ID NO: 17, SEQ ID NO: 26, SEQ ID NO: 40 or SEQ ID NO: 50; and/or
(n) a VH, wherein the VH comprises framework regions HFR1, HFR2, HFR3 and HFR4, the C-terminus of the HFR1 is directly or indirectly connected to the N-terminus of the HCDR1, the HFR2 is positioned between the HCDR1 and the HCDR2, the HFR3 is positioned between the HCDR2 and the HCDR3, and the N-terminus of the HFR4 is directly or indirectly connected to the C-terminus of the HCDR3; wherein the HFR1 comprises an amino acid sequence as set forth in SEQ ID NO: 14, the HFR2 comprises an amino acid sequence as set forth in SEQ ID NO: 15, the HFR3 comprises an amino acid sequence as set forth in SEQ ID NO: 16, and the HFR4 comprises an amino acid sequence as set forth in SEQ ID NO: 17; or
the HFR1 comprises an amino acid sequence as set forth in SEQ ID NO: 24, the HFR2 comprises an amino acid sequence as set forth in SEQ ID NO: 15, the HFR3 comprises an amino acid sequence as set forth in SEQ ID NO: 25, and the HFR4 comprises an amino acid sequence as set forth in SEQ ID NO: 26; or
the HFR1 comprises an amino acid sequence as set forth in SEQ ID NO: 24, the HFR2 comprises an amino acid sequence as set forth in SEQ ID NO: 28, the HFR3 comprises an amino acid sequence as set forth in SEQ ID NO: 25, and the HFR4 comprises an amino acid sequence as set forth in SEQ ID NO: 26; or
the HFR1 comprises an amino acid sequence as set forth in SEQ ID NO: 37, the HFR2 comprises an amino acid sequence as set forth in SEQ ID NO: 38, the HFR3 comprises an amino acid sequence as set forth in SEQ ID NO: 39, and the HFR4 comprises an amino acid sequence as set forth in SEQ ID NO: 40; or
the HFR1 comprises an amino acid sequence as set forth in SEQ ID NO: 47, the HFR2 comprises an amino acid sequence as set forth in SEQ ID NO: 48, the HFR3 comprises an amino acid sequence as set forth in SEQ ID NO: 49, and the HFR4 comprises an amino acid sequence as set forth in SEQ ID NO: 50; or
the HFR1 comprises an amino acid sequence as set forth in SEQ ID NO: 52, the HFR2 comprises an amino acid sequence as set forth in SEQ ID NO: 38, the HFR3 comprises an amino acid sequence as set forth in SEQ ID NO: 53, and the HFR4 comprises an amino acid sequence as set forth in SEQ ID NO: 26; and/or
(o) VH, wherein the VH comprises an amino acid sequence as set forth in SEQ ID NO: 18, SEQ ID NO: 66, SEQ ID NO: 41, SEQ ID NO: 67, SEQ ID NO: 95 or SEQ ID NO: 103.

11-24. (canceled)

25. The antigen-binding protein according to claim 1, comprising an antibody heavy chain constant region; optionally
(a) wherein the antibody heavy chain constant region comprises a constant region derived from human IgG; and/or
(b) wherein the antibody heavy chain constant region comprises a constant region derived from human IgG1, IgG2, IgG3 or IgG4; and/or
(c) the antibody heavy chain constant region comprises an amino acid sequence as set forth in SEQ ID NO: 70.

26-28. (canceled)

29. The antigen-binding protein according to claim 1, comprising an antibody heavy chain, wherein the antibody heavy chain comprises an amino acid sequence as set forth in SEQ ID NO: 72, SEQ ID NO: 85, SEQ ID NO: 80 or SEQ ID NO: 87.

30. The antigen-binding protein according to claim 1, comprising
(a) a VL, wherein the VL comprises an LCDR1, an LCDR2 and an LCDR3, and the LCDR1 comprises an amino acid sequence as set forth in SEQ ID NO: 5, SEQ ID NO: 11, SEQ ID NO: 92 or SEQ ID NO: 100; and/or
(b) a VL, wherein the VL comprises an LCDR1, an LCDR2 and an LCDR3, and the LCDR2 comprises an amino acid sequence as set forth in SEQ ID NO: 6, SEQ ID NO: 12, SEQ ID NO: 93 or SEQ ID NO: 101; and/or
(c) a VL, wherein the VL comprises an LCDR1, an LCDR2 and an LCDR3, and the LCDR3 comprises an amino acid sequence as set forth in SEQ ID NO: 7, SEQ ID NO: 13, SEQ ID NO: 94 or SEQ ID NO: 102; and/or
(d) a VL, wherein the VL comprises an LCDR1, an LCDR2 and an LCDR3, the LCDR1 comprises an amino acid sequence as set forth in SEQ ID NO: 5, the LCDR2 comprises an amino acid sequence as set forth in SEQ ID NO: 6, and the LCDR3 comprises an amino acid sequence as set forth in SEQ ID NO: 7; or
the LCDR1 comprises an amino acid sequence as set forth in SEQ ID NO: 11, the LCDR2 comprises an amino acid sequence as set forth in SEQ ID NO: 12, and the LCDR3 comprises an amino acid sequence as set forth in SEQ ID NO: 13; or
wherein the LCDR1 comprises the amino acid sequence as set forth in SEQ ID NO: 92, the LCDR2 comprises the amino acid sequence as set forth in SEQ ID NO: 93, and the LCDR3 comprises the amino acid sequence as set forth in SEQ ID NO: 94; or
wherein the LCDR1 comprises the amino acid sequence as set forth in SEQ ID NO: 100, the LCDR2 comprises the amino acid sequence as set forth in SEQ ID NO: 101, and the LCDR3 comprises the amino acid sequence as set forth in SEQ ID NO: 102.

31-33. (canceled)

34. The antigen-binding protein according to claim 1, comprising
(a) a VH and a VL, wherein the VH comprises an HCDR1, an HCDR2 and an HCDR3, and the VL comprises an LCDR1, an LCDR2 and an LCDR3; wherein the HCDR1 comprises an amino acid sequence as set forth in SEQ ID NO: 1, the HCDR2 comprises an amino acid sequence as set forth in SEQ ID NO: 3, the HCDR3 comprises an amino acid sequence as set forth in SEQ ID NO: 4, the LCDR1 comprises an amino acid sequence as set forth in SEQ ID NO: 5, the LCDR2 comprises an amino acid sequence as set forth in SEQ ID NO: 6, and the LCDR3 comprises an amino acid sequence as set forth in SEQ ID NO: 7; or
the HCDR1 comprises an amino acid sequence as set forth in SEQ ID NO: 1, the HCDR2 comprises an amino acid sequence as set forth in SEQ ID NO: 9, the HCDR3 comprises an amino acid sequence as set forth in SEQ ID NO: 10, the LCDR1 comprises an amino acid sequence as set forth in SEQ ID NO: 11, the LCDR2 comprises an amino acid sequence as set forth in SEQ ID NO: 12, and the LCDR3 comprises an amino acid sequence as set forth in SEQ ID NO: 13; or
(b) a VH and a VL, wherein the VH comprises an HCDR1, an HCDR2 and an HCDR3, and the VL comprises an LCDR1, an LCDR2 and an LCDR3; wherein the HCDR1 comprises an amino acid sequence as set forth in SEQ ID NO: 2, the HCDR2 comprises an amino acid sequence as set forth in SEQ ID NO: 3, the HCDR3 comprises an amino acid sequence as set forth in SEQ ID NO: 4, the LCDR1 comprises an amino acid sequence as set forth in SEQ ID NO: 5, the LCDR2 comprises an amino acid sequence as set forth in SEQ ID NO: 6, and the LCDR3 comprises an amino acid sequence as set forth in SEQ ID NO: 7; or
the HCDR1 comprises an amino acid sequence as set forth in SEQ ID NO: 8, the HCDR2 comprises an amino acid sequence as set forth in SEQ ID NO: 9, the HCDR3 comprises an amino acid sequence as set forth in SEQ ID NO: 10, the LCDR1 comprises an amino acid sequence as set forth in SEQ ID NO: 11, the LCDR2 comprises an amino acid sequence as set forth in SEQ ID NO: 12, and the LCDR3 comprises an amino acid sequence as set forth in SEQ ID NO: 13; or
wherein the HCDR1 comprises the amino acid sequence as set forth in SEQ ID NO: 89, the HCDR2 comprises the amino acid sequence as set forth in SEQ ID NO: 90, and the HCDR3 comprises the amino acid sequence as set forth in SEQ ID NO: 91, the LCDR1 comprises the amino acid sequence as set forth in SEQ ID NO: 92, the LCDR2 comprises the amino acid sequence as set forth in SEQ ID NO: 93, and the LCDR3 comprises the amino acid sequence as set forth in SEQ ID NO: 94; or
wherein the HCDR1 comprises the amino acid sequence as set forth in SEQ ID NO: 97, the HCDR2 comprises the amino acid sequence as set forth in SEQ ID NO: 98, and the HCDR3 comprises the amino acid sequence as set forth in SEQ ID NO: 99, the LCDR1 comprises the amino acid sequence as set forth in SEQ ID NO: 100, the LCDR2 comprises the amino acid sequence as set forth in SEQ ID NO: 101, and the LCDR3 comprises the amino acid sequence as set forth in SEQ ID NO: 102.

35. (canceled)

36. The antigen-binding protein according to claim 1, comprising
(a) a VL, wherein the VL comprises a framework region LFR1, the C-terminus of the LFR1 is directly or indirectly connected to the N-terminus of the LCDR1, and the LFR1 comprises an amino acid sequence as set forth in SEQ ID NO: 19, SEQ ID NO: 30, SEQ ID NO: 42, SEQ ID NO: 55, SEQ ID NO: 59 or SEQ ID NO: 64; and/or
(b) a VL, wherein the VL comprises a framework region LFR2, the LFR2 is positioned between the LCDR1 and the LCDR2, and the LFR2 comprises an amino acid sequence as set forth in SEQ ID NO: 20, SEQ ID NO: 31, SEQ ID NO: 43, SEQ ID NO: 56 or SEQ ID NO: 60; and/or
(c) a VL, wherein the VL comprises a framework region LFR3, the LFR3 is positioned between the LCDR2 and the LCDR3, and the LFR3 comprises an amino acid sequence as set forth in SEQ ID NO: 21, SEQ ID NO: 32, SEQ ID NO: 35, SEQ ID NO: 44, SEQ ID NO: 57 or SEQ ID NO: 61; and/or
(d) a VL, wherein the VL comprises a framework region LFR4, the N-terminus of the LFR4 is directly or indirectly connected to the C-terminus of the LCDR3, and the LFR4 comprises an amino acid sequence as set forth in SEQ ID NO: 22, SEQ ID NO: 33, SEQ ID NO: 45 or SEQ ID NO: 62; and/or
(e) a VL, wherein the VL comprises framework regions LFR1, LFR2, LFR3 and LFR4, wherein the C-terminus of the LFR1 is directly or indirectly connected to the N-terminus of the LCDR1, the LFR2 is positioned between the LCDR1 and the LCDR2, the LFR3 is positioned between the LCDR2 and the LCDR3, and the N-terminus of the LFR4 is directly or indirectly connected to the C-terminus of the LCDR3; wherein the LFR1 comprises an amino acid sequence as set forth in SEQ ID NO: 19, the LFR2 comprises an amino acid sequence as set forth in SEQ ID NO: 20, the LFR3 comprises an amino acid sequence as set forth in SEQ ID NO: 21, and the LFR4 comprises an amino acid sequence as set forth in SEQ ID NO: 22; or
the LFR1 comprises an amino acid sequence as set forth in SEQ ID NO: 30, the LFR2 comprises an amino acid sequence as set forth in SEQ ID NO: 31, the LFR3 comprises an amino acid sequence as set forth in SEQ ID NO: 32, and the LFR4 comprises an amino acid sequence as set forth in SEQ ID NO: 33; or
the LFR1 comprises an amino acid sequence as set forth in SEQ ID NO: 30, the LFR2 comprises an amino acid sequence as set forth in SEQ ID NO: 31, the LFR3 comprises an amino acid sequence as set forth in SEQ ID NO: 35, and the LFR4 comprises an amino acid sequence as set forth in SEQ ID NO: 33; or
the LFR1 comprises an amino acid sequence as set forth in SEQ ID NO: 42, the LFR2 comprises an amino acid sequence as set forth in SEQ ID NO: 43, the LFR3 comprises an amino acid sequence as set forth in SEQ ID NO: 44, and the LFR4 comprises an amino acid sequence as set forth in SEQ ID NO: 45; or
the LFR1 comprises an amino acid sequence as set forth in SEQ ID NO: 55, the LFR2 comprises an amino acid sequence as set forth in SEQ ID NO: 56, the LFR3 comprises an amino acid sequence as set forth in SEQ ID NO: 57, and the LFR4 comprises an amino acid sequence as set forth in SEQ ID NO: 45; or
the LFR1 comprises an amino acid sequence as set forth in SEQ ID NO: 59, the LFR2 comprises an amino acid sequence as set forth in SEQ ID NO: 60, the LFR3 comprises an amino acid sequence as set forth in SEQ ID NO: 61, and the LFR4 comprises an amino acid sequence as set forth in SEQ ID NO: 62; or
the LFR1 comprises an amino acid sequence as set forth in SEQ ID NO: 64, the LFR2 comprises an amino acid sequence as set forth in SEQ ID NO: 60, the LFR3 comprises an amino acid sequence as set forth in SEQ ID NO: 61, and the LFR4 comprises an amino acid sequence as set forth in SEQ ID NO: 62; and/or
(f) a VL, wherein the VL comprises an amino acid sequence as set forth in SEQ ID NO: 23, SEQ ID NO: 68, SEQ ID NO: 46, SEQ ID NO: 69, SEQ ID NO: 96 or SEQ ID NO: 104.

37-41. (canceled)

42. The antigen-binding protein according to claim 1, comprising
(a) a VH and a VL, wherein the VH comprises an amino acid sequence as set forth in SEQ ID NO: 18, and the VL comprises an amino acid sequence as set forth in SEQ ID NO: 23; or
(b) the VH comprises an amino acid sequence as set forth in SEQ ID NO: 41, and the VL comprises an amino acid sequence as set forth in SEQ ID NO: 46; or
(c) the VH comprises an amino acid sequence as set forth in SEQ ID NO: 66, and the VL comprises an amino acid sequence as set forth in SEQ ID NO: 68; or
(d) the VH comprises an amino acid sequence as set forth in SEQ ID NO: 67, and the VL comprises an amino acid sequence as set forth in SEQ ID NO: 69; or
(e) wherein the VH comprises the amino acid sequence as set forth in SEQ ID NO: 95, and the VL comprises the amino acid sequence as set forth in SEQ ID NO: 96; or
(f) wherein the VH comprises the amino acid sequence as set forth in SEQ ID NO: 103, and the VL comprises the amino acid sequence as set forth in SEQ ID NO: 104.

43. The antigen-binding protein according to claim 1, comprising an antibody light chain constant region; optionally,
(a) wherein the antibody light chain constant region comprises a human Igκ constant region or a human Igλ constant region; and/or
(b) wherein the antibody light chain constant region comprises an amino acid sequence as set forth in SEQ ID NO: 71.

44-45. (canceled)

46. The antigen-binding protein according to claim 1,
(a) comprising an antibody light chain, wherein the antibody light chain comprises an amino acid sequence as set forth in SEQ ID NO: 73, SEQ ID NO: 86, SEQ ID NO: 81 or SEQ ID NO: 88; and/or
(b) the antibody heavy chain comprises an amino acid sequence as set forth in SEQ ID NO: 72, and the antibody light chain comprises an amino acid sequence as set forth in SEQ ID NO: 73; or
the antibody heavy chain comprises an amino acid sequence as set forth in SEQ ID NO: 80, and the antibody light chain comprises an amino acid sequence as set forth in SEQ ID NO: 81; or
the antibody heavy chain comprises an amino acid sequence as set forth in SEQ ID NO: 85, and the antibody light chain comprises an amino acid sequence as set forth in SEQ ID NO: 86; or
the antibody heavy chain comprises an amino acid sequence as set forth in SEQ ID NO: 87, and the antibody light chain comprises an amino acid sequence as set forth in SEQ ID NO: 88.

47. (canceled)

48. A polypeptide, comprising the antigen-binding protein according to claim 1.

49. A nucleic acid molecule or molecules, encoding the antigen-binding protein according to claim 1 or a polypeptide comprising the antigen-binding protein.

50. A vector, comprising the nucleic acid molecule according to claim 49.

51. A cell, wherein the cell
(a) comprises a nucleic acid molecule encoding the antigen-binding protein according to claim 1 or a polypeptide comprising the antigen-binding protein; or
(b) comprises a vector comprising the nucleic acid molecule; or
(c) expresses the antigen-binding protein according to claim 1; or
(d) expresses a polypeptide comprising the antigen-binding protein

52. A method for preparing the antigen-binding protein according to claim 1, wherein the method comprises culturing a cell comprising a nucleic acid molecule encoding the antigen-binding protein according to claim 1 or a polypeptide comprising the antigen-binding protein under a condition that the antigen-binding protein according to claim 1 is expressed.

53. A pharmaceutical composition, comprising the antigen-binding protein according to claim 1, a polypeptide comprising the antigen-binding protein, the nucleic acid molecule encoding the antigen-binding protein or the polypeptide, the vector comprising the nucleic acid molecule and/or the cell comprising the nucleic acid molecule or the vector, and optionally a pharmaceutically acceptable carrier.

54. A kit, comprising the antigen-binding protein according to claim 1, a polypeptide comprising the antigen-binding protein or a pharmaceutical composition comprising the antigen-binding protein or the polypeptide.

55. A method for preventing and/or treating a disease or disorder related to CDH6, comprising administering the antigen-binding protein according to claim 1, a polypeptide comprising the antigen-binding protein, a nucleic acid molecule encoding the antigen-binding protein or the polypeptide, a vector comprising the nucleic acid molecule and/or a cell comprising the nucleic acid molecule or the vector, and/or a pharmaceutical composition comprising the antigen-binding protein or the polypeptide.

56. The method according to claim 55, wherein the disease or disorder related to CDH6 comprises a tumor, optionally
(a) wherein the tumor comprises a CDH6-expressing tumor; and/or
(b) wherein the tumor comprises renal cell carcinoma, renal clear cell carcinoma, papillary renal cell carcinoma, ovarian cancer, ovarian serous adenocarcinoma, thyroid cancer, bile duct cancer, lung cancer, small cell lung cancer, liver cancer, glioblastoma, mesothelioma, uterine cancer, pancreatic cancer, Wilm's tumor or neuroblastoma.

57-58. (canceled)

59. The method according to claim 55, wherein the method further comprises administering an additional therapeutic agent.

60. An immunoconjugate or a pharmaceutically acceptable salt or a solvate thereof comprising an antigen-binding protein according to claim 1 or a polypeptide comprising the antigen-binding protein.

61. The immunoconjugate or a pharmaceutically acceptable salt or a solvate thereof according to claim 60, further comprising an active moiety conjugated to the antibody or the antigen binding fragment thereof; optionally, wherein the active moiety comprises a drug moiety and/or a label; optionally,
(a) wherein the drug moiety is selected from the group consisting of a cytotoxic agent, a cytokine, a nucleic acid, a nucleic acid-associated molecule, a radionuclide, a chemokine, an immuno(co)-stimulatory molecule, an immunosuppressive molecule, a death ligand, an apoptosis-inducing protein, a kinase, a prodrug-converting enzyme, a RNase, an agonistic antibody or antibody fragment, an antagonistic antibody or antibody fragment, a growth factor, a hormone, a coagulation factor, a fibrinolytic protein, peptides mimicking these, and fragments, fusion proteins and derivatives thereof; and/or
(b) wherein the label is selected from the group consisting of a radiolabel, a fluorophore, a chromophore, an imaging agent, and a metal ion.

62-64.

65. The immunoconjugate or a pharmaceutically acceptable salt or a solvate thereof according to claim 61, wherein the cytotoxic agent comprises
(a) a microtubule disrupting drug and/or a DNA damaging agent;
(b) a tubulin inhibitor and/or a topoisomerase inhibitor;
(c) a topoisomerase I inhibitor;
(d) camptothecin or a derivative thereof; or
(e) the following structure of formula II or a tautomer, mesomer, racemate, enantiomer, or diastereomer thereof, or mixtures thereof, or a pharmaceutically acceptable salt or a solvate thereof:

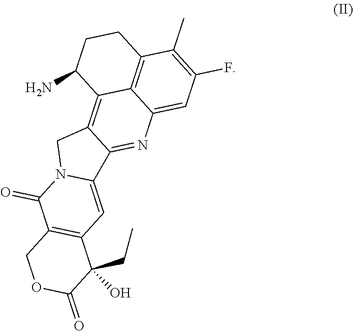 [SMILES: CC[C@@]1(O)C(=O)OCc2c1cc1n(c2=O)Cc2c-1nc1cc(F)c(C)c3c1c2[C@@H](N)CC3]

66-69. (canceled)

70. The immunoconjugate or a pharmaceutically acceptable salt or a solvate thereof according to claim 60, wherein the immunoconjugate is an antibody drug conjugate (ADC) of formula (I):
Ab−(L−(D)ₘ)ₙ, (I)
wherein Ab is an antigen-binding protein having one or more of the following properties:
i) being capable of binding to a CDH6 protein with a sensitivity of less than 12.5 ng/mL in an ELISA assay;
ii) being capable of binding to the CDH6 protein with a KD value below about 3.1×10⁻⁹M in an ELISA assay; and
iii) having the activity of being internalized into a CDH6-expressing cell by binding to CDH6;
L is a linker;
D is a drug moiety;
m is an integer from 1 to 8; and
n is any number from 1 to 10.

71. The immunoconjugate or a pharmaceutically acceptable salt or a solvate thereof according to claim 70, wherein the L is selected from: a cleavable linker and a non-cleavable linker.

72. The immunoconjugate or a pharmaceutically acceptable salt or a solvate thereof according to claim 70, wherein the L comprises a cleavable peptide; optionally, the cleavable peptide is cleavable by an enzyme; optionally, the enzyme comprises Cathepsin B; optionally, the cleavable peptide or L comprises an amino acid unit; optionally, the amino acid unit comprises a dipeptide, tripeptide, tetrapeptide or pentapeptide; optionally, the amino acid unit is selected from: Val-Cit, Val-Ala, Glu-Val-Cit, Ala-Ala-Asn, Gly-Val-Cit, Gly-Gly-Gly and Gly-Gly-Phe-Gly.

73-77. (canceled)

78. The immunoconjugate or a pharmaceutically acceptable salt or a solvate thereof according to claim 70, wherein the L comprises a spacer; optionally, the spacer comprises self-immolative spacers; optionally, the self-immolative spacer comprises p-aminobenzoxy carbonyl (PABC) or p-aminobenzyl (PAB); optionally, the p-aminobenzoxy carbonyl (PABC) or p-aminobenzyl (PAB) comprises a polysarcosine (poly-N-methylglycine) residue.

79-80. (canceled)

81. The immunoconjugate or a pharmaceutically acceptable salt or a solvate thereof according to claim 72, wherein the L comprises a spacer and wherein the cleavable peptide is directly spliced to the spacer; optionally, the spacer comprises self-immolative spacers; optionally, the self-immolative spacer comprises p-aminobenzoxy carbonyl (PABC) or p-aminobenzyl (PAB); optionally, the p-aminobenzoxy carbonyl (PABC) or p-aminobenzyl (PAB) comprises a polysarcosine (poly-N-methylglycine) residue.

82. The immunoconjugate or a pharmaceutically acceptable salt or a solvate thereof according to claim 70, wherein the L comprises: -Val-Cit-PABC-, -Val-Ala-PABC-, -Glu-Val-Cit-PABC-, -Ala-Ala-Asn-PABC-, -Gly-Val-Cit-PABC-, -Gly-Gly-Gly-PABC-, -Gly-Gly-Phe-Gly-PABC-, -Val-Cit-PAB-, -Val-Ala-PAB-, -Glu-Val-Cit-PAB-, -Ala-Ala-Asn-PAB-, -Gly-Val-Cit-PAB-, -Gly-Gly-Gly-PAB- or -Gly-Gly-Phe-Gly-PAB-.

83. The immunoconjugate or a pharmaceutically acceptable salt or a solvate thereof according to claim 70, wherein the spacer
(a) comprises the structure shown in —NH—(CH2)n1-La-Lb-Lc-, wherein La denotes —O— or a single bond; Lb denotes —CR2(—CR3)- or a single bond, wherein R2 and R3 each independently denote C1˜C6 alkyl, —(CH2)na-NH2, —(CH2)nb-COOH or —(CH2)nc-OH, n1 denotes an integer from 0 to 6, na, nb and nc each independently denote an integer from 1 to 4, but R2 and R3 are not the same when n^{a} is 0, and Lc denotes —C(═O)—; or
(b) comprises —NH—(CH2)3-C(═O)—, —NH—CH2-O—CH2-C(═O)— or —NH—(CH2)2-O—CH2-C(═O)—.

84. (canceled)

85. The immunoconjugate or a pharmaceutically acceptable salt or a solvate thereof according to claim 70, wherein the L
(a) comprises the structure shown in -L1-L2-L3-, where L1 denotes -(succinimidyl-3-yl-N)—(CH2)n2-C(═O)—, —CH2-C(═O)—NH—(CH2)n3-C(═O)— or —C(═O)—(CH₂)n4-C(═O)—, where n2 denotes an integer from 2 to 8, n3 denotes an integer from 1 to 8, and n4 denotes an integer from 1 to 8; L2 denotes amino acid unit; L3 denotes a self-degradable spacer, or
(b) is selected from:
-(succinimidyl-3-yl-N)—CH2CH2-C(═O)-GGFG-PABC-;
-(succinimidyl-3-yl-N)—CH2CH2CH2CH2CH2-C(═O)-GGFG-PABC-;
-(succinimidyl-3-yl-N)—CH2CH2CH2CH2CH2-C(═O)-GGFG-NH-PABC-;
-(succinimidyl-3-yl-N)—CH2CH2-C(═O)—NH—CH2CH2O—CH2CH2O—CH2CH2-C(═O)-GGFG-PABC-;
-(succinimidyl-3-yl-N)—CH2CH2-C(═O)—NH—CH2CH2O—CH2CH2O—CH2CH2O—CH2CH2O—CH2CH2-C(═O)-GGFG-PABC-;
—CH2-C(═O)—NH—CH2CH2-C(═O)-GGFG-PABC-;
—C(═O)—CH2CH2CH2CH2CH2CH2-C(═O)-GGFG-PABC-;
-(succinimidyl-3-yl-N)—CH2CH2-C(═O)-GGFG-NH—CH2CH2-C(═O)—;
-(succinimidyl-3-yl-N)—CH2CH2-C(═O)-GGFG-NH—CH2CH2CH2-C(═O)—;
-(succinimidyl-3-yl-N)—CH2CH2CH2CH2CH2-C(═O)-GGFG-NH—CH2CH2-C(═O)—;
-(succinimidyl-3-yl-N)—CH2CH2CH2CH2CH2-C(═O)-GGFG-NH—CH2CH2CH2-C(═O)—;
-(succinimidyl-3-yl-N)—CH2CH2CH2CH2CH2-C(═O)-GGFG-NH—CH2CH2CH2CH2CH2-C(═O)—;
-(succinimidyl-3-yl-N)—CH2CH2CH2CH2CH2-C(═O)-GGFG-NH—CH2-O—CH2-C(═O)—;
-(succinimidyl-3-yl-N)—CH2CH2CH2CH2CH2-C(═O)-GGFG-NH—CH2CH2-O—CH2-C(═O)—;
-(succinimidyl-3-yl-N)—CH2CH2-C(═O)—NH—CH2CH2O—CH2CH2O—CH2CH2-C(═O)-GGFG-NH—CH2CH2CH2-C(═O)—;
-(succinimidyl-3-yl-N)—CH2CH2-C(═O)—NH—CH2CH2O—CH2CH2O—CH2CH2-C(═O)-GGFG-NH—CH2CH2-C(═O)—;
-(succinimidyl-3-yl-N)—CH2CH2-C(═O)—NH—CH2CH2O—CH2CH2O—CH2CH2O—CH2CH2O—CH2CH2-C(═O)-GGFG-NH—CH2CH2CH2-C(═O)—;
-(succinimidyl-3-yl-N)—CH2CH2-C(═O)—NH—CH2CH2O—CH2CH2O—CH2CH2O—CH2CH2O—CH2CH2-C(═O)-GGFG-NH—CH2CH2-C(═O)—;
—CH2-C(═O)—NH—CH2CH2-C(═O)-GGFG-NH—CH2CH2CH2-C(═O)—;
—C(═O)—CH2CH2CH2CH2CH2CH2-C(═O)-GGFG-NH—CH2CH2CH2-C(═O)—;
-(succinimidyl-3-yl-N)—CH2CH2-C(═O)—VA-PABC-;
-(succinimidyl-3-yl-N)—CH2CH2CH2CH2CH2-C(═O)—VA-PABC-;
-(succinimidyl-3-yl-N)—CH2CH2CH2CH2CH2-C(═O)—VA-NH-PABC-;
-(succinimidyl-3-yl-N)—CH2CH2-C(═O)—NH—CH2CH2O—CH2CH2O—CH2CH2-C(═O)—VA-PABC-;
-(succinimidyl-3-yl-N)—CH2CH2-C(═O)—NH—CH2CH2o-CH2CH2O—CH2CH2O—CH2CH2O—CH2CH2-C(═O)—VA-PABC-;
—CH2-C(═O)—NH—CH2CH2-C(═O)—VA-PABC-;
—C(═O)—CH2CH2CH2CH2CH2CH2-C(═O)—VA-PABC-;
-(succinimidyl-3-yl-N)—CH2CH2-C(═O)—VA-NH—CH2CH2-C(═O)—;
-(succinimidyl-3-yl-N)—CH2CH2-C(═O)—VA-NH—CH2CH2CH2-C(═O)—;
-(succinimidyl-3-yl-N)—CH2CH2CH2CH2CH2-C(═O)—VA-NH—CH2CH2-C(═O)—;
-(succinimidyl-3-yl-N)—CH2CH2CH2CH2CH2-C(═O)—VA-NH—CH2CH2CH2-C(═O)—;
-(succinimidyl-3-yl-N)—CH2CH2CH2CH2CH2-C(═O)—VA-NH—CH2CH2CH2CH2CH2-C(═O)—;
-(succinimidyl-3-yl-N)—CH2CH2CH2CH2CH2-C(═O)—VA-NH—CH2-CH2-C(═O)—;
-(succinimidyl-3-yl-N)—CH2CH2CH2CH2CH2-C(═O)—VA-NH—CH2CH2-O—CH2-C(═O)—;
-(succinimidyl-3-yl-N)—CH2CH2-C(═O)—NH—CH2CH2O—CH2CH2O—CH2CH2-C(═O)—VA-NH—CH2CH2CH2-C(═O)—;
-(succinimidyl-3-yl-N)—CH2CH2-C(═O)—NH—CH2CH2O—CH2CH2O—CH2CH2-C(═O)—VA-NH—CH2CH2-C(═O)—;
-(succinimidyl-3-yl-N)—CH2CH2-C(═O)—NH—CH2CH2O—CH2CH2O—CH2CH2O—CH2CH2O—CH2CH2-C(═O)—VA-NH—CH2CH2CH2-C(═O)—;
-(succinimidyl-3-yl-N)—CH2CH2-C(═O)—NH—CH2CH2O—CH2CH2O—CH2CH2O—CH2CH2O—CH2CH2-C(═O)—VA-NH—CH2CH2-C(═O)—;
—CH2-C(═O)—NH—CH2CH2-C(═O)—VA-NH—CH2CH2CH2-C(═O)—; and
—C(═O)—CH2CH2CH2CH2CH2CH2-C(═O)—VA-NH—CH2CH2CH2-C(═O)—;
(c) is selected from the following structure: 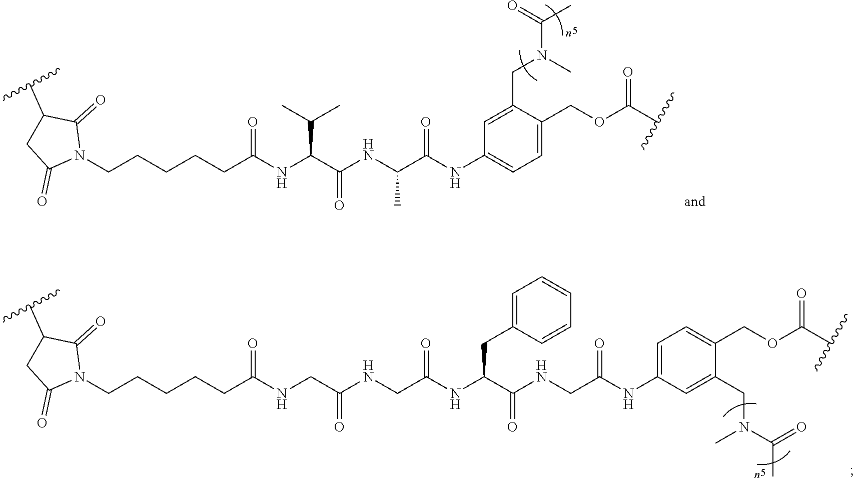
wherein n5 denotes an integer from 0 to 20; optionally, wherein n5 denotes an integer from 1 to 15; or
(d) is selected from the following structure:

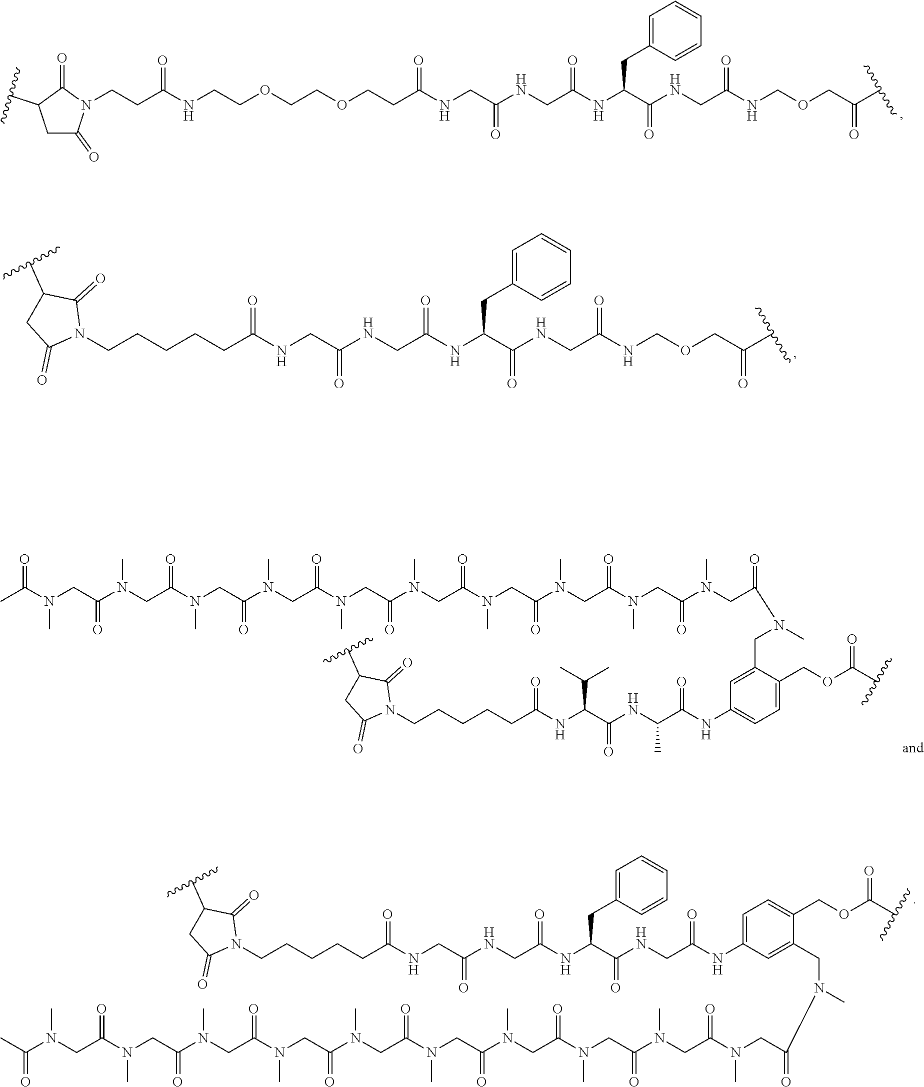

86-90. (canceled)

91. The immunoconjugate or a pharmaceutically acceptable salt or a solvate thereof according to claim 60, wherein the antibody drug conjugate is selected from the following structures: 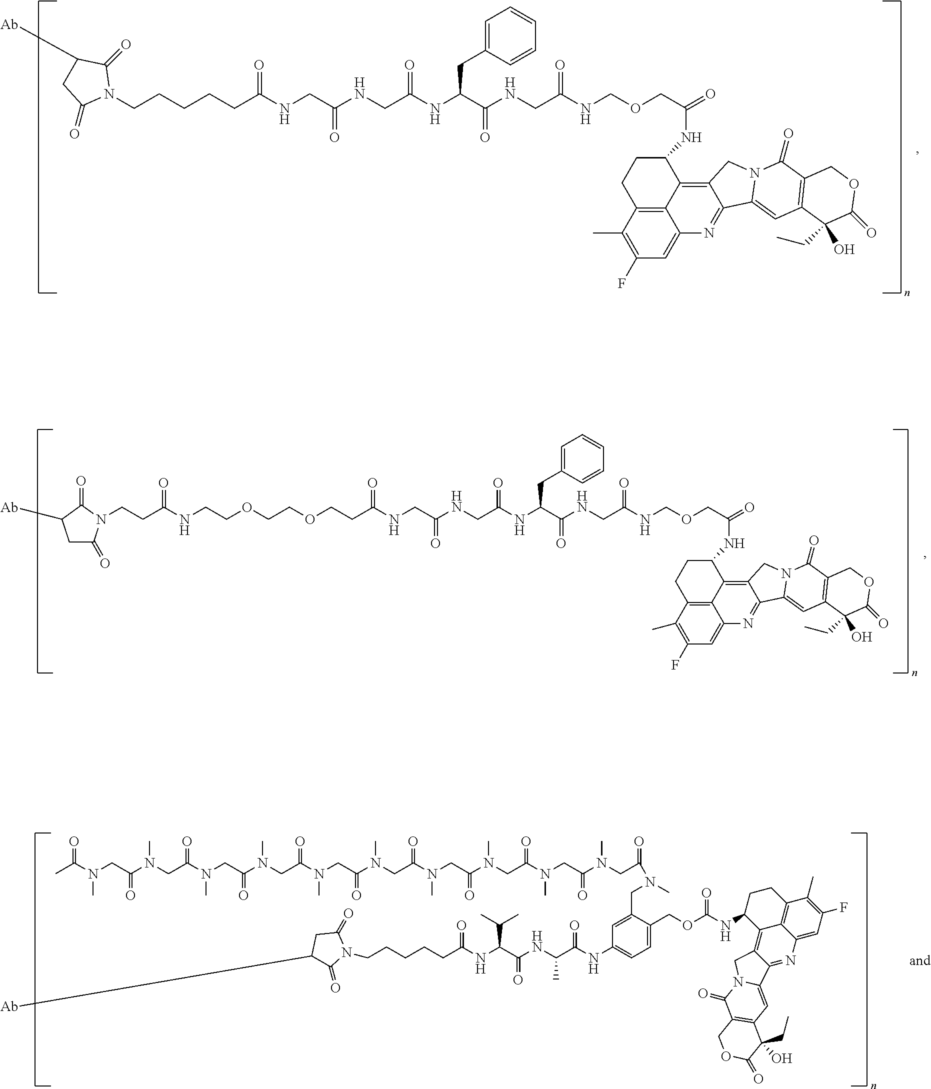 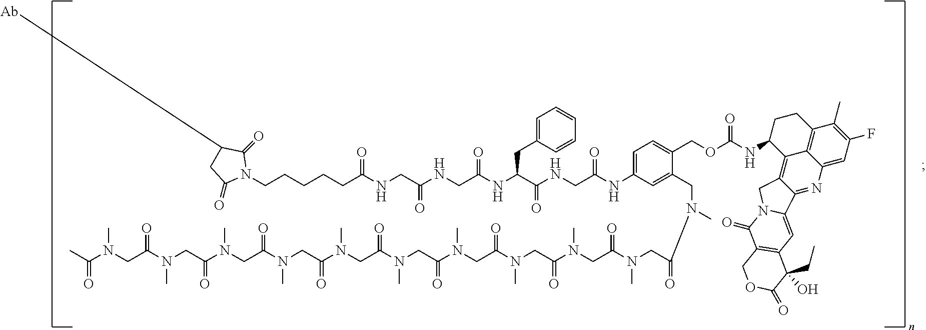
wherein n is any number from 1 to 10; optionally, n is any number from 2 to 9.

92. (canceled)

93. A method of preparing an immunoconjugate or a pharmaceutically acceptable salt or a solvate thereof according to claim 60, comprising the step of reacting an antigen-binding protein according to claim 1 with a drug-linker intermediate compound.

94. A pharmaceutical composition comprising the immunoconjugate or a pharmaceutically acceptable salt or a solvate thereof according to claim 60; optionally, the pharmaceutical composition further comprises a pharmaceutically acceptable carrier or excipient.

95-98. (canceled)

99. A method for treating a tumor, which comprises administering the immunoconjugate or a pharmaceutically acceptable salt or a solvate thereof according to claim 60; optionally, the tumor is a tumor expressing CDH6; optionally, the tumor comprises renal cell carcinoma, renal clear cell carcinoma, papillary renal cell carcinoma, ovarian cancer, ovarian serous adenocarcinoma, thyroid cancer, bile duct cancer, lung cancer, small-cell lung cancer, glioblastoma, mesothelioma, uterine cancer, pancreatic cancer, Wilms' tumor or neuroblastoma; optionally, the method further comprises administering at least one antitumor drug to a subject, simultaneously, separately or sequentially.

100-102. (canceled)


SEQUENCE LISTING (106 sequences)

SEQ ID NO: 1 - protein, 8 aa, synthetic construct
  REGION 1..8: note = CL069707 HCDR1; CL069463 HCDR1; Ch069707 HCDR1; Ch069463 HCDR1;
  VARIANT 5: note = misc_feature - Xaa = Asn or Thr
GFTFXTYA

SEQ ID NO: 2 - protein, 8 aa, synthetic construct
  REGION 1..8: note = CL069707 HCDR1; Ch069707 HCDR1;
GFTFTTYA

SEQ ID NO: 3 - protein, 10 aa, synthetic construct
  REGION 1..10: note = CL069707 HCDR2; Ch069707 HCDR2;
IRIKNNNYAT

SEQ ID NO: 4 - protein, 12 aa, synthetic construct
  REGION 1..12: note = CL069707 HCDR3; Ch069707 HCDR3;
VRPDYGNYGLAY

SEQ ID NO: 5 - protein, 7 aa, synthetic construct
  REGION 1..7: note = CL069707 LCDR1; Ch069707 LCDR1;
SSISSHY

SEQ ID NO: 6 - skipped

SEQ ID NO: 7 - protein, 8 aa, synthetic construct
  REGION 1..8: note = CL069707 LCDR3; Ch069707 LCDR3;
QQDIVPLT

SEQ ID NO: 8 - protein, 8 aa, synthetic construct
  REGION 1..8: note = CL069463 HCDR1; Ch069463 HCDR1;
GFTFNTYA

SEQ ID NO: 9 - protein, 10 aa, synthetic construct
  REGION 1..10: note = CL069463 HCDR2; Ch069463 HCDR2;
IKSKNKDYET

SEQ ID NO: 10 - protein, 10 aa, synthetic construct
  REGION 1..10: note = CL069463 HCDR3; Ch069463 HCDR3;
VRQNWNYFDY

SEQ ID NO: 11 - protein, 6 aa, synthetic construct
  REGION 1..6: note = CL069463 LCDR1; Ch069463 LCDR1;
ENIYYS

SEQ ID NO: 12 - skipped

SEQ ID NO: 13 - protein, 9 aa, synthetic construct
  REGION 1..9: note = CL069463 LCDR3; Ch069463 LCDR3;
QQAYDVPPT

SEQ ID NO: 14 - protein, 25 aa, synthetic construct
  REGION 1..25: note = CL069707 HFR1; Ch069707 HFR1;
EVHLVESGGGLVQPKGSLKLSCVAS

SEQ ID NO: 15 - protein, 17 aa, synthetic construct
  REGION 1..17: note = CL069707 HFR2; Ch069707 HFR2; CL069707-H1 HFR2
MNWVRQAPGKGLEWVAR

SEQ ID NO: 16 - protein, 38 aa, synthetic construct
  REGION 1..38: note = CL069707 HFR3; Ch069707 HFR3
YYADSVTDRFTISRDDSQSMLYLQMNNLKTEDTAMYYC

SEQ ID NO: 17 - protein, 11 aa, synthetic construct
  REGION 1..11: note = CL069707 HFR4; Ch069707 HFR4
WGQGTLVTVSA

SEQ ID NO: 18 - protein, 121 aa, synthetic construct
  REGION 1..121: note = CL069707 VH; Ch069707 VH
EVHLVESGGGLVQPKGSLKLSCVASGFTFTTYAMNWVRQAPGKGLEWVARIRIKNNNYATYYADSVTDRFTISRDDSQSMLYLQMNNLKTEDTAMYYCVRPDYGNYGLAYWGQGTLVTVSA

SEQ ID NO: 19 - protein, 26 aa, synthetic construct
  REGION 1..26: note = CL069707 LFR1; Ch069707 LFR1
EIVLTQSPTTMAASPGEKITITCSAS

SEQ ID NO: 20 - protein, 17 aa, synthetic construct
  REGION 1..17: note = CL069707 LFR2; Ch069707 LFR2
LHWYQQKPGFSPKLLIY

SEQ ID NO: 21 - protein, 36 aa, synthetic construct
  REGION 1..36: note = CL069707 LFR3; Ch069707 LFR3
YLASGVPLRFTGSGSGTSYSLTIGTMEAEDVATYYC

SEQ ID NO: 22 - protein, 10 aa, synthetic construct
  REGION 1..10: note = CL069707 LFR4; Ch069707 LFR4
FGAGTKLELK

SEQ ID NO: 23 - protein, 107 aa, synthetic construct
  REGION 1..107: note = CL069707 VL; Ch069707 VL
EIVLTQSPTTMAASPGEKITITCSASSSISSHYLHWYQQKPGFSPKLLIYRSFYLASGVPLRFTGSGSGTSYSLTIGTMEAEDVATYYCQQDIVPLTFGAGTKLELK

SEQ ID NO: 24 - protein, 25 aa, synthetic construct [sequence omitted]

SEQ ID NO: 25 - protein, 38 aa, synthetic construct
  REGION 1..38: note = CL069707-H1 HFR3; CL069707-H2 HFR3
YYADSVTDRFTISRDDSKNSLYLQMNSLKTEDTAVYYC

SEQ ID NO: 26 - protein, 11 aa, synthetic construct
  REGION 1..11: note = CL069707-H1 HFR4; CL069707-H2 HFR4; CL069463-H2 HFR4
WGQGTLVTVSS

SEQ ID NO: 27 - protein, 121 aa, synthetic construct
  REGION 1..121: note = CL069707-H1 VH
EVQLVESGGGLVQPGGSLRLSCAASGFTFTTYAMNWVRQAPGKGLEWVARIRIKNNNYATYYADSVTDRFTISRDDSKNSLYLQMNSLKTEDTAVYYCVRPDYGNYGLAYWGQGTLVTVSS

SEQ ID NO: 28 - protein, 17 aa, synthetic construct
  REGION 1..17: note = CL069707-H2 HFR2
MNWVRQAPGKGLEWVGR

SEQ ID NO: 29 - protein, 121 aa, synthetic construct
  REGION 1..121: note = CL069707-H2 VH
EVQLVESGGGLVQPGGSLRLSCAASGFTFTTYAMNWVRQAPGKGLEWVGRIRIKNNNYATYYADSVTDRFTISRDDSKNSLYLQMNSLKTEDTAVYYCVRPDYGNYGLAYWGQGTLVTVSS

SEQ ID NO: 30 - protein, 26 aa, synthetic construct [sequence omitted]

SEQ ID NO: 31 - protein, 17 aa, synthetic construct
  REGION 1..17: note = CL069707-L1 LFR2; CL069707-L2 LFR2
LHWYQQKPGKSPKLLIY

SEQ ID NO: 32 - protein, 36 aa, synthetic construct
  REGION 1..36: note = CL069707-L1 LFR3
YLASGVPSRFSGSGSGTDFTLTISSLQPEDFATYYC

SEQ ID NO: 33 - protein, 10 aa, synthetic construct
  REGION 1..10: note = CL069707-L1 LFR4; CL069707-L2 LFR4
FGQGTKLEIK

SEQ ID NO: 34 - protein, 107 aa, synthetic construct
  REGION 1..107: note = CL069707-L1 VL
DIQLTQSPSSLSASVGDRVTITCSASSSISSHYLHWYQQKPGKSPKLLIYRSFYLASGVPSRFSGSGSGTDFTLTISSLQPEDFATYYCQQDIVPLTFGQGTKLEIK

SEQ ID NO: 35 - protein, 36 aa, synthetic construct
  REGION 1..36: note = CL069707-L2 LFR3
YLASGVPLRFSGSGSGTDFTLTISSLQPEDFATYYC

SEQ ID NO: 36 - protein, 107 aa, synthetic construct
  REGION 1..107: note = CL069707-L2 VL
DIQLTQSPSSLSASVGDRVTITCSASSSISSHYLHWYQQKPGKSPKLLIYRSFYLASGVPLRFSGSGSGTDFTLTISSLQPEDFATYYCQQDIVPLTFGQGTKLEIK

SEQ ID NO: 37 - protein, 25 aa, synthetic construct
  REGION 1..25: note = CL069463 HFR1; Ch069463 HFR1;
EVQLVESGGGLVQPKGSLKLSCAAS

SEQ ID NO: 38 - protein, 17 aa, synthetic construct
  REGION 1..17: note = CL069463 HFR2; Ch069463 HFR2; CL069463-H2 HFR2
LTWVRQAPGKGLEWVGR

SEQ ID NO: 39 - protein, 38 aa, synthetic construct
  REGION 1..38: note = CL069463 HFR3; Ch069463 HFR3
YYADSVKDRFTISRNDSQSLLYLQMNNLKTEDTAIYYC

SEQ ID NO: 40 - protein, 11 aa, synthetic construct
  REGION 1..11: note = CL069463 HFR4; Ch069463 HFR4
WGQGTTLTVSS

SEQ ID NO: 41 - protein, 119 aa, synthetic construct
  REGION 1..119: note = CL069463 VH; Ch069463 VH
EVQLVESGGGLVQPKGSLKLSCAASGFTFNTYALTWVRQAPGKGLEWVGRIKSKNKDYETYYADSVKDRFTISRNDSQSLLYLQMNNLKTEDTAIYYCVRQNWNYFDYWGQGTTLTVSS

SEQ ID NO: 42 - protein, 26 aa, synthetic construct
  REGION 1..26: note = CL069463 LFR1; Ch069463 LFR1
DIQMTQSPASLAASVGETVTITCRAS

SEQ ID NO: 43 - protein, 17 aa, synthetic construct
  REGION 1..17: note = CL069463 LFR2; Ch069463 LFR2
LAWYQQRQGKSPQLLIY

SEQ ID NO: 44 - protein, 36 aa, synthetic construct
  REGION 1..36: note = CL069463 LFR3; Ch069463 LFR3
SLKDGVPSRFSGSGSGTQYSMKINSMQPEDTATYFC

SEQ ID NO: 45 - protein, 10 aa, synthetic construct
  REGION 1..10: note = CL069463 LFR4; Ch069463 LFR4 CL069463-L1 LFR4
FGGGTKLEIK

SEQ ID NO: 46 - protein, 107 aa, synthetic construct
  REGION 1..107: note = CL069463 VL; Ch069463 VL
DIQMTQSPASLAASVGETVTITCRASENIYYSLAWYQQRQGKSPQLLIYNANSLKDGVPSRFSGSGSGTQYSMKINSMQPEDTATYFCQQAYDVPPTFGGGTKLEIK

SEQ ID NO: 47 - protein, 25 aa, synthetic construct
  REGION 1..25: note = CL069463-H1 HFR1
EVQLVESGGGLVQPSGSLKLSCAAS

SEQ ID NO: 48 - protein, 17 aa, synthetic construct
  REGION 1..17: note = CL069463-H1 HFR2
LTWVRQASGKGLEWVGR

SEQ ID NO: 49 - protein, 38 aa, synthetic construct
  REGION 1..38: note = CL069463-H1 HFR3
YYADSVKDRFTISRDDSISTLYLQMNSLKTEDTALYYC

SEQ ID NO: 50 - protein, 11 aa, synthetic construct
  REGION 1..11: note = CL069463-H1 HFR4
WGQGTMLTVSS

SEQ ID NO: 51 - protein, 119 aa, synthetic construct
  REGION 1..119: note = CL069463-H1 VH
EVQLVESGGGLVQPSGSLKLSCAASGFTFNTYALTWVRQASGKGLEWVGRIKSKNKDYETYYADSVKDRFTISRDDSISTLYLQMNSLKTEDTALYYCVRQNWNYFDYWGQGTMLTVSS

SEQ ID NO: 52 - protein, 25 aa, synthetic construct
  REGION 1..25: note = CL069463-H2 HFR1
EVQLVESGGGLVQPGGSLKLSCAAS

SEQ ID NO: 53 - protein, 38 aa, synthetic construct
  REGION 1..38: note = CL069463-H2 HFR3
YYADSVKDRFTISRDDSKNTLYLQMNSLKTEDTAVYYC

SEQ ID NO: 54 - protein, 119 aa, synthetic construct
  REGION 1..119: note = CL069463-H2 VH
EVQLVESGGGLVQPGGSLKLSCAASGFTFNTYALTWVRQAPGKGLEWVGRIKSKNKDYETYYADSVKDRFTISRDDSKNTLYLQMNSLKTEDTAVYYCVRQNWNYFDYWGQGTLVTVSS

SEQ ID NO: 55 - protein, 26 aa, synthetic construct
  REGION 1..26: note = CL069463-L1 LFR1
DIQMTQSPSSLSASVGQRVTITCRAS

SEQ ID NO: 56 - protein, 17 aa, synthetic construct
  REGION 1..17: note = CL069463-L1 LFR2
LAWYQQKPGKPPRLLIY

SEQ ID NO: 57 - protein, 36 aa, synthetic construct
  REGION 1..36: note = CL069463-L1 LFR3
SLKDGVPSRFSGSGSGTQYTMTITSMQPEDFATYYC

SEQ ID NO: 58 - protein, 107 aa, synthetic construct
  REGION 1..107: note = CL069463-L1 VL
DIQMTQSPSSLSASVGQRVTITCRASENIYYSLAWYQQKPGKPPRLLIYNANSLKDGVPSRFSGSGSGTQYTMTITSMQPEDFATYYCQQAYDVPPTFGGGTKLEIK

SEQ ID NO: 59 - protein, 26 aa, synthetic construct [sequence omitted]

SEQ ID NO: 60 - protein, 17 aa, synthetic construct
  REGION 1..17: note = CL069463-L2 LFR2; CL069463-L3 LFR2
LAWYQQKPGKAPKLLIY

SEQ ID NO: 61 - protein, 36 aa, synthetic construct
  REGION 1..36: note = CL069463-L2 LFR3; CL069463-L3 LFR3
SLKDGVPSRFSGSGSGTDYTMTISSMQPEDFATYYC

SEQ ID NO: 62 - protein, 10 aa, synthetic construct
  REGION 1..10: note = CL069463-L2 LFR4; CL069463-L3 LFR4
FGGGTKVEIK

SEQ ID NO: 63 - protein, 107 aa, synthetic construct
  REGION 1..107: note = CL069463-L2 VL
DIQMTQSPSSLSASVGDRVTITCRASENIYYSLAWYQQKPGKAPKLLIYNANSLKDGVPSRFSGSGSGTDYTMTISSMQPEDFATYYCQQAYDVPPTFGGGTKVEIK

SEQ ID NO: 64 - protein, 26 aa, synthetic construct
  REGION 1..26: note = CL069463-L3 LFR1
DIQMTQSPSSLSASVGERVTITCRAS

SEQ ID NO: 65 - protein, 107 aa, synthetic construct
  REGION 1..107: note = CL069463-L3 VL
DIQMTQSPSSLSASVGERVTITCRASENIYYSLAWYQQKPGKAPKLLIYNANSLKDGVPSRFSGSGSGTDYTMTISSMQPEDFATYYCQQAYDVPPTFGGGTKVEIK

SEQ ID NO: 66 - protein, 121 aa, synthetic construct
  REGION 1..121: note = CL069707-H1 VH; CL069707-H2 VH
  VARIANT 49: note = misc_feature - Xaa = Ala or Gly
EVQLVESGGGLVQPGGSLRLSCAASGFTFTTYAMNWVRQAPGKGLEWVXRIRIKNNNYATYYADSVTDRFTISRDDSKNSLYLQMNSLKTEDTAVYYCVRPDYGNYGLAYWGQGTLVTVSS

SEQ ID NO: 67 - protein, 119 aa, synthetic construct
  REGION 1..119: note = CL069463-H1 VH; CL069463-H2 VH
  VARIANT 15: note = misc_feature - Xaa = Gly or Ser
  VARIANT 41: note = misc_feature - Xaa = Pro or Ser
  VARIANT 78: note = misc_feature - Xaa = Ile or Lys
  VARIANT 79: note = misc_feature - Xaa = Asn or Ser
  VARIANT 95: note = misc_feature - Xaa = Leu or Val
  VARIANT 114: note = misc_feature - Xaa = Leu or Met
  VARIANT 115: note = misc_feature - Xaa = Leu or Val
EVQLVESGGGLVQPXGSLKLSCAASGFTFNTYALTWVRQAXGKGLEWVGRIKSKNKDYETYYADSVKDRFTISRDDSXXTLYLQMNSLKTEDTAXYYCVRQNWNYFDYWGQGTXXTVSS

SEQ ID NO: 68 - protein, 107 aa, synthetic construct
  REGION 1..107: note = CL069707-L1 VL; CL069707-L2 VL
  VARIANT 61: note = misc_feature - Xaa = Leu or Ser
DIQLTQSPSSLSASVGDRVTITCSASSSISSHYLHWYQQKPGKSPKLLIYRSFYLASGVPXRFSGSGSGTDFTLTISSLQPEDFATYYCQQDIVPLTFGQGTKLEIK

SEQ ID NO: 69 - protein, 107 aa, synthetic construct
  REGION 1..107: note = CL069463-L1 VL; CL069463-L2 VL; CL069463-L3 VL
  VARIANT 17: note = misc_feature - Xaa = Asp, Glu or Gln
  VARIANT 43: note = misc_feature - Xaa = Ala or Pro
  VARIANT 45: note = misc_feature - Xaa = Lys or Arg
  VARIANT 70: note = misc_feature - Xaa = Asp or Gln
  VARIANT 76: note = misc_feature - Xaa = Ser or Thr
  VARIANT 104: note = misc_feature - Xaa = Leu or Val
DIQMTQSPSSLSASVGXRVTITCRASENIYYSLAWYQQKPGKXPXLLIYNANSLKDGVPSRFSGSGSGTXYTMTIXSMQPEDFATYYCQQAYDVPPTFGGGTKXEIK

SEQ ID NO: 70 - protein, 330 aa, synthetic construct [sequence omitted]

SEQ ID NO: 71 - protein, 107 aa, synthetic construct [sequence omitted]

SEQ ID NO: 72 - protein, 451 aa, synthetic construct
  REGION 1..451: note = Ch069707 HC
EVHLVESGGGLVQPKGSLKLSCVASGFTFTTYAMNWVRQAPGKGLEWVARIRIKNNNYATYYADSVTDRFTISRDDSQSMLYLQMNNLKTEDTAMYYCVRPDYGNYGLAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 214 aa, synthetic construct
  REGION 1..214: note = Ch069707 LC
EIVLTQSPTTMAASPGEKITITCSASSSISSHYLHWYQQKPGFSPKLLIYRSFYLASGVPLRFTGSGSGTSYSLTIGTMEAEDVATYYCQQDIVPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 451 aa, synthetic construct
  REGION 1..451: note = CL069707-H1
EVQLVESGGGLVQPGGSLRLSCAASGFTFTTYAMNWVRQAPGKGLEWVARIRIKNNNYATYYADSVTDRFTISRDDSKNSLYLQMNSLKTEDTAVYYCVRPDYGNYGLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 451 aa, synthetic construct
  REGION 1..451: note = CL069707-H2
EVQLVESGGGLVQPGGSLRLSCAASGFTFTTYAMNWVRQAPGKGLEWVGRIRIKNNNYATYYADSVTDRFTISRDDSKNSLYLQMNSLKTEDTAVYYCVRPDYGNYGLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 214 aa, synthetic construct
  REGION 1..214: note = CL069707-L1
DIQLTQSPSSLSASVGDRVTITCSASSSISSHYLHWYQQKPGKSPKLLIYRSFYLASGVPSRFSGSGSGTDFTLTISSLQPEDFATYYCQQDIVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 214 aa, synthetic construct
  REGION 1..214: note = CL069707-L2
DIQLTQSPSSLSASVGDRVTITCSASSSISSHYLHWYQQKPGKSPKLLIYRSFYLASGVPLRFSGSGSGTDFTLTISSLQPEDFATYYCQQDIVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 449 aa, synthetic construct
  REGION 1..449: note = CL069463-H1
EVQLVESGGGLVQPSGSLKLSCAASGFTFNTYALTWVRQASGKGLEWVGRIKSKNKDYETYYADSVKDRFTISRDDSISTLYLQMNSLKTEDTALYYCVRQNWNYFDYWGQGTMLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 449 aa, synthetic construct
  REGION 1..449: note = CL069463-H2
EVQLVESGGGLVQPGGSLKLSCAASGFTFNTYALTWVRQAPGKGLEWVGRIKSKNKDYETYYADSVKDRFTISRDDSKNTLYLQMNSLKTEDTAVYYCVRQNWNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 449 aa, synthetic construct
  REGION 1..449: note = Ch069463 HC
EVQLVESGGGLVQPKGSLKLSCAASGFTFNTYALTWVRQAPGKGLEWVGRIKSKNKDYETYYADSVKDRFTISRNDSQSLLYLQMNNLKTEDTAIYYCVRQNWNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 214 aa, synthetic construct
  REGION 1..214: note = Ch069463 LC
DIQMTQSPASLAASVGETVTITCRASENIYYSLAWYQQRQGKSPQLLIYNANSLKDGVPSRFSGSGSGTQYSMKINSMQPEDTATYFCQQAYDVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 214 aa, synthetic construct
  REGION 1..214: note = CL069463-L1
DIQMTQSPSSLSASVGQRVTITCRASENIYYSLAWYQQKPGKPPRLLIYNANSLKDGVPSRFSGSGSGTQYTMTITSMQPEDFATYYCQQAYDVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 214 aa, synthetic construct
  REGION 1..214: note = CL069463-L2
DIQMTQSPSSLSASVGDRVTITCRASENIYYSLAWYQQKPGKAPKLLIYNANSLKDGVPSRFSGSGSGTDYTMTISSMQPEDFATYYCQQAYDVPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 214 aa, synthetic construct
  REGION 1..214: note = CL069463-L3
DIQMTQSPSSLSASVGERVTITCRASENIYYSLAWYQQKPGKAPKLLIYNANSLKDGVPSRFSGSGSGTDYTMTISSMQPEDFATYYCQQAYDVPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 451 aa, synthetic construct
  REGION 1..451: note = CL069707-H1; CL069707-H2
  VARIANT 49: note = misc_feature - Xaa = Ala or Gly
EVQLVESGGGLVQPGGSLRLSCAASGFTFTTYAMNWVRQAPGKGLEWVXRIRIKNNNYATYYADSVTDRFTISRDDSKNSLYLQMNSLKTEDTAVYYCVRPDYGNYGLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 214 aa, synthetic construct
  REGION 1..214: note = CL069707-L1; CL069707-L2
  VARIANT 61: note = misc_feature - Xaa = Leu or Ser
DIQLTQSPSSLSASVGDRVTITCSASSSISSHYLHWYQQKPGKSPKLLIYRSFYLASGVPXRFSGSGSGTDFTLTISSLQPEDFATYYCQQDIVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 449 aa, synthetic construct
  REGION 1..449: note = CL069463-H1; CL069463-H2
  VARIANT 15: note = misc_feature - Xaa = Gly or Ser
  VARIANT 41: note = misc_feature - Xaa = Pro or Ser
  VARIANT 78: note = misc_feature - Xaa = Ile or Lys
  VARIANT 79: note = misc_feature - Xaa = Asn or Ser
  VARIANT 95: note = misc_feature - Xaa = Leu or Val
  VARIANT 114: note = misc_feature - Xaa = Leu or Met
  VARIANT 115: note = misc_feature - Xaa = Leu or Val
EVQLVESGGGLVQPXGSLKLSCAASGFTFNTYALTWVRQAXGKGLEWVGRIKSKNKDYETYYADSVKDRFTISRDDSXXTLYLQMNSLKTEDTAXYYCVRQNWNYFDYWGQGTXXTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 214 aa, synthetic construct
  REGION 1..214: note = CL069463-L3; CL069463-L2; CL069463-L1
  VARIANT 17: note = misc_feature - Xaa = Asp, Glu or Gln
  VARIANT 43: note = misc_feature - Xaa = Ala or Pro
  VARIANT 45: note = misc_feature - Xaa = Lys or Arg
  VARIANT 70: note = misc_feature - Xaa = Asp or Gln
  VARIANT 76: note = misc_feature - Xaa = Ser or Thr
  VARIANT 104: note = misc_feature - Xaa = Leu or Val
DIQMTQSPSSLSASVGXRVTITCRASENIYYSLAWYQQKPGKXPXLLIYNANSLKDGVPSRFSGSGSGTXYTMTIXSMQPEDFATYYCQQAYDVPPTFGGGTKXEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 8 aa, synthetic construct
  REGION 1..8: note = CL069066 HCDR1; Ch069066 HCDR1
GFDFSRYW

SEQ ID NO: 90 - protein, 8 aa, synthetic construct
  REGION 1..8: note = CL069066 HCDR2; Ch069066 HCDR2
INPDSSTI

SEQ ID NO: 91 - protein, 12 aa, synthetic construct
  REGION 1..12: note = CL069066 HCDR3; Ch069066 HCDR3
TRRPHYYGYFDS

SEQ ID NO: 92 - protein, 10 aa, synthetic construct
  REGION 1..10: note = CL069066 LCDR1; Ch069066 LCDR1
ESVEYYGTTL

SEQ ID NO: 93 - skipped

SEQ ID NO: 94 - protein, 9 aa, synthetic construct
  REGION 1..9: note = CL069066 LCDR3; Ch069066 LCDR3
QQSRKVPWT

SEQ ID NO: 95 - protein, 119 aa, synthetic construct
  REGION 1..119: note = CL069066 VH; Ch069066 VH
EVKLLESGGGLVQPGGSLKLSCAASGFDFSRYWMTWVRQAPGKGLEWIGEINPDSSTINYTPSLKDKFIISRDNAKNTLYLQMNKVTSEDTALYYCTRRPHYYGYFDSWGQGTTLTVSS

SEQ ID NO: 96 - protein, 111 aa, synthetic construct
  REGION 1..111: note = CL069066 VL; Ch069066 VL
DIVLTQSPASLAVSLGQRATISCRASESVEYYGTTLMQWYQQKPGQPPQLLIYAGSNVESGVPARFSGSGSGTEFSLNIHPVEEDDIGMYFCQQSRKVPWTFGGGTKLEIK

SEQ ID NO: 97 - protein, 8 aa, synthetic construct
  REGION 1..8: note = CL069439 HCDR1; Ch069439 HCDR1
GYSFTGYY

SEQ ID NO: 98 - protein, 8 aa, synthetic construct
  REGION 1..8: note = CL069439 HCDR2; Ch069439 HCDR2
ISCFNGDT

SEQ ID NO: 99 - protein, 13 aa, synthetic construct
  REGION 1..13: note = CL069439 HCDR3; Ch069439 HCDR3
VRGRYGNFNYFDY

SEQ ID NO: 100 - protein, 10 aa, synthetic construct
  REGION 1..10: note = CL069439 LCDR1; Ch069439 LCDR1
ESVEFYGTSL

SEQ ID NO: 101 - skipped

SEQ ID NO: 102 - protein, 9 aa, synthetic construct
  REGION 1..9: note = CL069439 LCDR3; Ch069439 LCDR3
QQSRRIPWT

SEQ ID NO: 103 - protein, 120 aa, synthetic construct
  REGION 1..120: note = CL069439 VH; Ch069439 VH
EVQLQQSGTELVRTGASVKISCKASGYSFTGYYIHWIKQSHGESLEWIGYISCFNGDTSYNQNFKDRATFNVDTSSSTAYMQFISLTSEDSAVYYCVRGRYGNFNYFDYWGQGTTLTVSS

SEQ ID NO: 104 - protein, 111 aa, synthetic construct
  REGION 1..111: note = CL069439 VL; Ch069439 VL
DIVLTQSPASLAVSLGQRATISCRASESVEFYGTSLMQWFQQKPGHPPQLLIYATSNVDSGVPARFSASGSGTDFSLNIHPVEEADIAMYFCQQSRRIPWTFGGGTKLEIK

SEQ ID NO: 105 - protein, 122 aa, synthetic construct
  REGION 1..122: note = H01L02 VH
EVQLVQSGAEVKKPGASVKVSCKASGYTFTRNFMHWVRQAPGQGLEWMGWIYPGDGETEYAQKFQGRVTITADTSTSTAYMELSSLRSEDTAVYYCARGVYGGFAGGYFDFWGQGTLVTVSS

SEQ ID NO: 106 - protein, 106 aa, synthetic construct
  REGION 1..106: note = H01L02 VL
DIQMTQSPSSLSASVGDRVTITCKASQNIYKNLAWYQQKPGKAPKLLIYDANTLQTGVPSRFSGSGSGSDFTLTISSLQPEDFATYFCQQYYSGWAFGQGTKVEIK